(19) United States
(12) Patent Application Publication
(10) Pub. No.: US 2006/0053516 A1
(43) Pub. Date: Mar. 9, 2006

(54) Genetically modified plants comprising SARS-CoV viral nucleotide sequences and methods of use thereof for immunization against SARS

(75) Inventors: Mee Len Chye, Pokfulam (HK); Hong Ye Li, Guangzhou (CN); Sathiskumar Ramalingam, Hong Kong (HK); Leo Lit Man Poon, Tsuen Wan (HK); Joseph Sriyal Malik Peiris, Pokfulam (HK)

Correspondence Address: COOPER & DUNHAM, LLP, 1185 AVENUE OF THE AMERICAS, NEW YORK, NY, 10036, US

(73) Assignee: The University of Hong Kong

(21) Appl. No.: 11/004,399

(22) Filed: Dec. 3, 2004

Related U.S. Application Data
(60) Provisional application No. 60/527,637, filed on Dec. 5, 2003.

(51) Int. Cl. A61K 39/12; C12N 5/04; A01H 1/00; C12N 15/82; A61K 36/18

(52) U.S. Cl. 800/288; 435/419; 435/468; 424/186.1

(57) ABSTRACT

The invention relates to genetically modified plants and progeny thereof which constitute edible plant-derived mucosal vaccines and injectable plant-derived mucosal vaccines against Severe Acute Respiratory Syndrome (SARS). The invention relates to a recombinant vector that transforms specifically, but not limited to, the nuclei and/or plastids of tobacco, tomato and lettuce plants for antigen production. In specific embodiments, the plastid transformation vector expressing the nucleotide sequences are pCV1, pCV6 and pCV8, and their derivatives containing an S1 with nucleotide (but not amino acid) changes to optimize codon usage for expression in plants. The present invention relates to methods of making the modified plants which comprises transformation of plants with vectors for nuclear expression and/or plastid expression of nucleotide sequences of the SARS-CoV virus, fragments, derivatives, analogs, or variants thereof. The present invention also relates to methods of immunization against SARS and methods of antibody detection using the SARS-CoV antigens generated by the plastid and/or nuclear vector(s) transformed plants.

This application claims priority of U.S. Ser. No. 60/527,637, filed Dec. 5, 2003, the contents of which are hereby incorporated by reference into this application.

Throughout this application, certain publications are referenced. Full citations for these publications, as well as additional related references, may be found immediately preceding the claims. The disclosures of these publications are hereby incorporated by reference into this application in order to more fully describe the state of the art as of the date of the invention described and claimed herein.

### BACKGROUND OF THE INVENTION

Recently, there has been an outbreak of atypical pneumonia in Guangdong province in mainland China. Between November 2002 and March 2003, there were 792 reported cases with 31 fatalities (WHO, Severe Acute Respiratory Syndrome (SARS), Weekly Epidemiol. Rec. (2003) Vol. 78, page 86). In response to this crisis, the Hospital Authority in Hong Kong has increased the surveillance on patients with severe atypical pneumonia. In the course of this investigation, a number of clusters of health care workers with the disease were identified. In addition, there were clusters of pneumonia incidents among persons in close contact with those infected. The disease was unusual in its severity and its progression in spite of the antibiotic treatment typical for the bacterial pathogens that are known to be commonly associated with atypical pneumonia. Some of the present inventors were one of the groups involved in the investigation of these patients. The disease was given the acronym Severe Acute Respiratory Syndrome (“SARS”), which is caused by a novel coronavirus (Peiris et al., 2003, Lancet Vol. 61, pages 1319-25; Fouchier et al., 2003, Nature Vol. 423, page 240) known as the SARS-CoV virus. The SARS-associated coronavirus is distinct from previously characterized members of the family Coronaviridae that cause respiratory and enteric diseases in animals including humans.

Plants play a critical role as nutrients for animals, including humans, and for the production of substances useful as pharmaceuticals, cosmetics and the like. The generation of transgenic plants by plant nuclear transformation has successfully produced mucosal vaccines against cholera, Norwalk virus, Hepatitis B, foot-and-mouth disease (Walmsley and Arntzen, 2003, Curr. Opin. Biotech. Vol. 14, pages 145-150). Viral antigens expressed in transgenic plants are effective in inducing mucosal and serum immune responses in animals, irrespective of parenteral or oral delivery (Daniell et al., 2001, Trends Plant Sci. Vol. 6, pages 219-226).

Plastid transformation offers great benefits for recombinant DNA technology. In the plant cell, the mere presence of up to 10,000 more copies of the plastid genome as compared to a nuclear genome, ensures enhanced yield of the plastid-expressed foreign protein, or antigen in the case of vaccine production. Further, maternal inheritance of plastids would result in foreign gene containment due to the lack of pollen transmission (Daniell et al., 2002, Trends Plant Sci. Vol. 7, pages 84-91). Human somatotropin accumulates to 7% total soluble protein, which is 300-fold greater than from nuclear-transformed tobacco (Staub et al., 2000, Plant Journal Vol. 6, pages 547-553), while human serum albumin accumulates to 11.1% total soluble protein, which is 500-fold more than nuclear-transformed leaves (Fernandez-San Millan et al., 2003, Plant Biotech Vol. 1, pages 71-79). The cholera toxin B subunit, the first plant-derived vaccine arising from plastid transformation, accumulates to 4.1% total soluble protein in tobacco leaves (Daniell et al., 2001, J. Mol. Biol. Vol. 311, pages 1001-1009). In comparison, the yield of foreign protein from nuclear-transformed plants rarely exceeds 1% total soluble protein (Daniell et al., 2001, Trends Plant Sci. Vol. 6, pages 219-226).

### SUMMARY OF THE INVENTION

The present invention is based on the observation of the present inventors that genetically modified plants and progeny thereof expressing SARS-CoV viral antigens can be used as vaccines against SARS. When the genetically modified plants or progeny thereof are ingested by animals, or extracts from genetically modified plants or progeny thereof are injected into or ingested by animals, preferably human, antibodies are generated against the SARS-CoV viral antigens. An edible vaccine against the SARS-CoV virus, deliverable as fruits (e.g., tomato), leaves (e.g., lettuce), tubers (e.g., potato), seeds (e.g., rice or corn), flowers, stems or roots, would obliterate costly purification procedures required of recombinant vaccines from microorganisms. It has further advantages in easy storage, transport and administration by direct ingestion. Also, edible vaccines in the form of fruits, leaves, tubers, seeds, flowers, stems or roots can be grown and easily distributed in developing countries, cutting the costs of immunization programs, and omitting the need for refrigeration and delivery by injection which requires trained health personnel.

In accordance with the present invention, plant transformation vectors are engineered to provide SARS-CoV viral antigens for eliciting an immune response of an animal for the prevention and treatment of SARS. The antigen includes proteinaceous agent or molecules of the SARS-CoV virus, natural or artificial variants, or mutants thereof. The plant vectors comprising the SARS-CoV viral sequences may be engineered to provide one, two, three or more nucleotide sequences of the SARS-CoV virus. In accordance with the present invention, the antigenic sequences may be derived from the SARS-CoV virus, natural or artificial variants, or mutants thereof.

The present invention provides plant transformation vectors comprising SARS-CoV nucleotide sequences which encode fragments, derivatives, analogs, or variants of polypeptides of SARS-CoV virus. In a specific embodiment, the invention provides for transgenic and/or transplastomic tobacco plants. The present invention provides modified plants that comprise SARS-CoV antigens, including SARS-CoV viral polypeptides or fragments, derivatives, analogs, or variants thereof, or molecules having similar activities as the polypeptides of the SARS-CoV virus. These activities include functional activities as well as immunogenic abilities. The present invention also provides a method of producing the modified plants which comprises transforming a plant with plastid and/or nuclear transformation vector comprising SARS-CoV viral sequences, fragments, derivatives, analogs, or variants thereof. In preferred embodiments, the present invention provides for modified plants that express less than the entire genome of the SARS-CoV virus. In preferred embodiments, the modified plants provide less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, or less than 1% of the entire genome of SARS-CoV virus. In preferred embodiments, the modified plants provide at least 1, 2, 3, 4, 5, 6, 7, 8, 9, 10, 11, 12 polypeptides, fragments, derivatives, analogs and variants of the SARS-CoV virus.

In another specific embodiment, the invention provides a vaccine against SARS. The vaccine comprises the modified plant, its proteins, or extracts. The present invention also provides a method of immunization against SARS. The method comprises administering the modified plants to an animal so as to elicit the production of antibodies in the animal against the SARS-CoV viral antigens, which include SARS-CoV viral polypeptides, or fragments, derivatives, analogs, or variants thereof, or molecules having the similar activities as the polypeptide of the SARS-CoV virus.

In another specific embodiment, plant transformation vectors are engineered to provide SARS-CoV viral antigens for use as reagents in serological tests. One aspect of the invention relates to methods of detecting an antibody to a SARS-CoV viral antigen in a sample by incubating plant-derived SARS-CoV viral antigens with the sample and detecting the presence of antibodies bound to the plant-derived SARS-CoV viral antigens. The sample may be a biological fluid, such as blood, serum, plasma, saliva, urine, stool, sputum, nasopharyngeal aspirates, cells and tissues. Such plant-derived antigens would be cheaper alternative to those generated by infected cell lines for antibody detection.

In one embodiment, the invention relates to compositions comprising a SARS-CoV viral antigen produced by a transformed plant cell or plant. Methods of using the compositions to detect an antibody to a SARS-CoV viral antigen are also encompassed. In a specific embodiment, a method comprises contacting a sample with a composition comprising a SARS-CoV viral antigen produced by a transformed plant cell or plant, and detecting the presence of an antibody bound to the SARS-CoV viral antigen.

In one specific embodiment, the invention provides for a plastid transformation vector comprising the nucleotide sequences of the SARS-CoV virus encoding fragments, derivatives, analogs, or variants of polypeptides of SARS-CoV virus. In another embodiment, the invention provides for a plastid transformation vector which expresses SARS-CoV viral polypeptides, fragments, derivatives, analogs, or variants thereof, or molecules having the similar activities as the polypeptide of the SARS-CoV virus. In a specific embodiment, the vector devoid of viral DNA is pMLVHisA. In a specific embodiment, the present invention provides a method of producing SARS-CoV viral antigens via the plastid transformation vector pCV1, pCV6 or pCV8, and its derivatives containing an S1 with nucleotide (but not amino acid) changes to optimize codon usage for expression in plants.

In another specific embodiment, the invention provides for a nuclear transformation vector comprising the nucleotide sequences of the SARS-CoV virus, fragments, derivatives, analogs, or variants thereof. In another embodiment, the invention provides for a nuclear transformation vector which expresses SARS-CoV viral polypeptides, fragments, derivatives, analogs, or variants thereof, or molecules having the similar activities as the polypeptide of the SARS-CoV virus. In a specific embodiment, the vector devoid of viral DNA is pSa7. In a specific embodiment, the present invention provides a method of producing SARS-CoV viral antigens via the nuclear transformation vector pCV2, pCV4 or pCV12, and its derivatives containing an S1 with nucleotide (but not amino acid) changes to optimize codon usage for expression in plants.

In a specific embodiment, the present invention further provides transplastomic tobacco plants having therein a vector comprising a promoter derived from the rice plastid gene psbA, and a terminator. Plant cells containing a vector comprising a nucleotide sequence of the SARS-CoV virus is also an aspect of this invention. Plant parts of the modified plants, such as for example, fruits, leaves, tubers, seeds, flowers, stems or roots, which comprise cells expressing the SARS-CoV polypeptides, derivatives, analogs, or variants thereof are provided in the invention. The plant parts include parts that are separated from the whole plant or attached onto the whole plant. In a specific embodiment, the present invention further utilizes a selectable marker gene aadA, which specifies spectinomycin-resistance. In another embodiment, the present invention utilizes a start codon for recombinant (His)₅-tagged protein and an rbcL terminator. In a preferred embodiment, the modified plant further comprises heterologous nucleotide sequences that express protease inhibitor protein.

In another specific embodiment, the invention provides for the construction of a nuclear transformation vector for expression of SARS-CoV antigens including polypeptides of the SARS-CoV virus, or fragments, derivatives, analogs, or variants thereof, or molecules having similar activities as the polypeptides of the SARS-CoV virus. In a specific embodiment, the present invention provides plant nuclear transformation vectors pCV2, pCV4 and pCV12.

In another specific embodiment, the invention provides for the construction of a plastid transformation vector for expression of SARS-CoV antigens including polypeptides of the SARS-CoV virus, or fragments, derivatives, analogs, or variants thereof, or molecules having similar activities as the polypeptides of the SARS-CoV virus. In a specific embodiment, the present invention provides plant plastid transformation vectors pCV1, pCV6 and pCV8.

In a specific embodiment, a nuclear transformation vector is used to express one or more SARS-CoV antigens, including polypeptides of the SARS-CoV virus, or fragments, derivatives, analogs, or variants thereof, or molecules having similar activities as the polypeptides of the SARS-CoV virus.

In a specific embodiment, a plastid transformation vector is used to express one or more SARS-CoV antigens, including polypeptides of the SARS-CoV virus, or fragments, derivatives, analogs, or variants thereof, or molecules having similar activities as the polypeptides of the SARS-CoV virus.

In a specific embodiment, a plastid transformation vector and a nuclear transformation vector are used to express one or more SARS-CoV antigens, including polypeptides of the SARS-CoV virus, or fragments, derivatives, analogs, or variants thereof, or molecules having similar activities as the polypeptides of the SARS-CoV virus.

The present invention provides a method of producing SARS-CoV viral antigens in plants, the antigens include polypeptides of the SARS-CoV virus, or fragments, derivatives, analogs, or variants thereof, or molecules having similar activities as the polypeptides of the SARS-CoV virus. The method comprises transforming a plant with a vector which comprises a promoter, operably associated with a coding sequence for one or more SARS-CoV viral polypeptides, and a terminator. Plant cells containing a vector which comprises one or more nucleic acid sequences encoding for the SARS-CoV viral antigens, including polypeptides of the SARS-CoV virus, or fragments, derivatives, analogs, or variants thereof, or molecules having the similar activities as the polypeptides of the SARS-CoV virus, are also an aspect of this invention. Alternatively, the plant cells may contain one or more vectors of the present invention. The present invention provides plant parts, such as for example, fruits, leaves, tubers, seeds, flowers, stems, roots, and all other anatomical parts of the modified plant.

The present invention provides for a vaccine that elicits an immune response against SARS-CoV antigens, including SARS-CoV viral polypeptides, or fragments, derivatives, analogs, or variants thereof, or molecules having similar activities as the polypeptides of the SARS-CoV virus. In one specific embodiment, the invention provides a method of immunization against SARS. The method comprises ingesting an edible transformed plant parts comprising nucleic acid molecules of the SARS-CoV viral sequences encoding the SARS-CoV viral polypeptides. In another embodiment, the method comprises the consumption of the modified plant or intravenous injection or ingestion of an extract of the modified plant, which plant expresses SARS-CoV viral antigens, including SARS-CoV viral polypeptides, or fragments, derivatives, analogs, or variants thereof, or molecules having similar activities as the polypeptides of the SARS-CoV virus. In another specific embodiment, the present invention provides a method of immunization against SARS by feeding the modified plant of the present invention or injecting to or ingestion of an extract of the modified plant of the present invention by a rabbit, goat, cow, pig, sheep, horse, primate, civet, rodent, raccoon, raccoon dog, dog, ferret, ferret Badger, cat, avian, or any other species of animal, including human. The present invention further provides extracting antibodies from an immunized animal. The antibodies are immunospecific to SARS-CoV antigens, including viral polypeptides of the SARS-CoV virus, or fragments, derivatives, analogs, or variants thereof, or molecules having similar activities as the polypeptides of the SARS-CoV virus.

### BRIEF DESCRIPTION OF THE FIGURES

FIGS. 1A-1W show restriction sites of pCV1.

FIGS. 2A-2C show the nucleic acid sequence of pCV1 (SEQ ID NO:1).

FIGS. 3A-3B show restriction maps of pCV1 with specific restriction enzymes.

FIG. 4 shows the pMLVHisA plastid transformation vector from which pCV1, pCV6 and pCV8 were derived. The flanking regions rbcL and accD are derived from the tobacco plastid genome for homologous recombination during plastid transformation. P_{psbA} represents the promoter for the expression of the inserted gene. T_{psbA} represents the terminator. Pᵣᵣₙ represents the promoter driving expression of the spectinomycin-resistance marker aadA. T_{rbcL} represents the rbcL terminator. Met represents the start codon for recombinant (His)₅-tagged protein. (A) shows the sequence (SEQ ID NO:2) and restriction sites of pCV8. (B) shows a restriction map of pCV8 with specific restriction enzymes. (C) shows a restriction map of pCV8 with specific restriction enzymes. (D) shows a restriction map of pCV8 with specific restriction enzymes.

FIG. 5 shows the pSa7 nuclear transformation vector from which pCV2 and pCV4 were derived. RB and LB represent the right and left borders of T(transferred)-DNA for random insertion into the plant nuclear genome. NOS-Pro represents the nopaline synthase (NOS) promoter. NOS-ter represents the NOS-terminator. NPTII (Kan^{R}) represents neomycin phosphotransferase specifying kanamycin-resistance. CaMV35S-Pro represents the Cauliflower Mosaic Virus 35S promoter. SaPIN2a cDNA represents the cDNA encoding Solanum americanum proteinase inhibitor IIA.

FIGS. 6A-6I show the entire nucleic acid sequence of the SARS virus (SEQ ID NO:7).

FIGS. 7A-7LL show restriction sites of pCV8.

FIGS. 8A-8D show the nucleic acid sequence of pCV8 (SEQ ID NO:2).

FIG. 9 shows a restriction map of pCV8 with specific restriction enzymes.

FIG. 10 shows a restriction map of pCV8 with specific restriction enzymes.

FIG. 11 shows a restriction map of pCV8 with specific restriction enzymes.

FIG. 12 shows the pCV12 nuclear transformation vector for expression of a protein fusion consisting of the SARS-CoV S1 protein fused with green fluorescent protein (GFP). RB and LB represent the right and left borders of T(transferred)-DNA for random insertion into the plant nuclear genome. NOS-ter represents the NOS-terminator. CaMV35S-Pro represents the Cauliflower Mosaic Virus 35S promoter. S1 represents the SARS-CoV S1 protein and GFP, the green fluorescent protein.

FIG. 13A-13B show the genomic DNA sequence of the M-gene (SEQ ID NO: 3), and the amino acid sequence of the M protein (SEQ ID NO:4), respectively.

FIGS. 14A-14B show the genomic DNA sequence of the S-gene (SEQ ID NO:5).

FIG. 15 show the amino acid sequence of the S protein (SEQ ID NO:6).

FIGS. 16A-16NN show the deduced amino acid sequences obtained from SEQ ID NO:7 in three frames. An aster (*) indicates a stop codon which marks the end of a peptide.

FIGS. 17A-17NN show the deduced amino acid sequences obtained from the complement of SEQ ID NO:7 in three frames. An aster (*) indicates a stop codon which marks the end of a peptide.

FIGS. 18A-D show transient expression of S1:GFP in agroinfiltrated tobacco leaves. Representative tobacco leaf epidermal cells are shown by confocal microscopy 2 days following agroinfiltration (Yang Y. et al., In vivo analysis of plant promoters and transcription factors by agroinfiltration of tobacco leaves. Plant J. 2000; 22: 543-551) of Agrobacterium tumefaciens LBA4404 harboring plasmid pCV12 expressing S1:GFP fusion protein (A, C) or LBA4404 harboring pGDG expressing GFP alone (B, D). Bar represents 20 μm.

FIGS. 19A-19XX show restriction sites of pCV2.

FIGS. 20A-20E show the nucleic acid sequence of pCV2 (SEQ ID NO:8).

FIGS. 21A-21NN show restriction sites of pCV6.

FIGS. 22A-22D show the nucleic acid sequence of pCV6 (SEQ ID NO:9).

FIGS. 23A-23I show plants obtained following transformation and their analysis. FIGS. 23A-23E show the regenerated plantlets obtained following Agrobacterium-mediated transformation of tobacco and lettuce using nuclear transformation vector pCV2 and analysis of these plantlets by PCR using primers 35S and NOS-ter followed by Southern blot analysis with a ³²P-radiolabeled S1 probe. FIGS. 23F-23I show the regenerated plantlets obtained following plastid transformation of tobacco using plastid transformation vector pCV1, PCR analysis of these plantlets using S1 primers and Northern blot analysis with a ³²P-radiolabeled S1 probe. FIG. 23A shows the plants regenerated from tobacco leaves after Agrobacterium-mediated transformation. FIG. 23B shows the regenerated tobacco shoot used in the PCR analysis. FIG. 23C shows the plants regenerated from lettuce cotyledons after Agrobacterium-mediated transformation. FIG. 23D shows the regenerated lettuce shoot used in the PCR analysis. FIG. 23E shows the presence of a 2.1-kb S1 hybridizing band (arrowed) in two independent tobacco lines (lanes 2 and 3), and two independent lettuce lines (lanes 4 and 5), and the absence of this band in wild-type tobacco (lane 6) and wild-type lettuce (lane 7). One other tobacco line tested negative (lane 1). FIGS. 23F and 23G show the plantlets regenerated from tobacco after particle bombardment. FIG. 23H shows the specific 0.7-kb PCR band in regenerated tobacco (lane 2) which is absent in wild-type (lane 1). FIG. 231 shows the presence of a 2.1-kb hybridizing S1 mRNA band (arrowed) in a tobacco line (lane 2) which is absent in wild-type (lane 1).). FIG. 23J shows western blot analysis of transplastomic tobacco expressing S1 using Ni-NTA conjugate in detection of His-tagged S1 protein. The arrow indicates the expected 73-kDa (His)₅—S1 band.

FIG. 24 shows a Western blot analysis using antibodies against GFP show transient expression of S1:GFP in tobacco leaves following agroinfiltration. Total protein (200 μg) extracted from tobacco leaves, infiltrated with plasmid pGDG expressing GFP alone (lane 1) or plasmid pCV12 expressing S1:GFP fusion (lane 2), were separated on a 8% SDS-PAGE gel, blotted onto Hybond-C filters according to Sambrook et al. (1989. Molecular Cloning: A Laboratory Manual. Cold Spring Harbor Laboratory Press, Cold Spring Harbor), and cross-reacted with antibodies against GFP (Clontech). Arrow indicates cross-reacting S1:GFP band (calculated size 99.1 kDa). M, molecular mass marker.

### DETAILED DESCRIPTION OF THE INVENTION

Definitions of Terms:

As used herein, the term “modified plant or plant parts” refers to a plant or plant part, whether it is attached or detached from the whole plant. It also includes progeny of the modified plant or plant parts that are produced through sexual or asexual reproduction.

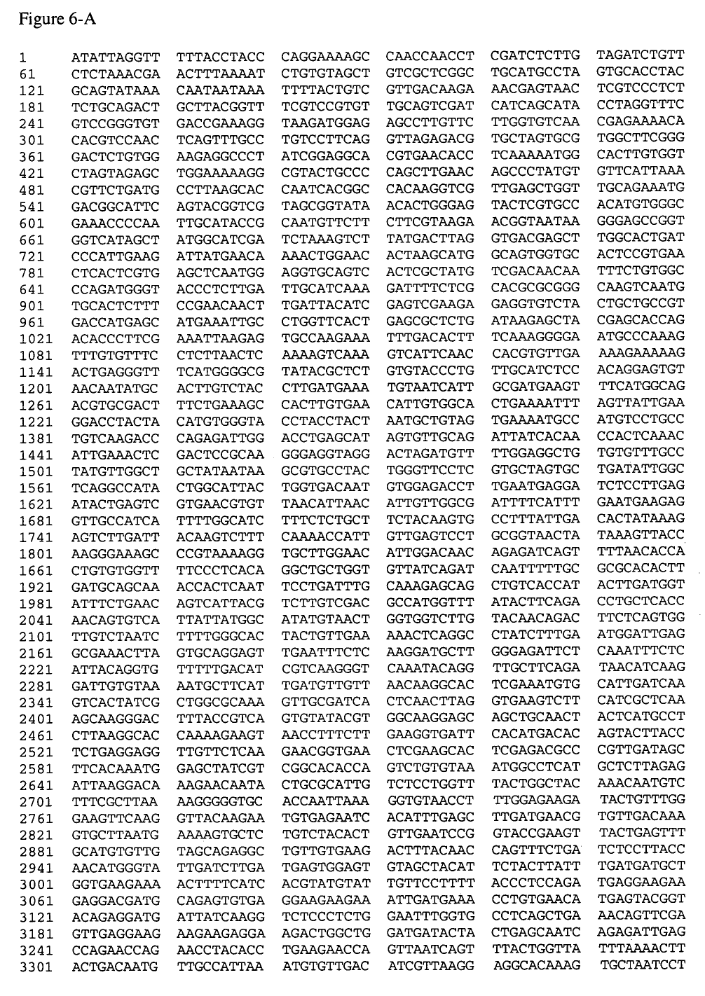

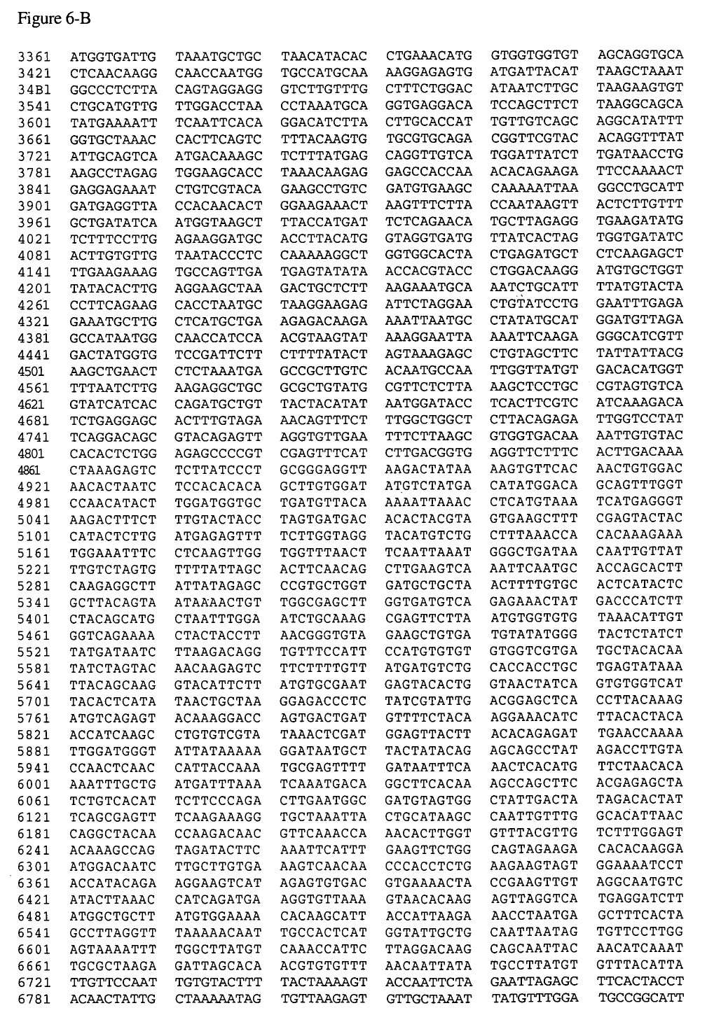

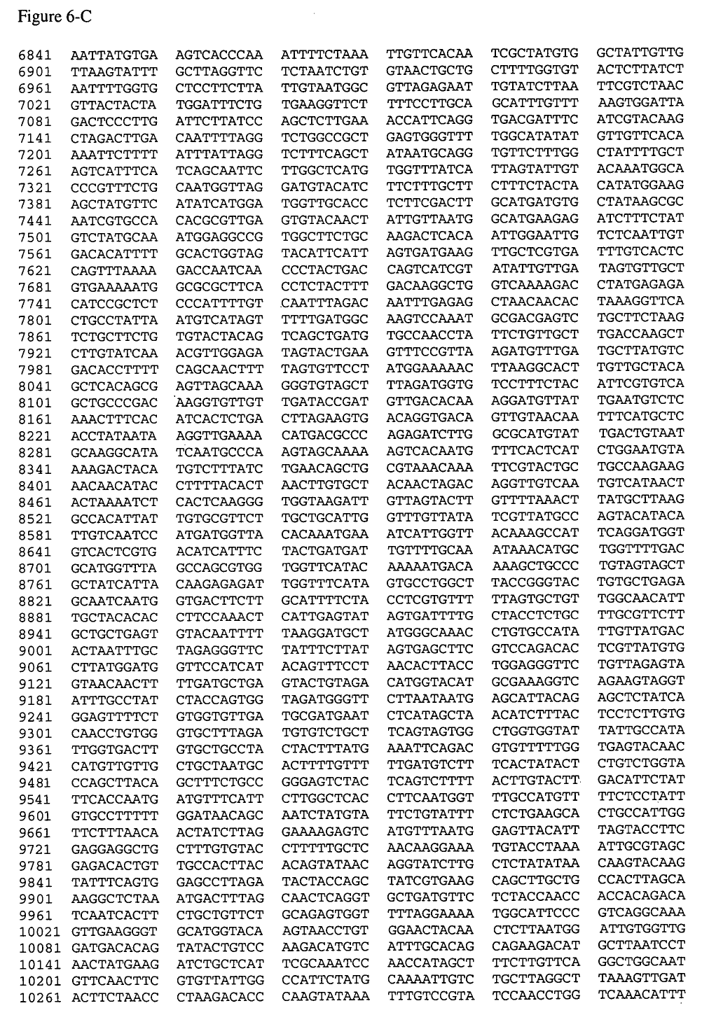

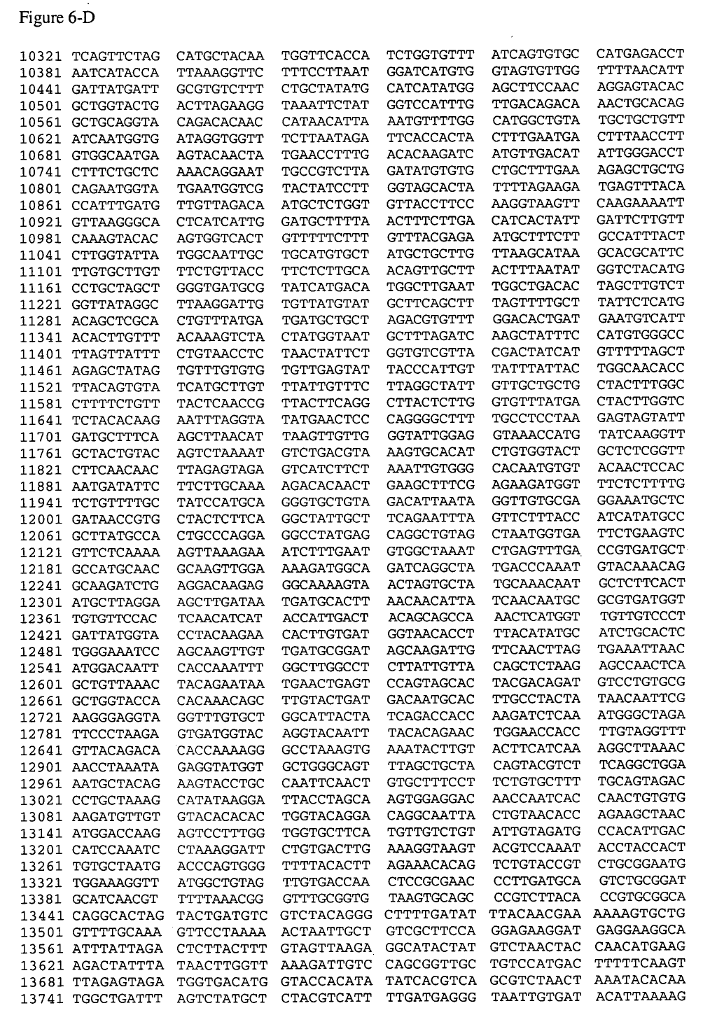

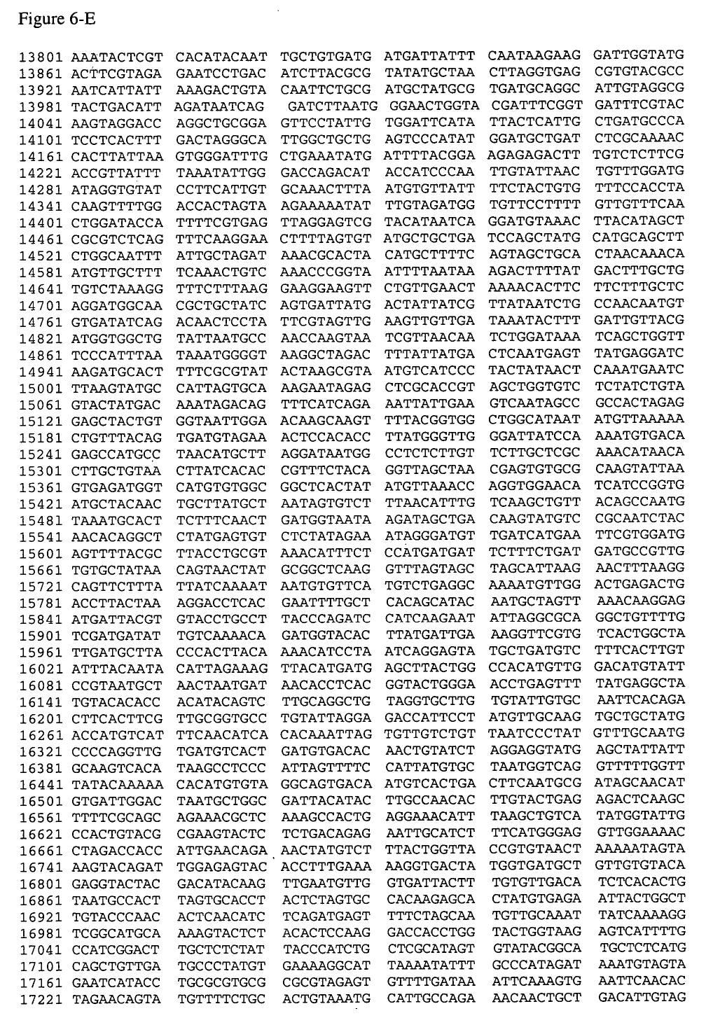

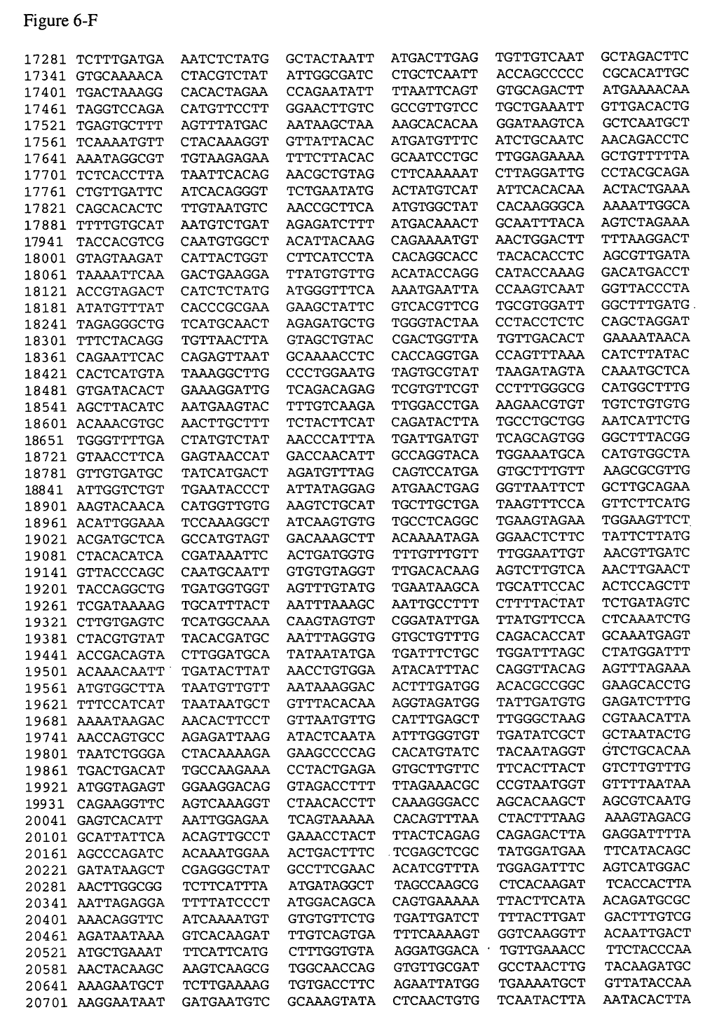

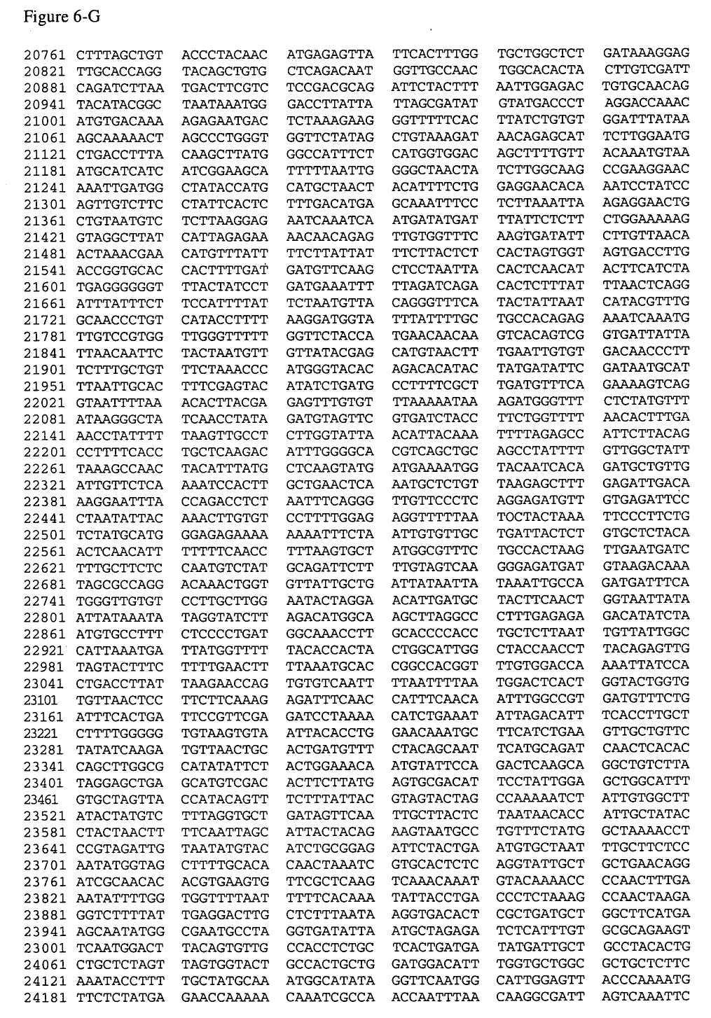

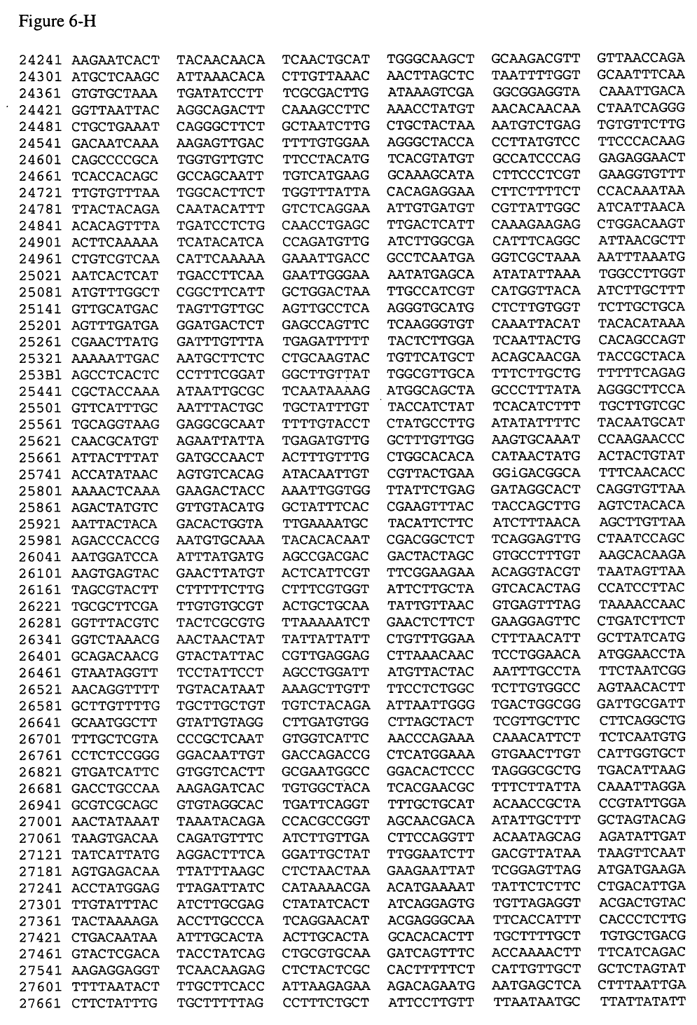

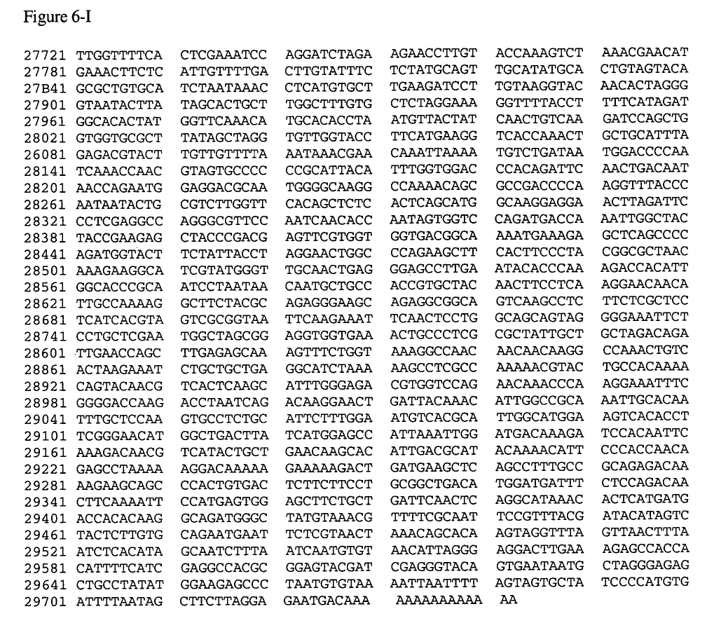

As used herein, the term “variant” refers either to a naturally occurring genetic mutant of SARS-CoV or a recombinantly prepared variation of the SARS-CoV virus, each of which contain one or more mutations in its genome compared to the SARS-CoV virus of a deposited virus, CCTCC-V200303, which sequence is shown in FIG. 6A-6I (SEQ ID NO:7). The term “variant” may also refer to either a naturally occurring variation of a given peptide or a recombinantly prepared variation of a given peptide or protein in which one or more amino acid residues have been modified by amino acid substitution, addition, or deletion. A natural variant of SARS-CoV has a sequence that is different from the genomic sequence of the SARS-CoV virus due to one or more naturally occurred mutations, including, but not limited to, point mutations, rearrangements, insertions, deletions, etc., to the genomic sequence that may or may not result in a phenotypic change. Preferably, the variants include less than 25, less than 20, less than 15, less than 10, less than 5, less than 4, less than 3, or less than 2 amino acid substitutions, rearrangements, insertions, and/or deletions relative to the SARS-CoV virus.

In preferred embodiments, the variants have conservative amino acid substitutions that are made at one or more predicted non-essential amino acid residues (i.e., amino acid residues which are not critical for the expression of the biological activities of the virus, e.g., infectivity, replication ability, protein synthesis ability, assembling ability, and cytotoxic effect). A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine). In another embodiment, the variants have non-conservative amino acid substitution, i.e., amino acid residues are replaced by an amino acid that does not have a side chain with a similar charge.

In a specific embodiment, the SARS-CoV sequences that may be used in the present invention include those deposited with GenBank® having accession nos. NC_{—}004718, AY304495, AY304494, AY304493, AY304492, AY304491, AY304490, AY304489, AY304488, AY304487, AY304486, AY360146, AY278491, AY310120, AY278489, AY362699, AY362698, AY283798, AY283797, AY283796, AY283795, AY283794, AY268070, AY278741, AY340092, AY351680, AP006561, AP006560, AP006559, AP006558, AP006557, AY278554, AY348314, AY338175, AY338174, AY323977, AY322199, AY322198, AY322197, AH013000, AY322208, AY322207, AY322206, AY322205, AH012999, AY321118, AY323976, AY323975, AY323974, AY286320, AY290752, AY291315, AY307165, AY279354, AY278490, AY278487, AY297028, AY286402, AY274119, AY291451, AY271716, AY282752, AY278488, AY268049, AY269391, all of which are incorporated herein by reference in their entireties.

The SARS-CoV viral nucleotide sequences used in the present invention may be derived from a mutant SARS-CoV virus. Mutations can be introduced randomly along all or part of the coding sequence of the SARS-CoV virus or variants thereof, such as by saturation mutagenesis, and the resultant mutants can be screened for biological activity to identify mutants that retain activity. In preferred embodiments, the mutant polypeptides do not retain activity of the wild-type polypeptide. In specific embodiments, the mutant polypeptides do not retain the virulent activity of the SARS-CoV virus. Techniques for mutagenesis known in the art can also be used, including but not limited to, point-directed mutagenesis, chemical mutagenesis, in vitro site-directed mutagenesis, using, for example, the QuikChange® Site-Directed Mutagenesis Kit (Stratagene), etc. Non-limiting examples of such modifications include substitutions of amino acids to cysteines toward the formation of disulfide bonds; substitution of amino acids to tyrosine and subsequent chemical treatment of the polypeptide toward the formation of dityrosine bonds, as disclosed in detail herein; one or more amino acid substitutions and/or biological or chemical modification toward generating a binding pocket for a small molecule (substrate or inhibitor), and/or the introduction of side-chain specific tags (e.g., to characterize molecular interactions or to capture protein-protein interaction partners). Biological modifications that are useful in the present invention comprises alkylation, phosphorylation, sulfation, oxidation or reduction, ADP-ribosylation, hydroxylation, glycosylation, glucosylphosphatidylinositol addition, ubiquitination, etc. Chemical modifications that are useful in the present invention comprise, e.g., altering the charge of the recombinant virus. A positive or negative charge is chemically added to an amino acid residue where a charged amino acid residue is modified to an uncharged residue.

As used herein, the terms “antibody” and “antibodies” refer to monoclonal antibodies, bispecific antibodies, multispecific antibodies, human antibodies, humanized antibodies, chimeric antibodies, camelised antibodies, single domain antibodies, single-chain Fvs (scFv), single chain antibodies, Fab fragments, F(ab′) fragments, disulfide-linked Fvs (sdFv), and anti-idiotypic (anti-Id) antibodies (including, e.g., anti-Id antibodies to antibodies of the invention), and epitope-binding fragments of any of the above. In particular, antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass.

As used herein, the term “an antibody that immunospecifically binds a polypeptide of the SARS-CoV virus” refers to an antibody that immunospecifically binds to the polypeptide encoded by SARS-CoV virus and does not non-specifically bind to other polypeptides. An antibody that immunospecifically binds to the polypeptide of the SARS-CoV virus does not cross-react with other antigens. Preferably, an antibody that immunospecifically binds to a polypeptide of the SARS-CoV virus does not cross-react with other antigens. An antibody that immunospecifically binds to the polypeptide of the SARS-CoV virus can be identified by, for example, immunoassays or other techniques known to those skilled in the art.

As used herein, the term “epitope” refers to a fragment of a SARS-CoV virus, polypeptide or protein having antigenic or immunogenic activity in an animal, preferably a mammal, and most preferably in a human. An epitope having immunogenic activity is a fragment of a polypeptide that elicits an antibody response in an animal. An epitope having antigenic activity is a fragment of a polypeptide or protein to which an antibody immunospecifically binds as determined by any method well known in the art, for example, by the immunoassays described herein. Antigenic epitopes need not necessarily be immunogenic.

As used herein, the term “antigenicity” refers to the ability of a substance (e.g., foreign objects, microorganisms, drugs, antigens, proteins, peptides, polypeptides, nucleic acids, DNA, RNA, etc.) to trigger an immune response in a particular organism, tissue, and/or cell. Sometimes, the term “antigenic” is synonymous with the term “immunogenic”.

As used herein, the term “immunogenicity” refers to the property of a substance (e.g., foreign objects, microorganisms, drugs, antigens, proteins, peptides, polypeptides, nucleic acids, DNA, RNA, etc.) being able to evoke an immune response within an organism. Immunogenicity depends partly upon the size of the substance in question and partly upon how unlike the host molecules is the substance. Highly conserved proteins tend to have rather low immunogenicity.

As used herein, the term “hybridizes under stringent conditions” describes conditions for hybridization and washing under which nucleotide sequences having at least 30%, at least 35%, at least 40%, at least 45%, at least 50%, at least 55%, at least 60%, at least 65%, at least 70%, at least 75%, at least 80%, at least 85%, at least 90%, or at least 95% identity to each other typically remain hybridized to each other. Such hybridization conditions are described in, for example but not limited to, Current Protocols in Molecular Biology, John Wiley & Sons, N.Y. (1989), 6.3.1-6.3.6; Basic Methods in Molecular Biology, Elsevier Science Publishing Co., Inc., N.Y. (1986), pp. 75-78, and 84-87; and Molecular Cloning, Cold Spring Harbor Laboratory, N.Y. (1982), pp. 387-389, and are well known to those skilled in the art. The conditions under which hybridization and/or washing can be carried out can range from 42-68° C. and the washing buffer can comprise from 0.1× sodium chloride/sodium citrate (SSC), 0.5% SDS to 6×SSC, 0.5% SDS. Typically, hybridization can be carried out overnight at 65° C. (high stringency conditions), 60° C. (medium stringency conditions), or 55° C. (low stringency conditions). The filters can be washed for 2×15 minutes with 0.1×SSC, 0.5% SDS at 65° C. (high stringency washing). The filters were washed for 2×15 minutes with 0.1×SSC, 0.5% SDS at 63° C. (medium stringency washing). For low stringency washing, the filters were washed at 60° C. for 2×15 minutes at 2×SSC, 0.5% SDS. A preferred, non-limiting example of stringent hybridization conditions is hybridization in 6×SSC, 0.5% SDS at about 68° C. followed by one or more washes in 2×SSC, 0.5% SDS at room temperature. Another preferred, non-limiting example of stringent hybridization conditions is hybridization in 6×SSC at about 45° C. followed by one or more washes in 0.2×SSC, 0.1% SDS at about 50-65° C.

An “isolated” or “purified” antibody is substantially free of cellular material or other contaminating proteins from the biological fluid from which the antibody is derived. The language “substantially free of cellular material” includes preparations of an antibody in which the antibody is separated from cellular components of the cells from which it is isolated. Thus, an antibody that is substantially free of cellular material includes preparations of the antibody having less than about 30%, 20%, 10%, 5%, 2.5%, or 1%, (by dry weight) of contaminating protein. In a preferred embodiment of the present invention, the antibody is isolated or purified.

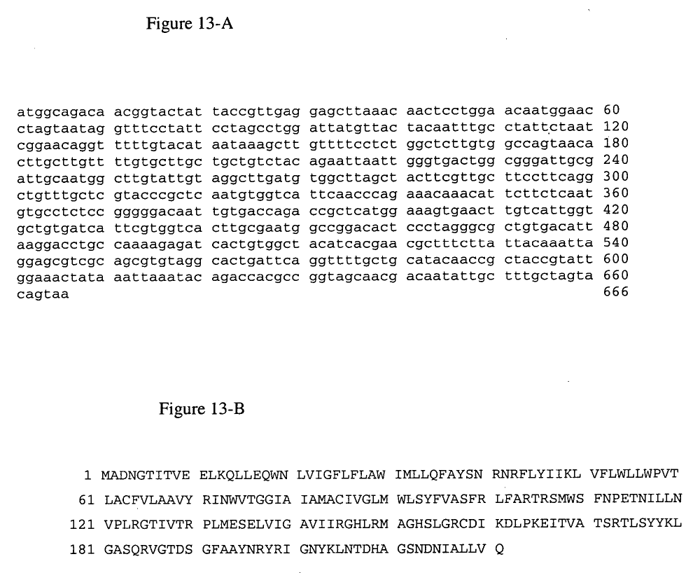

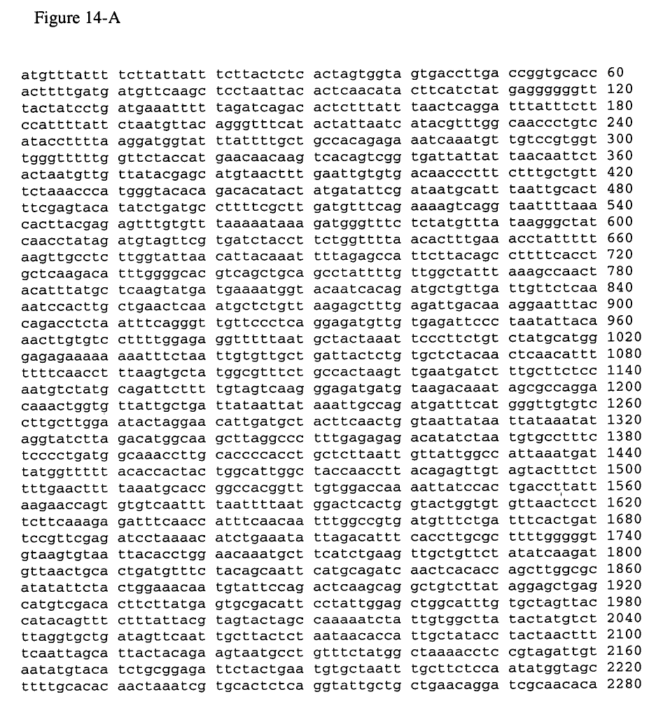

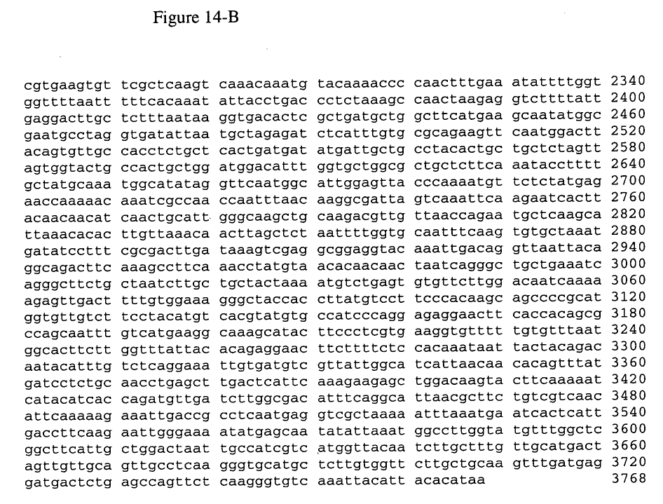

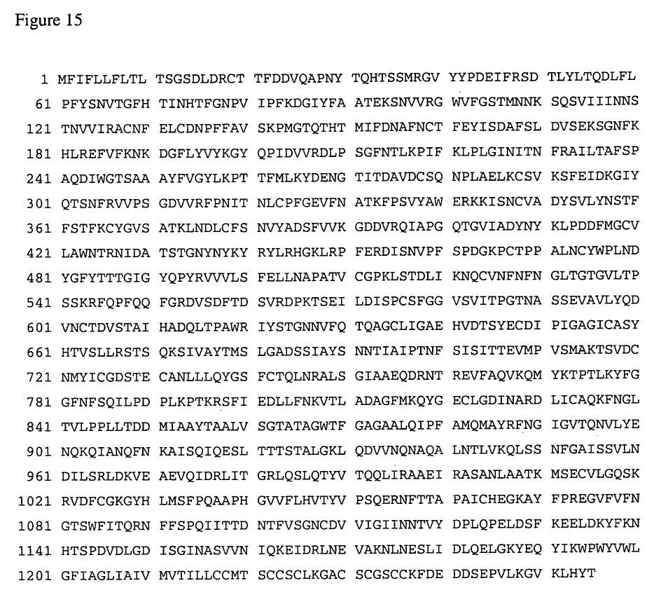

As used herein, the term “having a biological activity of the polypeptides of the SARS-CoV virus” refers to the characteristics of the polypeptides or proteins having a common biological activity similar or identical structural domain and/or having sufficient amino acid identity to SARS polypeptides or the polypeptides having the amino acid sequences as shown in FIGS. 13B, 15, 16A-16NN, and 17A-17NN, or a fragment thereof. Such common biological activities of the polypeptides of the invention include antigenicity and immunogenicity.

As used herein, the term “molecules having the similar activities as the polypeptide of the SARS-CoV virus” refers to polypeptides or proteins having similar biological activities and similar or identical structural domain and/or having sufficient amino acid identity to the polypeptides of the SARS-CoV virus. Such biological activities can include, but are not limited to, antigenicity, immunogenicity, cytotoxicity, hormonal activities, binding properties and affinities, pharmacological activities, stimulation or inhibition of growth proliferation and differentiation, induction of changes in cells, antiviral, antibacterial, antifungal and antiparasitic activities, etc. In preferred embodiments, polypeptides that are useful for the present invention may retain at least one, two, three, four, five, or more biological activities of the wild-type SARS-CoV virus (e.g., infectivity, replication ability, protein synthesis ability, assembling ability, and cytotoxic effect).

As used herein, the term “portion” or “fragment” refers to a fragment of a nucleic acid molecule containing at least about 25, 30, 35, 40, 45, 100, 150, 200, 250, 300, 350, 400, 450, 500, 550, 600, 650, 700, 750, 800, 850, 900, 950, 1,000, 1,050, 1,100, 1,150, 1,200, 1,500, 2,000, 2,500, 3,000, 3,500, 4,000, 5,000, 6,000, 7,000, 8,000, 9,000, 10,000, 15,000, 20,000, 25,000, or more contiguous nucleic acids in length of the relevant nucleic acid molecule and having at least one functional feature of the nucleic acid molecule (or the encoded protein has one functional feature of the protein encoded by the nucleic acid molecule); or a fragment of a protein or a polypeptide containing at least 5, 10, 15, 20, 25, 30, 35, 40, 45, 50, 55, 60, 65, 70, 75, 80, 90, 100, 120, 140, 160, 180, 200, 220, 240, 260, 280, 300, 320, 340, 360, 380, 400, 450, 500, 600, 700, 800, 900, 1,000, 2,000, 3,000, 4,000, 5,000, 6,000, 7,000, 8,000, or more contiguous amino acid residues in length of the relevant protein or polypeptide and having at least one functional feature of the protein or polypeptide.

As used herein, the term “analog” in the context of proteinaceous agent (e.g., proteins, polypeptides, peptides, and antibodies) refers to a proteinaceous agent that possesses a similar or identical function as a second proteinaceous agent but does not necessarily comprise a similar or identical amino acid sequence of the second proteinaceous agent, or possess a similar or identical structure of the second proteinaceous agent. In a specific embodiment, antibody analogs immunospecifically bind to the same epitope as the original antibodies from which the analogs were derived. In an alternative embodiment, antibody analogs immunospecifically bind to different epitopes than the original antibodies from which the analogs were derived. A proteinaceous agent that has a similar amino acid sequence refers to a second proteinaceous agent that satisfies at least one of the following: (a) a proteinaceous agent having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of a second proteinaceous agent; (b) a proteinaceous agent encoded by a nucleotide sequence that hybridizes under stringent conditions to a nucleotide sequence encoding a second proteinaceous agent of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, or at least 150 contiguous amino acid residues; and (c) a proteinaceous agent encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% identical to the nucleotide sequence encoding a second proteinaceous agent. A proteinaceous agent with similar structure to a second proteinaceous agent refers to a proteinaceous agent that has a similar secondary, tertiary or quaternary structure to the second proteinaceous agent. The structure of a proteinaceous agent can be determined by methods known to those skilled in the art, including but not limited to, peptide sequencing, X ray crystallography, nuclear magnetic resonance, circular dichroism, and crystallographic electron microscopy.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can also be accomplished using a mathematical algorithm. A preferred, non limiting example of a mathematical algorithm utilized for the comparison of two sequences is the algorithm of Karlin and Altschul, 1990, Proc. Natl. Acad. Sci. U.S.A. Vol. 87, pages 2264-2268, modified as in Karlin and Altschul, 1993, Proc. Natl. Acad. Sci. U.S.A. Vol. 90, pages 5873-5877. Such an algorithm is incorporated into the NBLAST and XBLAST programs of Altschul et al., 1990, J. Mol. Biol. Vol. 215, page 403. BLAST nucleotide searches can be performed with the NBLAST nucleotide program parameters set, e.g., for score=100, wordlength=12 to obtain nucleotide sequences homologous to a nucleic acid molecules of the present invention. BLAST protein searches can be performed with the XBLAST program parameters set, e.g., to score 50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. Vol. 25, pages 3389-3402. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., the NCBI website). Another preferred, non limiting example of a mathematical algorithm utilized for the comparison of sequences is the algorithm of Myers and Miller, 1988, CABIOS Vol. 4, pages 11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

As used herein, the term “analog” in the context of a non-proteinaceous analog refers to a second organic or inorganic molecule which possesses a similar or identical function as a first organic or inorganic molecule and is structurally similar to the first organic or inorganic molecule.

As used herein, the term “derivative” in the context of proteinaceous agent (e.g., proteins, polypeptides, peptides, and antibodies) refers to a proteinaceous agent that comprises an amino acid sequence which has been altered by the introduction of amino acid residue substitutions, deletions, and/or additions. The term “derivative” as used herein also refers to a proteinaceous agent which has been modified, i.e., by the covalent attachment of any type of molecule to the proteinaceous agent. For example, but not by way of limitation, an antibody may be modified, e.g., by glycosylation, acetylation, pegylation, phosphorylation, amidation, derivatization by known protecting/blocking groups, proteolytic cleavage, linkage to a cellular ligand or other protein, etc. A derivative of a proteinaceous agent may be produced by chemical modifications using techniques known to those of skill in the art, including, but not limited to specific chemical cleavage, acetylation, formylation, metabolic synthesis of tunicamycin, etc. Further, a derivative of a proteinaceous agent may contain one or more non-classical amino acids. A derivative of a proteinaceous agent possesses a similar or identical function as the proteinaceous agent from which it was derived.

As used herein, the term “derivative” in the context of a non-proteinaceous derivative refers to a second organic or inorganic molecule that is formed based upon the structure of a first organic or inorganic molecule. A derivative of an organic molecule includes, but is not limited to, a molecule modified, e.g., by the addition or deletion of a hydroxyl, methyl, ethyl, carboxyl or amine group. An organic molecule may also be esterified, alkylated and/or phosphorylated.

As used herein, the terms “subject” and “patient” are used interchangeably. As used herein, the terms “subject” and “subjects” refer to an animal, preferably a mammal including a non-primate (e.g., rabbits, goats, cows, pigs, sheep, horses, civets, rodents, raccoons, raccoon dogs, dogs, ferrets, ferret Badger, cats, and avian species) and a primate (e.g., monkeys such as a cynomolgous monkey and humans), and more preferably a human.

As used herein, the terms “SARS” or “SARS related symptoms” include various clinical indications and classifications. As described herein, SARS include (1) asymptomatic or mild respiratory illness; (2) moderate respiratory illness; or (3) severe respiratory illness.

As used herein, the term “carrier” is a substance used to support or convey another substance such as a pigment, catalyst, or radioactive material.

As used herein, the term “vehicle” is a substance that facilitates the use of a drug, pigment, or other material mixed with it.

As used herein, the term “excipients” refers to inert substances which are commonly used as a diluent, vehicle, preservatives, binders, or stabilizing agent for drugs and includes, but not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). Also see Remington's Pharmaceutical Sciences (by Joseph P. Remington, 18th ed., Mack Publishing Co., Easton, Pa.), which is hereby incorporated in its entirety.

As used herein, the term “operably associated” or “operably linked” refers to an association in which the regulatory regions (e.g., promoter, enhancer) and the nucleic acid sequence to be expressed are covalently joined and positioned in such a way as to permit transcription, and under the appropriate condition, translation.

### PREFERRED EMBODIMENTS AND EXPERIMENTAL DETAILS

The present invention provides transgenic and transplastomic plants and their progeny that are generated by plastid and/or nuclear transformation vectors thereby producing a mucosal vaccine against SARS-CoV viral antigens. Viral antigens expressed in transgenic plants are effective in inducing mucosal and serum immune responses in animals, irrespective of parenteral or oral delivery. Viral antigens expressed in transgenic plants are also useful as reagents for antibody detection in serological tests. The present invention seeks to transform various types of plants with plant vectors comprising nucleotide sequences from the SARS-CoV virus including but not limited to nucleotide sequences of the SARS-CoV virus which encodes fragments, derivatives, analogs, or variants of polypeptides of SARS-CoV virus, or that it encodes an epitope or a proteinaceous molecule having similar activities as the polypeptides, fragments, derivatives, analogs, or variants thereof, of the SARS-CoV virus.

Analysis of the genome sequence of the SARS-associated coronavirus revealed that four of its eleven predicted open reading frames encode structural proteins including a spike glycoprotein (S) of 1255 deduced amino acids and a membrane glycoprotein (M) of 221 deduced amino acids; S and M presumably associate to form the viral envelope. The S glycoprotein, a type I membrane protein, functions in attachment to host receptors and has an N-terminal signal sequence (S1; amino acids 1-658) and a C-terminal transmembrane region followed by a cytoplasmic tail (Marra et al., 2003; Rota et al, 2003). The N-terminal region of M also includes an uncleavable signal sequence and is exposed to the surface of the virus while its C-terminus resides within the viral membrane. While both S and M are believed to be targeted to the plasma membrane, M was also believed to be localized at the endoplasmic reticulum in plant cells. In preferred embodiments, the modified plant or plant parts or progeny of the modified plant comprise SARS-CoV viral sequences that encode the spike protein (S or S1) of the SARS-CoV virus, or fragments, derivatives, analogs, or variants thereof. In a preferred embodiment, the modified plant or plant parts or progeny of the modified plant comprises nucleotide sequences that encode the N-terminal region (S1) of the spike protein (S) and/or the membrane protein (M) of the SARS-CoV virus, or fragments, derivatives, analogs, or variants thereof, or molecule having the similar activities as the polypeptide of the SARS-CoV virus. In preferred embodiments, the plant or plant parts comprises the nucleotide sequence of SEQ ID NO:3, 5, or 7, or a fragment thereof, or that immunospecifically binds to the polypeptide having the nucleotide sequence of SEQ ID NO:3, 5, or 7, or an analog, derivative, or fragment thereof, and/or polypeptides encoded by the nucleotide sequence of SEQ ID NO:3, 5, or 7, or a fragment thereof. The present invention also relates to a method of producing modified plants comprising one or more plastid and/or nuclear transformation vectors comprising one or more SARS-CoV nucleotide sequences which encode fragments, derivatives, analogs or variants of polypeptides of SARS-CoV virus. All varieties of plants may be used for the present invention. In a preferred embodiment, the plant is tobacco, lettuce, potato, tomato, banana, corn, rice, cereals, wheat, maize, barley, apple, pear, strawberry, carrot, sugar beets, yam, kiwifruit, or spinach.

Construction of Transformation Vectors

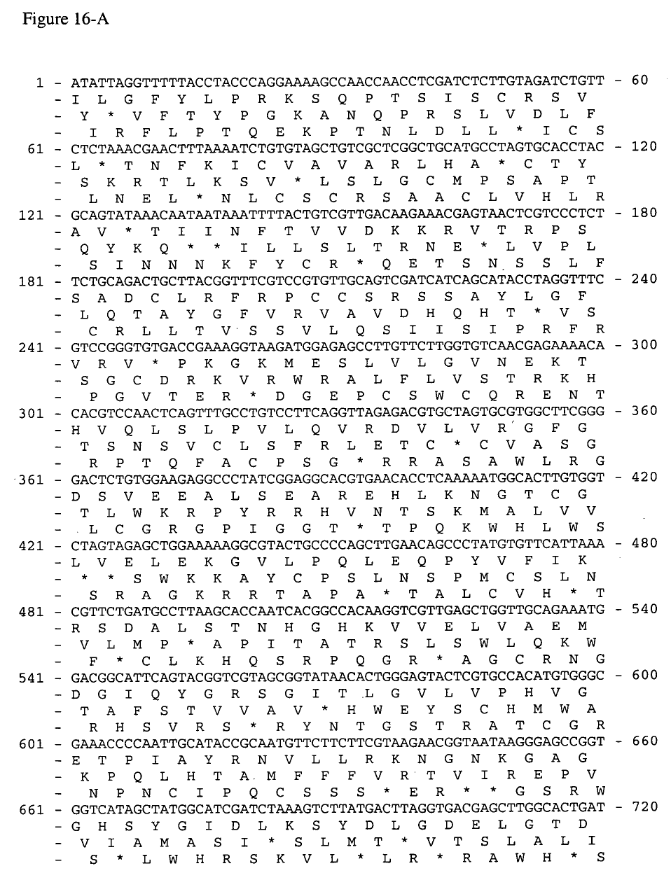

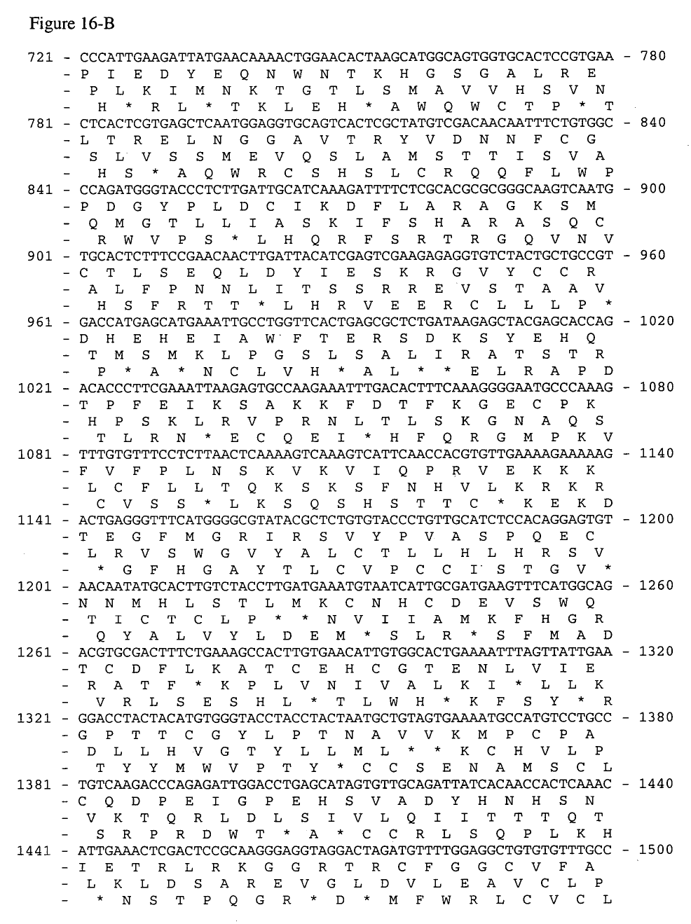

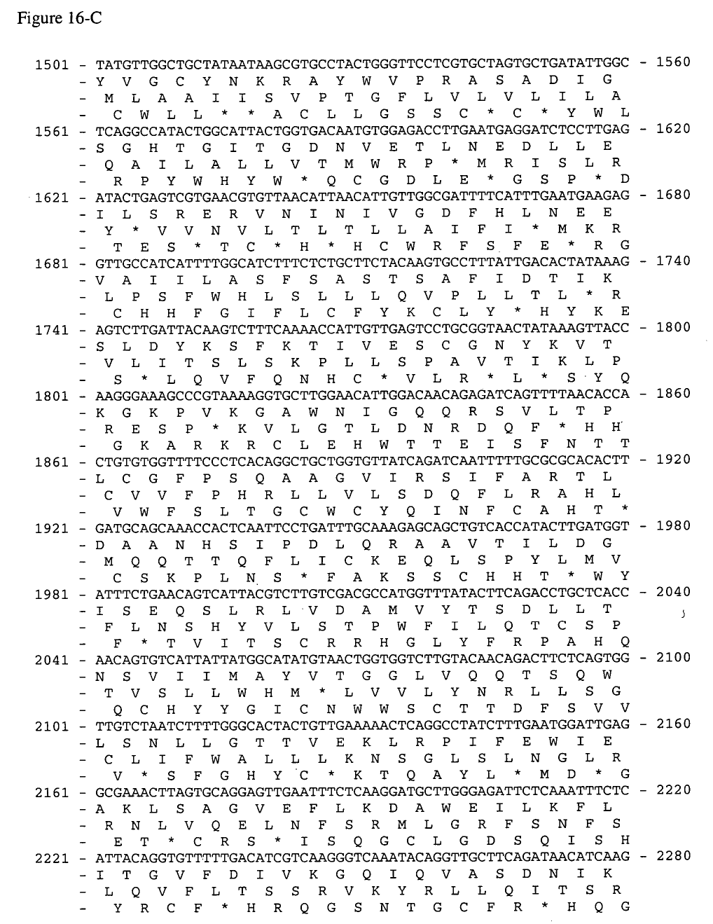

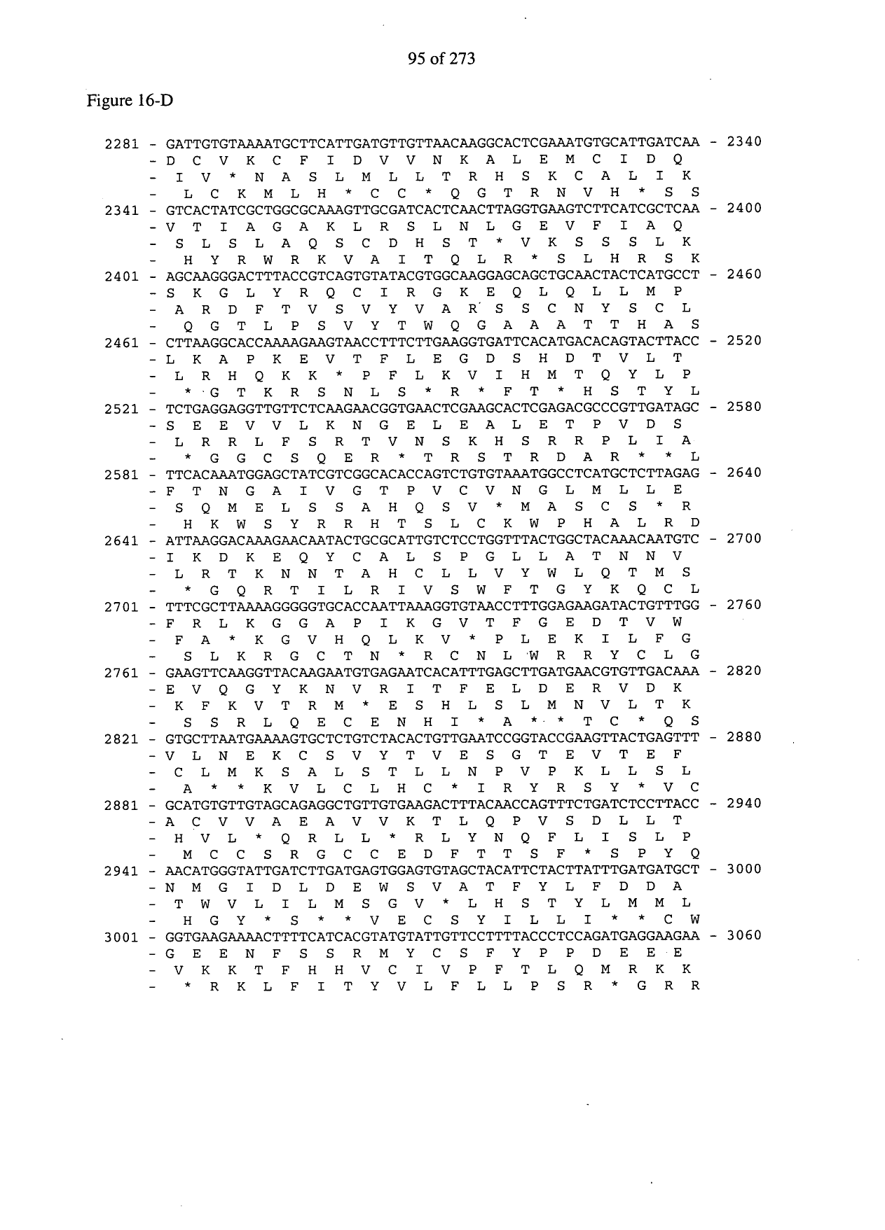

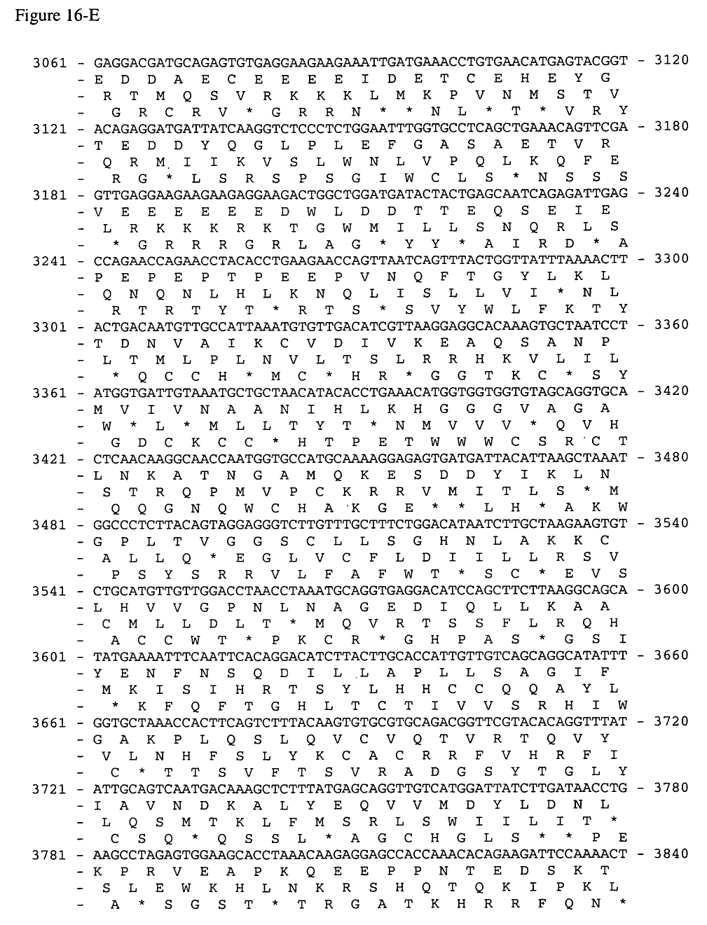

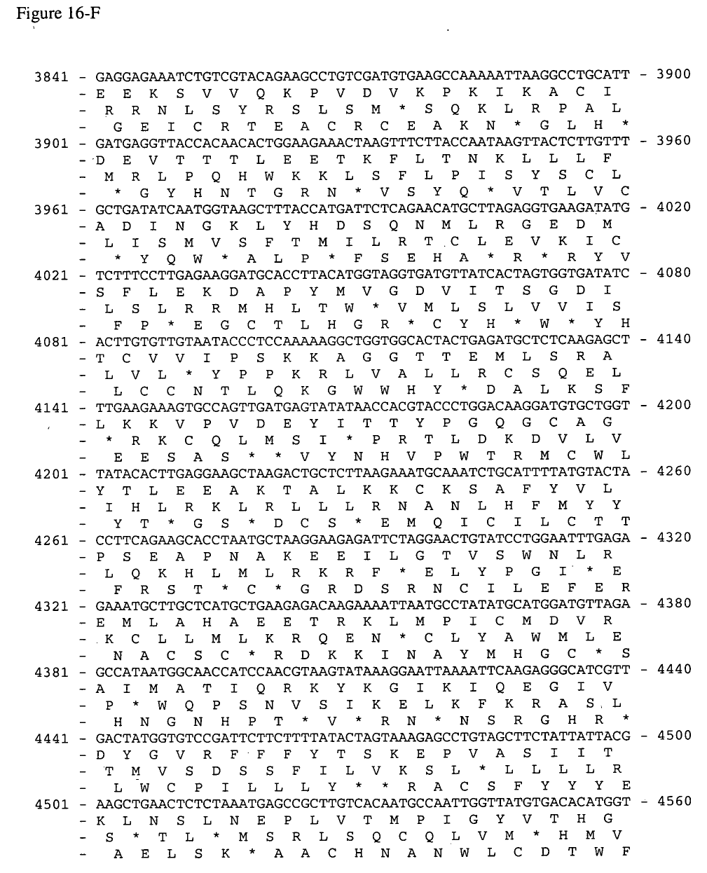

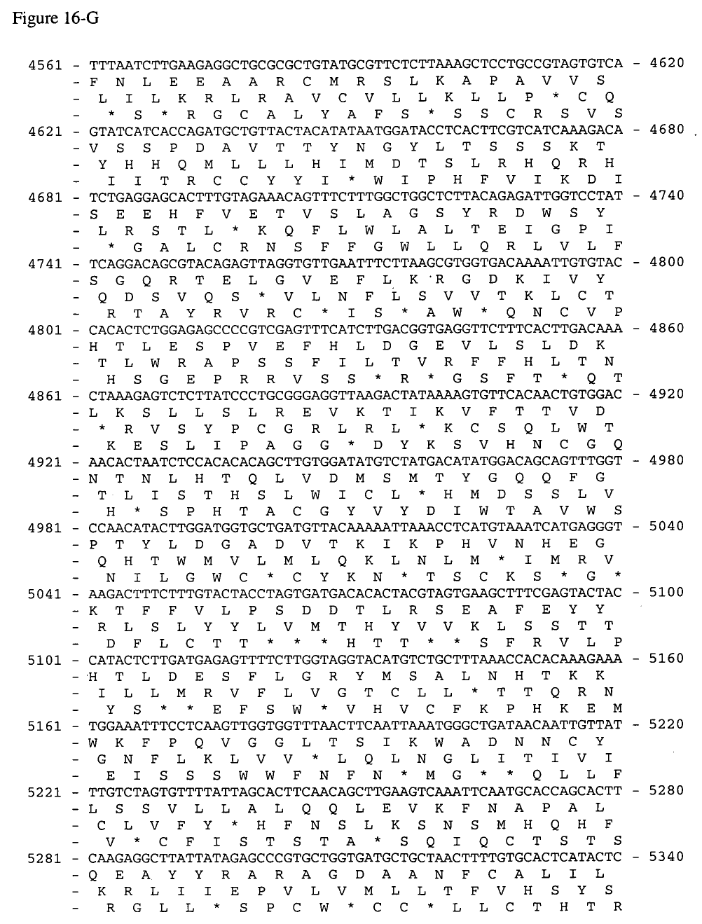

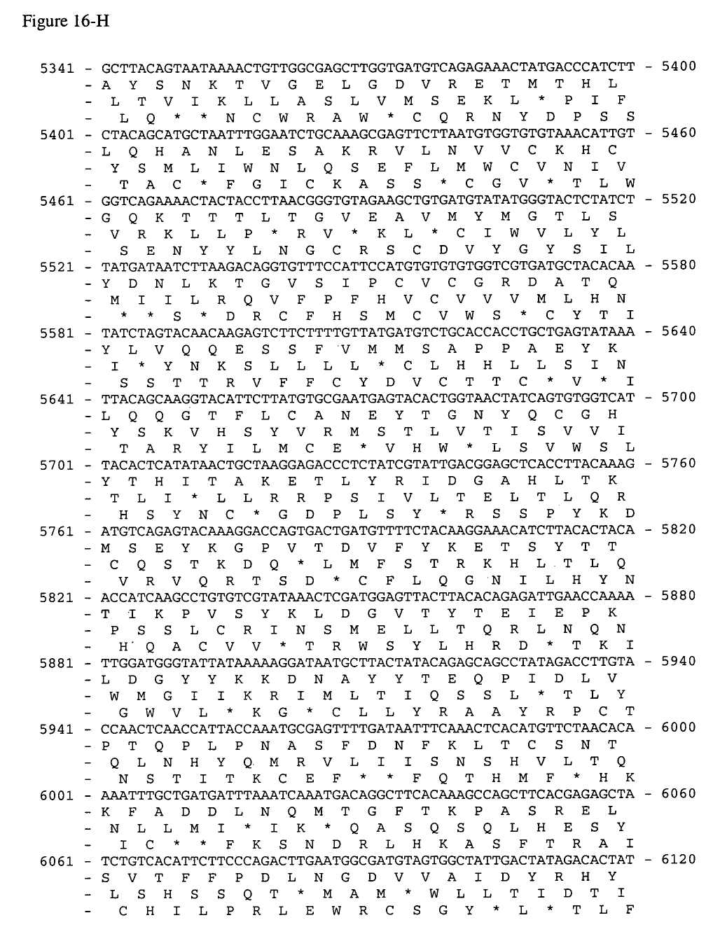

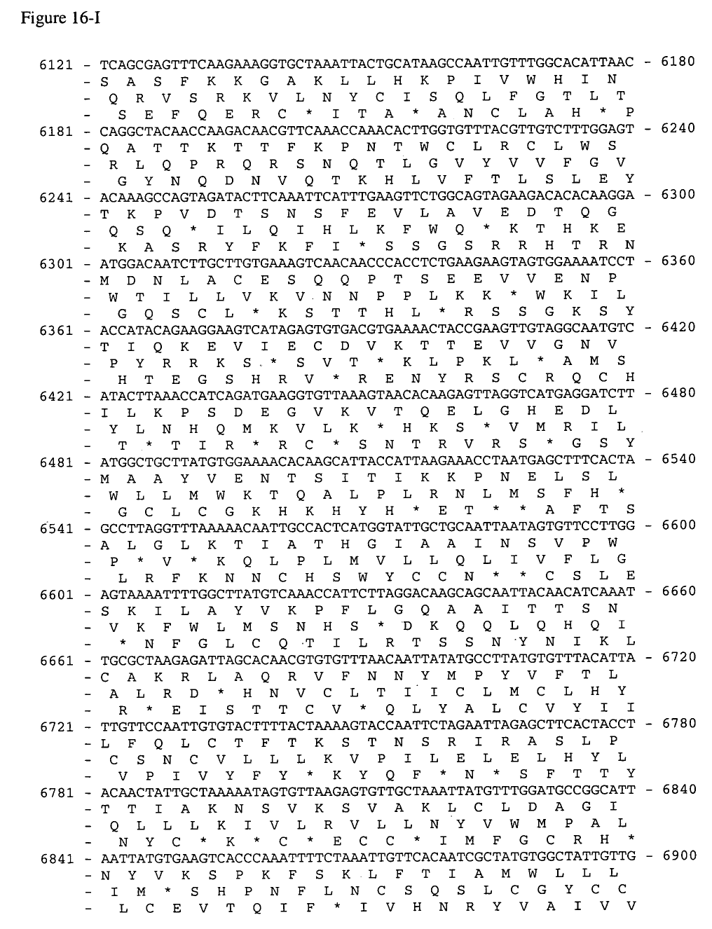

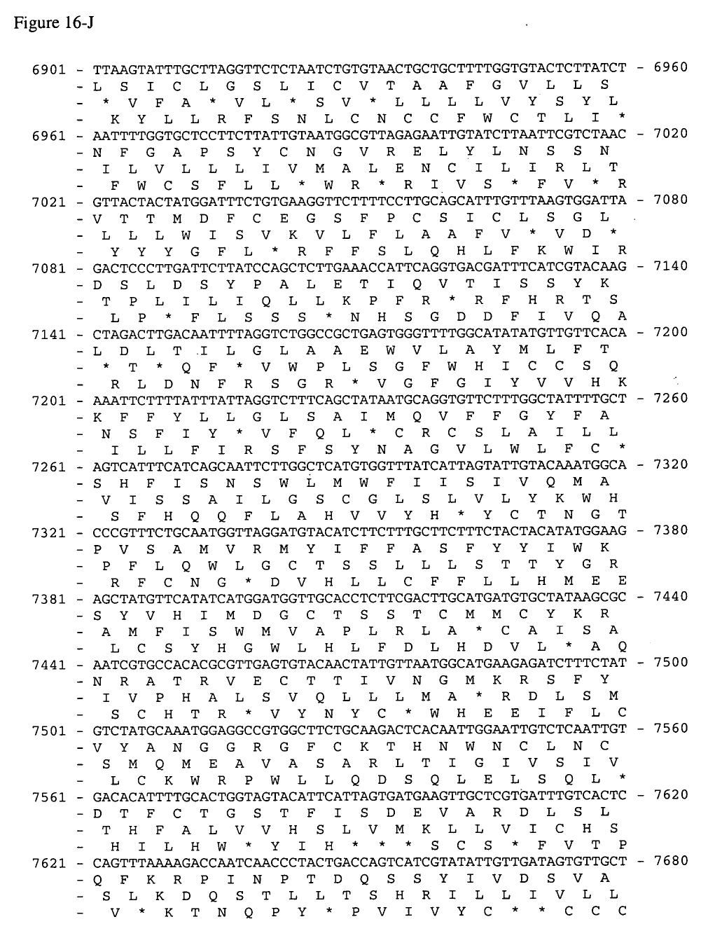

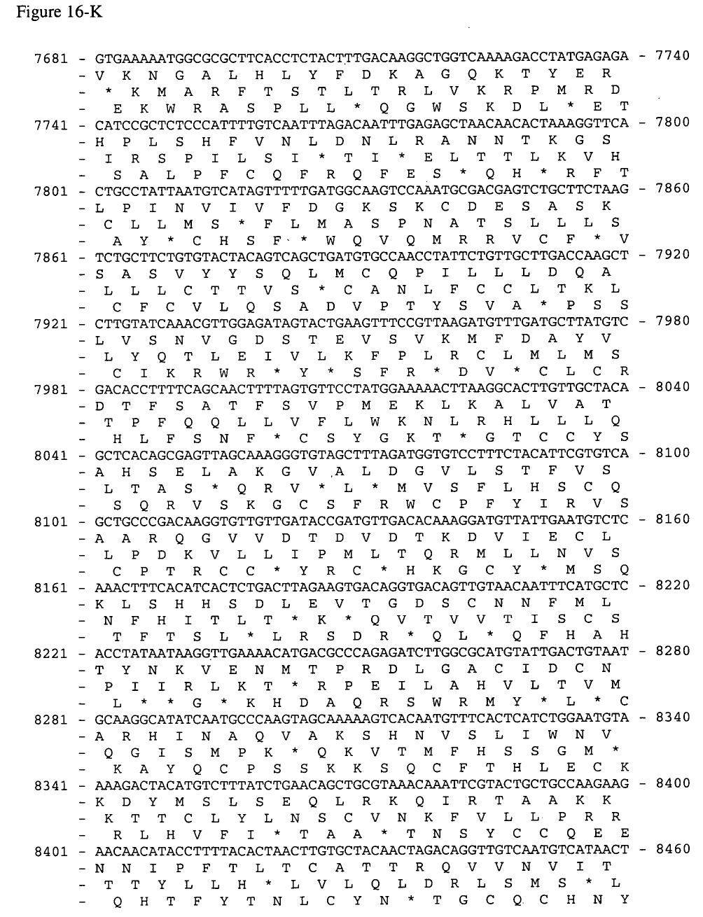

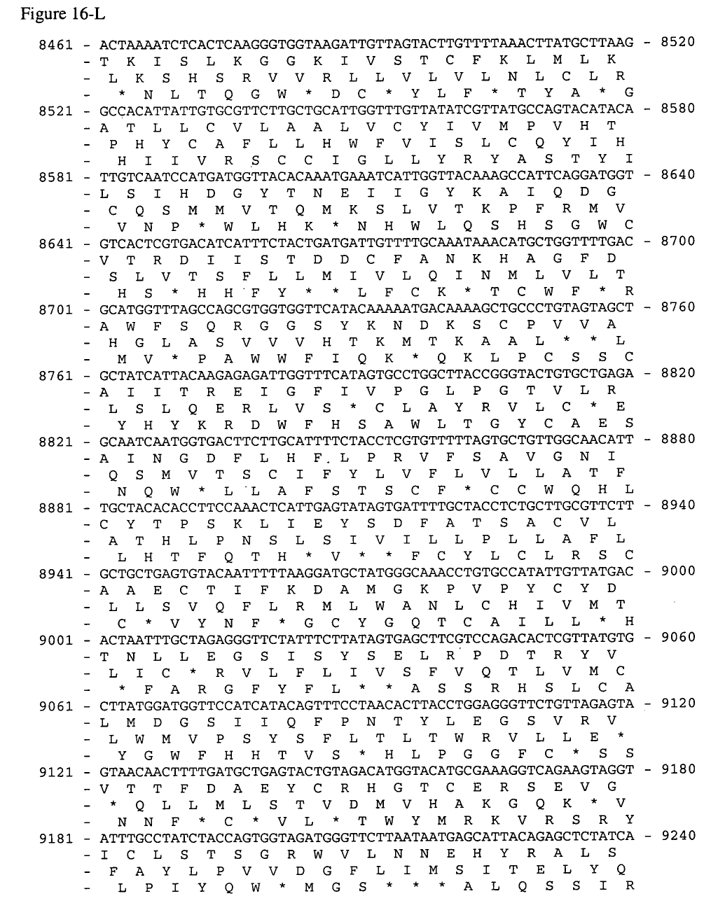

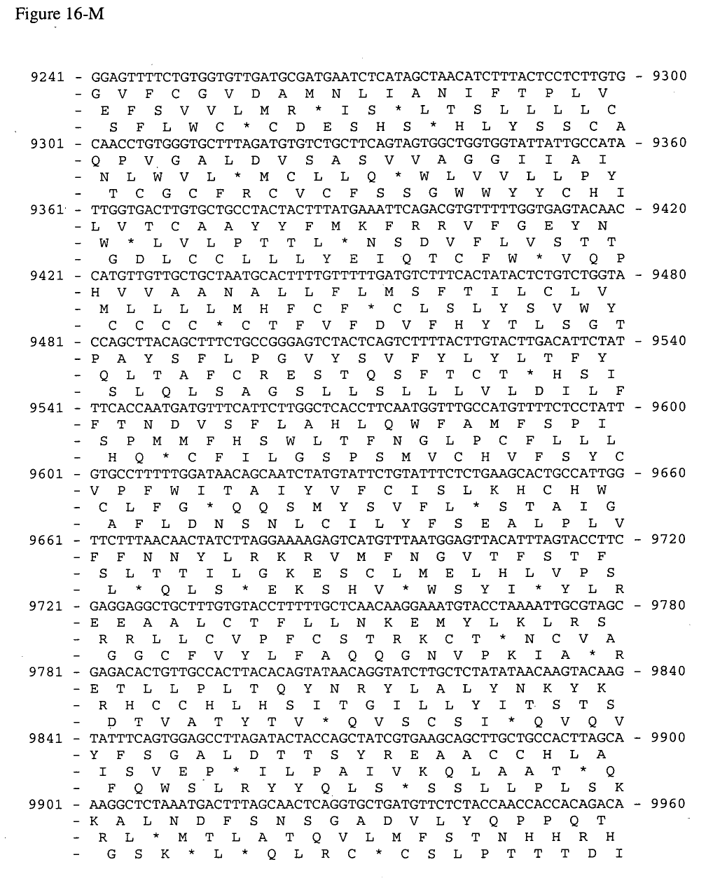

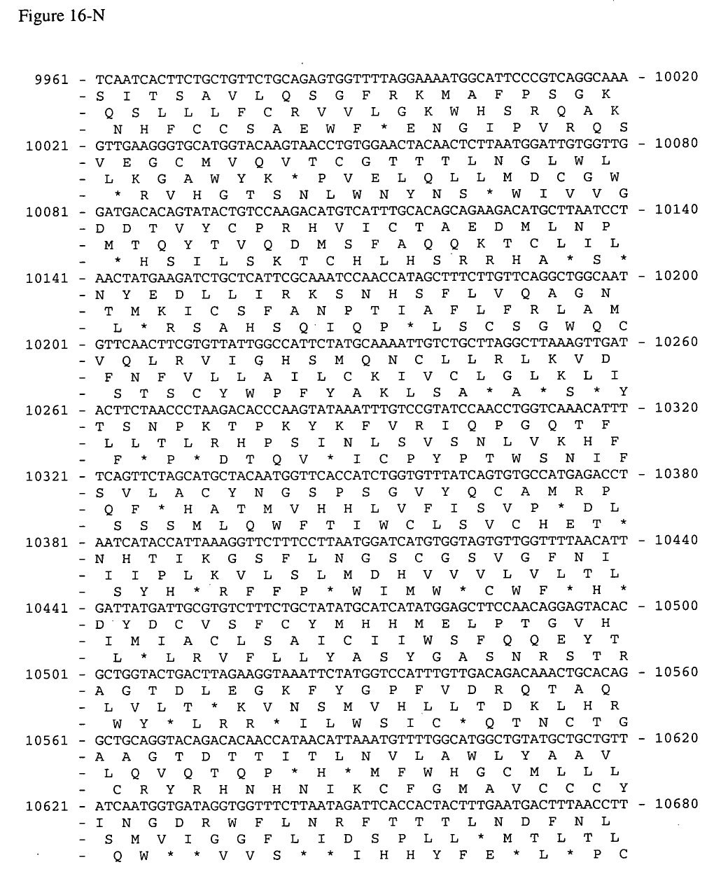

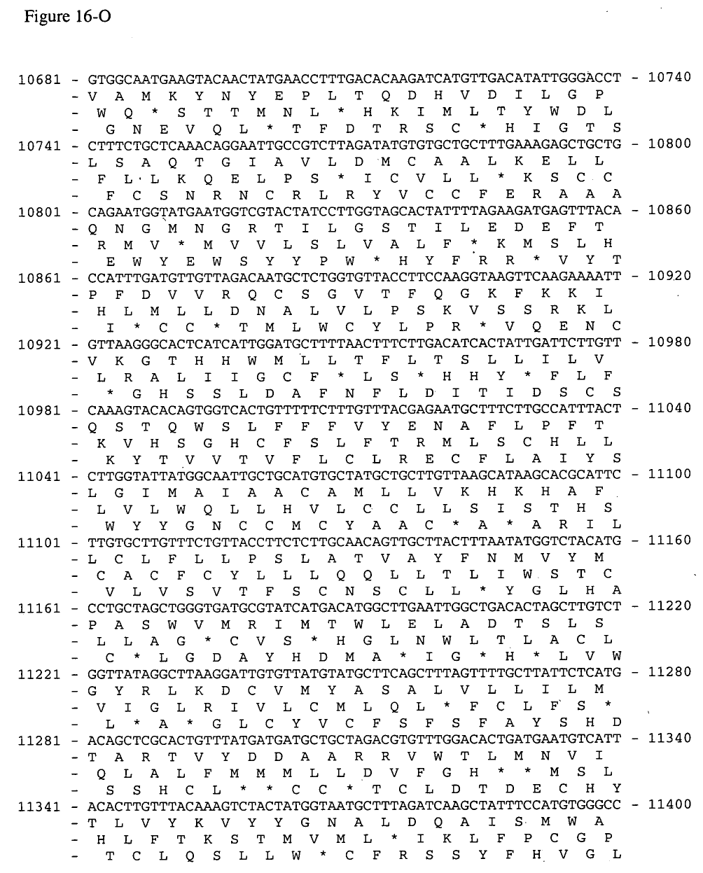

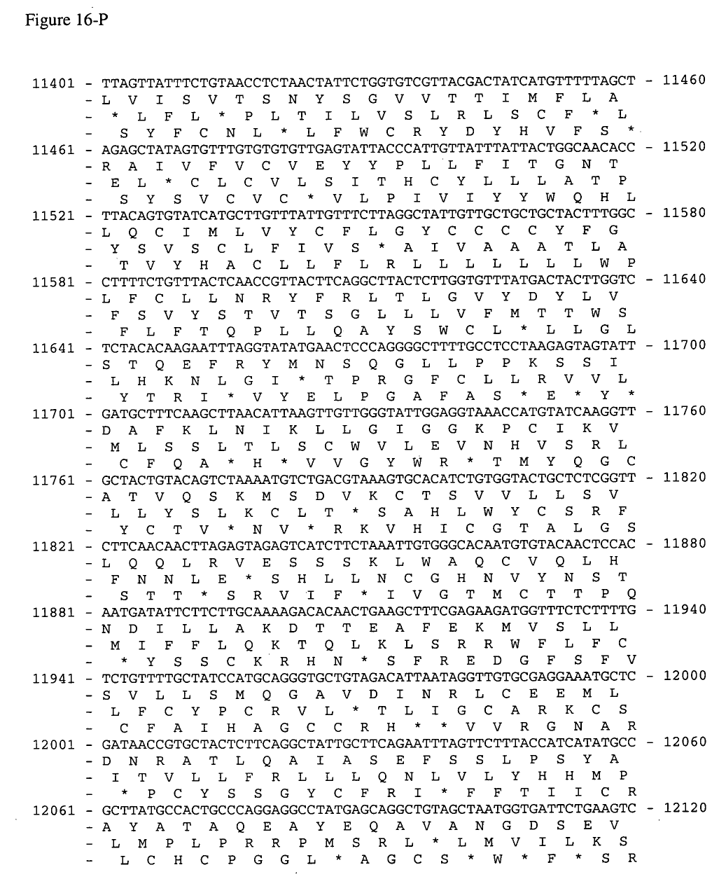

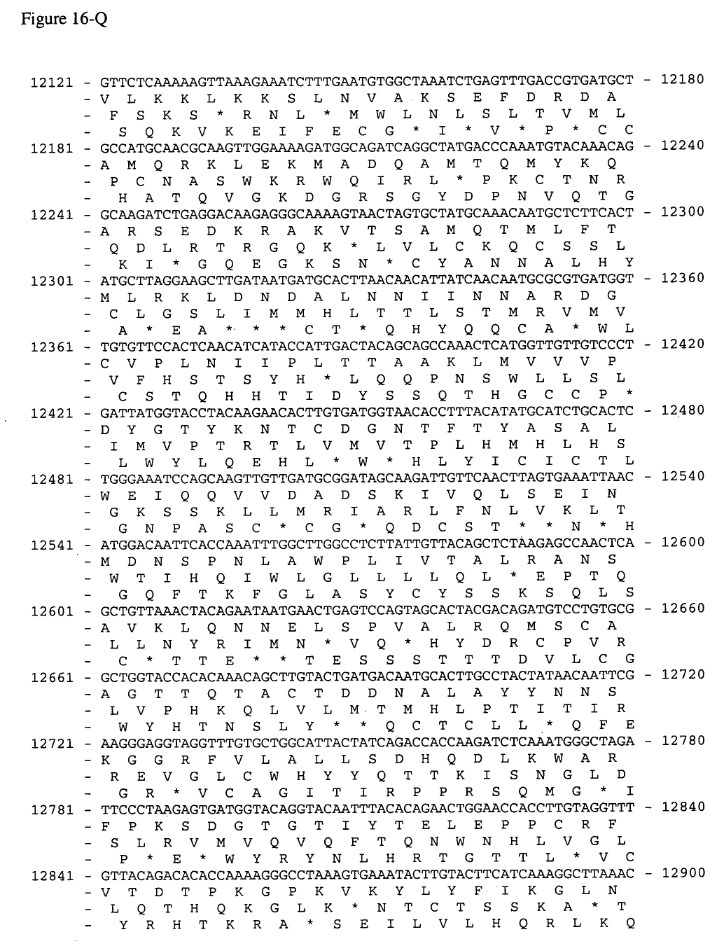

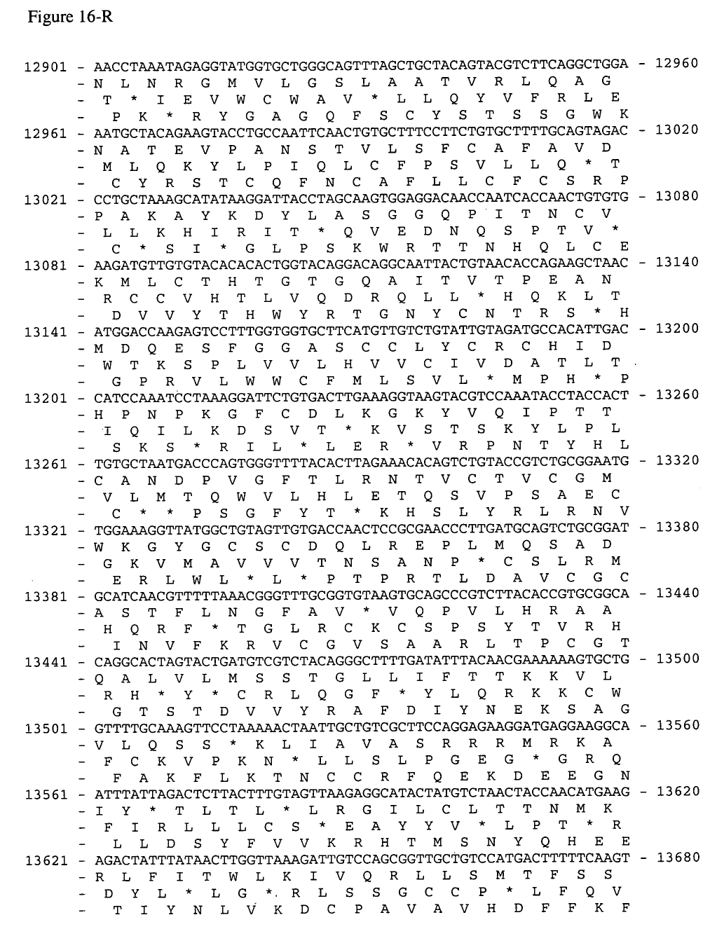

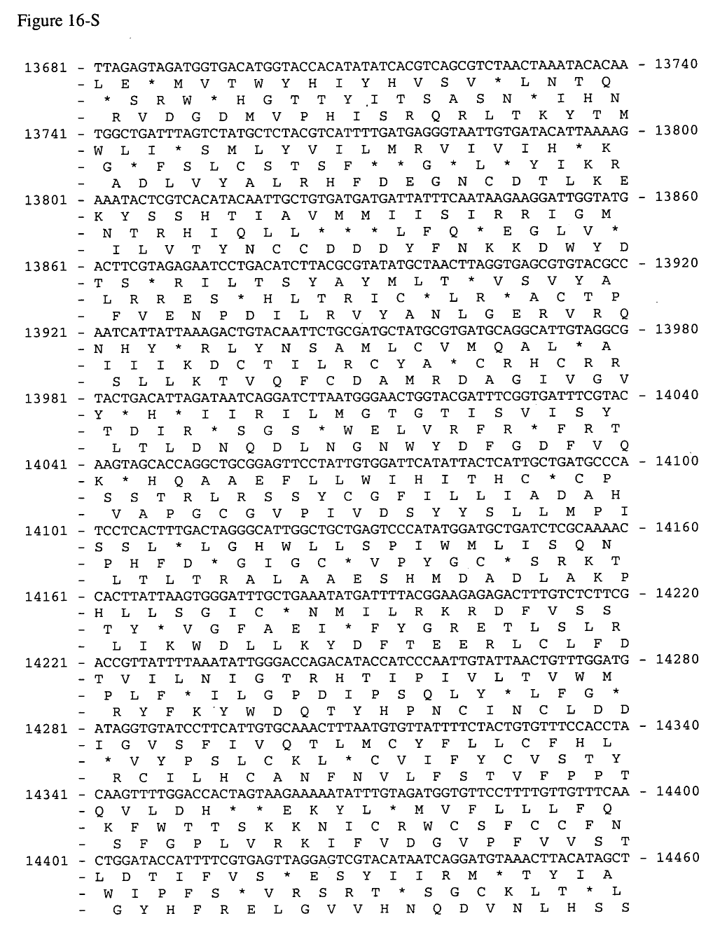

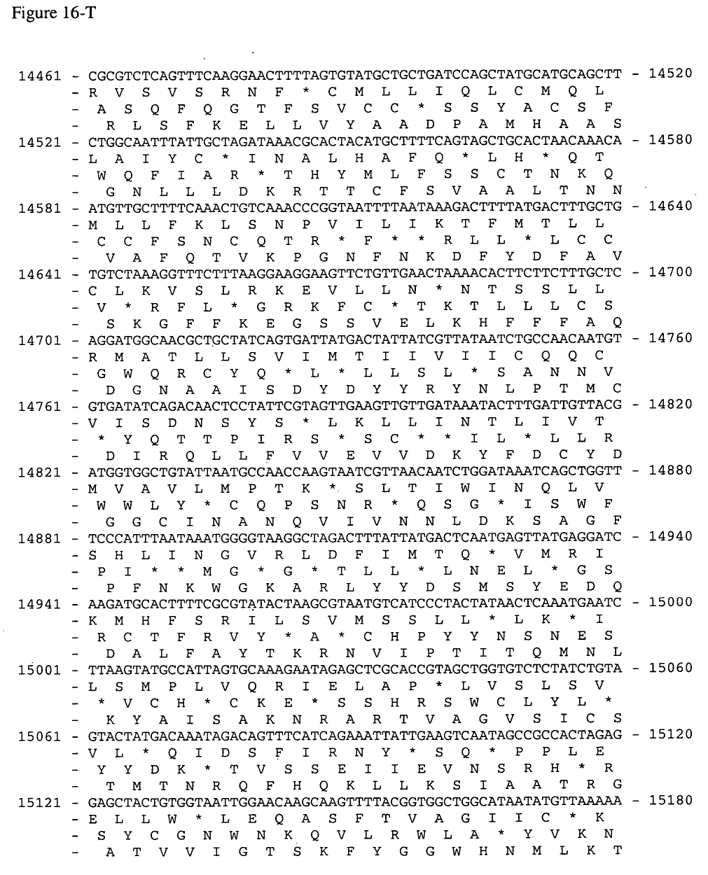

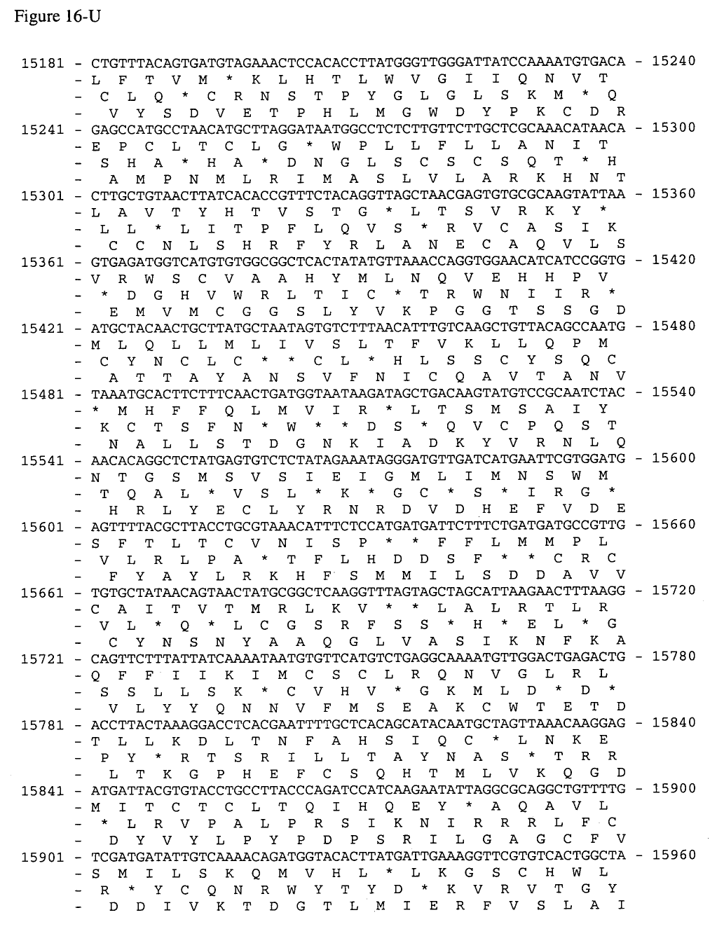

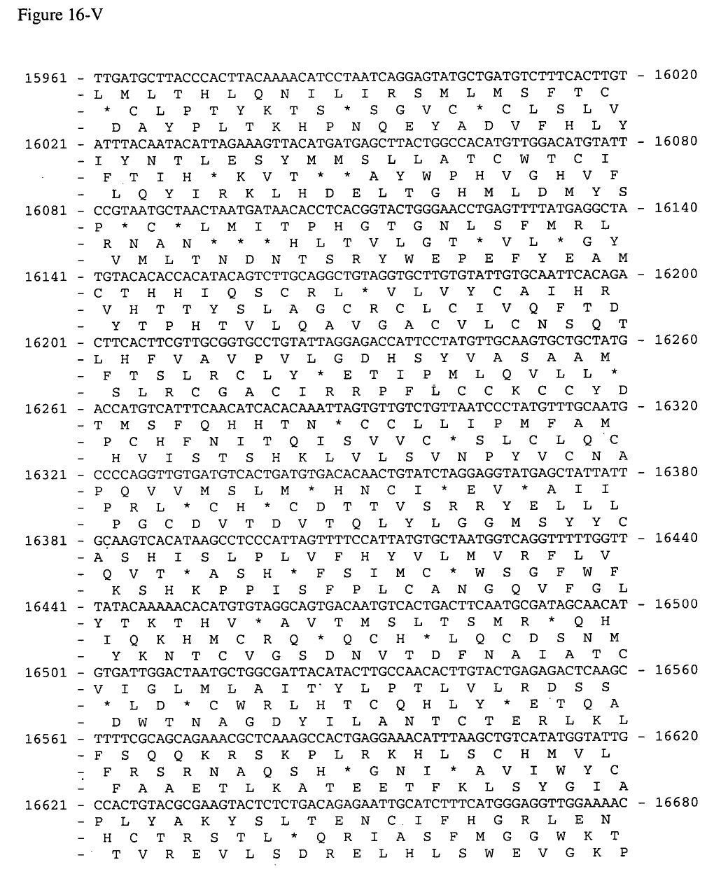

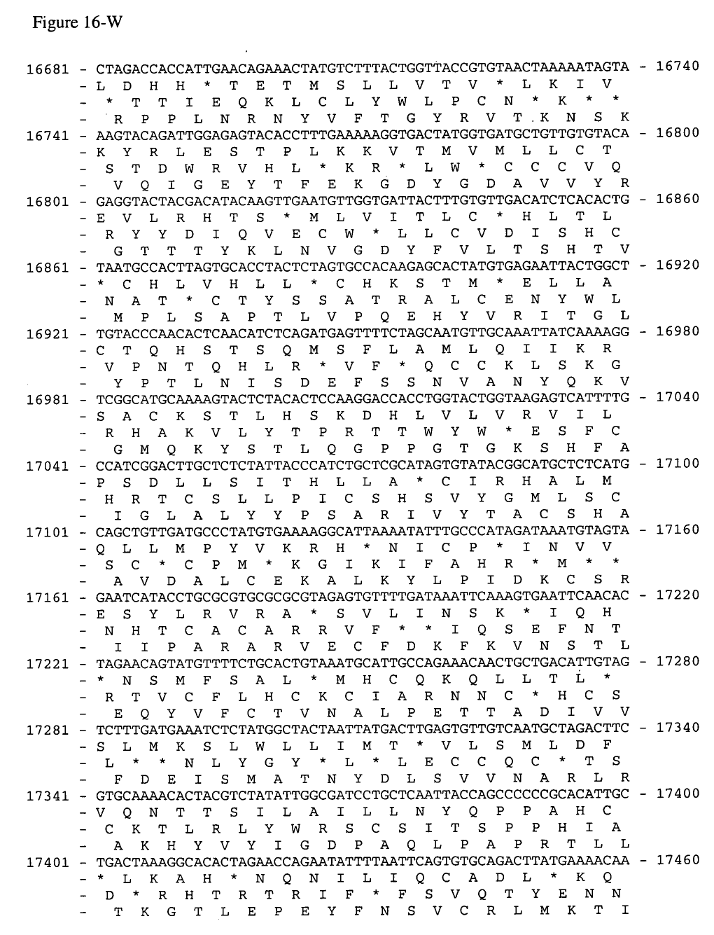

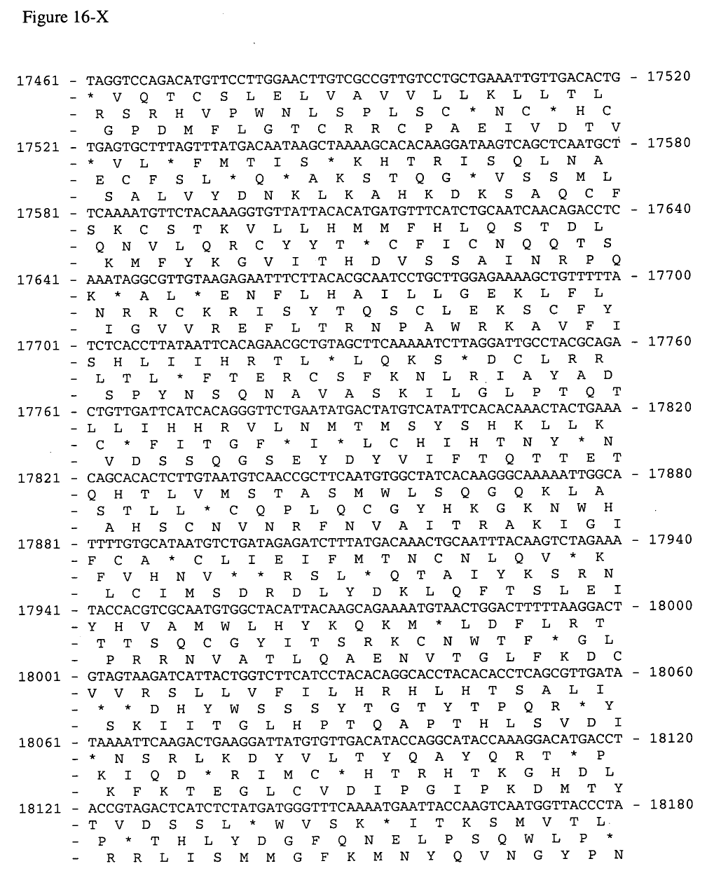

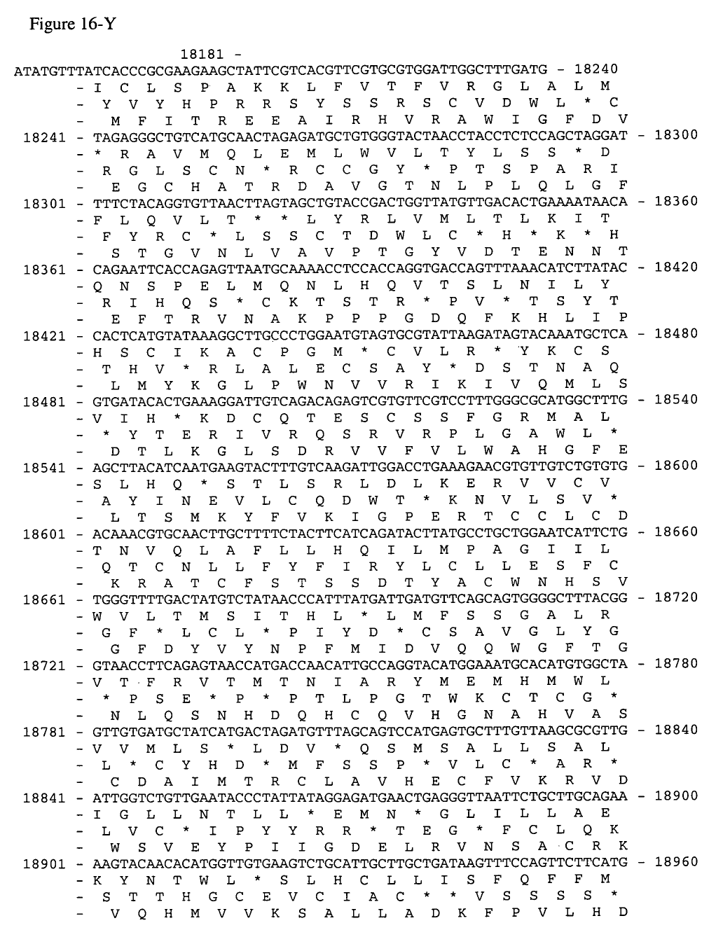

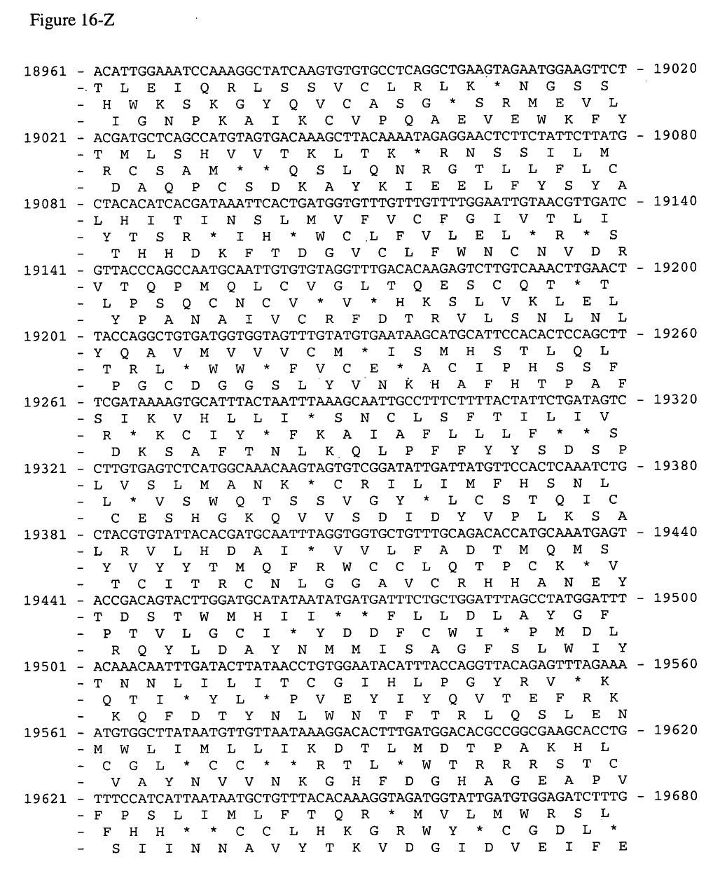

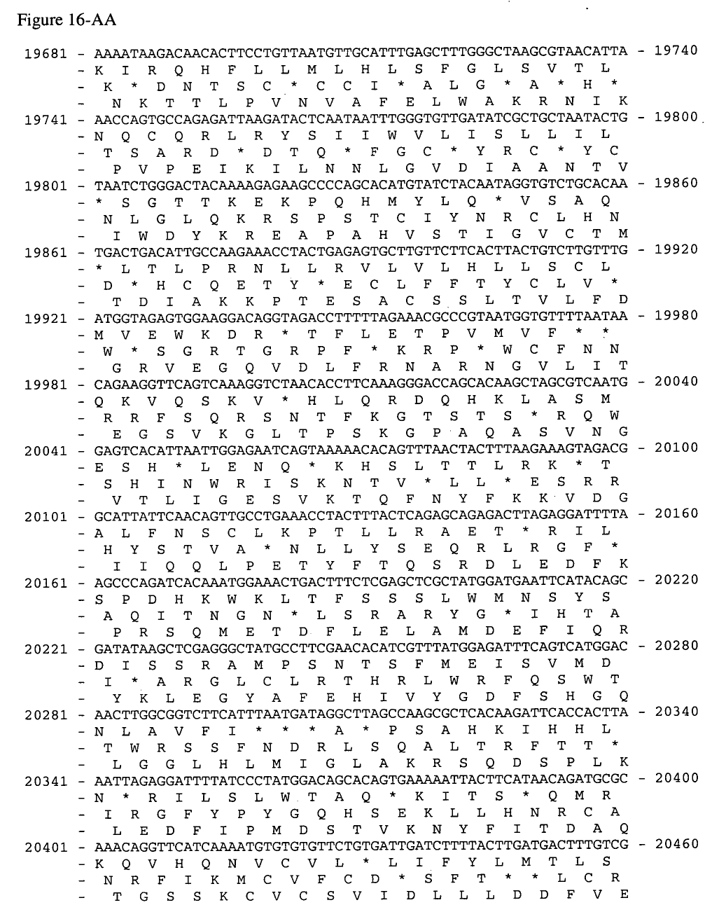

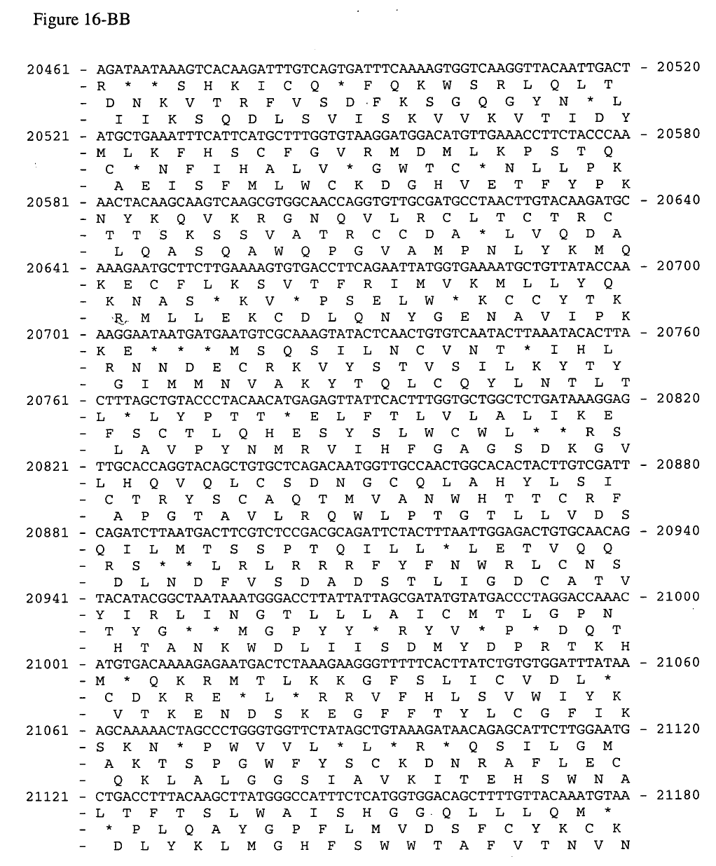

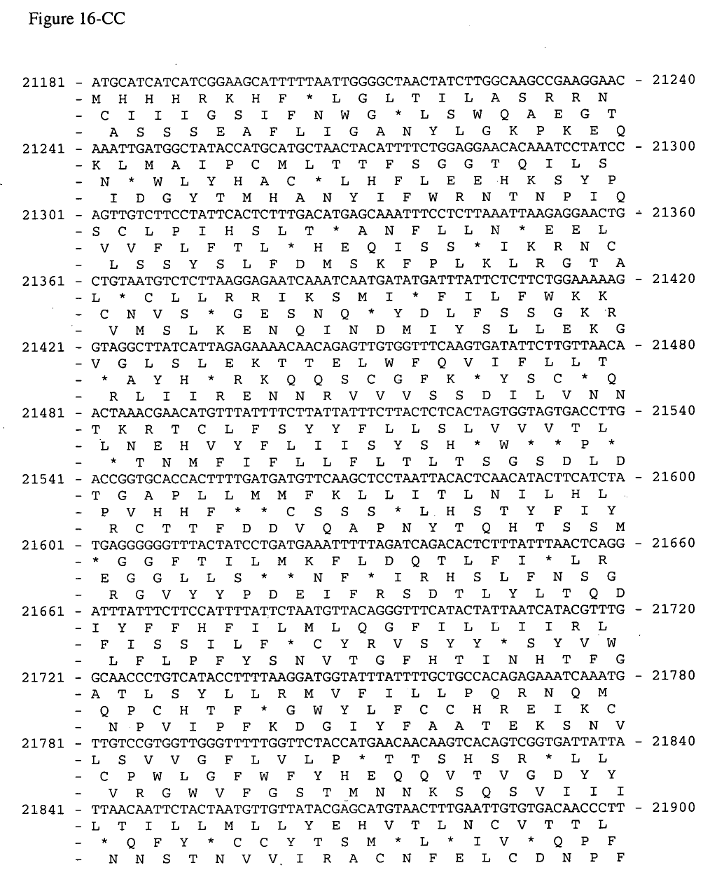

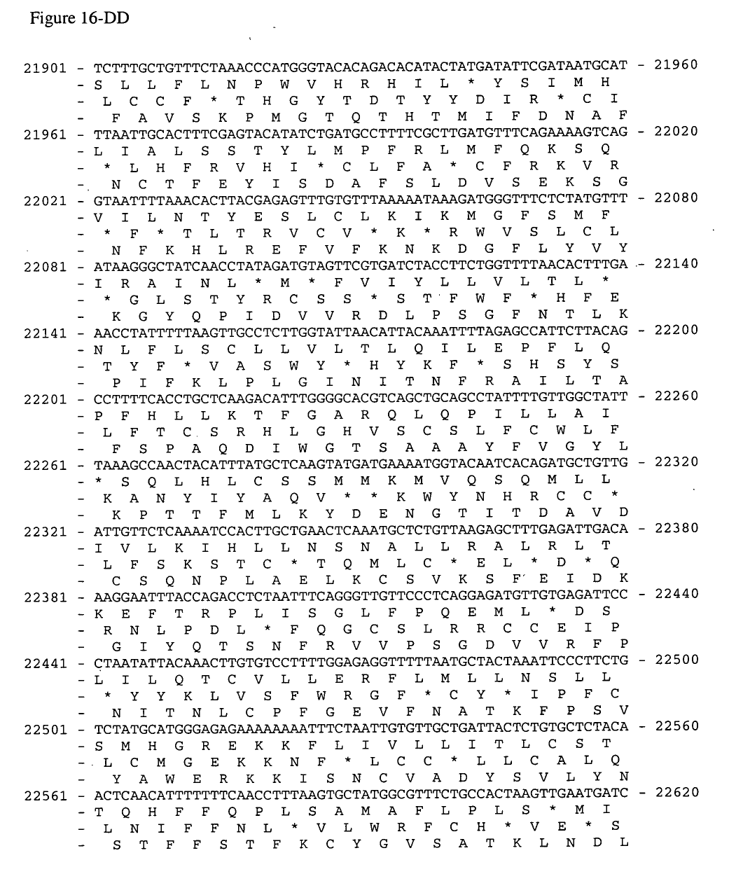

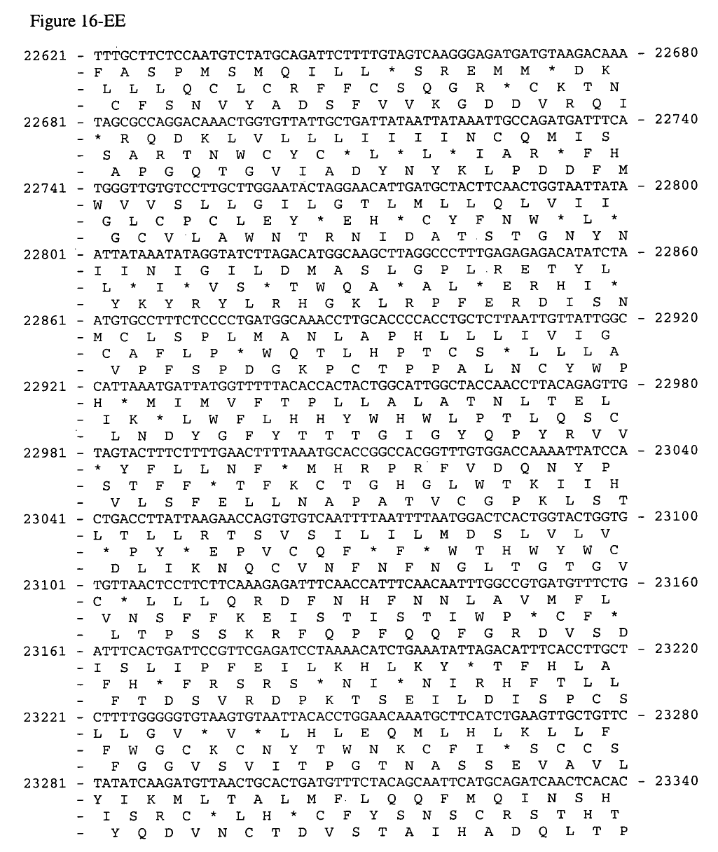

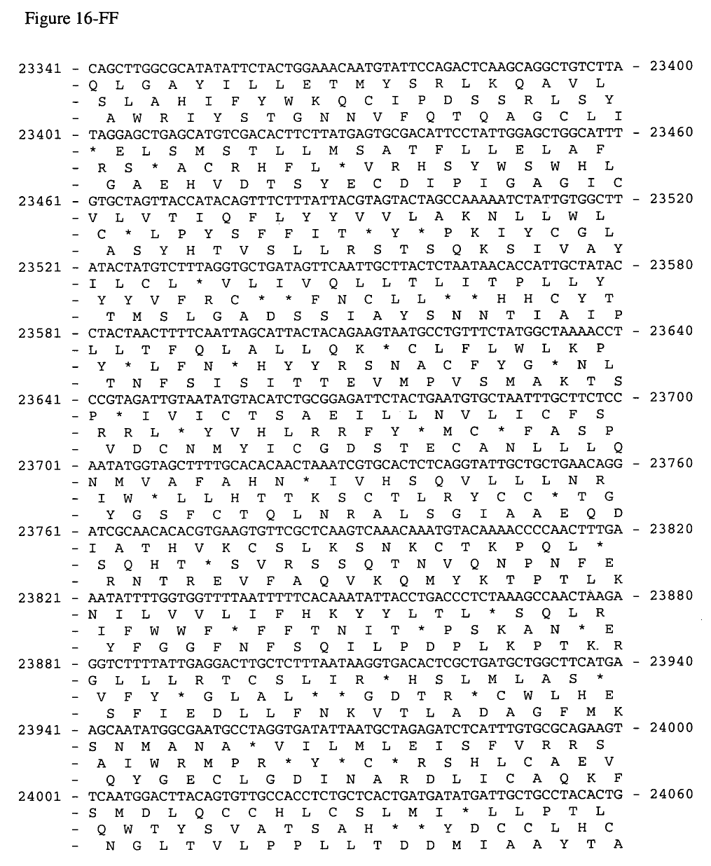

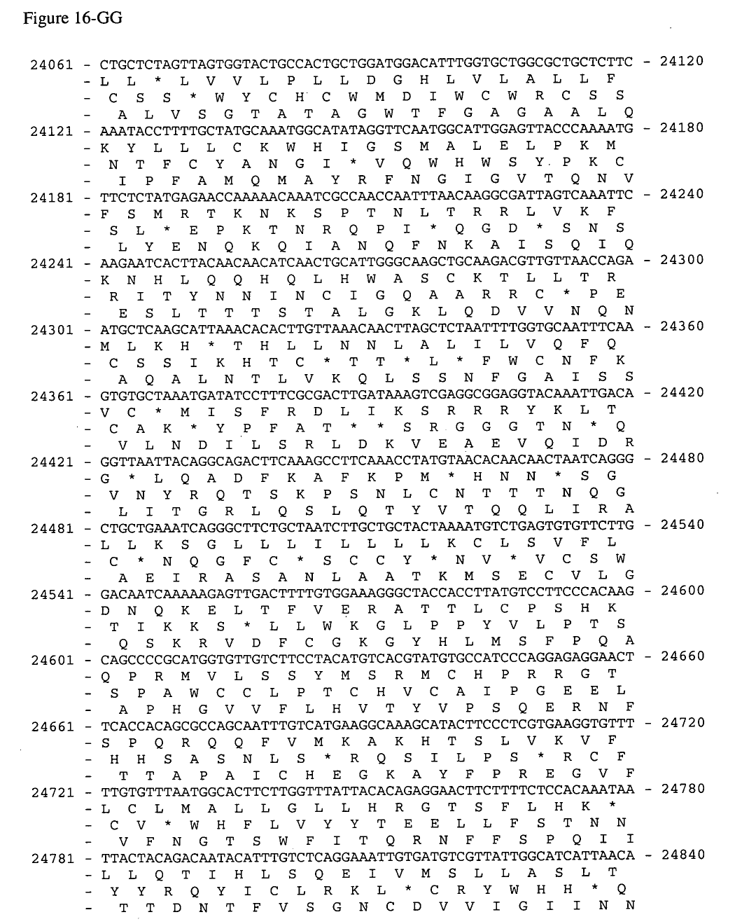

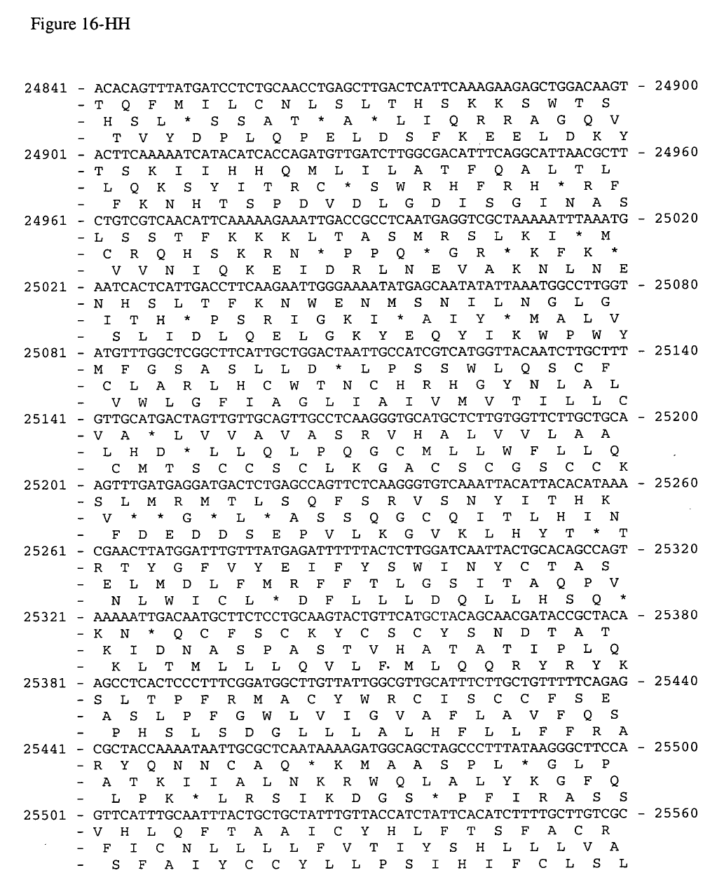

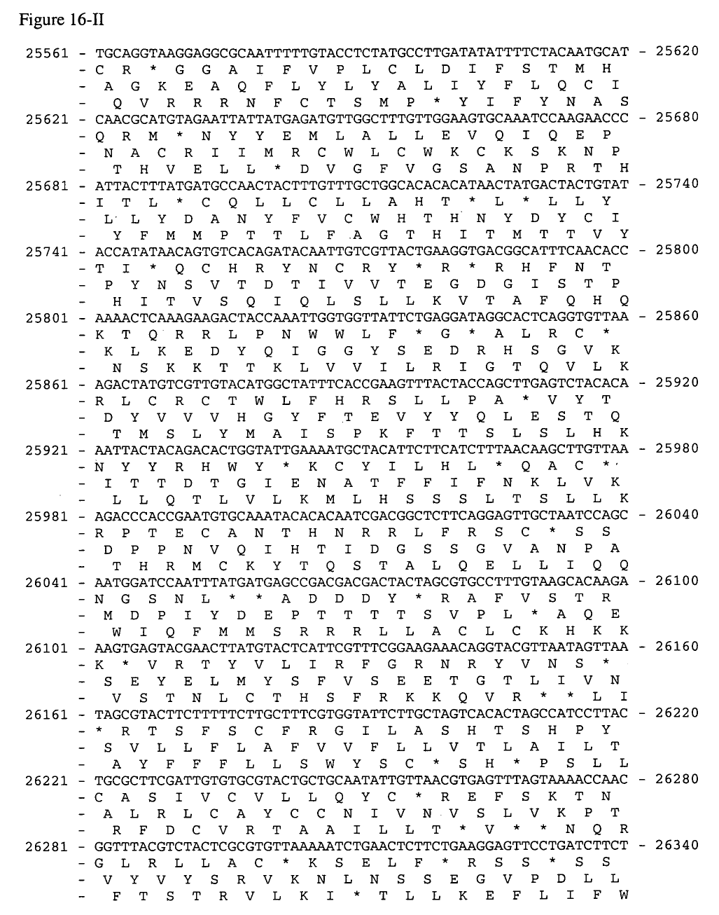

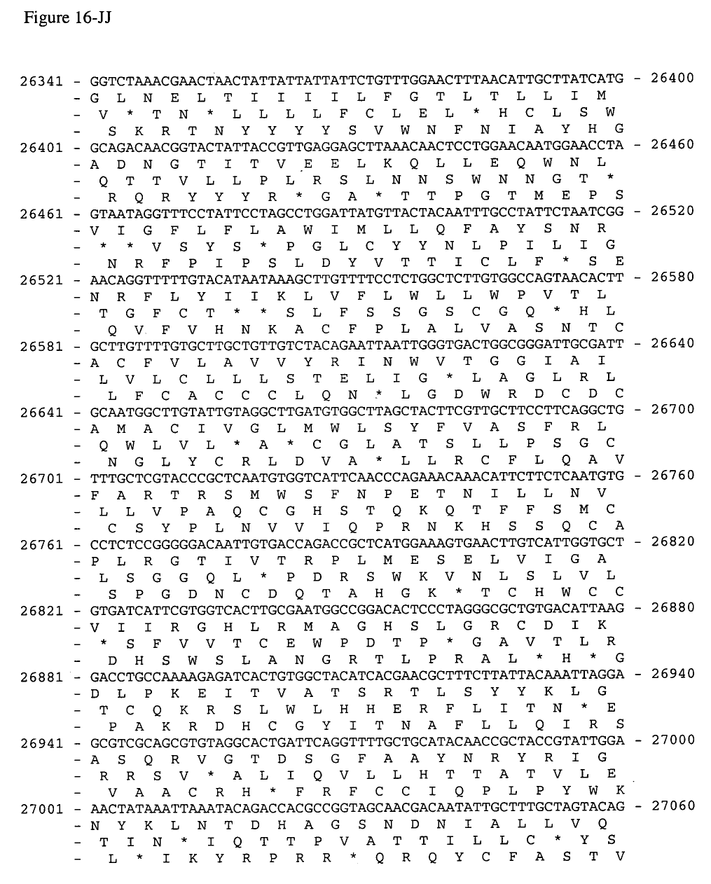

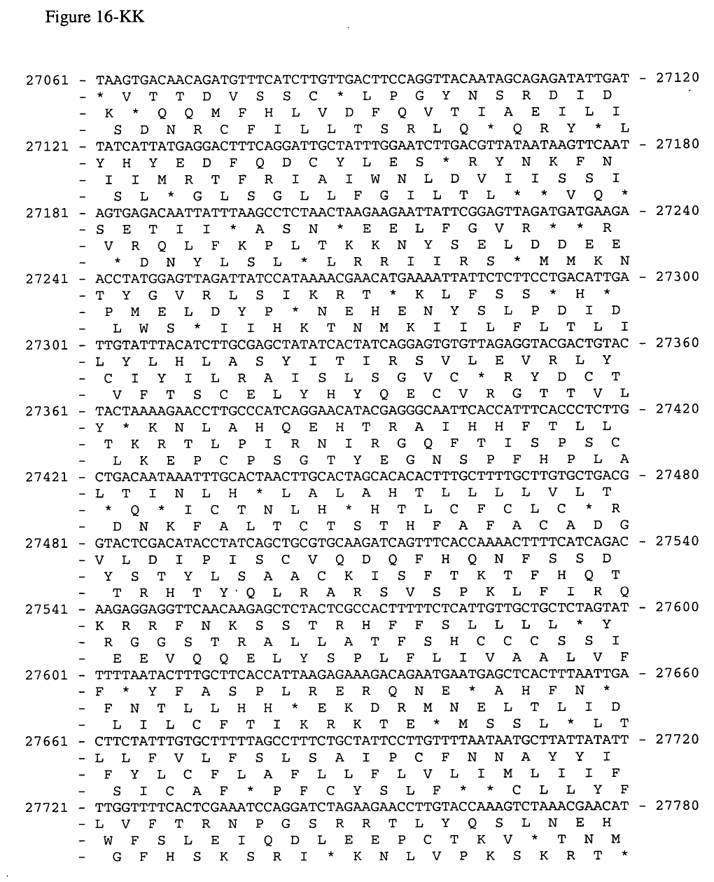

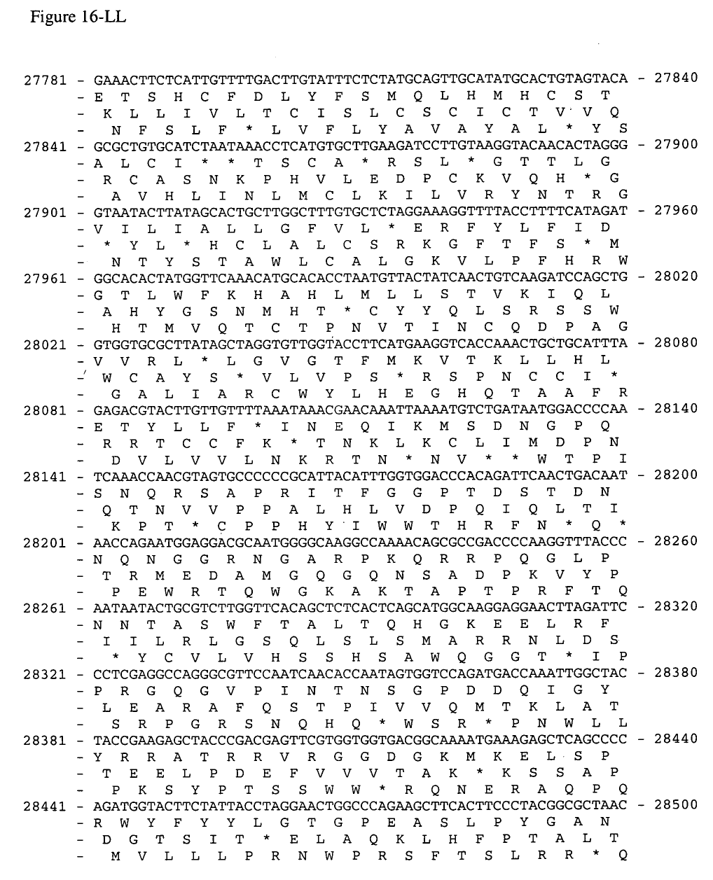

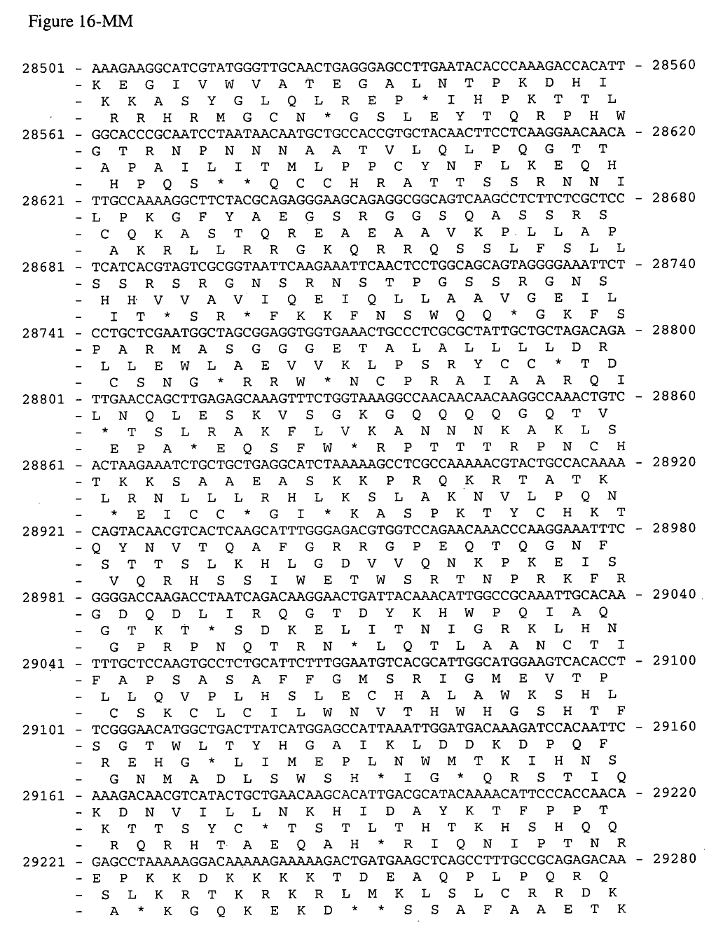

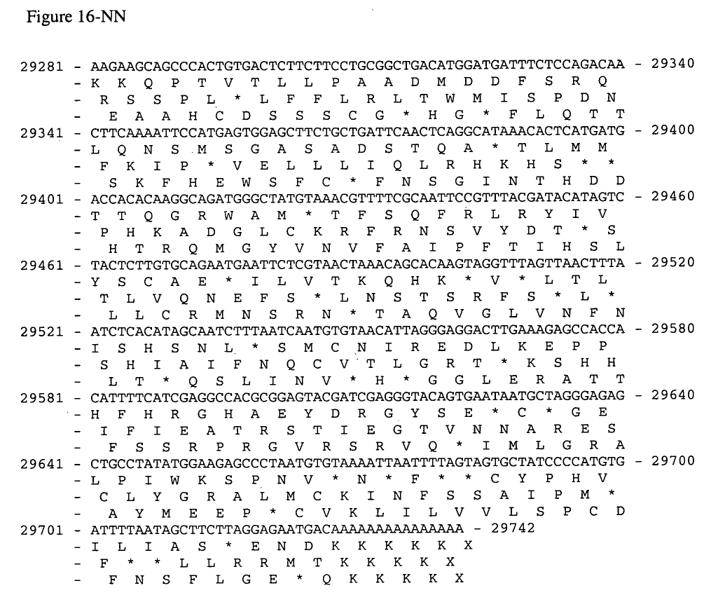

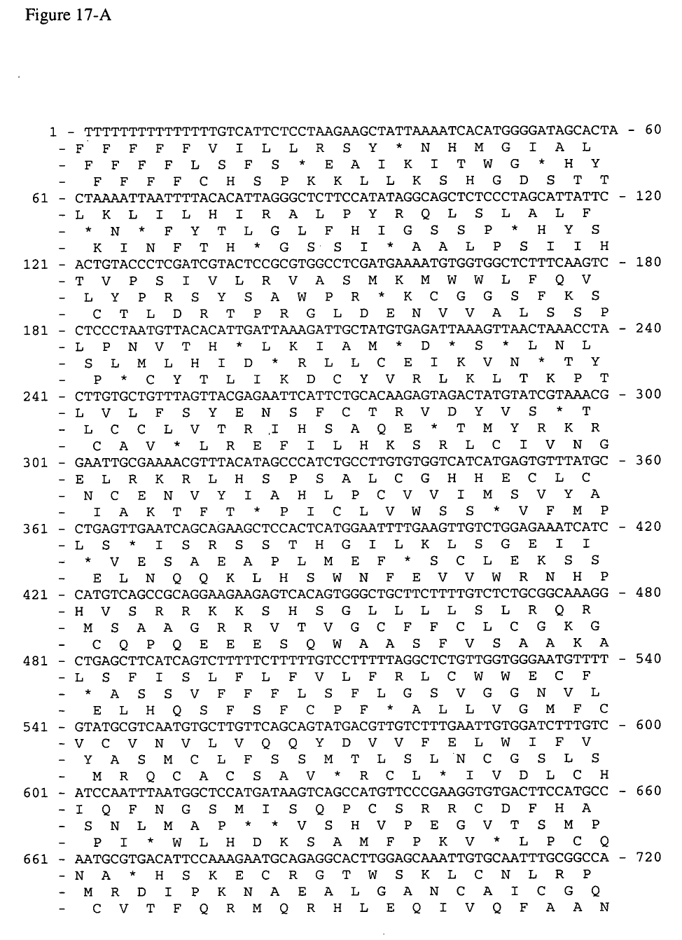

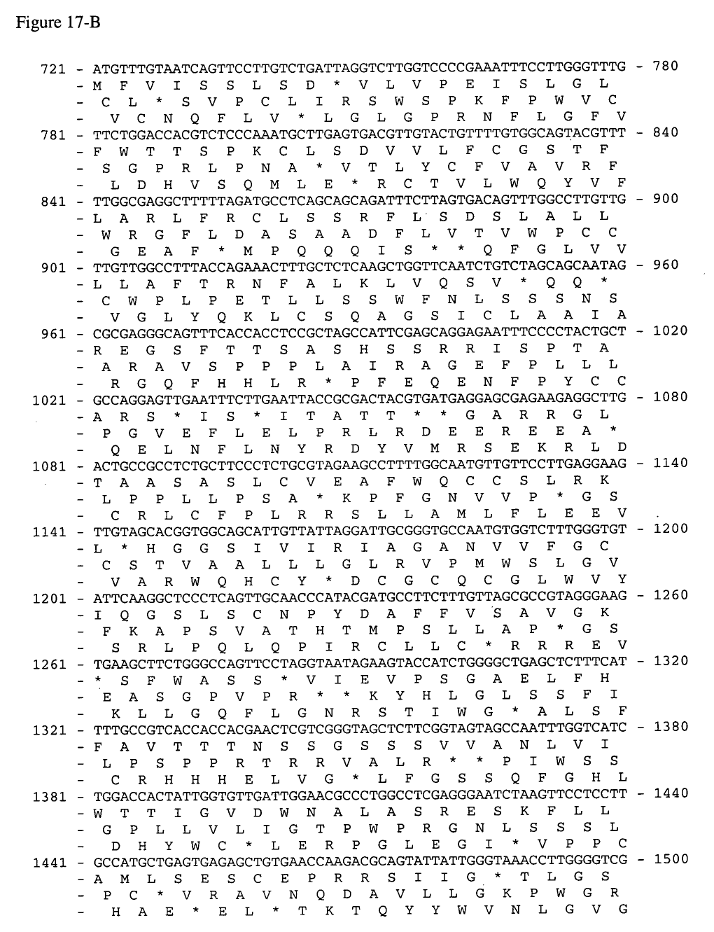

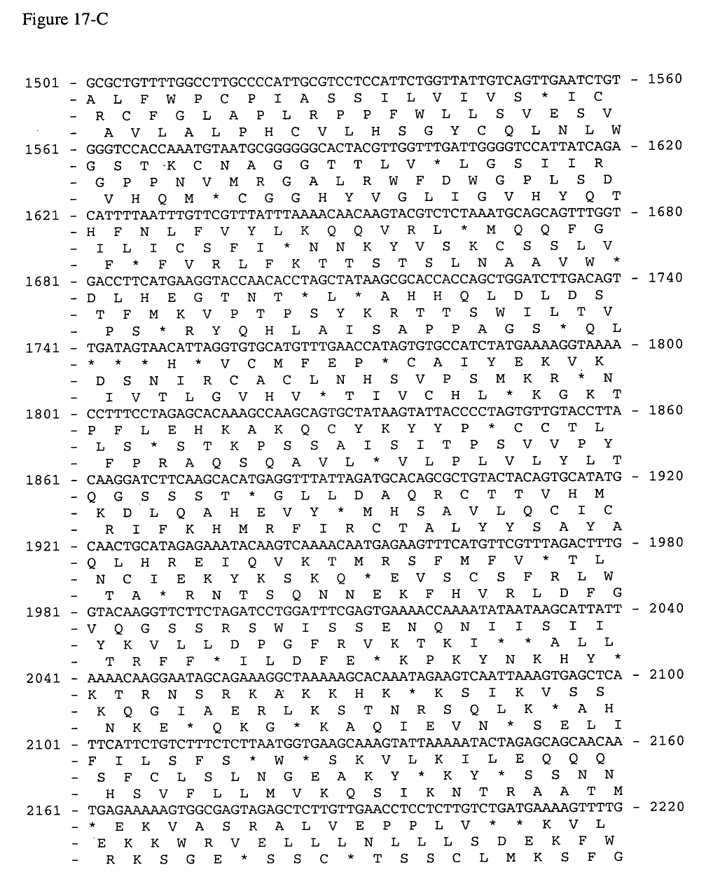

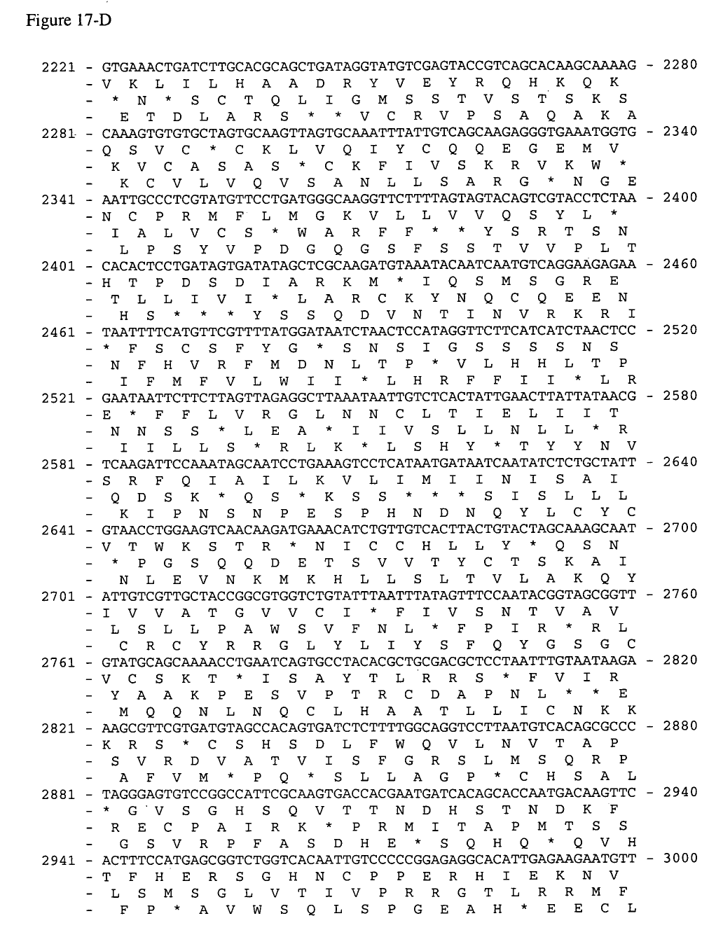

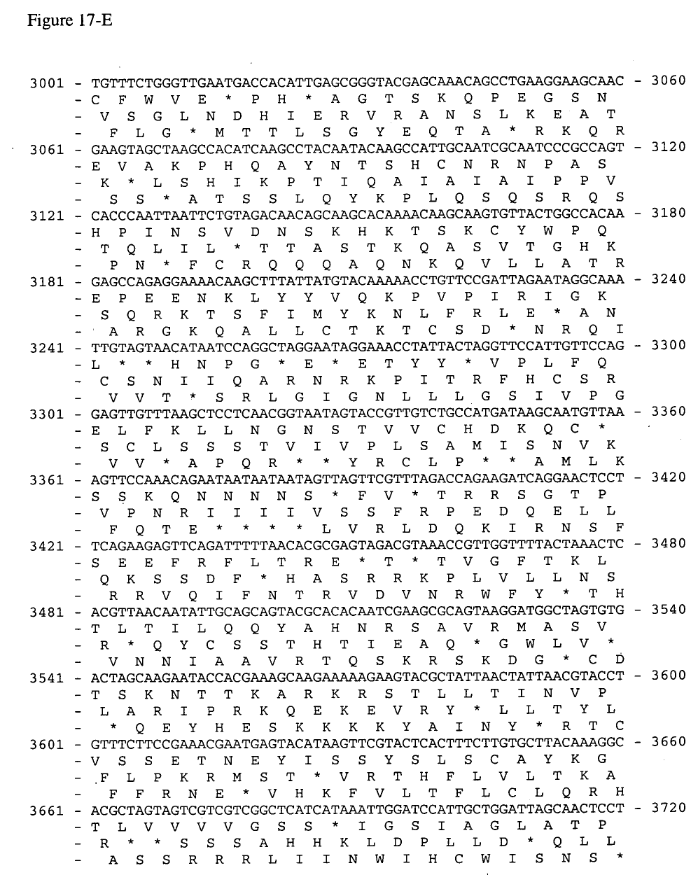

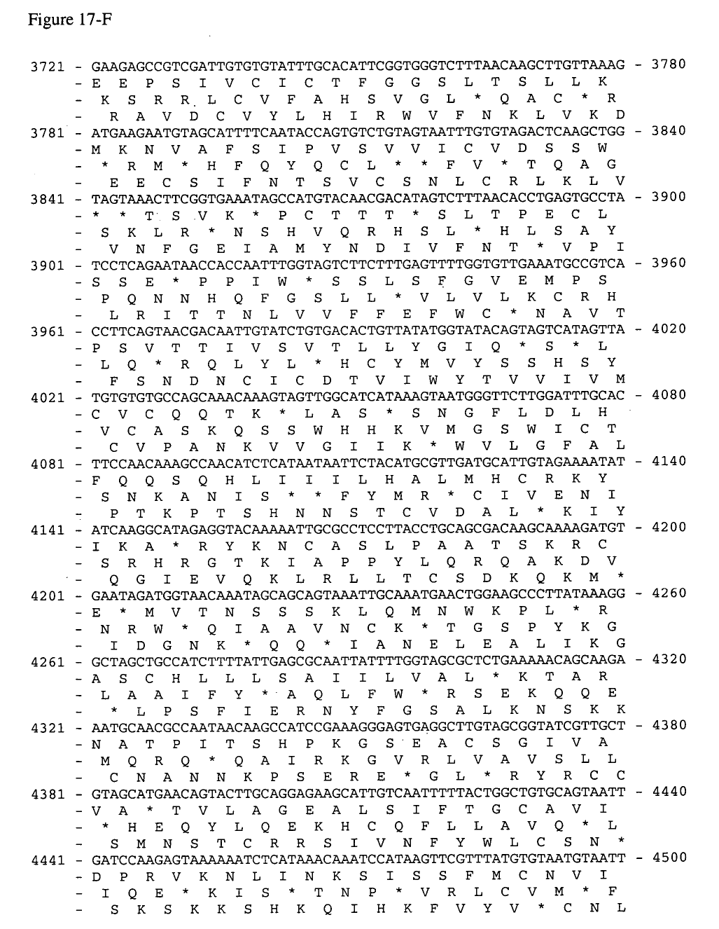

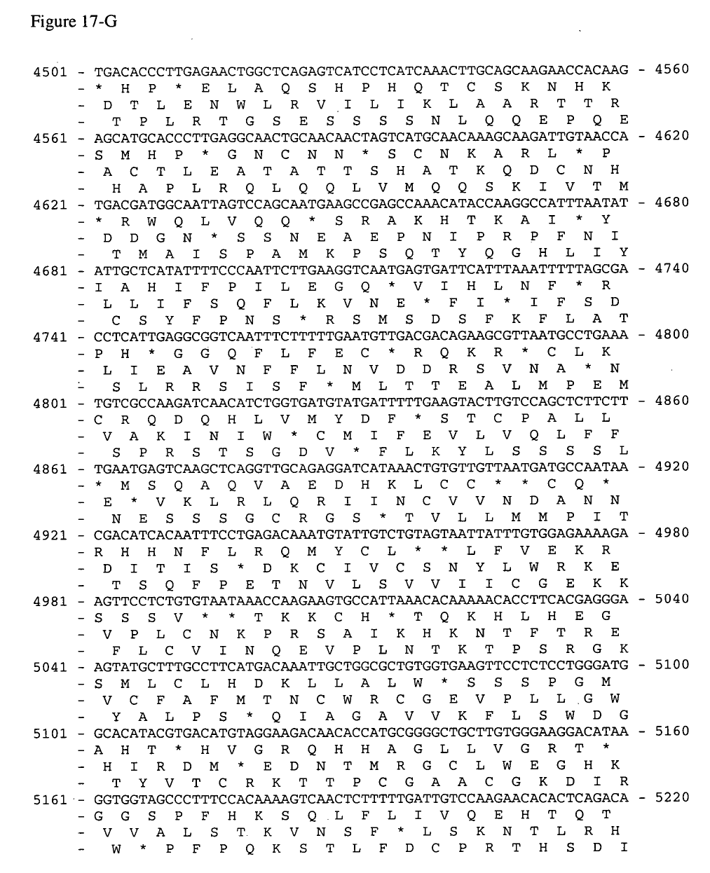

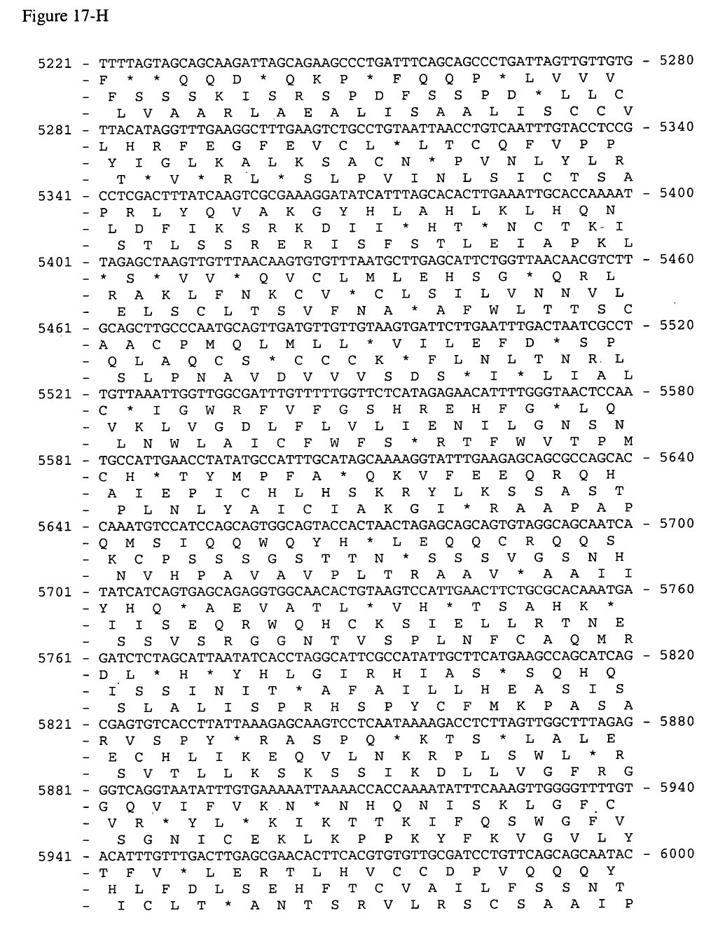

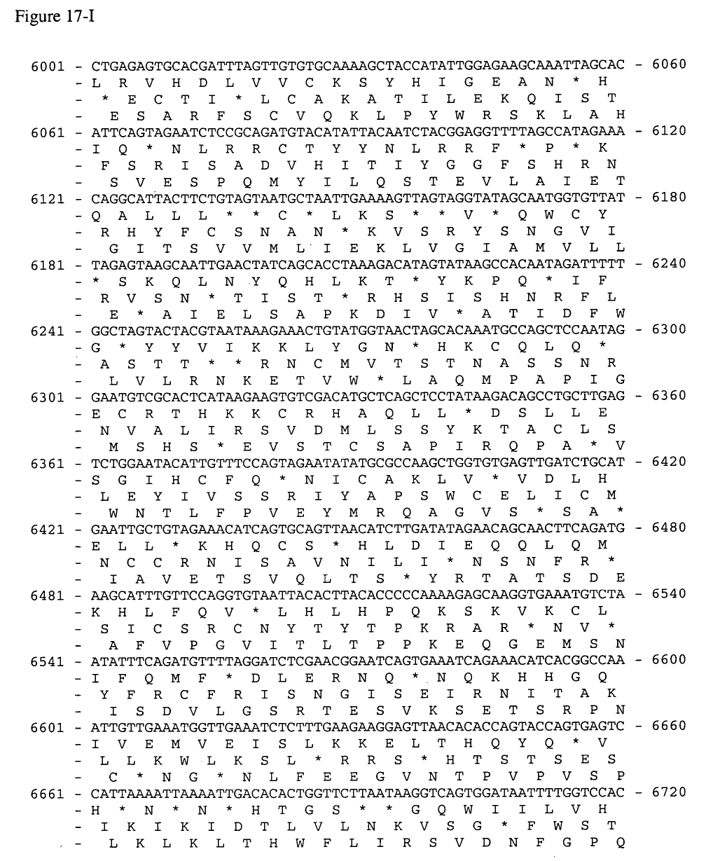

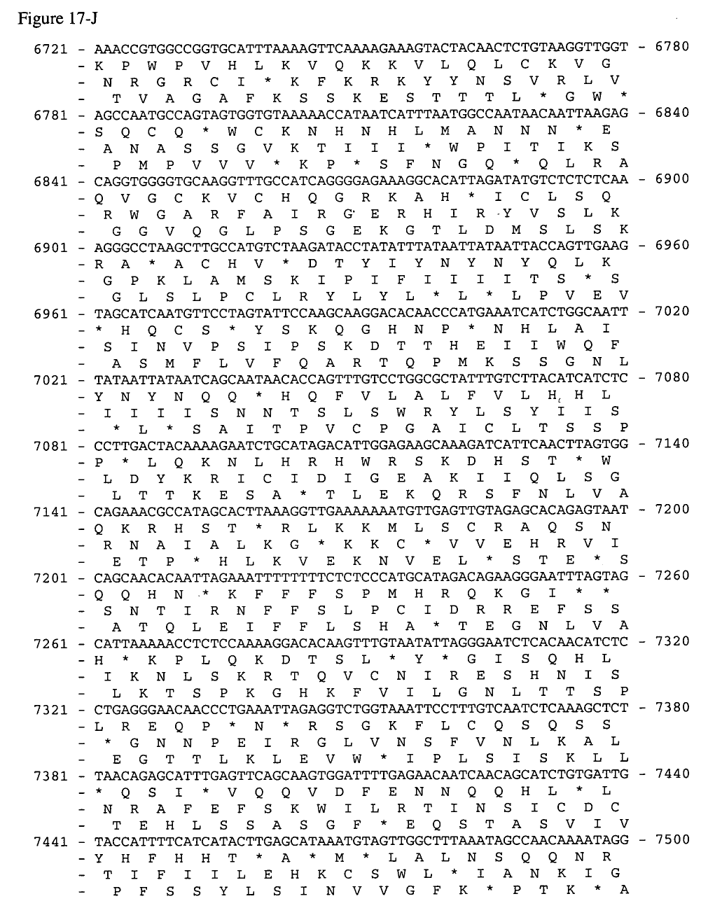

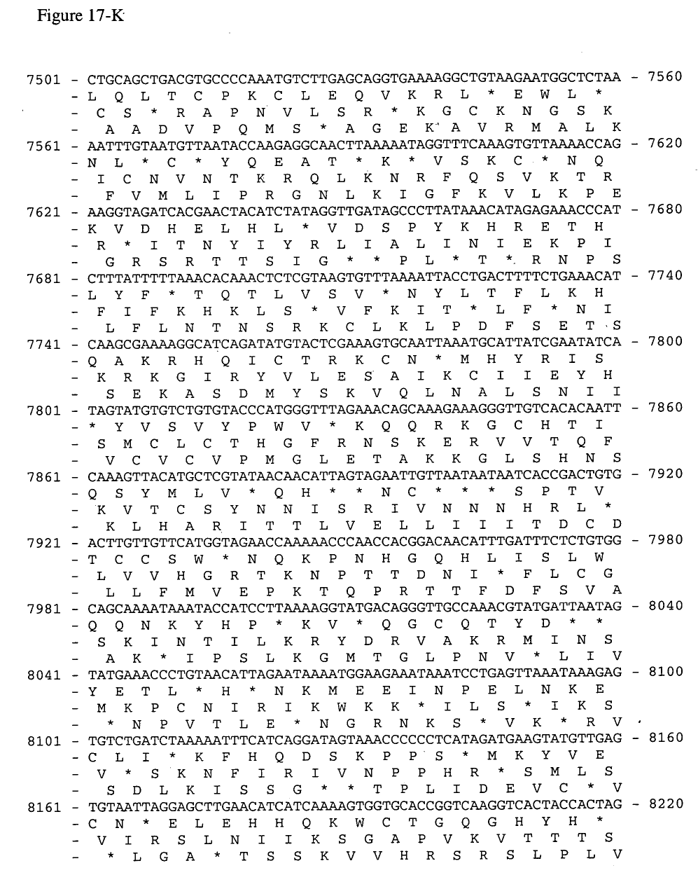

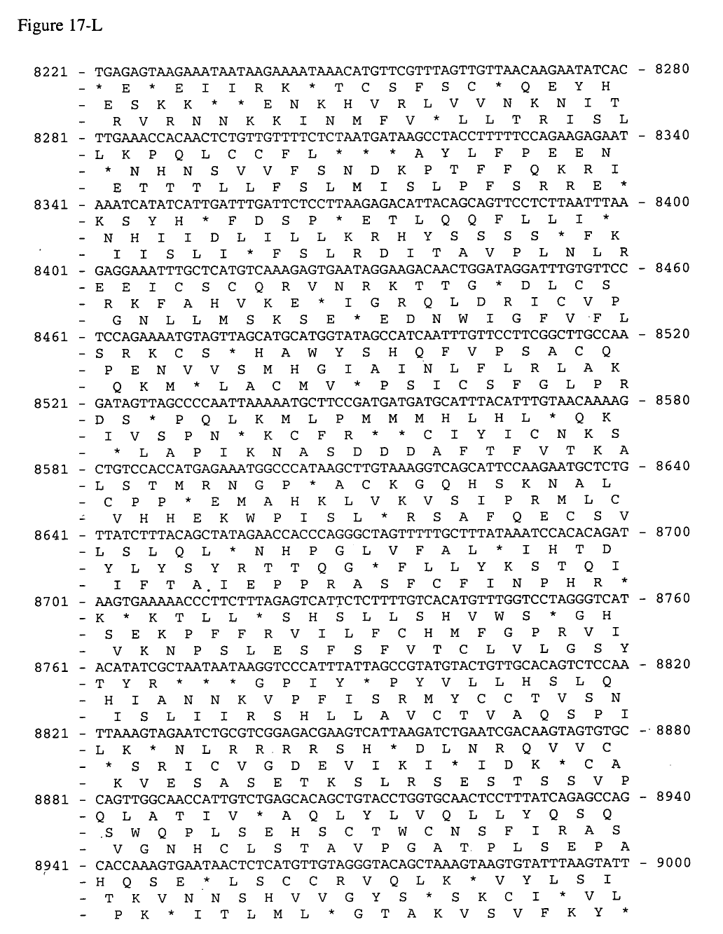

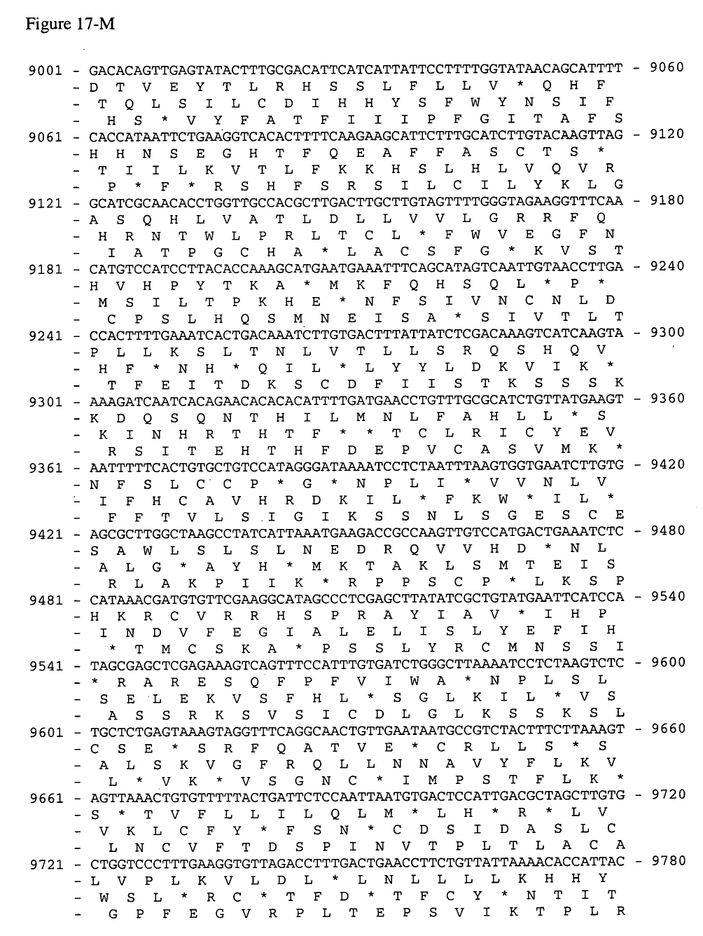

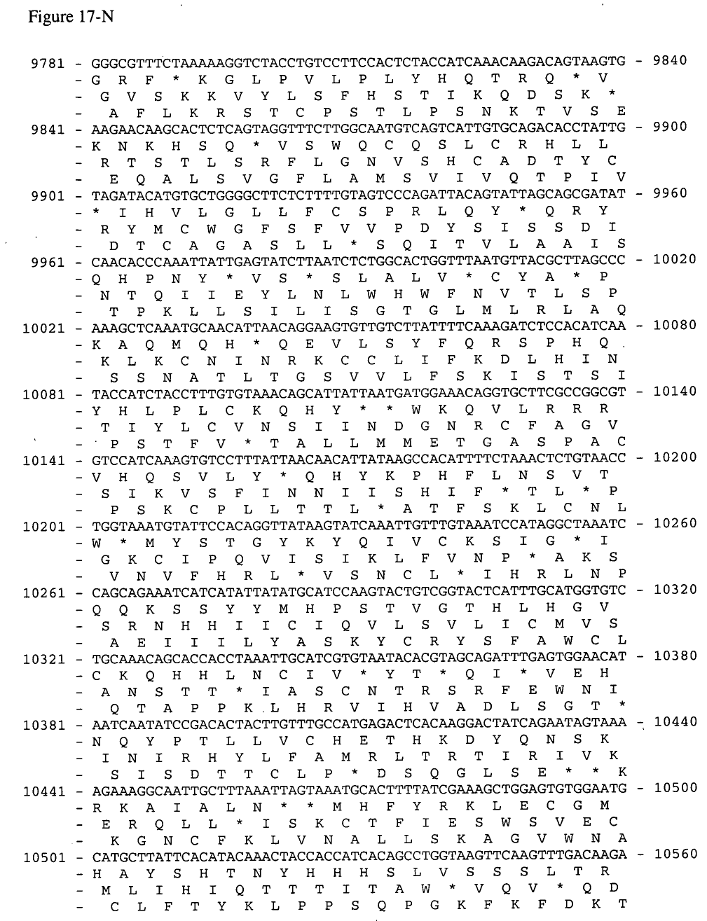

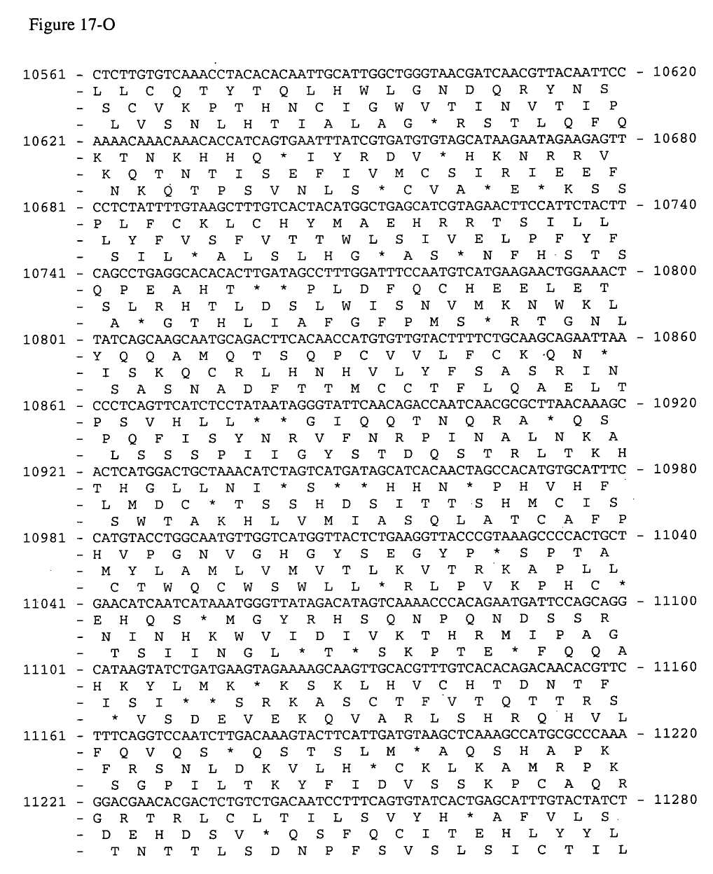

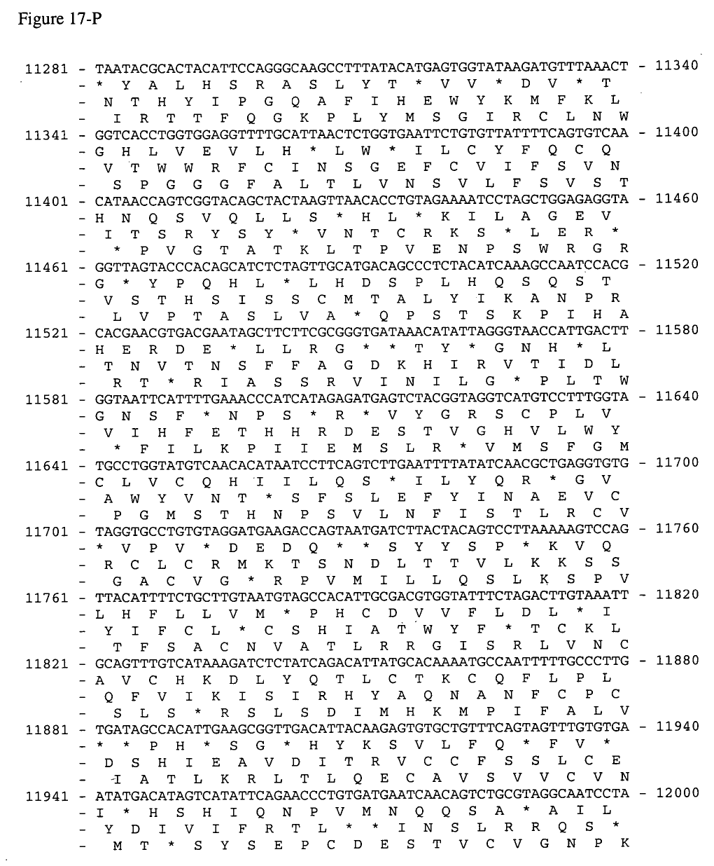

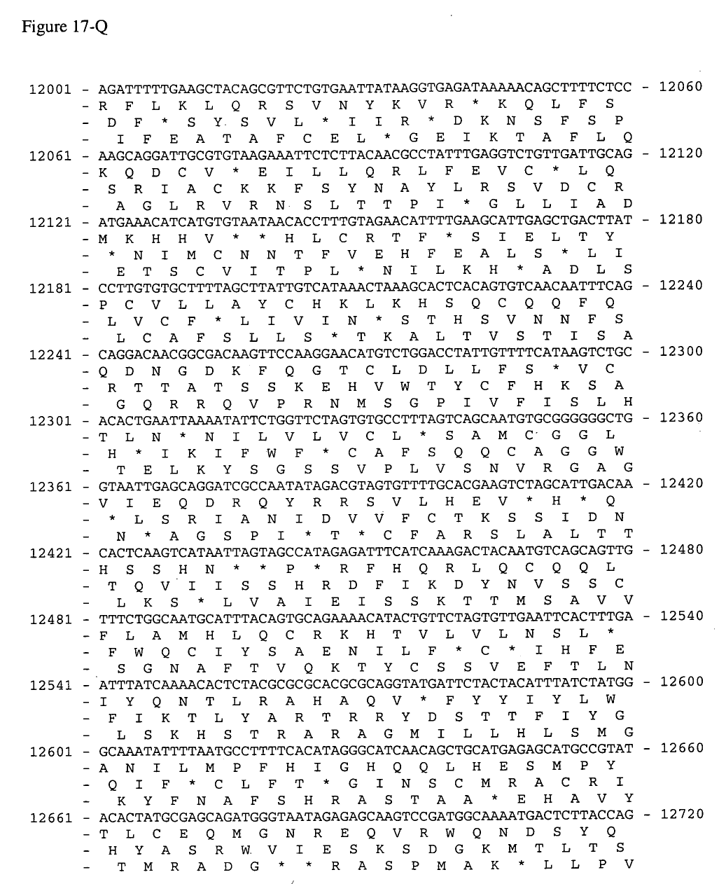

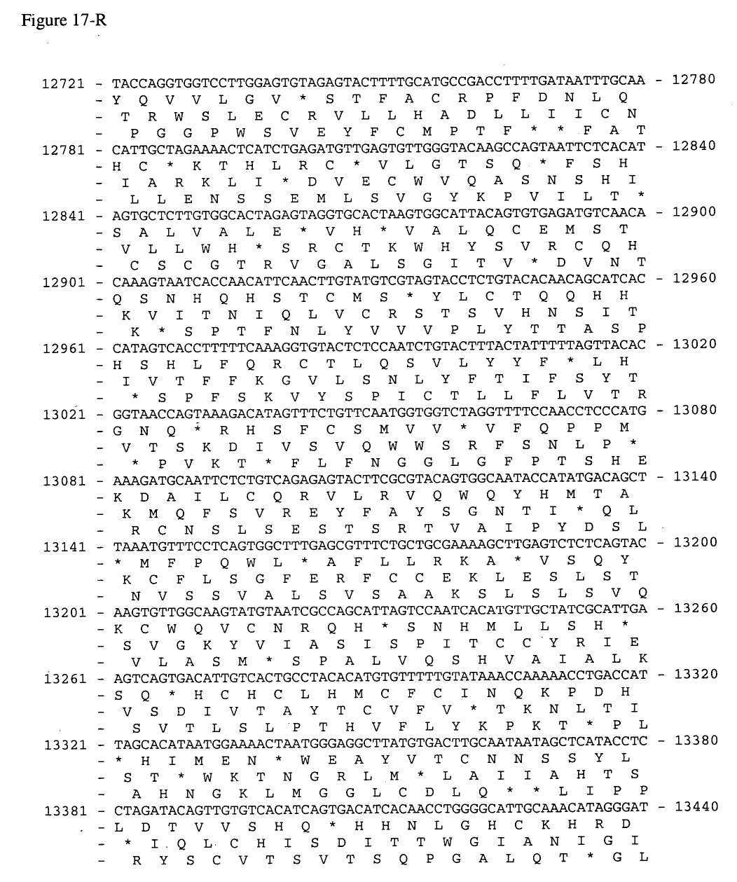

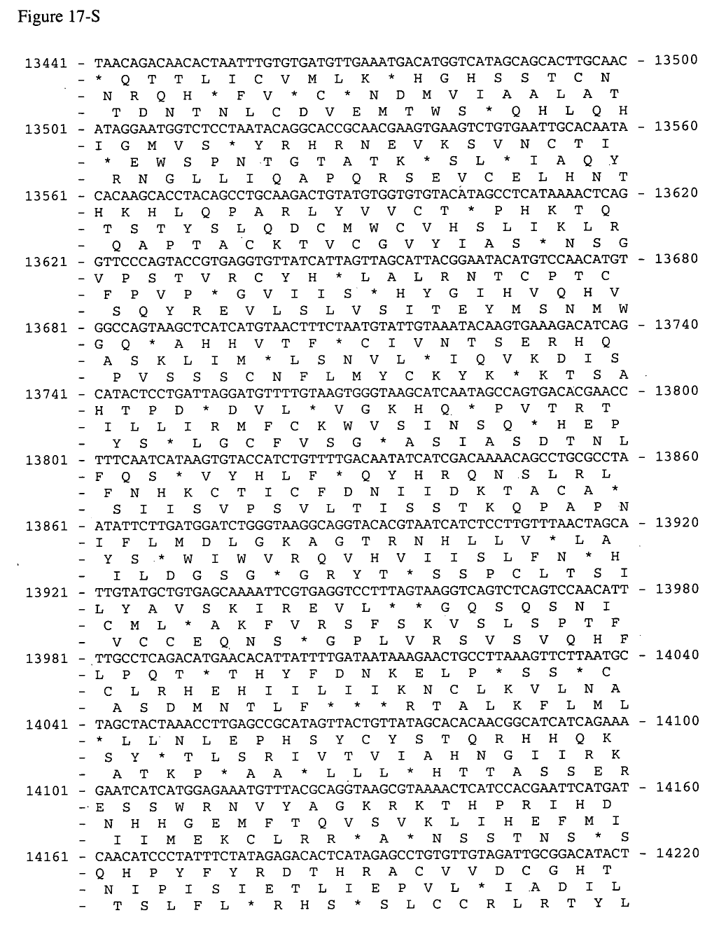

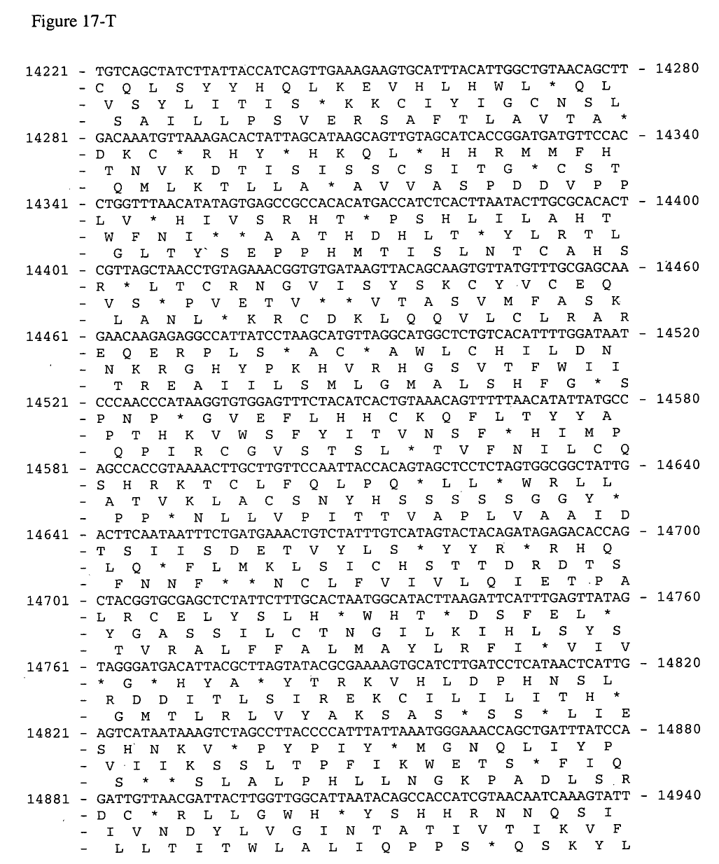

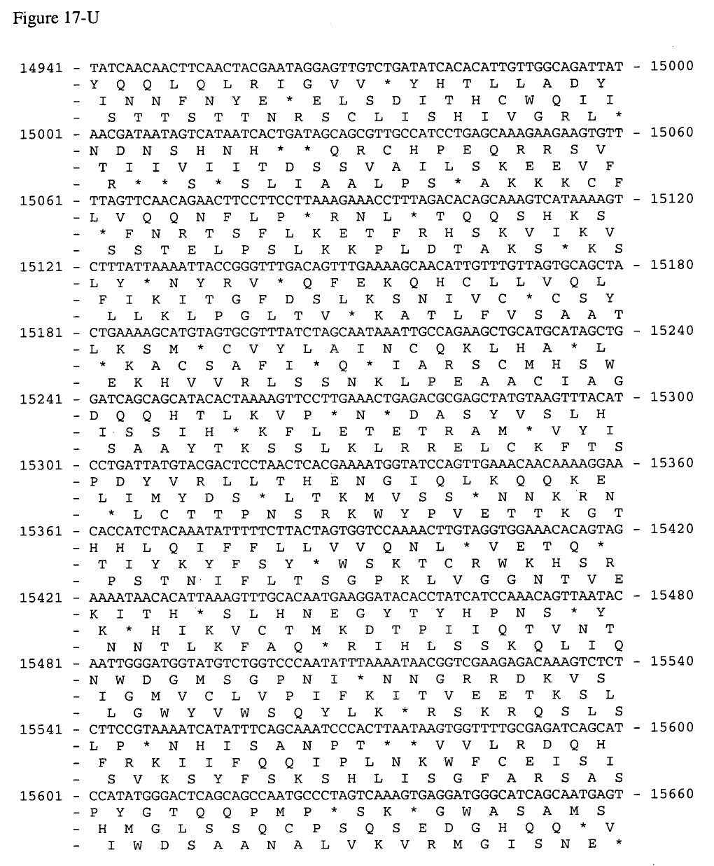

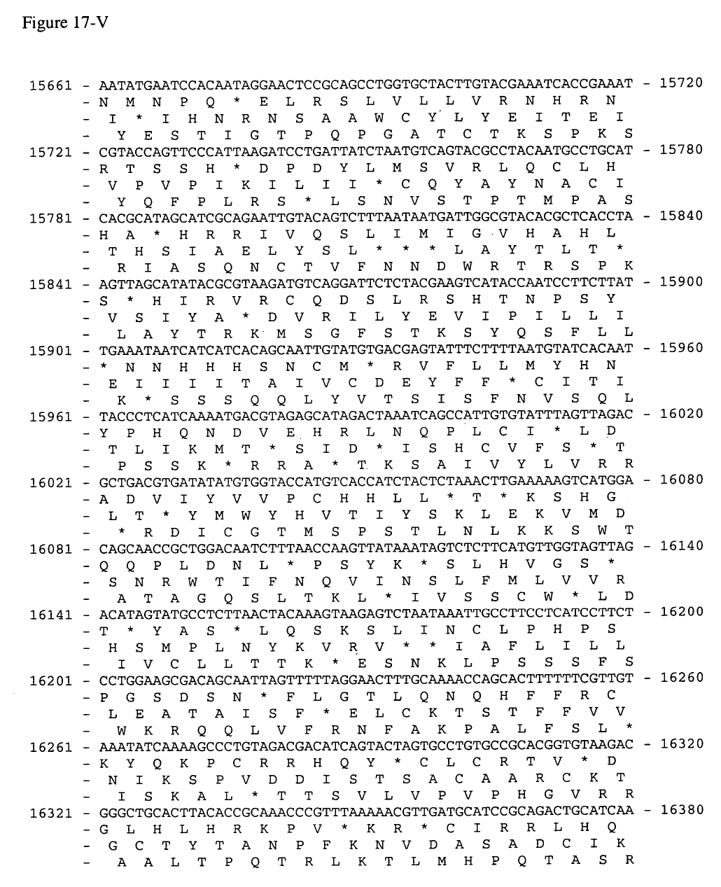

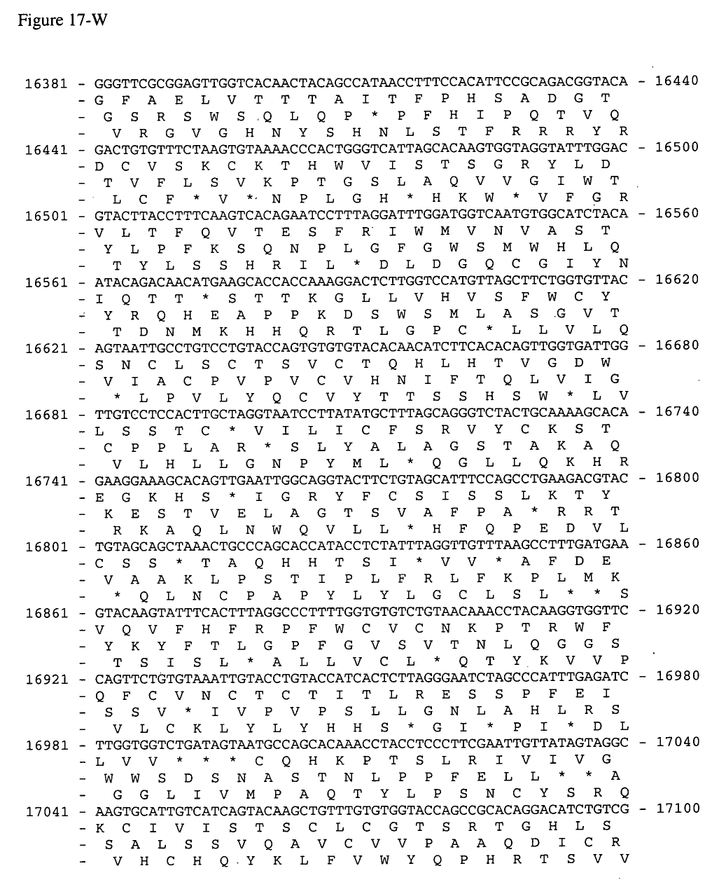

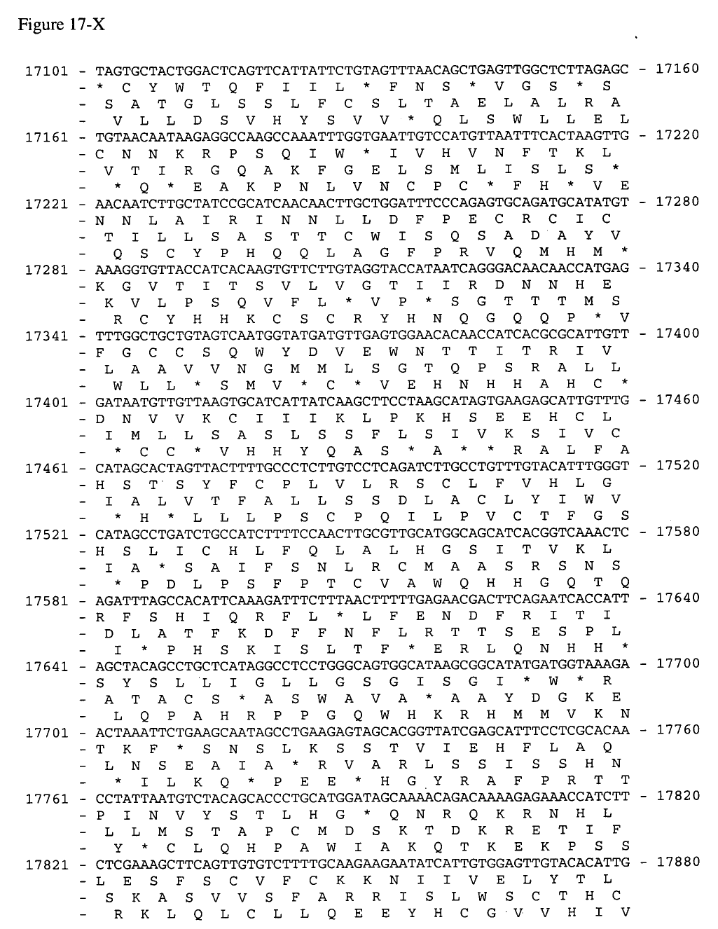

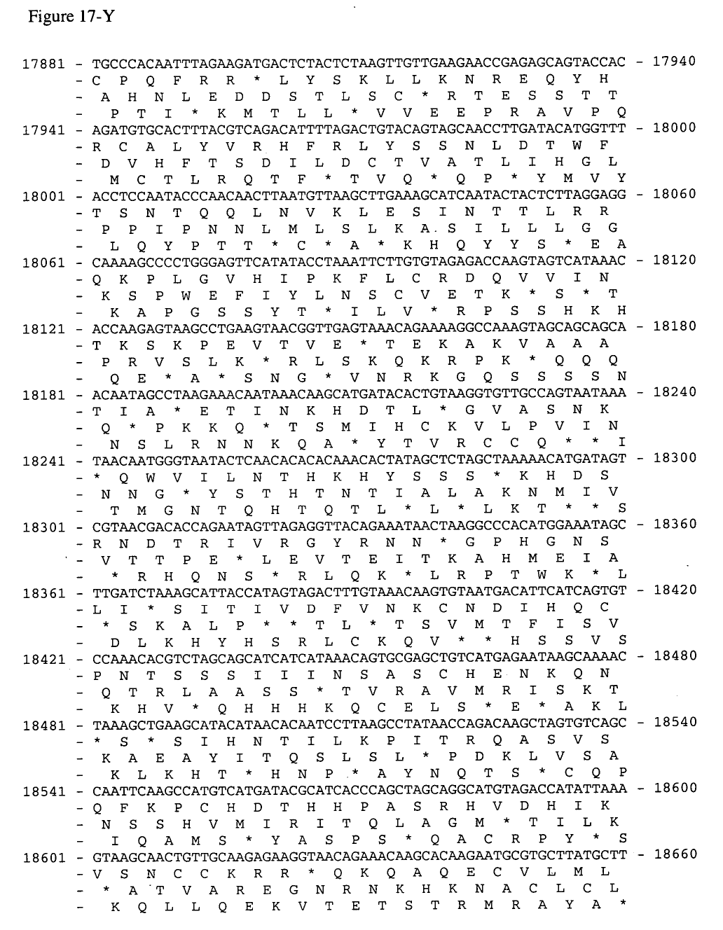

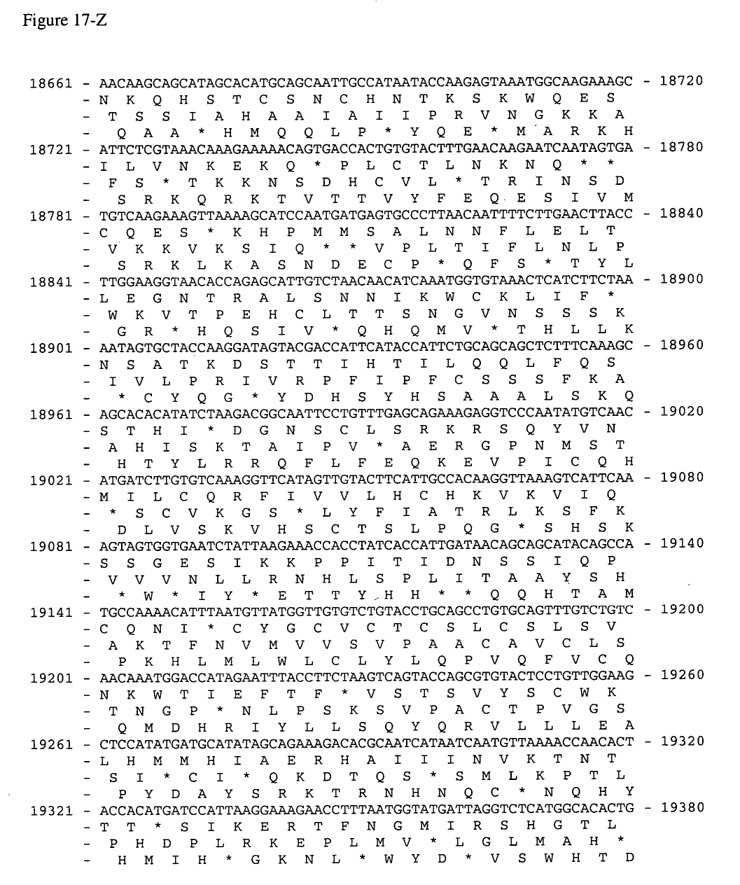

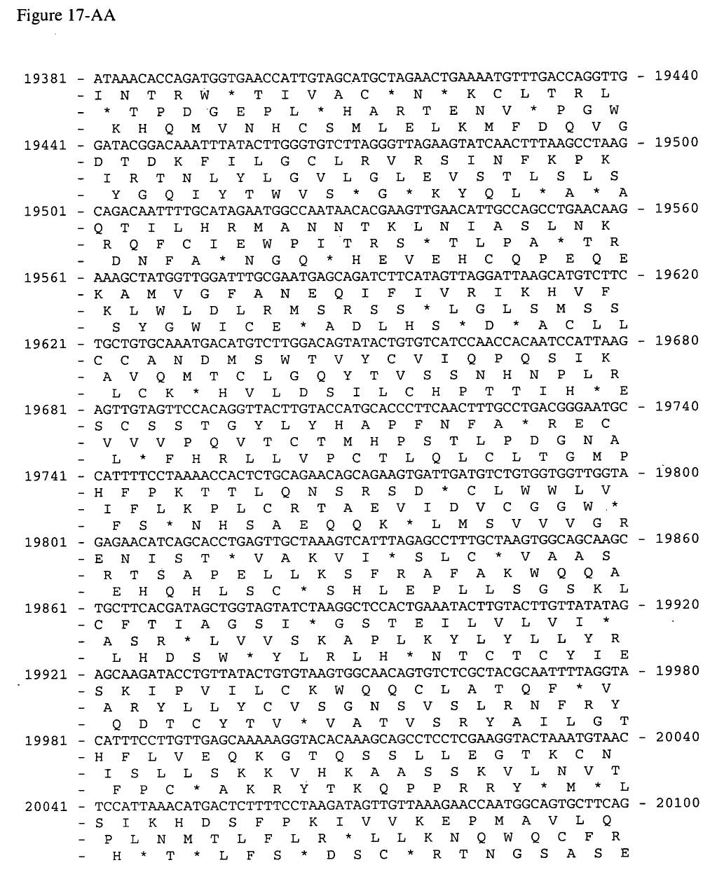

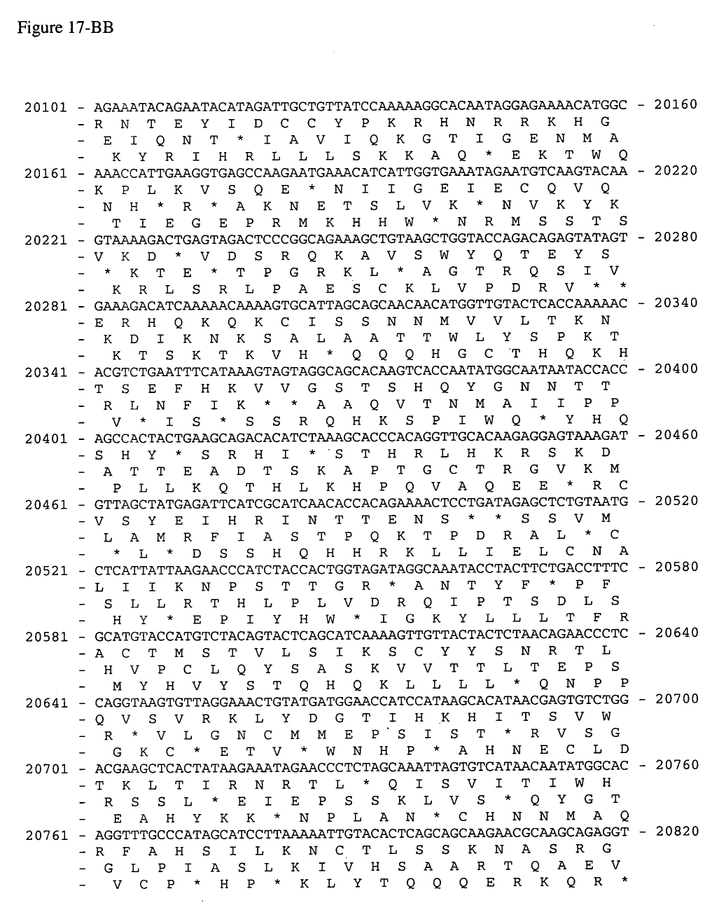

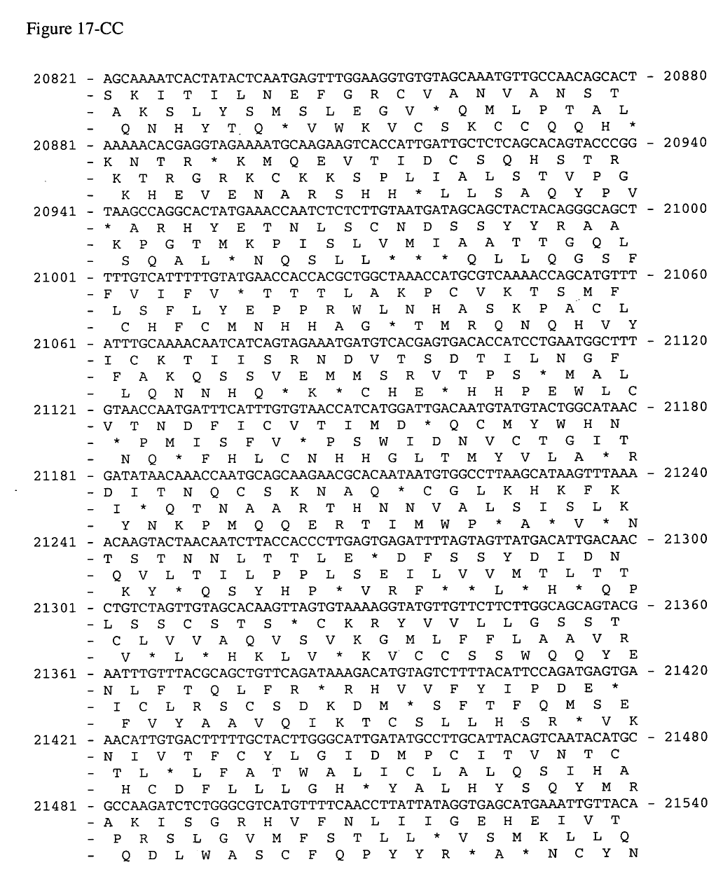

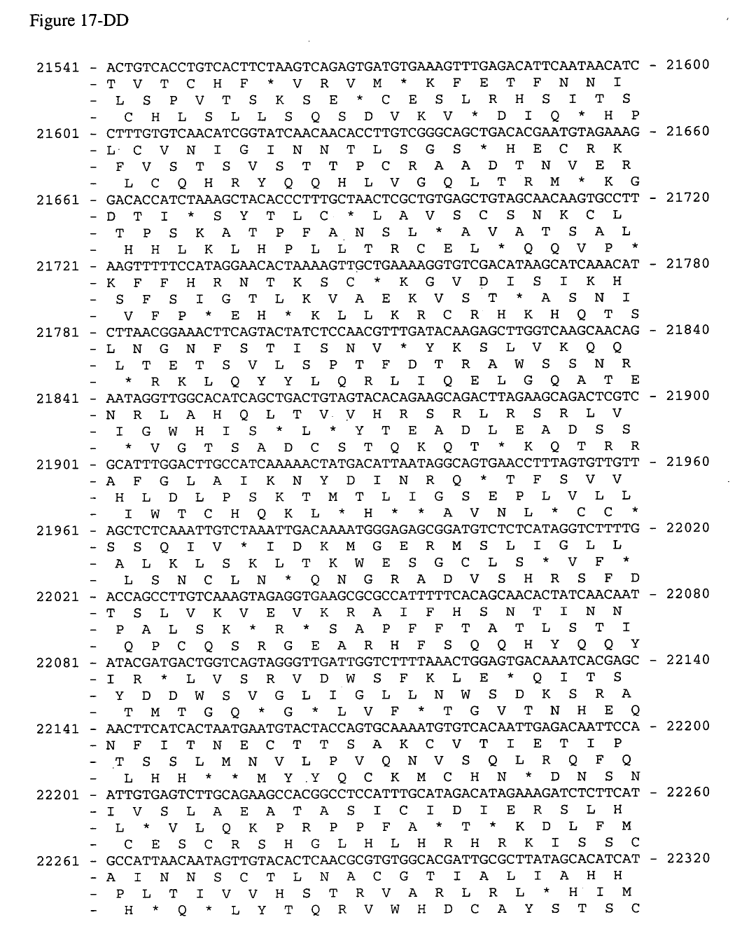

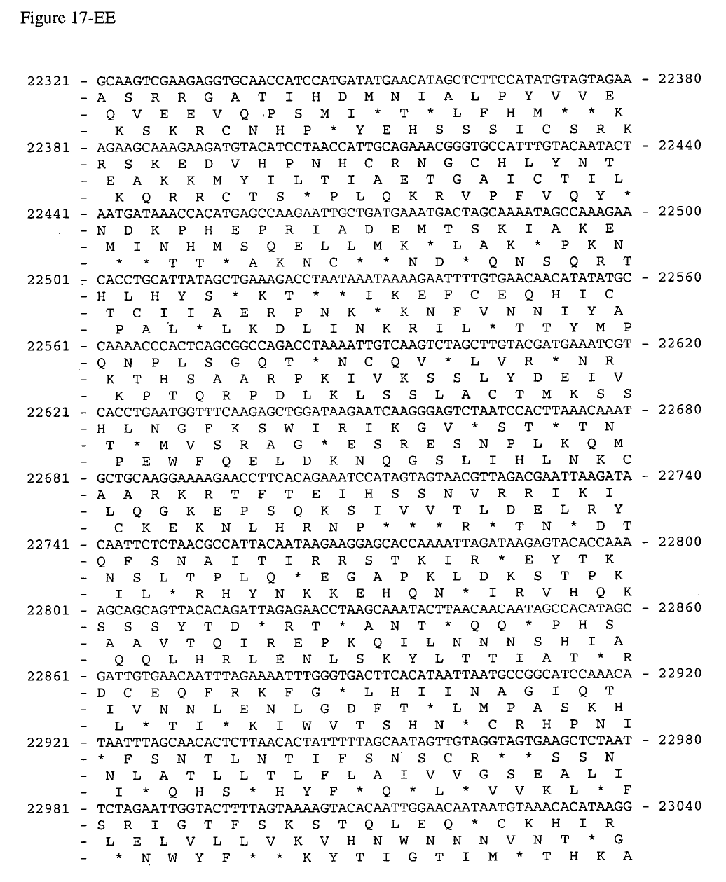

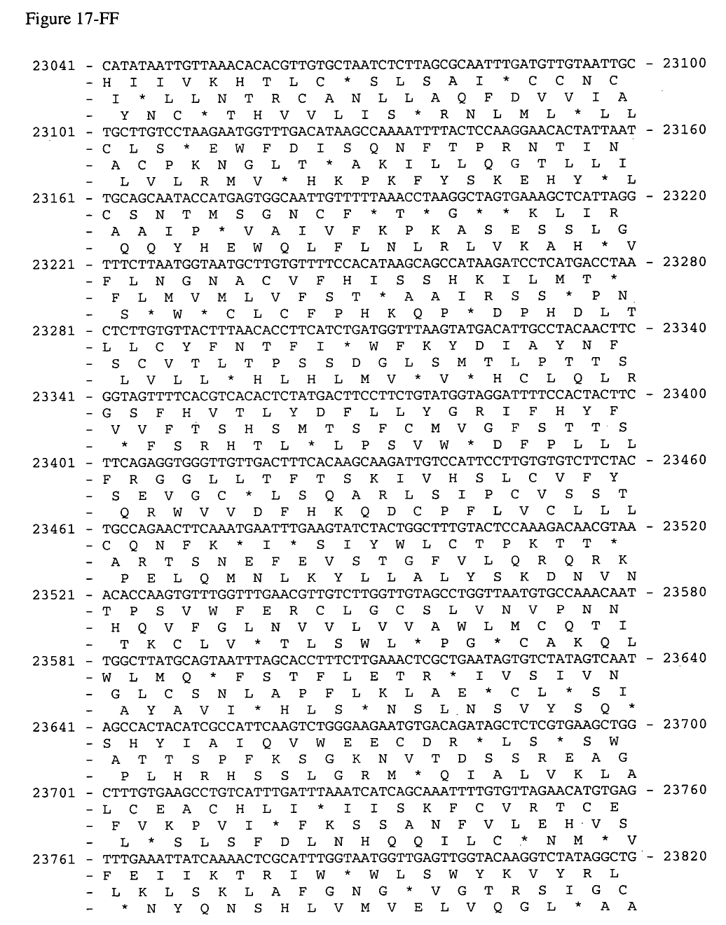

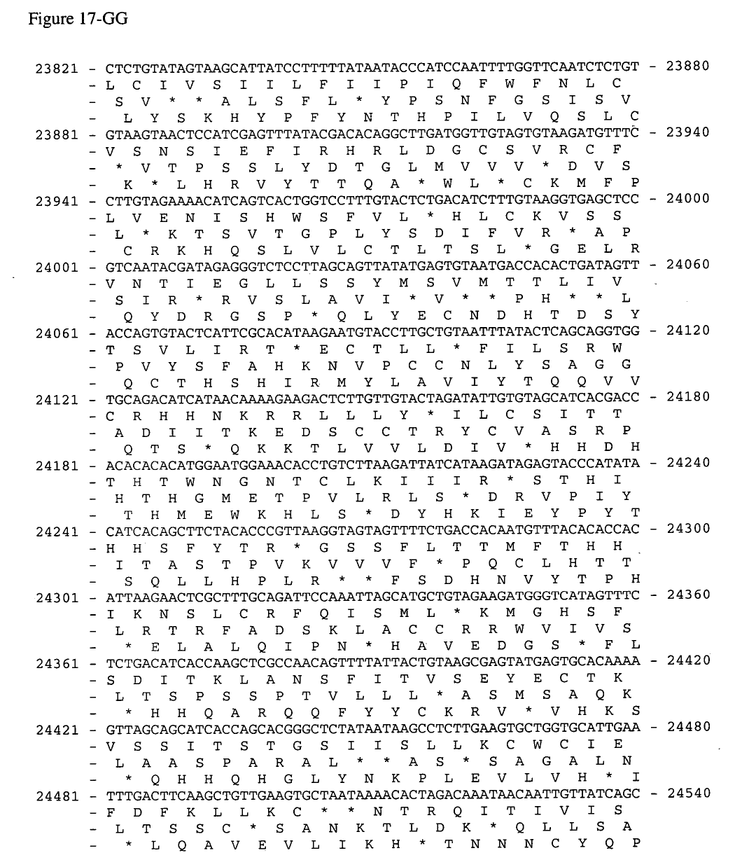

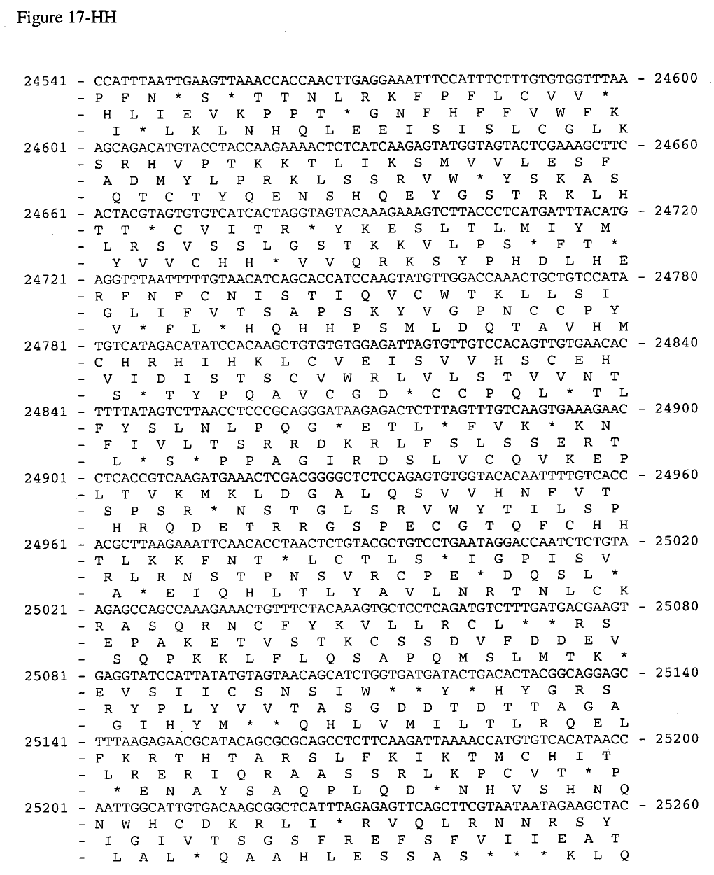

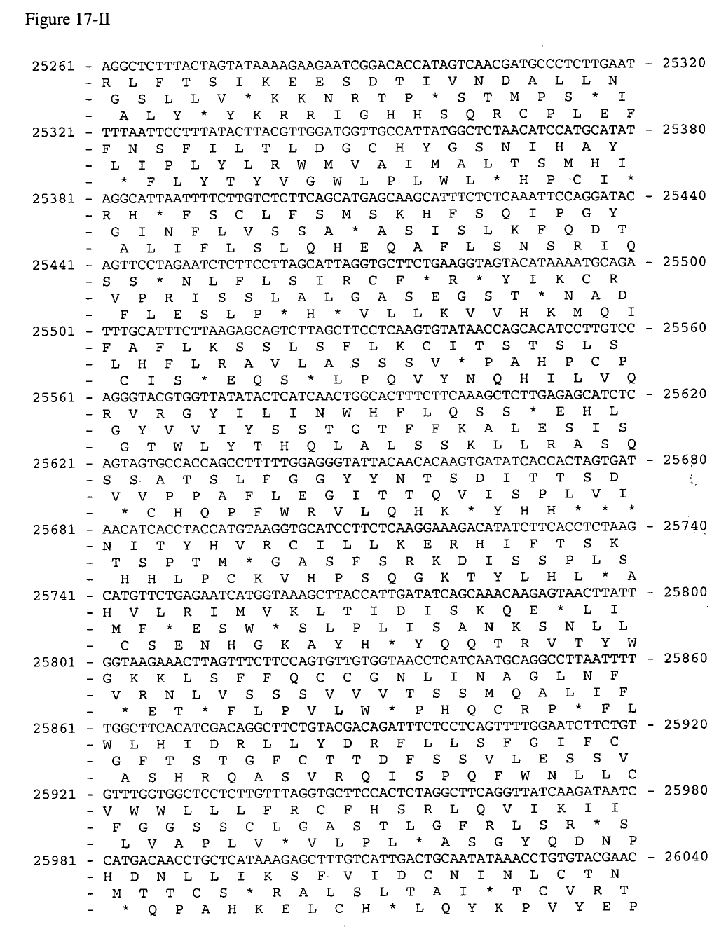

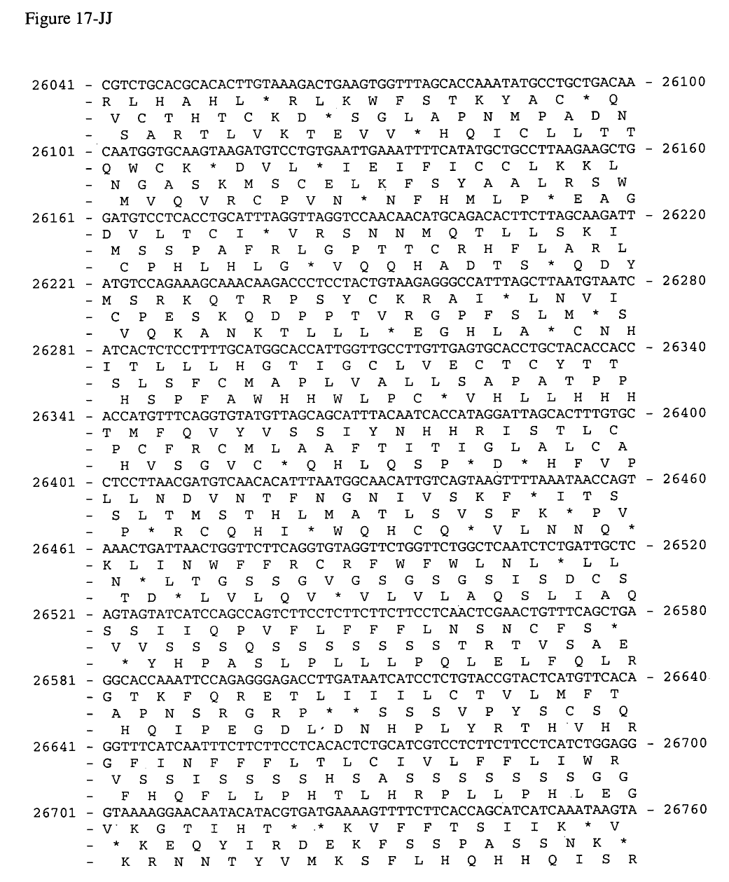

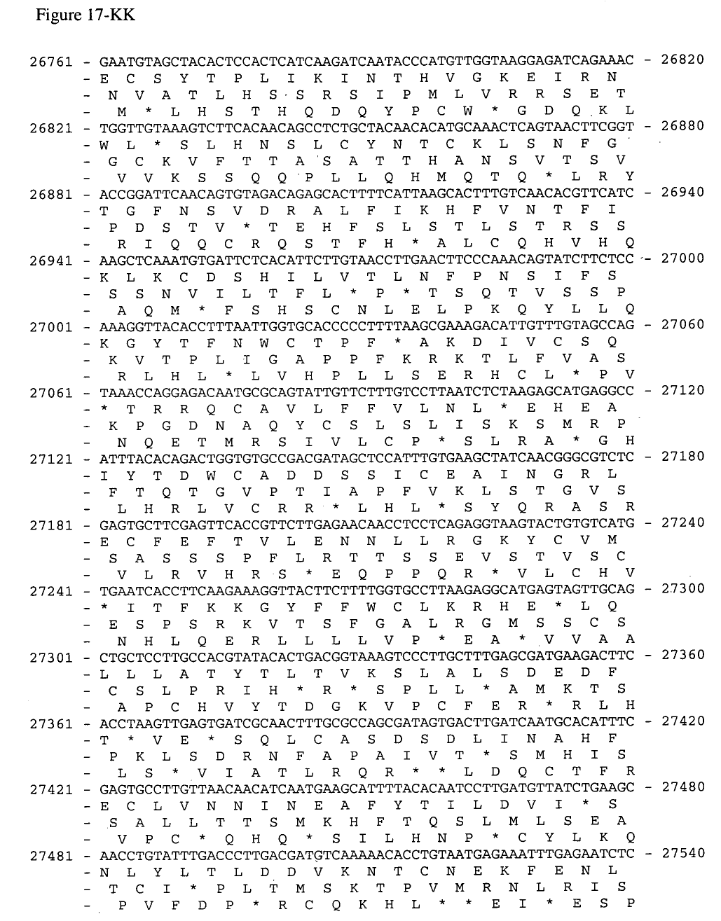

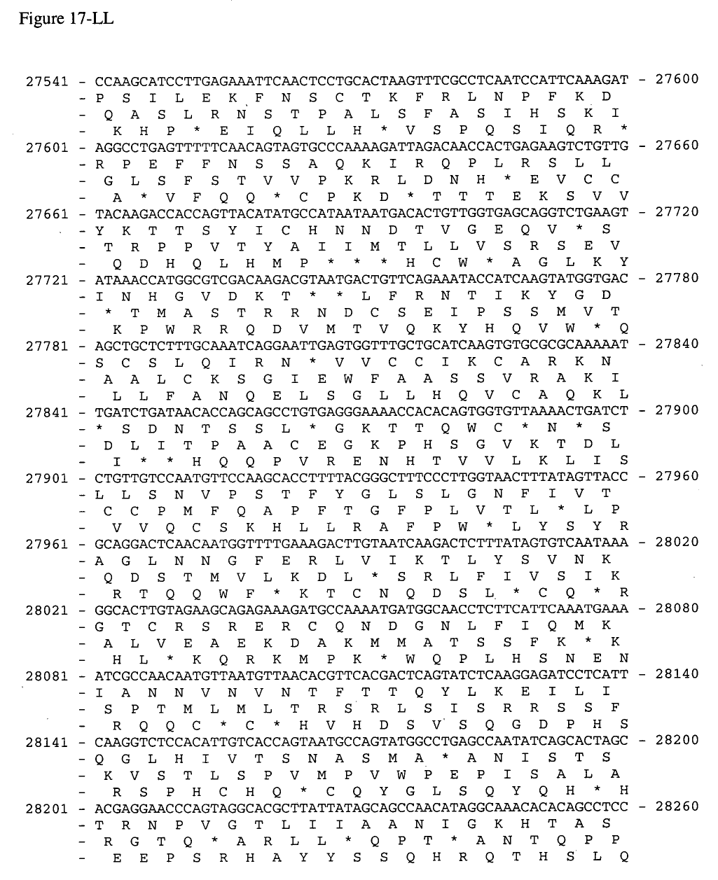

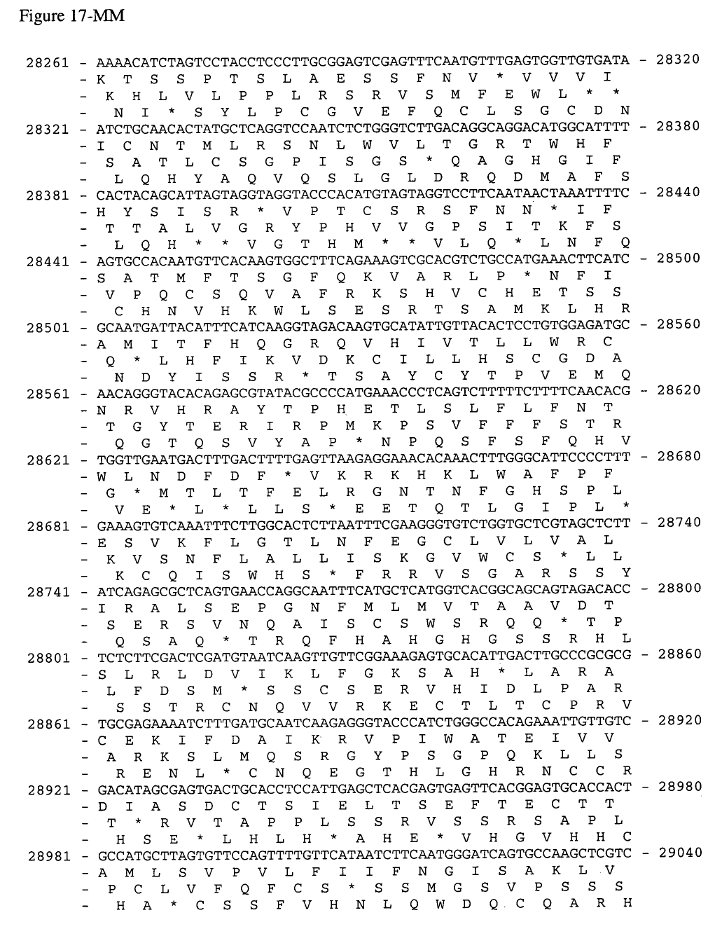

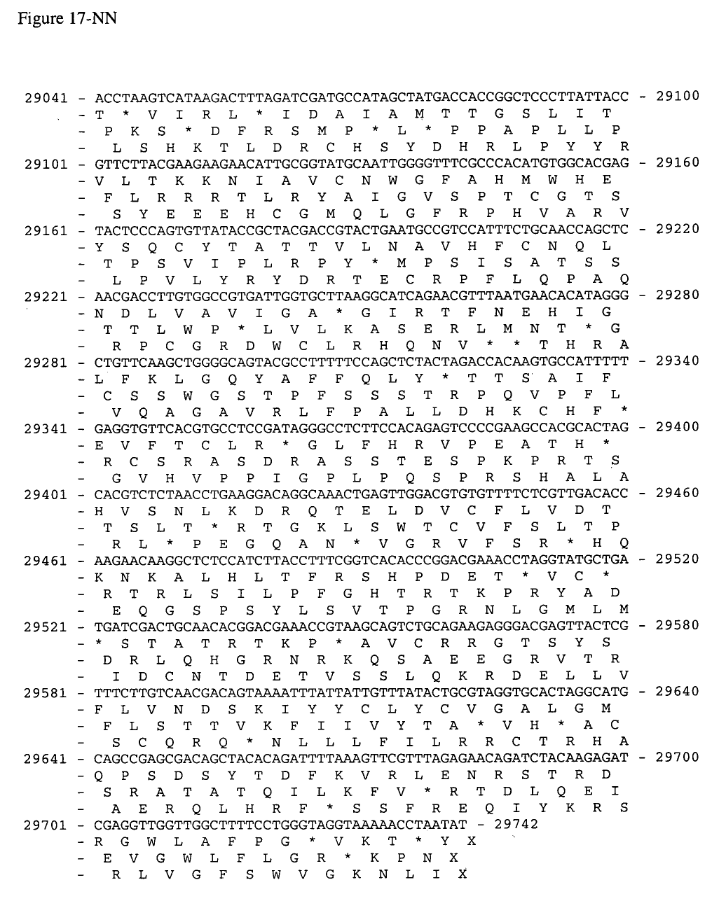

The present invention relates to plant vectors comprising one or more nucleotide sequences of the SARS-CoV virus, or fragments, derivatives, analogs or variants thereof. In a preferred embodiment, the plant vectors are plastid and nuclear transformation vectors. In preferred embodiments, the nucleotide sequence is the M-gene and/or S-gene of the SARS-CoV virus. The present invention also relates to the construction of plastid and nuclear transformation vectors comprising one or more SARS-CoV viral nucleotide sequences, or fragments, derivatives, analogs or artificial or natural variants thereof. In specific embodiments, the invention provides plant transformation vectors that comprise one or more nucleotide sequences that are at least 5, 10, 15, 20, 25, 30, 35, 40, 45, 100, 150, 200, 300, 350, 400, 450, 500, 550, 600, 650, 700, 750, 800, 850, 900, 950, 1,000, 1,050, 1,100, 1,150, 1,200, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, 26,000, 27,000, 28,000, 29,000, or more contiguous nucleotides of SEQ ID NO:7, or a complement thereof, or a nucleotide comprising a nucleotide sequence that hybridizes to the nucleotide sequences of the SARS-CoV virus under stringent conditions. In specific embodiments, the invention provides plant transformation vectors that comprise one or more nucleotide sequences that are at least 5, 10, 15, 20, 25, 30, 35, 40, 45, 100, 150, 200, 300, 350, 400, 450, 500, 550, 600, 650, or more contiguous nucleotides of SEQ ID NO:3, or a nucleotide comprising a nucleotide sequence that hybridizes to the nucleotide sequences of the SARS-CoV virus under stringent conditions. In specific embodiments, the invention provides plant transformation vectors that comprise one or more nucleotide sequences that are at least 5, 10, 15, 20, 25, 30, 35, 40, 45, 100, 150, 200, 300, 350, 400, 450, 500, 550, 600, 650, 700, 750, 800, 850, 900, 950, 1,000, 1,050, 1,100, 1,150, 1,200, 2,000, 3,000, 3,500, or more contiguous nucleotides of SEQ ID NO:5, or a nucleotide comprising a nucleotide sequence that hybridizes to the nucleotide sequences of the SARS-CoV virus under stringent conditions. In specific embodiments, the invention provides plant transformation vectors that comprise one or more nucleotide sequences that encode polypeptides that are at least 5, 10, 15, 20, 25, 30, 35, 40, 45, 100, 150, 200, or more contiguous amino acids of SEQ ID NO:4. In specific embodiments, the invention provides plant transformation vectors that comprise one or more nucleotide sequences that encode polypeptides that are at least 5, 10, 15, 20, 25, 30, 35, 40, 45, 100, 150, 200, 300, 350, 400, 450, 500, 550, 600, 650, 700, 750, 800, 850, 900, 950, 1,000, 1,050, 1,100, 1,150, 1,200, or more contiguous amino acids of SEQ ID NO:6. In one embodiment, the plant vector comprises nucleotide sequences that encode amino acids 14-1195 of S. In preferred embodiments, the plant vector comprises nucleotide sequences that encode at least 20, 30, 40, 50, 60, 80, 100, 200, 300, 400, 500, or more contiguous amino acids in length of amino acids 14-1195 of S. In specific embodiments, the polypeptides are those shown in FIGS. 16A-16NN and 17A-17NN, or fragments, derivatives or analogs thereof. In specific embodiments, the invention provides plant transformation vectors that comprise one or more nucleotide sequences that encode any of the genes, or portions thereof, or variants, fragments, analogs, or derivatives, from the SARS-CoV virus. These genes include, but are not limited to, envelop protein (E protein), integral membrane protein (M protein), spike protein (S protein), nucleocapsid protein (N protein), hemagglutinin esterase (HE protein), and RNA-dependent RNA polymerase. Particularly useful are also those proteinaceous substances that are encoded by recombinant nucleic acid fragments of the SARS-CoV genome. Preferred are those that are within the open reading frames (ORFs), in particular, for eliciting SARS-CoV specific antibody or T cell responses, whether in vivo (e.g., for protective or therapeutic purposes or for providing diagnostic antibodies) or in vitro (e.g., by phage display technology or another technique useful for generating synthetic antibodies).

The viral vectors can be engineered to provide antigenic molecules, including nucleotide sequences, or polypeptides of the SARS-CoV virus, including recombinant and chimeric forms of the virus, or subunits of the virus, or fragments, derivatives, analogs, or variants thereof, or molecules having similar activities as the polypeptides of the SARS-CoV virus. The present invention further provides methods of preparing recombinant or chimeric forms of SARS-CoV antigen.

In another specific embodiment, the present invention provides methods for treating, ameliorating, managing, or preventing SARS by administering a vaccine preparations or antibodies of SARS-CoV virus alone or in combination with antivirals (e.g., amantadine, rimantadine, zanamivir, abacavir, combivir, emtricitabine, tenofovir, trizivir, enfuvirtide, gancyclovir, acyclovir, ribavirin, penciclovir, oseltamivir, foscamet zidovudine (AZT), didanosine (ddI), lamivudine (3TC), zalcitabine (ddC), stavudine (d4T), nevirapine, delavirdine, efavirenz, fosamprenavir, amprenavir, atazanavir, kaletra, indinavir, ritonavir, vidarabine, nelfinavir, saquinavir, relenza, tamiflu, pleconaril, interferons, etc.), steroids and corticosteroids such as prednisone, cortisone, fluticasone and glucocorticoid, antibiotics, analgesics, bronchodialaters, or other treatments for respiratory and/or viral infections. In another specific embodiment, the methods of the present invention provides the use of herbs, herbal extracts, Chinese medicine and other remedies in combination with the modified plants of the present invention for the prevention and treatment of SARS.

Furthermore, the present invention provides pharmaceutical compositions comprising anti-viral agents of the present invention and a pharmaceutically acceptable carrier, vehicle or excipient. In addition, the present invention provides pharmaceutical compositions comprising liposomally encapsulated plant extracts, preferably purified, or anti-viral agents of the present invention. The present invention also provides kits comprising the pharmaceutical compositions of the present invention.

The present invention encompasses the use of a nucleotide sequence that encodes a chimeric polypeptide of the SARS-CoV virus. In a specific embodiment, the chimeric polypeptide comprises amino acid sequences from two or more different strains of the SARS-CoV virus. In accordance with the present invention, the modified plants of the present invention further comprise a nucleotide sequence that is non-native to the viral genome. The nucleotide sequence that may be useful for the present invention includes a portion of the SARS-CoV viral sequence which further comprises a heterologous nucleotide sequence. A heterologous nucleotide sequence may be from a virus, bacteria, animal, or plant. In a preferred embodiment, the heterologous nucleotide sequence encodes a proteinase inhibitor. The heterologous nucleotide sequence renders the expressed SARS-CoV polypeptide more stable and reduces programmed cell death and increases shelf life of the modified plant of the present invention. In a specific embodiment, the heterologous nucleotide sequence (e.g., SaPIN2a or SaPIN2b) renders the expressed SARS-CoV polypeptides more stable so that the modified plants that express the SARS-CoV polypeptide may be processed before ingestion, injection, or other methods of administration. SaPIN2a and SaPIN2b, which are proteinase inhibitor II genes isolated from Solanum americanum, a weed belonging to the Solanaceae family, encode serine proteinase inhibitor II proteins which confer insect resistance in transgenic plants (See U.S. Provisional Application No. 60/429,992 filed Nov. 29, 2002; and U.S. application Ser. No. 10/725,829, Attorney Docket No. 9661-043-999, filed Dec. 1, 2003, each of which is incorporated by reference herein in its entirety). SaPIN2a and SaPIN2b are highly expressed in the phloem and have possible involvement in regulating proteolysis in the sieve elements (See, Xu et al., 2001, Plant Mol. Biol. Vol. 47, pages 727-738; and Xu et al., 2003, Planta Vol. 218, pages 623-629, each of which is incorporated by reference herein in its entirety).

In certain embodiments, the present invention relates to vectors and nucleic acid molecules comprising the nucleotide sequence that encodes a chimeric polypeptide of the SARS-CoV virus. In a specific embodiment, a vector comprises a heterologous nucleotide sequence of SARS-CoV. In another embodiment, such heterologous nucleotide sequences have been added, inserted or substituted for native or non-native sequences. In accordance with the present invention, the nucleotide sequence of the SARS-CoV virus may be derived from different strains or variants of SARS-CoV virus.

A plant vector comprising chimeric SARS-CoV viral sequences may be of particular use for the generation of recombinant vaccines protecting against two or more viruses (Tao et al., J. Virol. 72: 2955-2961; Durbin et al., 2000, J. Virol. 74: 6821-6831; Skiadopoulos et al., 1998, J. Virol. 72: 1762-1768 (1998); Teng et al., 2000, J. Virol. 74: 9317-9321). For example, it can be envisaged that a plant vector comprising the SARS-CoV viral nucleotide sequences may express one or more peptides from variants of the SARS-CoV virus, and will protect a subject against infections by both the native SARS-CoV and the variant.

In accordance with the present invention, the plant vectors can be engineered to provide antigenic sequences which confer protection against infection by the SARS-CoV and natural variants thereof when ingested by a subject. The plant vectors may be engineered to provide one or more antigenic sequences of SARS-CoV virus. In accordance with the present invention, the antigenic sequences may be derived from the same virus, from different strains or variants of the same type of virus, or from different viruses.

The invention provides a host cell comprising a vector according to the invention. Plant plastid or nuclear transformation vector containing the nucleotide sequences of the SARS-CoV virus such as containing the full-length, portions or fragments of the SARS-CoV genome for the expression of SARS-CoV viral nucleic acids. These plant vectors may contain other sequences for the generation of chimeric SARS-CoV viral polypeptides which may contain mutations, deletions or insertions of the SARS-CoV polypeptides. Nucleotide sequences which encode SARS-CoV virus, fragments, derivatives, analogs, or variants of polypeptides of SARS-CoV virus include, but are not limited to, those deposited with GenBank® having accession nos. NC_{—}004718, AY304495, AY304494, AY304493, AY304492, AY304491, AY304490, AY304489, AY304488, AY304487, AY304486, AY360146, AY278491, AY310120, AY278489, AY362699, AY362698, AY283798, AY283797, AY283796, AY283795, AY283794, AY268070, AY278741, AY340092, AY351680, AP006561, AP006560, AP006559, AP006558, AP006557, AY278554, AY348314, AY338175, AY338174, AY323977, AY322199, AY322198, AY322197, AH013000, AY322208, AY322207, AY322206, AY322205, AH012999, AY321118, AY323976, AY323975, AY323974, AY286320, AY290752, AY291315, AY307165, AY279354, AY278490, AY278487, AY297028, AY286402, AY274119, AY291451, AY271716, AY282752, AY278488, AY268049, AY269391, all of which are incorporated herein by reference in their entireties.

In one specific embodiment, plant cells may be transiently or stably expressing one or more nucleotide sequences of the SARS-CoV virus. Plants cells are modified by transfection (proteins or nucleic acid vectors), infection (viral vectors) or transduction (viral vectors). The modified plant may be used to modulate a subject's immune system by stimulating a humoral immune response, a cellular immune response or by stimulating tolerance to an antigen. As used herein, a subject means: humans, primates, rabbits, goats, cows, pigs, sheep, horses, civets, rodents, raccoons, raccoon dogs, dogs, ferrets, ferret Badger, cat, cats, avian species, or other non-human animals.

The nucleotide molecules encoding the SARS-CoV viral antigen proteins may be cloned by amplification. The term “amplified” refers to the process of making multiple copies of the nucleic acid from a single polynucleotide molecule. The amplification of polynucleotides can be carried out in vitro by biochemical processes known to those of skill in the art. The amplification agent may be any compound or system that will function to accomplish the synthesis of primer extension products, including enzymes. Suitable enzymes for this purpose include, for example, E. coli DNA polymerase I, Taq polymerase, Klenow fragment of E. coli DNA polymerase I, T4 DNA polymerase, other available DNA polymerases, polymerase mutants, reverse transcriptase, ligase, and other enzymes, including heat-stable enzymes (i.e., those enzymes that perform primer extension after being subjected to temperatures sufficiently elevated to cause denaturation). Suitable enzymes will facilitate combination of the nucleotides in the proper manner to form the primer extension products that are complementary to each mutant nucleotide strand. Generally, the synthesis will be initiated at the 3′-end of each primer and proceed in the 5′-direction along the template strand, until synthesis terminates, producing molecules of different lengths. There may be amplification agents, however, that initiate synthesis at the 5′-end and proceed in the other direction, using the same process as described above. In any event, the method of the invention is not to be limited to the embodiments of amplification described herein.

One method of in vitro amplification, which can be used according to this invention, is the polymerase chain reaction (PCR) described in U.S. Pat. Nos. 4,683,202 and 4,683,195. The term “polymerase chain reaction” refers to a method for amplifying a DNA base sequence using a heat-stable DNA polymerase and two oligonucleotide primers, one complementary to the (+)-strand at one end of the sequence to be amplified and the other complementary to the (−)-strand at the other end. Because the newly synthesized DNA strands can subsequently serve as additional templates for the same primer sequences, successive rounds of primer annealing, strand elongation, and dissociation produce rapid and highly specific amplification of the desired sequence. The polymerase chain reaction is used to detect the presence of polynucleotides encoding cytokines in the sample. Many polymerase chain methods are known to those of skill in the art and may be used in the method of the invention. For example, DNA can be subjected to 30 to 35 cycles of amplification in a thermocycler as follows: 95° C. for 30 sec, 52-60° C. for 1 min, and 72° C. for 1 min, with a final extension step of 72° C. for 5 min. For another example, DNA can be subjected to 35 polymerase chain reaction cycles in a thermocycler at a denaturing temperature of 95° C. for 30 sec, followed by varying annealing temperatures ranging from 54-58° C. for 1 min, an extension step at 70° C. for 1 min and a final extension step at 70° C.

The primers for use in amplifying the nucleotide sequences that encodes the proteinaceous molecules of the SARS-CoV virus, or fragments, derivatives, analogs, or variants thereof, or molecules having the similar activities as the polypeptides of the SARS-CoV virus may be prepared using any suitable method, such as conventional phosphotriester and phosphodiester methods or automated embodiments thereof so long as the primers are capable of hybridizing to the polynucleotides of interest. One method for synthesizing oligonucleotides on a modified solid support is described in U.S. Pat. No. 4,458,066. The exact length of primer will depend on many factors, including temperature, buffer, and nucleotide composition. The primer must prime the synthesis of extension products in the presence of the inducing agent for amplification.

Primers used according to the method of the invention are complementary to each strand of nucleotide sequence to be amplified. The term “complementary” means that the primers must hybridize with their respective strands under conditions, which allow the agent for polymerization to function. In other words, the primers that are complementary to the flanking sequences hybridize with the flanking sequences and permit amplification of the nucleotide sequence. Preferably, the 3′ terminus of the primer that is extended has perfectly base paired complementarity with the complementary flanking strand. Primers and probes for the nucleotide sequence encoding the antigens, polypeptides of the SARS-CoV or fragments, derivatives, analogs, or variants thereof, or molecules having the similar activities as the polypeptides of the SARS-CoV virus can be developed using known methods combined with the present disclosure.

Those of ordinary skill in the art will know of various amplification methodologies that can also be utilized to increase the copy number of target nucleic acid. The polynucleotides that may be used for the present invention can be further evaluated, detected, cloned, sequenced, and the like, either in solution or after binding to a solid support, by any method usually applied to the detection of a specific nucleic acid sequence such as another polymerase chain reaction, oligomer restriction (Saiki et al., 1985, Bio/Technology Vol. 3, pages 1008-1012), allele-specific oligonucleotide (ASO) probe analysis (Conner et al., 1983, Proc. Natl. Acad. Sci. USA Vol. 80, page 278), oligonucleotide ligation assays (OLAs) (Landegren et al., 1988, Science Vol. 241, page 1077), RNase Protection Assay (RPA) and the like. Molecular techniques for DNA analysis have been reviewed (Landegren et al, 1988, Science Vol. 242, pages 229-237). Following DNA amplification, the reaction product may be detected by Southern blot analysis, without using radioactive probes. In such a process, for example, a small sample of DNA containing the polynucleotides obtained from the tissue or subject are amplified, and analyzed via a Southern blotting technique. The use of non-radioactive probes or labels is facilitated by the high level of the amplified signal.

The size of the primers used to amplify nucleotide sequences that encode antigens, polypeptides of the SARS-CoV or fragments, derivatives, analogs, or variants thereof, or molecules having the similar activities as the polypeptides of the SARS-CoV virus is at least 10, 15, 20, 25, 30 nucleotide in length. In particular, primers that amplify the M-gene or S-gene is most preferred. Preferably, the G:C ratio should be above 30%, 35%, 40%, 45%, 50%, 55%, 60% so as to prevent hair-pin structure on the primer. Furthermore, the amplicon should be sufficiently long enough to be detected by standard molecular biology methodologies. Preferably, the amplicon is at least 40, 60, 100, 200, 300, 400, 500, 600, 800, 1000, or more base pair in length.

The polynucleotides that may be used in the present invention include polynucleotides having the DNA sequences presented herein, and additionally include any nucleotide sequences encoding an epitope comprising contiguous and functional antigens, polypeptides of the SARS-CoV or fragments, derivatives, analogs, or variants thereof, or molecules having the similar activities as the polypeptides of the SARS-CoV virus encoding open reading frame (ORF) that hybridizes to a complement of the DNA sequences presented herein under highly stringent conditions. By way of example and not limitation, high stringency hybridization conditions can be defined as follows: The filter-bound DNA were hybridized in a solution containing 50% deionized formamide, 6×SSC, 5× Denhardt's, 1% SDS, 100 μg/ml denatured salmon sperm DNA at 42° C. overnight (about 4-16 hours), and washing in 0.1× SSC, 0.1% SDS at 65° C. (Ausubel F. M. et al., eds., 1989, Current Protocols in Molecular Biology, Vol. 1, Green Publishing Associates, Inc., and John Wiley & sons, Inc., New York) and encodes a functionally equivalent gene product. For oligonucleotide probes, by way of example and not limitation, highly stringent conditions may refer, e.g., to washing in 6×SSC/0.05% sodium pyrophosphate at 37° C. (for 14-base oligos), 48° C. (for 17-base oligos), 55° C. (for 20-base oligos), and 60° C. (for 23-base oligos).

Additionally contemplated polynucleotides that may be used in the present invention include any nucleotide sequences that hybridize under moderately stringent conditions to the complement of the DNA sequences that encode antigens, polypeptides of the SARS-CoV or fragments, derivatives, analogs, or variants thereof, or molecules having the similar activities as the polypeptides of the SARS-CoV virus. By way of example but not limitation, such moderately stringent conditions may include, e.g., washing in 0.2×SSC/0.1% SDS at 42° C. (Ausubel et al., 1989, supra).

Additionally contemplated polynucleotides that may be used in the present invention include any nucleotide sequences that hybridize under low stringency conditions to the complement of the DNA sequences that encode antigens, polypeptides of the SARS-CoV or fragments, derivatives, analogs, or variants thereof, or molecules having the similar activities as the polypeptides of the SARS-CoV virus. By way of example and not limitation, procedures using such conditions of low stringency are described in Shilo and Weinberg, 1981, Proc. Natl. Acad. Sci. USA Vol. 78, pages 6789-6792. A variant may comprise one or more changes in the amino acid sequence of the protein, e.g., by way of addition, substitution, or deletion of one or more amino acids, compared with the wild type protein. Any change should not abolish the ability of the protein to perform its function, though it may increase or decrease this ability depending on the nature of the changes. Preferably, the amino acid changes are conservative.

In various embodiments, antigen, polypeptide of the SARS-CoV virus or fragment, variant, analog, or derivative may be expressed as a fusion, or chimeric protein product (comprising the enzyme, fragment, analog, or derivative joined via a peptide bond to a heterologous protein sequence (of a different protein)). Such a chimeric gene product can be made by ligating the appropriate nucleic acid sequences encoding the desired amino acid sequences to each other by methods known in the art, in the proper coding frame, and expressing the chimeric product by methods commonly known in the art. Alternatively, such a chimeric product may be made by protein synthetic techniques, e.g., by use of a peptide synthesizer. Preferably, the fragment, analog, and derivative of the enzyme in the fusion protein retains the ability to perform the enzyme's function.

For the construction of plant nuclear transformation vectors, the expression of the SARS-CoV nucleotide sequences, genes, fragments, derivatives, analogs or variants thereof may be driven by any of a number of regulatory elements. For example, viral promoters such as the 35S RNA and 19S RNA promoters of CaMV (Brisson et al., 1984, Nature Vol. 310, pages 511-514), or the coat protein promoter of TMV (Takamatsu et al., 1987, EMBO J. Vol. 6, pages 307-311) may be used; alternatively, plant promoters such as the small subunit of RUBISCO (Coruzzi et al., 1984, EMBO J. Vol. 3, pages 1671-1680; Broglie et al., 1984, Science Vol. 224, pages 838-843); or heat shock promoters, e.g., soybean hsp17.5-E or hsp17.3-B (Gurley et al., 1986, Mol. Cell. Biol. Vol. 6, pages 559-565) may be used. These constructs can be introduced into plant cells using Ti plasmids, R1 plasmids, plant virus vectors, direct DNA transformation, biolistics/particle bombardment, microinjection, electroporation, etc. For reviews of such techniques see, for example, Weissbach & Weissbach, 1988, Methods for Plant Molecular Biology, Academic Press, New York, Section VIII, pp. 421-463; and Grierson & Corey, 1988, Plant Molecular Biology, 2d Ed., Blackie, London, Ch. 7-9. As used herein, regulatory elements include but are not limited to inducible and non-inducible promoters, enhancers, operators and other elements known to those skilled in the art that drive and regulate expression. Preferably the promoter is capable of directing expression in a particular tissue of the plant and/or at particular stages of development of the plant. The promoter may be heterologous or homologous to the plant. Preferably the promoter directs expression to the fruit, e.g., tomato, the leaves, e.g., lettuce, the endosperm of the plant seed or to the roots or tuber of the plant. A preferred promoter is the high molecular weight glutenin (HMWG) gene of wheat. Other suitable promoters will be known to the skilled man, such as the promoters of gliadin, branching enzyme, ADPG pyrophosphorylase, starch synthase and actin, for example.

A transformed plant with the ability to express SARS-CoV antigens including polypeptides of the SARS-CoV virus, or fragments, derivatives, analogs, or variants thereof, or molecules having the similar activities as the polypeptides of the SARS-CoV virus may be engineered by transforming a plant cell with a vector comprising a sequence encoding antigens, including polypeptides of the SARS-CoV or fragments, derivatives, analogs, or variants thereof, or molecules having the similar activities as the polypeptides of the SARS-CoV virus. In one embodiment, a plant promoter is operably associated with a sequence encoding the desired antigens. As used herein, the term “Operably associated” or “operably linked” refers to an association in which the regulatory regions (e.g., promoter, enhancer) and the nucleic acid sequence to be expressed are covalently joined and positioned in such a way as to permit transcription, and under the appropriate condition, translation. In a preferred embodiment of the present invention, the associated promoter for nuclear transformation is a strong and non tissue- or developmental-specific plant promoter (e.g., a promoter that strongly expresses in many or all plant tissue types). Examples of such strong, “constitutive” promoters include, but are not limited to, the CaMV 35S promoter (Odell et al., 1985, Nature 313:810-812), the T-DNA mannopine synthetase promoter, and their various derivatives. In another preferred embodiment, an inducible or repressible promoter is used to express the SARS-CoV virus of interest in a plant, for example, a tet operator promoter as described in Weinmann et al., 1994, The Plant Journal 5: 559-569; or a glucocorticoid-inducible promoter as described in McNellis et al., 1998, The Plant Journal 14: 247-257; or an ethanol inducible promoter as described in Caddick et al., 1998, Nature Biotechnology 16: 177-180. See, also, Gatz, 1995, Methods In Cell Biology 50: 411-424, which describes inducible and repressible gene expression systems for plants.

The promoters used for plastid transformation include strong and constitutive promoters in plastid expression including the psbA promoter (the psbA gene encodes the photosystem II 32 kD protein) and the 16S rRNA operon (rrn) promoter, or modifications thereof of these promoters which have enhanced expression (Suzuki et al., 2003, Plant Cell 15: 195-205; see also PCT publication no. WO 00/03012).

In one embodiment of the invention, SARS-CoV antigens will be localized in the apoplastic space from nuclear expression. The SARS-CoV antigens may be directed to the apoplastic space, when expressed in a plant, by expressing the antigens as fusion proteins together with a peptide that acts as a signal or transporter so that the antigen is localized in the apoplastic space of the transgenic plant. A variety of signal or transporter peptides can be used, for example, the PR1b signal sequence as described in Lund et al., 1992, Plant Molecular Biology 18: 47-53; or the PR-1a, b and c signal sequences as described in Pfitzner et al., 1987, Nucleic Acids Research 15: 4449-4465. A fusion protein comprising a signal or transporter peptide and an SARS-CoV antigen may be constructed by linking polynucleotides specific for each component to each other (e.g., the polynucleotides are linked in frame) so that the desired fusion protein is made when the fusion polynucleotide is expressed in a transgenic plant. A skilled artisan would know how to construct a polynucleotide useful for expressing SARS-CoV antigens, including polypeptides of the SARS-CoV or fragments, derivatives, analogs, or variants thereof, or molecules having the similar activities as the polypeptides of the SARS-CoV virus in the apoplastic space of a transgenic plant.

In another embodiment of the present invention, it may be advantageous to engineer a plant with a vector comprising a sequence SARS-CoV antigens, polypeptides of the SARS-CoV or fragments, derivatives, analogs, or variants thereof, or molecules having the similar activities as the polypeptides of the SARS-CoV virus operably associated with a tissue- or developmental-specific promoter, such as, but not limited to, the tomato E8 fruit-specific promoter, the chalcone synthase (CHS) promoter, the patatin promoter.

In yet another embodiment of the present invention, it may be advantageous to transform a plant with a vector comprising a sequence encoding SARS-CoV antigens, polypeptides of the SARS-CoV or fragments, derivatives, analogs, or variants thereof, or molecules having the similar activities as the polypeptides of the SARS-CoV virus operably linked to a modified or artificial promoter. Typically, such promoters, constructed by recombining structural elements of different promoters, have unique expression patterns and/or levels not found in natural promoters. See, e.g., Salina et al., 1992, Plant Cell 4: 1485-1493, for examples of artificial promoters constructed from combining cis-regulatory elements with a promoter core.

In yet an additional embodiment of the present invention, the expression of SARS-CoV antigens, polypeptides of the SARS-CoV or fragments, derivatives, analogs, or variants thereof, or molecules having the similar activities as the polypeptides of the SARS-CoV virus may be engineered by increasing the copy number of the gene encoding the desired protein or polypeptide using techniques known in the art.

The present invention provides a vector capable of directing the expression of SARS-CoV viral nucleotide sequences, fragments, derivatives, analogs or variants thereof, in a genetically modified plant or progeny thereof including, for example, transgenic and transplastomic plants. The plant vector is constructed using general recombinant DNA and cloning techniques known in the art of biotechnology, see, e.g., Sambrook et al., supra; Ausubel et al., supra. Such a polynucleotide construct typically comprises a polynucleotide sequence that encodes an engineered gene product and one or more regulatory polynucleotide sequence. Regulatory sequences useful for the polynucleotide construct of the invention include, but are not limited to, a promoter, an enhancer, an intron, a splice donor, a splice acceptor, a polyadenylation sequence, a RNA stability regulating sequence, or an element of any one of the above (e.g., promoter elements including, but not limited to, a TATA box). The regulatory elements useful for the present invention are capable of directing expression in a plant species in which expression of the nucleotide sequences that encode the SARS-CoV viral antigens is desired. In another preferred aspect, the regulatory elements are capable of directing expression in a cell type in which expression of the engineered gene product is desired in the plant species of interest.

Regulatory elements useful for the present invention are known to those of skill in the art, for example, promoter and enhancer elements of genes known to be expressed in the cell type and plant species of interest. A promoter useful for expression of the SARS-CoV viral antigens in a plant species of interest may also be isolated using routine experimentation, for example, by isolating a promoter region of a gene known to be expressed in the desired fashion. For example, one may screen a genomic library with a cDNA probe specific for the 5′ end of a messenger RNA known to be expressed in the cell type of interest of the plant species of interest. Such a 5′ end cDNA probe should preferably be only about 100 base pairs to about 300 base pairs so that the clones identified in the genomic library are likely to include the 5′ end of the gene possibly including the promoter region of the gene for which the probe is specific. The promoter region typically includes about 1,000 to about 2,000 base pairs upstream of the transcription initiation site. Thus, a promoter useful for the expression of the engineered SARS-CoV nucleotide sequences, genes, fragments, derivatives, analogs or variants thereof of the present invention is a polynucleotide from about 2,000 base pairs upstream to about 50 base pairs downstream of the transcription initiation site of a gene known to be expressed in the cell type of interest in the plant species of interest, or is a portion of the polynucleotide.

In order to facilitate the proper processing of the SARS-CoV viral antigens, it may be necessary to include a nucleotide sequence that encodes a peptide sequence necessary for such processing. For example, a peptide sequence which is recognized by and functional in the transgenic host plant, for example, to facilitate the entry of the antigen into the endoplasmic reticulum may be necessary, i.e., signal Sequence.

Transformation of Plants and Plant Cells

Plants and plant cells nuclei and plastids may be transformed using any method known in the art. In an embodiment of the present invention, Agrobacterium is employed to introduce the vector of the present invention in nuclear transformation. Such transformation preferably uses binary Agrobacterium T-DNA vectors (Bevan, 1984, Nuc. Acid Res. Vol. 12, pages 8711-8721) and the co-cultivation procedure (Horsch et al., 1985, Science Vol. 227, pages 1229-1231). Generally, the Agrobacterium transformation system is used to engineer dicotyledonous plants (Bevan et al., 1982, Ann. Rev. Genet Vol. 16, pages 357-384; Rogers et al., 1986, Methods Enzymol. Vol. 118, pages 627-641). The Agrobacterium transformation system may also be used to transform, as well as transfer, DNA to monocotyledonous plants and plant cells (see Hernasteen et al., 1984, EMBO J. Vol. 3: pages 3039-3041; Hooykaas-Van Slogteren et al., 1984, Nature Vol. 311, pages 763-764; Grimsley et al., 1987, Nature Vol. 325, pages 1677-179; Boulton et al., 1989, Plant Mol. Biol. Vol. 12, pages 31-40; and Gould et al., 1991, Plant Physiol. Vol. 95, pages 426-434).

In other embodiments, various alternative methods for introducing recombinant nucleic acid constructs into plants and plant cells may also be utilized. These other methods are particularly useful where the target is a monocotyledonous plant or plant cell. Alternative gene transfer and transformation methods include, but are not limited to, particle gun bombardment (biolistics), protoplast transformation through calcium-, polyethylene glycol (PEG)- or electroporation-mediated uptake of naked DNA (see Paszkowski et al., 1984, EMBO J. Vol. 3, pages 2717-2722; Potrykus et al. 1985, Molec. Gen. Genet. Vol. 199, pages 169-177; Fromm et al., 1985, Proc. Nat. Acad. Sci. USA Vol. 82, pages 5824-5828; and Shimamoto, 1989, Nature Vol. 338, pages 274-276) and electroporation of plant tissues (D'Halluin et al., 1992, Plant Cell Vol. 4, pages 1495-1505). Additional methods for plant cell transformation include microinjection, silicon carbide mediated DNA uptake (Kaeppler et al., 1990, Plant Cell Reporter Vol. 9, pages 415-418), and microprojectile bombardment (see Klein et al., 1988, Proc. Nat. Acad. Sci. USA Vol. 85, pages 4305-4309; and Gordon-Kamm et al., 1990, Plant Cell Vol. 2, pages 603-618). In any methods, selectable markers may be used, at least initially, in order to determine whether transformation has actually occurred. Useful selectable markers include enzymes which confer resistance to an antibiotic, such as gentamycin, hygromycin, kanamycin and the like. Alternatively, markers which provide a compound identifiable by a color change, such as GUS, or luminescence, such as luciferase, may be used.

The chimeric gene may also comprise a gene switch mechanism which determines under what conditions or when the coding sequence is to be expressed. The gene switch may be a chemically induced promoter or a temperature controlled promoter, for example.

In a specific embodiment, each plastid transformation construct (pCV1, pCV6 or pCV8, and its derivatives containing an S1 with nucleotide (but not amino acid) changes to optimize codon usage for expression in plants) was introduced by particle gun bombardment into tobacco (Staub and Maliga, 1994, Plant Journal Vol. 6, pages 547-553) and tomato (Ruf et al., 2001, Nature Biotech Vol. 19, pages 870-875). In a specific embodiment, production of transgenic tobacco and transgenic lettuce expressing S1 or M antigen by nuclear transformation was carried out using Agrobacterium-mediated transformation (Horsch et al., 1985, Science Vol. 227, pages 1227-1231) with plasmids pCV2 [and its derivative containing an S1 with nucleotide (but not amino acid) changes to optimize codon usage for expression in plants] and pCV4, respectively. In another specific embodiment, production of transgenic tomato expressing S1 or M antigen by nuclear transformation is carried out.

According to the present invention, a wide variety of plants and plant cell systems may be engineered for the desired physiological and agronomic characteristics described herein using the nucleic acid constructs of the present invention and the various transformation methods mentioned above. In preferred embodiments, target plants and plant cells for engineering include, but are not limited to, those monocotyledonous and dicotyledonous plants, such as crops including grain crops (e.g., wheat, maize, rice, millet, barley), tobacco, fruit crops (e.g., tomato, apple, pear, strawberry, orange), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); plants used in phytoremediation (e.g., heavy metal accumulating plants); oil crops (e.g., sunflower, rape seed); and plants used for experimental purposes (e.g., Arabidopsis).

Screening of Transformed Plants and Plant Cells

According to the present invention, desired plants may be obtained by engineering one or more of the vectors expressing SARS-CoV antigens as described herein into a variety of plant cell types, including but not limited to, protoplasts, tissue culture cells, tissue and organ explants, pollens, embryos, as well as whole plants. In an embodiment of the present invention, the engineered plant material is selected or screened for transformants (those that have incorporated or integrated the introduced gene construct(s)) following the approaches and methods described below. An isolated transformant may then be regenerated into a plant and progeny thereof via sexual or asexual reproduction or growth. Alternatively, the engineered plant material may be regenerated into a plant before subjecting the derived plant to selection or screening for the marker gene traits. Procedures for regenerating plants from plant cells, tissues or organs, either before or after selecting or screening for marker gene(s), are well known to those skilled in the art.

A transformed plant cell, callus, tissue or plant may be identified and isolated by selecting or screening the engineered plant material for traits encoded by the marker genes present on the transforming DNA. For instance, selection may be performed by growing the engineered plant material on media containing inhibitory amount of the antibiotic or herbicide to which the transforming gene construct confers resistance. Further, transformed plants and plant cells may also be identified by screening for the activities of any visible marker genes (e.g., the β-glucuronidase, luciferase, B or Cl genes) that may be present on the vector of the present invention. Such selection and screening methodologies are well known to those skilled in the art.

Physical and biochemical methods may also be used to identify plant or plant cell transformants containing the gene constructs of the present invention. These methods include but are not limited to: 1) Southern analysis or PCR amplification for detecting and determining the structure of the recombinant DNA insert; 2) Northern blot, S1 RNase protection, primer-extension or reverse transcriptase-PCR amplification for detecting and examining RNA transcripts of the gene constructs; 3) enzymatic assays for detecting enzyme activity, where such gene products are encoded by the gene construct; 4) protein gel electrophoresis (PAGE), Western blot techniques, immunoprecipitation, or enzyme-linked immunoassays, where the gene construct products are proteins. Additional techniques, such as in situ hybridization, enzyme staining, and immunostaining, also may be used to detect the presence or expression of the recombinant construct in specific plant organs and tissues. The methods for doing all these assays are well known to those skilled in the art.

In a specific embodiment, the selectable marker gene aadA, which specifies spectinomycin-resistance, is driven from the rice plastid 16S rRNA operon (rrn) promoter in plastid transformation. In a specific embodiment, the selectable marker gene nptII, which specifies kanamycin-resistance, is driven by the NOS (nopaline synthase) promoter in nuclear transformation.

The present invention relates to transgenic and transplastomic plants that express one or more epitopes of the SARS-CoV virus. A transgenic or transplastomic plant expressing a SARS-CoV antigen elicits the production of antibodies against SARS-CoV by the subject after ingestion of the modified plant or plant parts by a subject; or administration of plant extract to a subject using methods known in the art.

Examples of plants are monocots, dicots, crop plants (i.e., any plant species grown for purposes of agriculture, food production for animals including humans, plants that are typically grown in groups of more than about 10 plants in order to harvest the entire plant or a part of the plant, e.g., a fruit, a flower or a crop, e.g., tobacco, grain, that the plants bear, etc.), trees (i.e., fruit trees, trees grown for wood production, trees grown for decoration, etc.), flowers of any kind (i.e., plants grown for purposes of decoration, for example, following their harvest), cactuses

Further examples of plants in which the SARS-CoV viral antigens may be expressed include Viridiplantae, Streptophyta, Embryophyta, Tracheophyta, Euphyllophytes, Spermatophyta, Magnoliophyta, Liliopsida, Commelinidae, Poales, Poaceae, Oryza, Oryza sativa, Zea, Zea mays, Hordeum, Hordeum vulgare, Triticum, Triticum aestivum, Eudicotyledons, Core eudicots, Asteridae, Euasterids, Rosidae, Eurosids II, Brassicales, Brassicaceae, Arabidopsis, Magnoliopsida, Solananae, Solanales, Solanaceae, Solanum, and Nicotiana.

Also included are, for example, crops of particular interest including Solanaceae, including processing and fresh market tomatoes, pepper and eggplant; leafy plants, including lettuce and spinach; Brassicas, including broccoli, brussels sprouts, calabrese, cale, cauliflower, red cabbage and white cabbage; cucurbits, including cucumber, melon, watermelon, zucchini and squash; large seeded plants, including peas, beans and sweetcorn; rooted plants, including carrots and onions; vegetatively propagated plants, including berries, grapes, banana, pineapple, kiwifruit, and rosaceous fruit and nut crops; and tropical crops, including mango and papaya.

Thus, the invention has use over a broad range of plants including, but not limited to, species from the genera Anacardium, Arachis, Asparagus, Atropa, Avena, Brassica, Citrus, Citrullus, Capsicum, Carthamus, Cocos, Coffea, Cucumis, Cucurbita, Daucus, Elaeis, Fragaria, Glycine, Gossypium, Helianthus, Heterocallis, Hordeum, Hyoscyamus, Lactuca, Linum, Lolium, Lupinus, Lycopersicon, Malus, Manihot, Majorana, Medicago, Nicotiana, Olea, Oryza, Panieum, Panneserum, Persea, Phaseolus, Pistachia, Pisum, Pyrus, Prunus, Raphanus, Ricinus, Secale, Senecio, Sinapis, Solanum, Sorghum, Theobromus, Trigonella, Titicum, Vicia, Vitis, Vigna, and Zea.

Vaccine Formulations and Administration

The invention also provides vaccine formulations containing extracts of the modified plants of the present invention, which are suitable for administration to elicit a protective immune (humoral and/or cell mediated) response against certain antigens, e.g., for the treatment and prevention of SARS.

Suitable preparations of such vaccines include injectables, either as liquid solutions or suspensions; solid forms suitable for solution in, or suspension in, liquid prior to injection, may also be prepared. The preparation may also be emulsified, or the proteinaceous molecules encapsulated in liposomes. The active immunogenic ingredients are often mixed with carriers, vehicles or excipients which are pharmaceutically acceptable and compatible with the active ingredient. Suitable excipients are, for example, water, saline, buffered saline, dextrose, glycerol, ethanol, sterile isotonic aqueous buffer or the like and combinations thereof. In addition, if desired, the vaccine preparation may also include minor amounts of auxiliary substances such as wetting or emulsifying agents, pH buffering agents, and/or adjuvants which enhance the effectiveness of the vaccine.

Examples of adjuvants which may be effective, include, but are not limited to: aluminum hydroxide, N-acetyl-muramyl-L-threonyl-D-isoglutamine (thr-MDP), N-acetyl-nor-muramyl-L-alanyl-D-isoglutamine, N-acetylmuramyl-L-alanyl-D-isoglutaminyl-L-alanine-2-(1′-2′-dipalmitoyl-sn-glycero-3-hydroxyphosphoryloxy)-ethylamine.

The effectiveness of an adjuvant may be determined by measuring the induction of anti-idiotype antibodies directed against the injected modified plant extract formulated with the particular adjuvant.

The composition can be a liquid solution, suspension, emulsion, tablet, pill, capsule, sustained release formulation, or powder. Oral formulation can include standard carriers such as pharmaceutical grades of mannitol, lactose, starch, magnesium stearate, sodium saccharine, cellulose, magnesium carbonate.

In another embodiment, the composition can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

Generally, the ingredients are supplied either separately or mixed together in unit dosage form, for example, as a dry lyophilized powder or water-free concentrate in a hermetically sealed container such as an ampoule or sachette indicating the quantity of active agent. Where the composition is administered by injection, an ampoule of sterile diluent can be provided so that the ingredients may be mixed prior to administration.

In a specific embodiment, the lyophilized modified immunoglobulin of the invention is provided in a first container; a second container comprises diluent consisting of an aqueous solution of 50% glycerin, 0.25% phenol, and an antiseptic (e.g., 0.005% brilliant green).

The invention also provides a pharmaceutical pack or kit comprising one or more containers filled with one or more of the ingredients of the vaccine formulations of the invention. Associated with such container(s) can be a notice in the form prescribed by a governmental agency regulating the manufacture, use or sale of pharmaceuticals or biological products, which notice reflects approval by the agency of manufacture, use or sale for human administration.

The compositions may, if desired, be presented in a pack or dispenser device which may contain one or more unit dosage forms containing the active ingredient. The pack may for example comprise metal or plastic foil, such as a blister pack or transdermal patch. The pack or dispenser device may be accompanied by instructions for administration. Composition comprising an extract of the invention formulated in a compatible pharmaceutical carrier may also be prepared, placed in an appropriate container, and labeled for treatment of an indicated condition.

The subject to which the vaccine is administered is preferably a mammal, most preferably a human, but can also be a non-human animal including but not limited to rabbits, goats, cows, pigs, sheep, horses, civets, rodents, raccoons, raccoon dogs, dogs, ferrets, ferret Badger, cats, and avian species.

Many methods may be used to introduce the vaccine formulations of the invention; these include but are not limited to oral, intracerebral, intradermal, intramuscular, intraperitoneal, intravenous, subcutaneous, intranasal routes, and via scarification (scratching through the top layers of skin, e.g., using a bifurcated needle) or any other standard routes of immunization. In a specific embodiment, scarification is employed.

The precise dose of the extract of the modified plant to be employed in the formulation will also depend on the route of administration, and the nature of the patient, and should be decided according to the judgment of the practitioner and each patient's circumstances according to standard clinical techniques. An effective immunizing amount is that amount sufficient to produce an immune response to the SARS-CoV viral epitope in the host (i.e., an anti-idiotype reaction) to which the vaccine preparation is administered. Effective doses may also be extrapolated from dose-response curves derived from animal model test systems.

The present invention provides methods of vaccination against SARS. The methods comprise administering whole modified plants or plant parts, extracts, to a subject for the production of antibodies against SARS-CoV viral antigens. In preferred embodiments, the vaccine is administered by ingestion of the modified plants or injection of the modified plant extracts. In certain embodiments, a first vaccine comprising a modified plant extract transformed by a vector for expression of a S antigen may be administered prior to, simultaneously with, or after administration of a second vaccine comprising a modified plant extract transformed by a vector for expression of a M antigen. The term “concurrently” is not limited to the administration of prophylactic or therapeutic composition at exactly the same time, but rather it is meant that the composition of the present invention and the other agent are administered to a mammal in a sequence and within a time interval such that the composition comprising the polynucleotides can act together with the other composition to provide an increased benefit than if they were administered otherwise. In various embodiments, the prophylactic or therapeutic compositions are administered less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart or no more than 48 hours apart. In preferred embodiments, two or more components are administered within the same patient visit.

In other embodiments, the prophylactic or therapeutic compositions are administered at about 30 minutes, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 1 to 2 days apart, at about 2 to 4 days apart, at about 4 to 6 days apart, at about 1 week part, at about 1 to 2 weeks apart, or more than 2 weeks apart. One skilled in the art would be able to determine such a time frame by determining the half life of the administered compositions.

In certain embodiments, the prophylactic or therapeutic compositions of the invention are cyclically administered to a subject. Cycling therapy involves the administration of a first composition for a period of time, followed by the administration of a second composition and/or third composition for a period of time and repeating this sequential administration. Cycling therapy can reduce the development of resistance to one or more of the therapies, avoid or reduce the side effects of one of the therapies, and/or improves the efficacy of the treatment.

In certain embodiments, prophylactic or therapeutic compositions are administered in a cycle of less than about 3 weeks, about once every two weeks, about once every 10 days or about once every week. One cycle can comprise the administration of a therapeutic or prophylactic composition by infusion over about 90 minutes every cycle, about 1 hour every cycle, about 45 minutes every cycle. Each cycle can comprise at least 1 week of rest, at least 2 weeks of rest, at least 3 weeks of rest. The number of cycles administered is from about 1 to about 12 cycles, more typically from about 2 to about 10 cycles, and more typically from about 2 to about 8 cycles.

In yet other embodiments, the therapeutic and prophylactic compositions of the invention are administered in metronomic dosing regiments, either by continuous infusion or frequent administration without extended rest periods. Such metronomic administration can involve dosing at constant intervals without rest periods. The dosing regimens encompass the chronic daily administration of relatively low doses for extended periods of time. In preferred embodiments, the use of lower doses can minimize toxic side effects and eliminate rest periods. In certain embodiments, the therapeutic and prophylactic compositions are delivered by chronic low-dose or continuous infusion ranging from about 24 hours to about 2 days, to about 1 week, to about 2 weeks, to about 3 weeks to about 1 month to about 2 months, to about 3 months, to about 4 months, to about 5 months, to about 6 months. The scheduling of such dose regimens can be optimalized by the skilled physician.

The dosage amounts and frequencies of administration provided herein are encompassed by the terms therapeutically effective and prophylactically effective. The dosage and frequency further will typically vary according to factors specific for each patient depending on the specific therapeutic or prophylactic composition administered, the severity and type of disease or disorder, the route of administration, as well as age, body weight, response, and the past medical history of the patient. Suitable regimens can be selected by one skilled in the art by considering such factors and by following, for example, dosages reported in the literature and recommended in the Physician's Desk Reference (56^{th} ed., 2002).

Various delivery systems are known and can be used to administer the therapeutic or prophylactic composition of the present invention, e.g., encapsulation in liposomes, microparticles, microcapsules, recombinant cells capable of expressing the antibody or antibody fragment, receptor-mediated endocytosis (see, e.g., Wu and Wu, J. Biol. Chem. 262:4429-4432 (1987)), construction of a nucleic acid as part of a retroviral or other vector, etc. Methods of administering a prophylactic or therapeutic composition of the invention include, but are not limited to, parenteral administration (e.g., intradermal, intramuscular, intraperitoneal, intravenous and subcutaneous), epidural, and mucosal (e.g., intranasal and oral routes). In a specific embodiment, prophylactic or therapeutic composition of the invention is administered intramuscularly, intravenously, or subcutaneously. The prophylactic or therapeutic composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the prophylactic or therapeutic composition of the invention locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers.

In yet another embodiment, the prophylactic or therapeutic composition can be delivered in a controlled release or sustained release system. In one embodiment, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of the therapeutic or prophylactic composition of the invention (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. No. 5,679,377; U.S. Pat. No. 5,916,597; U.S. Pat. No. 5,912,015; U.S. Pat. No. 5,989,463; U.S. Pat. No. 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a sustained release formulation is inert, free of leachable impurities, stable on storage, sterile, and biodegradable. In yet another embodiment, a controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one skilled in the art can be used to produce sustained release formulations comprising one or more therapeutic composition of the invention. See, e.g., U.S. Pat. No. 4,526,938, PCT publication WO 91/05548, PCT publication WO 96/20698, Ning et al., 1996, “Intratumoral Radioimmunotheraphy of a Human Colon Cancer Xenograft Using a Sustained-Release Gel,” Radiotherapy & Oncology 39:179-189, Song et al., 1995, “Antibody Mediated Lung Targeting of Long-Circulating Emulsions,” PDA Journal of Pharmaceutical Science & Technology 50:372-397, Cleek et al., 1997, “Biodegradable Polymeric Carriers for a bFGF Antibody for Cardiovascular Application,” Pro. Int'l. Symp. Control. Rel. Bioact. Mater. 24:853-854, and Lam et al., 1997, “Microencapsulation of Recombinant Humanized Monoclonal Antibody for Local Delivery,” Proc. Int'l. Symp. Control Rel. Bioact. Mater. 24:759-760, each of which is incorporated herein by reference in their entireties.

Antibodies may be isolated from a subject after administration of the vaccine of the present invention. Methods of production of antibodies against SARS comprise administering an effective amount of modified plant to a subject and isolating antibodies from a sample of the subject after a period of time. The sample may be a biological fluid, such as blood, serum, plasma, saliva, urine, stool, sputum, nasopharyngeal aspirates, cells and tissues.

Demonstration of Prophylactic and Therapeutic Utility

The present invention provides methods for preventing, treating, ameliorating, and/or managing SARS by administration of the modified plants of the present invention to a subject in need thereof. Modified plants that are useful for the methods of the invention can be screened or assayed in a variety of ways for efficacy in treating or preventing SARS.

First, the immunopotency of a vaccine formulation containing the modified plant or extracts of the plant of the invention can be determined by monitoring the immune response of test animals following immunization with the vaccine. Generation of a humoral response may be taken as an indication of a generalized immune response, other components of which, particularly cell-mediated immunity, may be important for protection against a disease. Test animals may include mice, rabbits, chimpanzees and eventually human subjects. A vaccine made in this invention can be made to infect chimpanzees experimentally. However, since chimpanzees are a protected species, the antibody response to a vaccine of the invention can first be studied in a number of smaller, less expensive animals, with the goal of finding one or two best candidate modified plants that produce the best combinations of SARS-CoV viral epitopes to use be used in chimpanzee efficacy studies.

The safety of the vaccine can also be determined by observing or monitoring symptoms of subjects after administration of vaccine to a test subject. In a specific embodiment, the entire genome of SARS-CoV is used in the vaccine. In preferred embodiment, the plant express less than 10%, less than 20%, less than 25%, less than 30%, less than 35%, less than 40%, less than 45%, less than 50%, less than 55%, less than 60%, less than 65%, less than 70%, less than 80%, less than 90% of the SARS-CoV polypeptides.

The immune response of the test subjects can be analyzed by various approaches such as the reactivity of the resultant immune serum to antigens, as assayed by known techniques, e.g., enzyme linked immunosorbent assay (ELISA), immunoblots, radioimmunoprecipitations, etc.; or protection from infection and/or attenuation of disease symptoms in immunized hosts.

As one example of suitable animal testing, the vaccine composition of the invention may be tested in rabbits for the ability to induce an immune response to the SARS viral antigens. For example, male specific-pathogen-free (SPF) young adult New Zealand White rabbits may be used. The test group of rabbits each receives an effective amount of the vaccine, via any route of administration, e.g., ingestion of modified plant or injection of modified plant extract. A control group of rabbits receives an injection in 1 mM Tris-HCl pH 9.0 of the vaccine containing the same kind of plant extract from an unmodified plant of the same type. Blood samples may be drawn from the rabbits every one or two weeks, and serum analyzed for antibodies specific for the viral antigen was directed using, e.g., a radioimmunoassay (Abbott Laboratories). The presence of antibodies may be assayed using an ELISA. Because rabbits may give a variable response due to their outbred nature, it may also be useful to test the vaccines in mice.

In one embodiment, the invention relates to biological material collected from subjects to which the modified plants and/or plant extracts were administered, either by injection or ingestion. The biological material can be tested for the presence of SARS-CoV polypeptides or fragments thereof. Biological materials include, but are not limited to, blood, serum, plasma, saliva, urine, stool, sputum, nasopharyngeal aspirates, cells and tissues. The biological material can be collected 1 hour, 2 hours, 6 hours, 12 hours, 24 hours, 2 days, 3 days, or 1 week after administration of the modified plant and/or plant extracts of the present invention.

In addition, a modified plant of the invention may be tested by first administering the modified plant to a test subject, either animal or human, and then isolating the anti-anti-idiotypic antibodies (i.e., the Ab3 antibodies) generated as part of the anti-idiotype response to the viral antigens. The isolated Ab3 may then be tested for the ability to bind the particular viral antigen (e.g., by any immunoassays known in the art, for example, but not limited to, radioimmunoassays, ELISA, “sandwich” immunoassay, gel diffusion precipitin reactions, immunodiffusion assays, western blots, precipitation reactions, agglutination assays, complement fixation assays, immunofluorescence assays, protein A assays, immunoelectrophoresis assays, etc.)

In one aspect where the vaccine is directed against SARS-CoV viral antigen, the efficacy of the isolated Ab3 for treating SARS is screened by culturing SARS-CoV virus infected cells from a culture or a patient, contacting the cells with the Ab3 antibody to be tested, and comparing the proliferation or survival of the contacted cells with the proliferation or survival of cells not so contacted with the Ab3 antibody, wherein a lower level of proliferation or survival of the contacted cells indicates that the Ab3 antibody (which was elicited by immunization with the SARS-CoV viral antigen) is effective to treat the SARS in the patient. Many assays standard in the art can be used to assess such survival and/or growth; for example, cell proliferation can be assayed by measuring ³H-thymidine incorporation, by direct cell count, by detecting changes in transcriptional activity of known genes such as proto-oncogenes (e.g., fos, myc) or cell cycle markers; cell viability can be assessed by trypan blue staining, differentiation can be assessed visually based on changes in morphology.

The present invention also provides antibodies against the SARS-CoV virus subsequent to the administration of the modified plant of the invention to a subject in need thereof. An antibody that immunospecifically binds an antigen of the SARS-CoV virus may also be tested directly in vivo. To monitor the effect of an antibody of the invention, the level of the antigen is measured at suitable time intervals before, during, or after administration of the vaccine. Any change or absence of change in the amount of the antigen can be identified and correlated with the effect of the treatment on the subject.

In particular, in the case of SARS, the serum levels of a SARS-CoV antigen bears a direct relationship with severity of SARS. Generally, a decrease in the level of antigen is associated with efficacious treatment. The serum levels of SARS-CoV antigens in a treated subject can be measured every 1 hour, 2 hours, 3 hours, 6 hours, 12 hours, 24 hours, 2 days, 3 days, or 1 week after administration of the modified plant and/or plant extracts of the present invention to said subject.

In a preferred aspect, the approach that can be taken is to determine the levels of antigen at different time points and to compare these values with a baseline level. The baseline level can be either the level of the marker present in normal, disease free individuals; and/or the levels present prior to treatment, or during periods of stability. These levels can then be correlated with the disease course or treatment outcome.

The levels of antigen can be determined by any method well known in the art. For example, SARS-CoV viral antigen can be quantitated by known immunodiagnostic methods such as western blotting immunoprecipitation using any antibody against SARS-CoV antigen.

The strength of the immune response in vivo to the viral antigen may be determined by any method known in the art, for example, but not limited to, delayed hypersensitivity skin tests and assays of the activity of cytolytic T-lymphocytes in vitro.

Delayed hypersensitivity skin tests are of great value in the testing of the overall immunocompetence and cellular immunity to an antigen. Proper technique of skin testing requires that the antigens be stored sterile at 4° C., protected from light and reconstituted shortly before use. A 25- or 27-gauge need ensures intradermal, rather than subcutaneous, administration of antigen. Twenty-four and 48 hours after intradermal administration of the antigen, the largest dimensions of both erythema and induration are measured with a ruler. Hypoactivity to any given antigen or group of antigens is confirmed by testing with higher concentrations of antigen or, in ambiguous circumstances, by a repeat test with an intermediate test.

To test the activity of cytolytic T-lymphocytes, T-lymphocytes isolated from the immunized subject, e.g., by the Ficoll-Hypaque centrifugation gradient technique, are re-stimulated with cells bearing the antigen against SARS was directed in 3 ml RPMI medium containing 10% fetal calf serum. In some experiments, 33% secondary mixed lymphocyte culture supernatant or IL-2 is included in the culture medium as a source of T cell growth factors. In order to measure the primary response of cytolytic T-lymphocytes after immunization, the isolated T cells are cultured with or without the cells bearing the antigen. After six days, the cultures are tested for cytotoxity in a 4 hour ⁵¹Cr-release assay. The spontaneous ⁵¹Cr-release of the targets should reach a level less than 20% if immunization was effective (Heike et al., J. Immunotherapy Vol. 15, pages 15-174).

The efficacy of the antibody can be assayed by administering the antibody to a subject (either a human subject or an animal model for the disease) and then monitoring either the levels of the SARS-CoV viral antigens or symptoms of the particular infectious disease. The levels of the SARS-CoV viral antigens may be determined by any method known in the art for assaying the levels of SARS-CoV viral antigens, e.g., the viral titer, in the case of a virus, or bacterial levels (for example, by culturing of a sample from the patient), etc. A decrease in the levels of the viral antigens or an elimination, amelioration or reduction in the number of symptoms of SARS indicates that the antibody is effective. Symptoms of SARS include, but are not limited to, temperature of greater than 100.4° F. (>38° C.) (according to the CDC), chills, headache, malaise, body aches, cough, shortness of breath, difficulty breathing, and hypoxia. Radiographic evidence of pneumonia, respiratory distress syndrome, and autopsy findings are also useful to determine whether a subject is inflicted with, recovering from, or free of SARS.

Efficacy of the Prophylactic and Therapeutic Utilities

Toxicity and efficacy of the prophylactic and/or therapeutic protocols of the instant invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

### EXAMPLES

The following examples illustrate the generation of transgenic or transplastomic tobacco plants that comprise the M and/or S1 proteins of the SARS-CoV virus.

Plasmid Construction

The backbone for construction of these plastid transformation vectors is derived from plasmid pVSR326 (Reddy et al., 2002, Mol Breeding 9: 259-269; PCT international application no. PCT/EP00/12446, international publication no. WO 01/42441). Plasmid pVSR326 utilizes the tobacco plastid genome sequences spanning rbcL-accD to target the reporter gene encoding β-glucuronidase (GUS) into the chloroplast genome by homologous recombination (Reddy et al., 2002). The promoter and terminator for GUS are derived from the rice plastid gene psbA; the psbA gene encoding the photosystem II 32 kD protein. The selectable marker gene aadA, which specifies spectinomycin-resistance, is driven from the rice plastid 16S rRNA operon (rrn) promoter and aadA is located adjacent to GUS (Reddy et al., 2002). In the pMLVHis vectors, other than unique restriction sites at this region, a start codon plus a (His)₅-tag is incorporated to facilitate the cloning and expression of (His)₅-tagged recombinant proteins.

Construction of pCV1, a Plastid Transformation Vector for Expression of the S1 Antigen

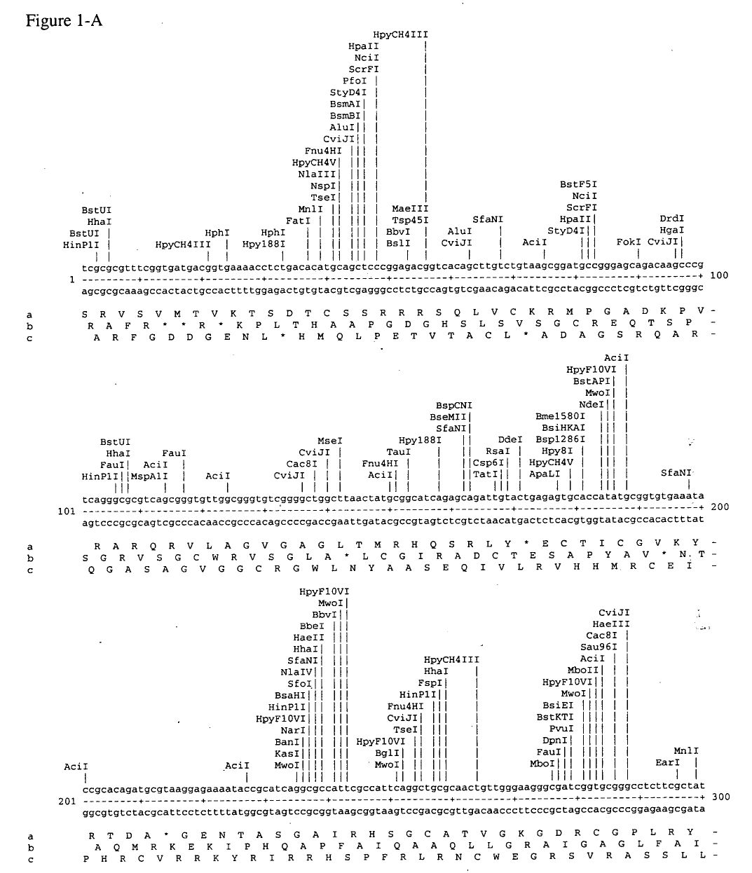

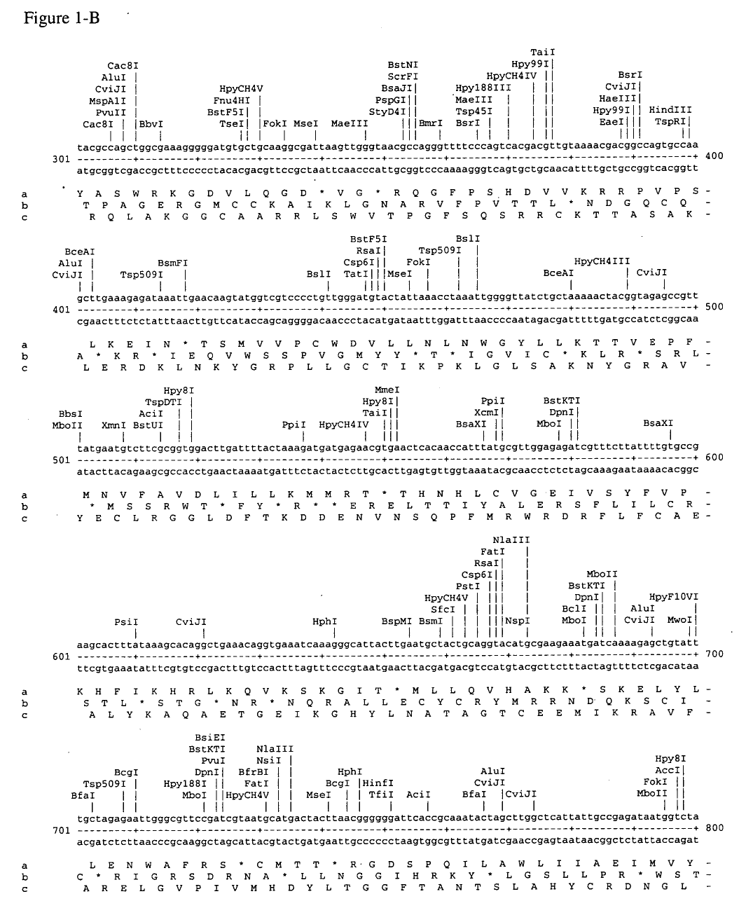

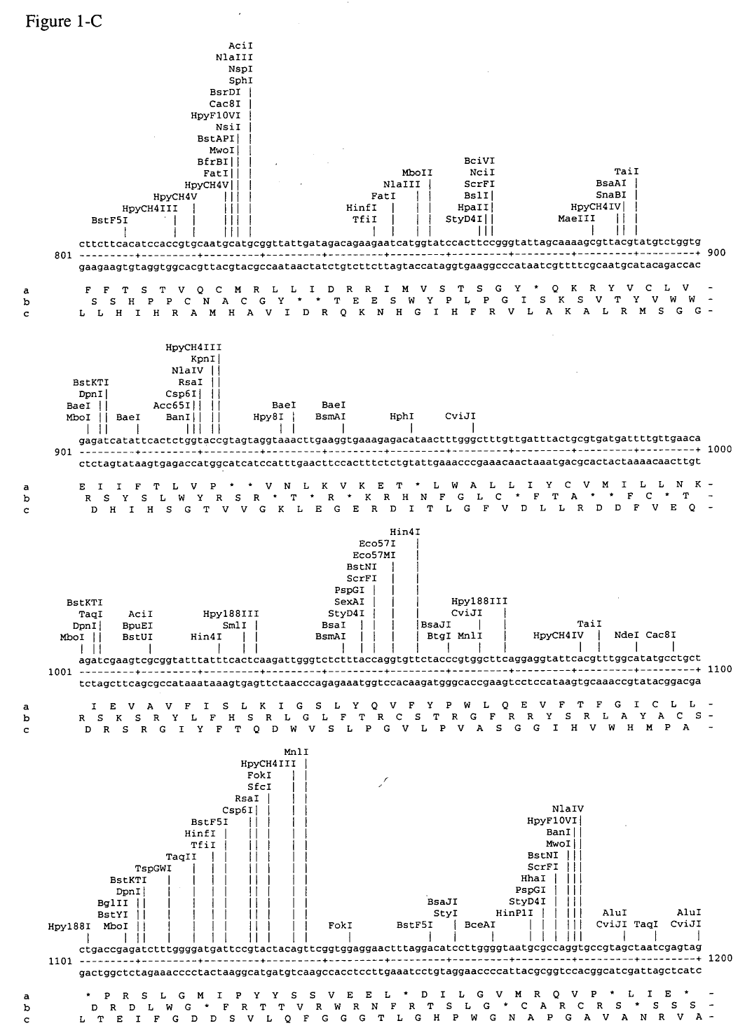

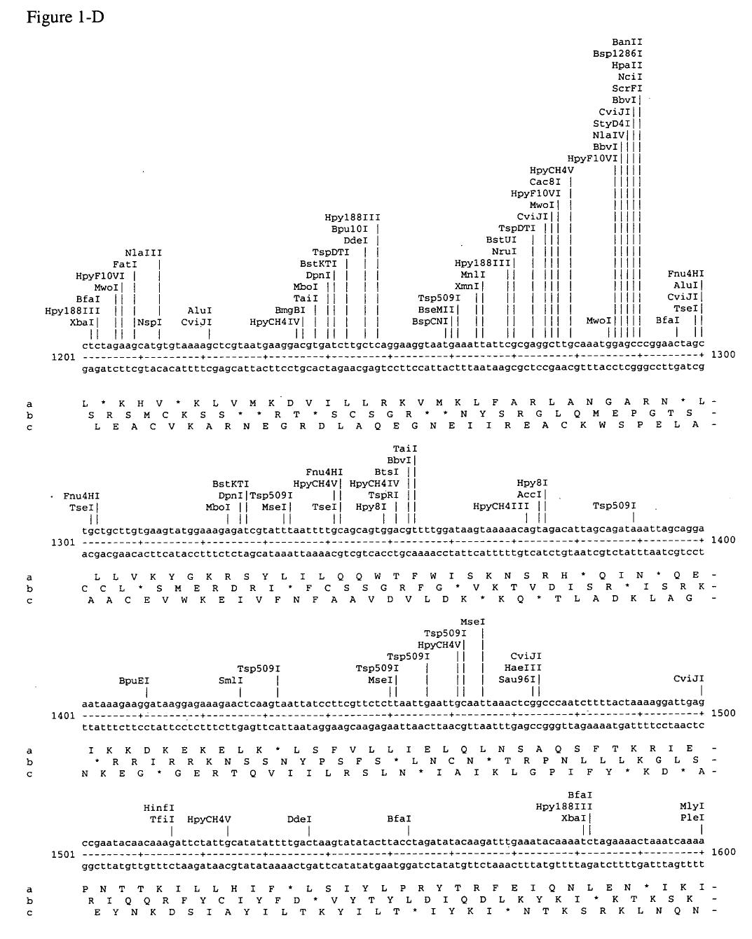

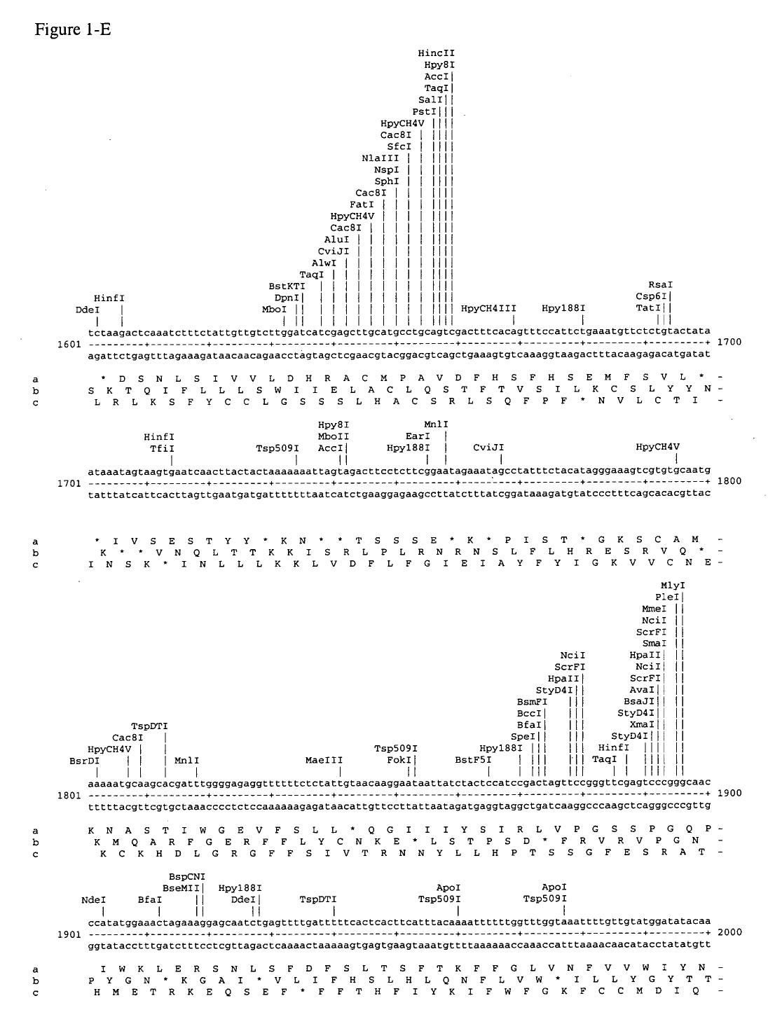

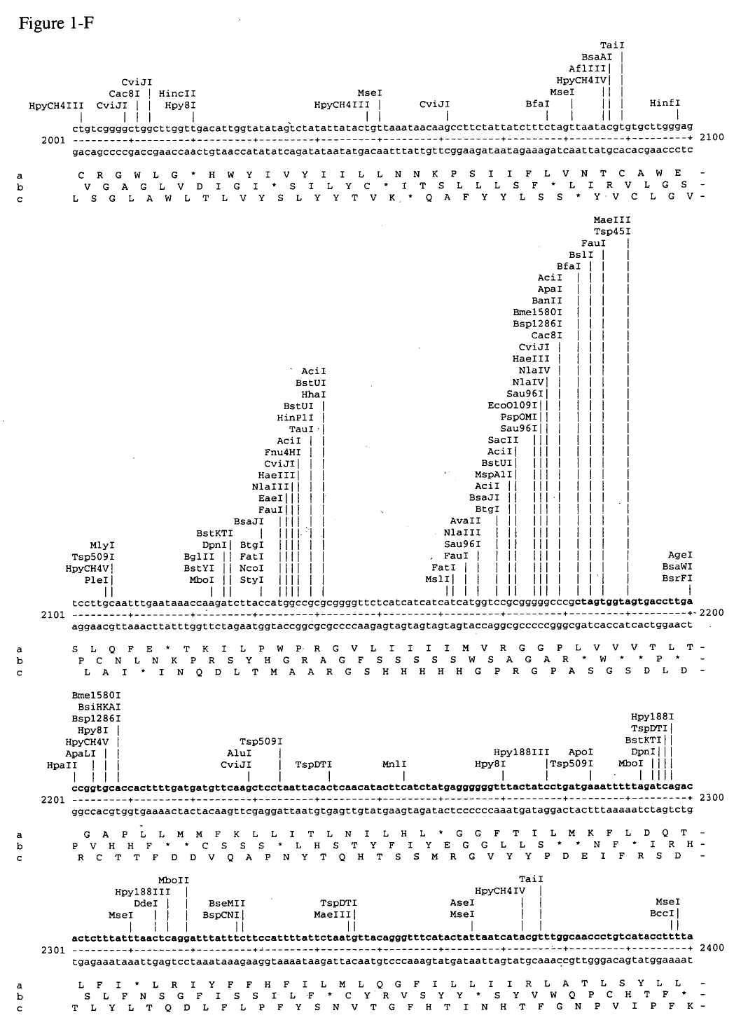

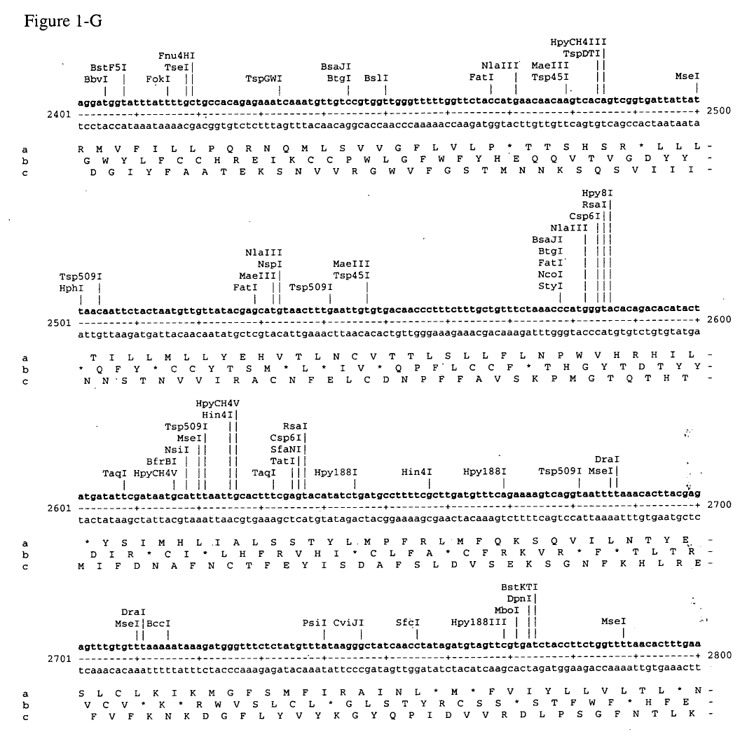

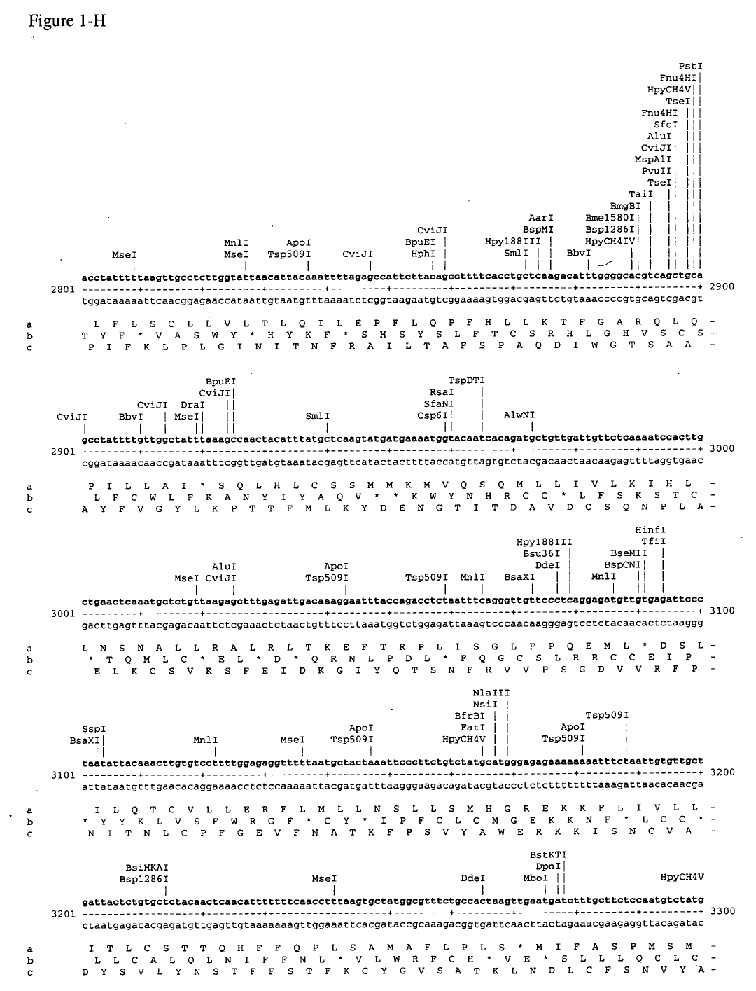

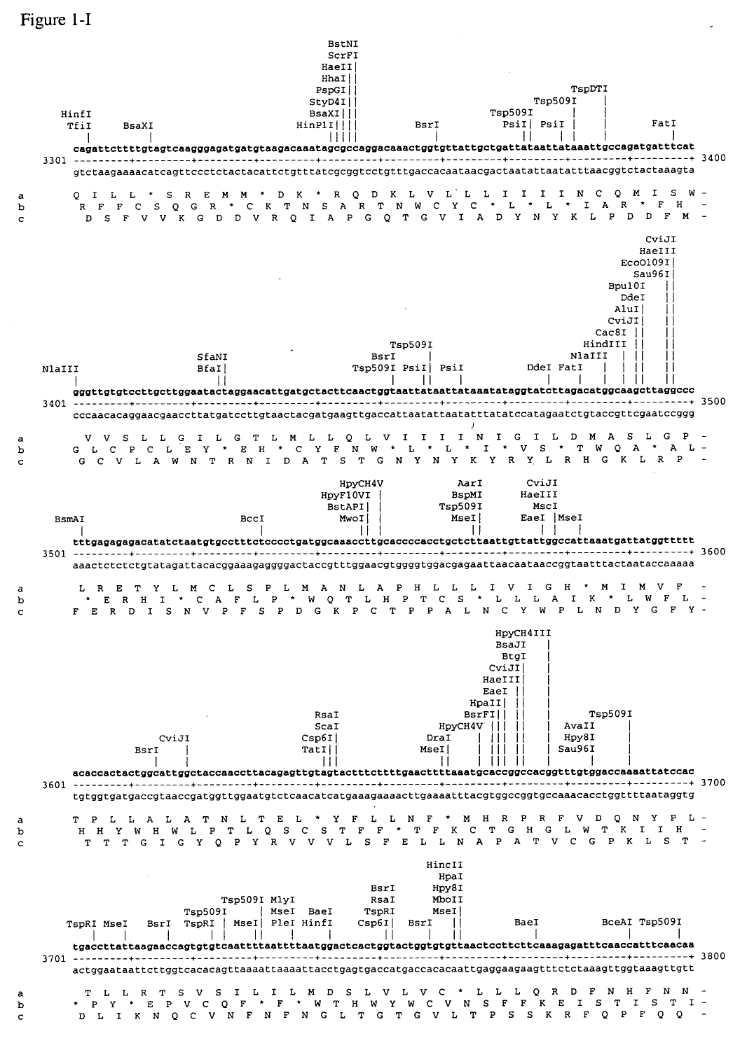

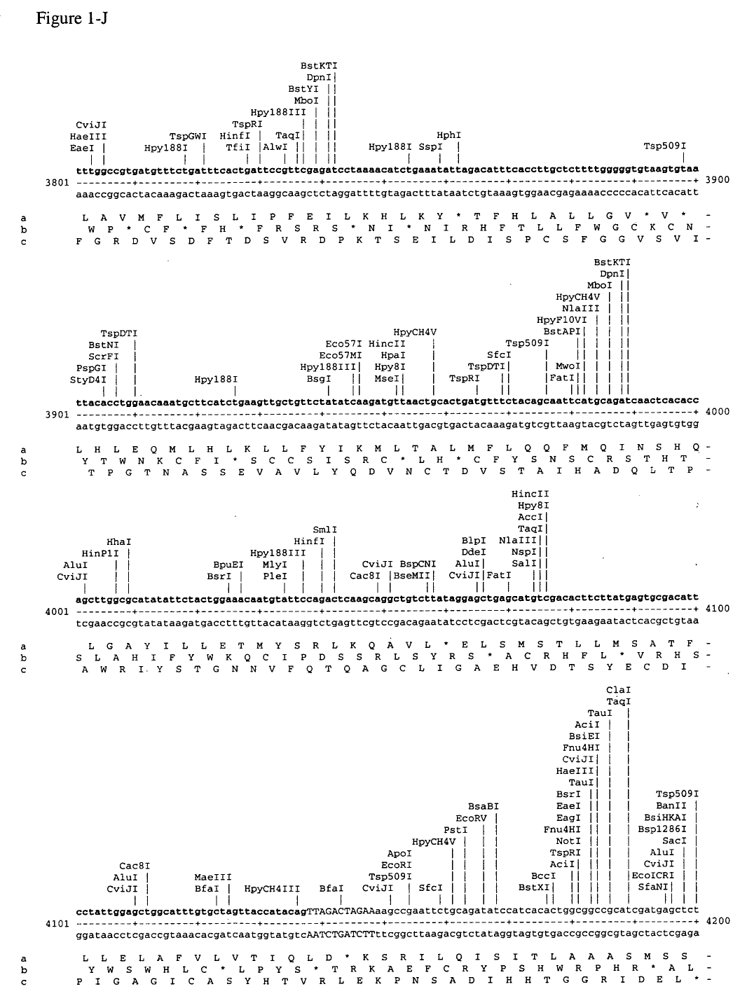

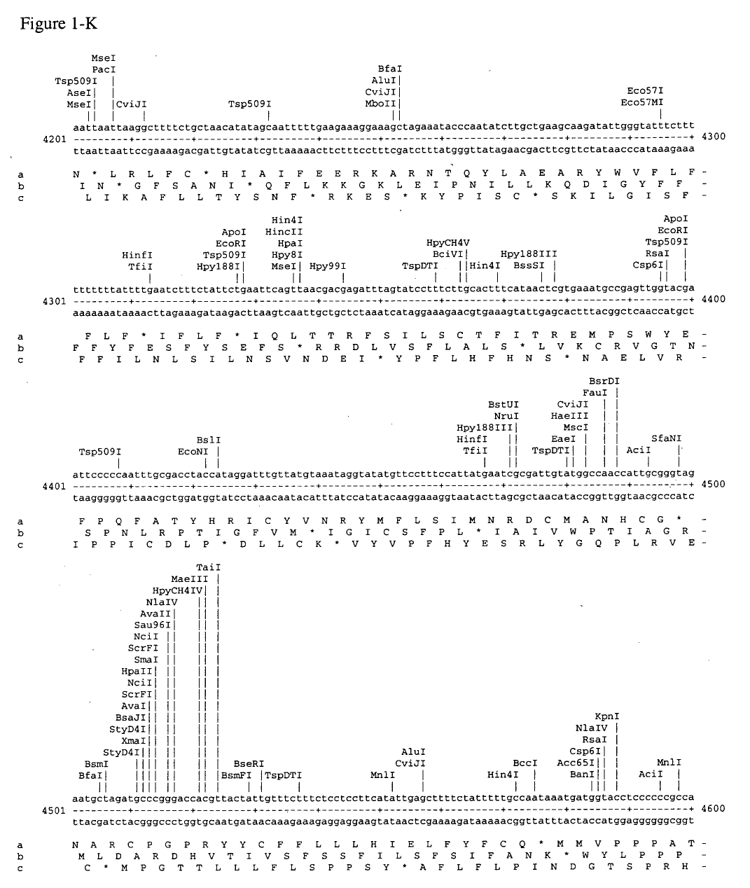

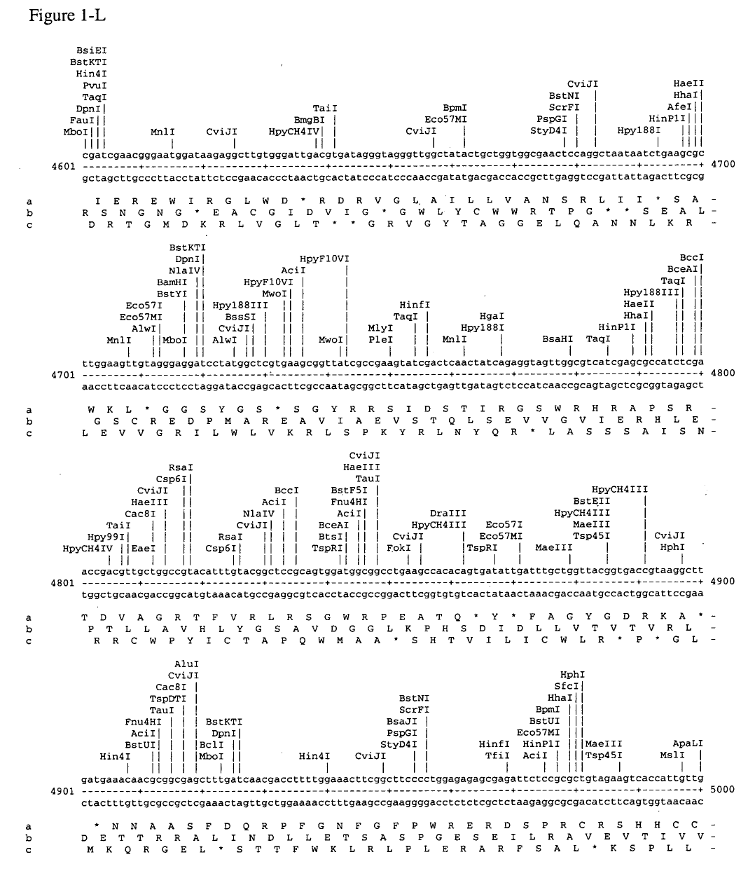

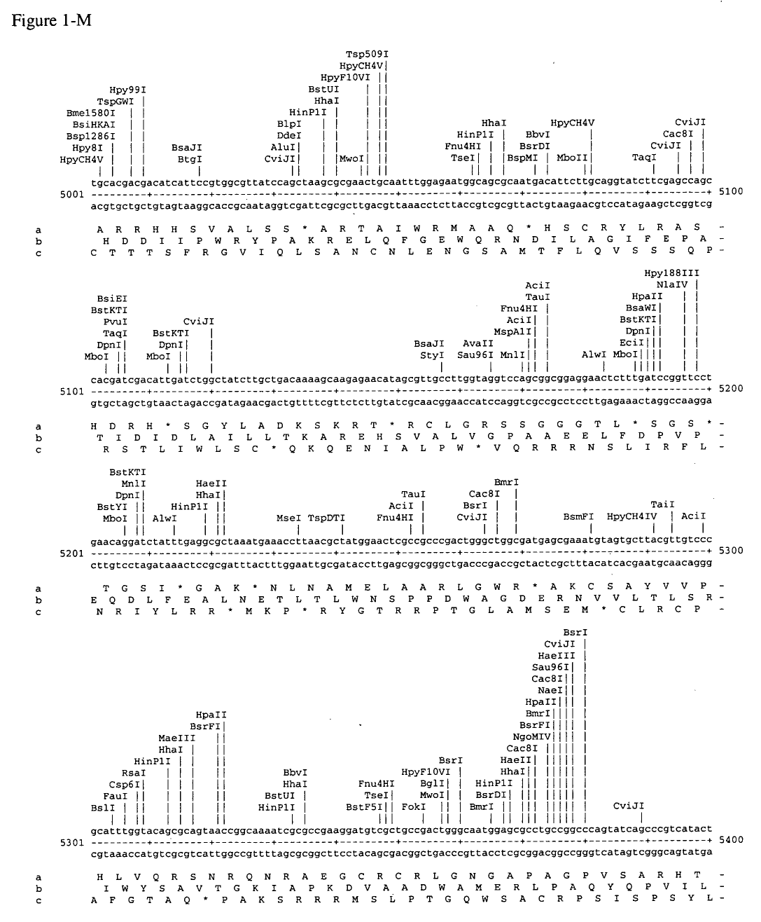

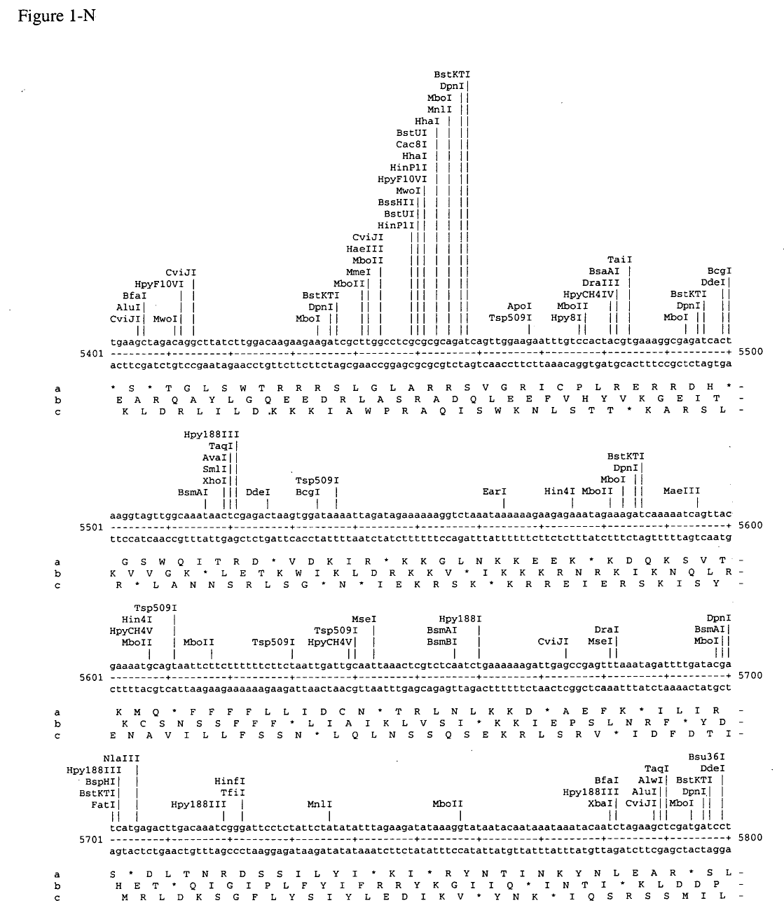

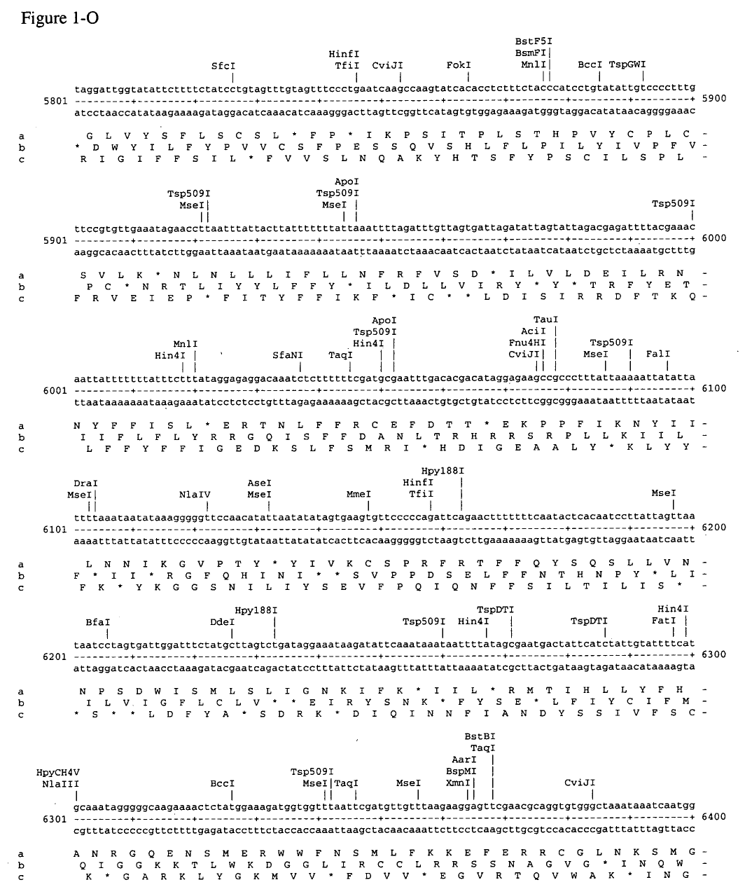

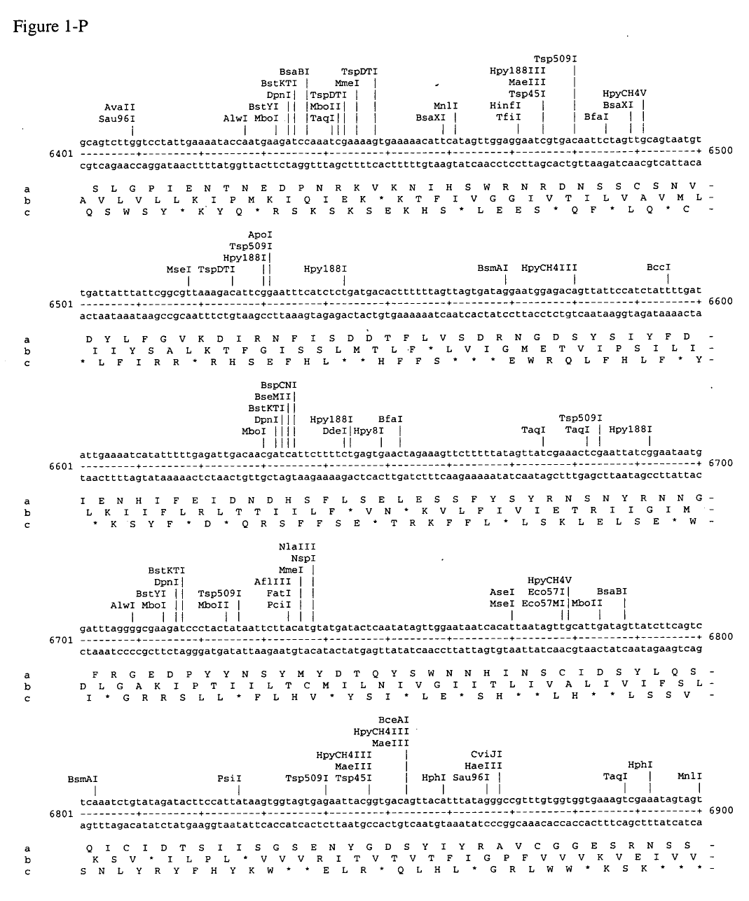

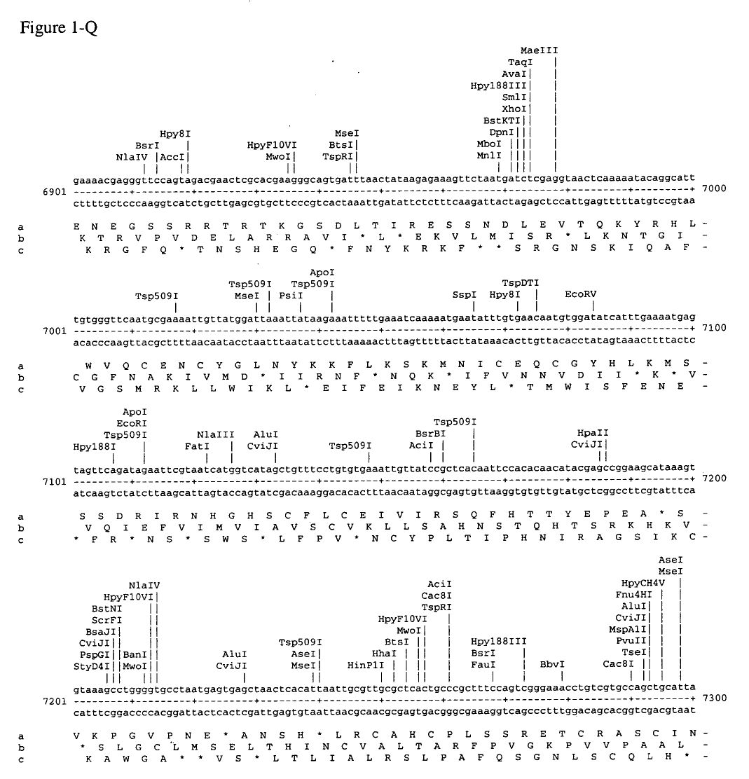

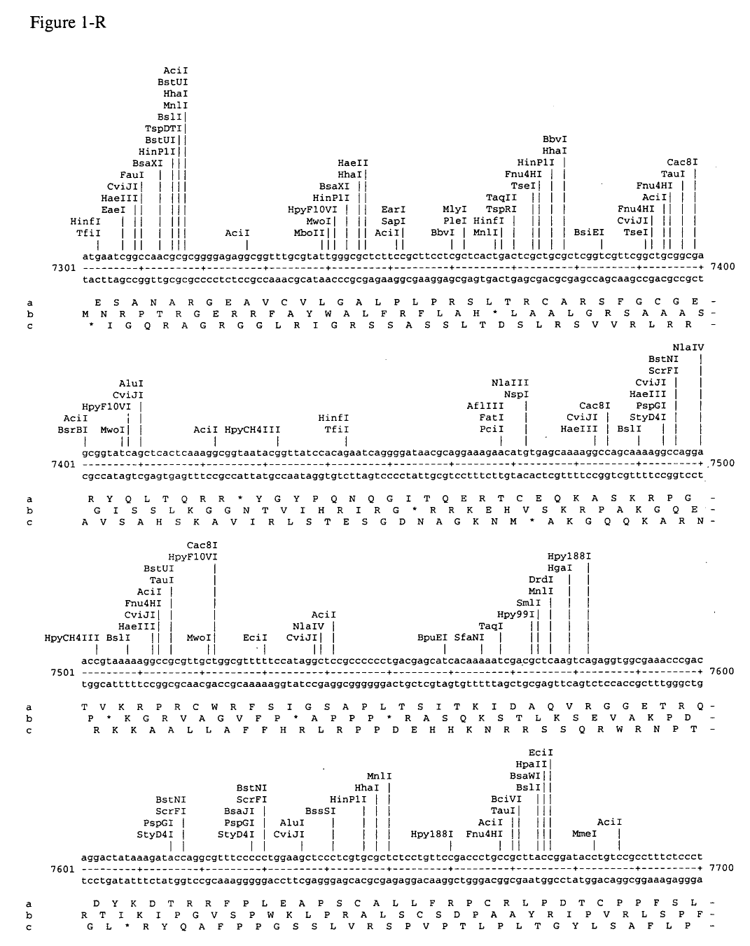

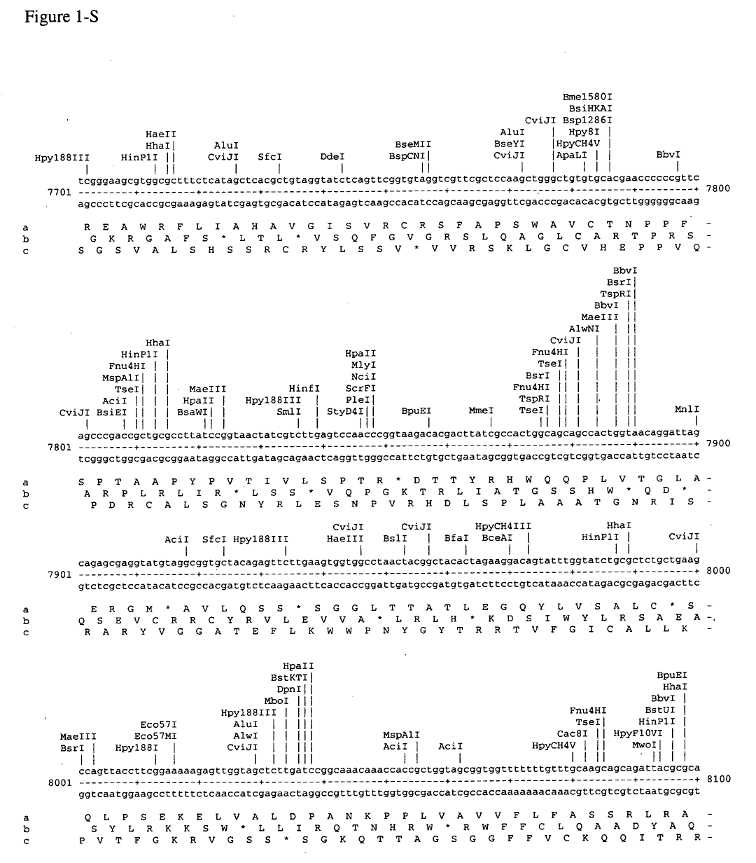

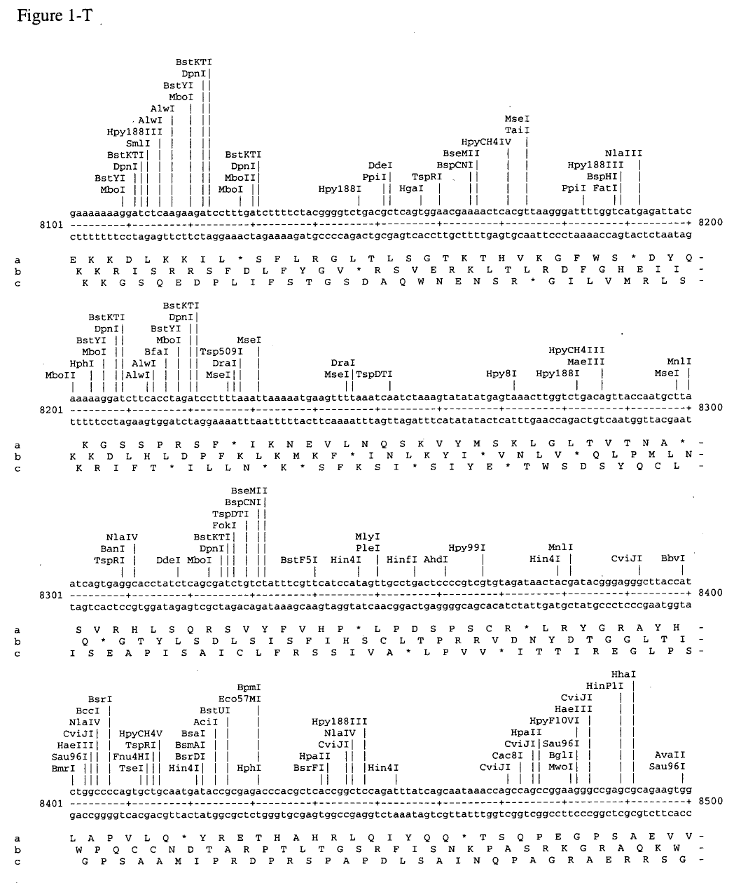

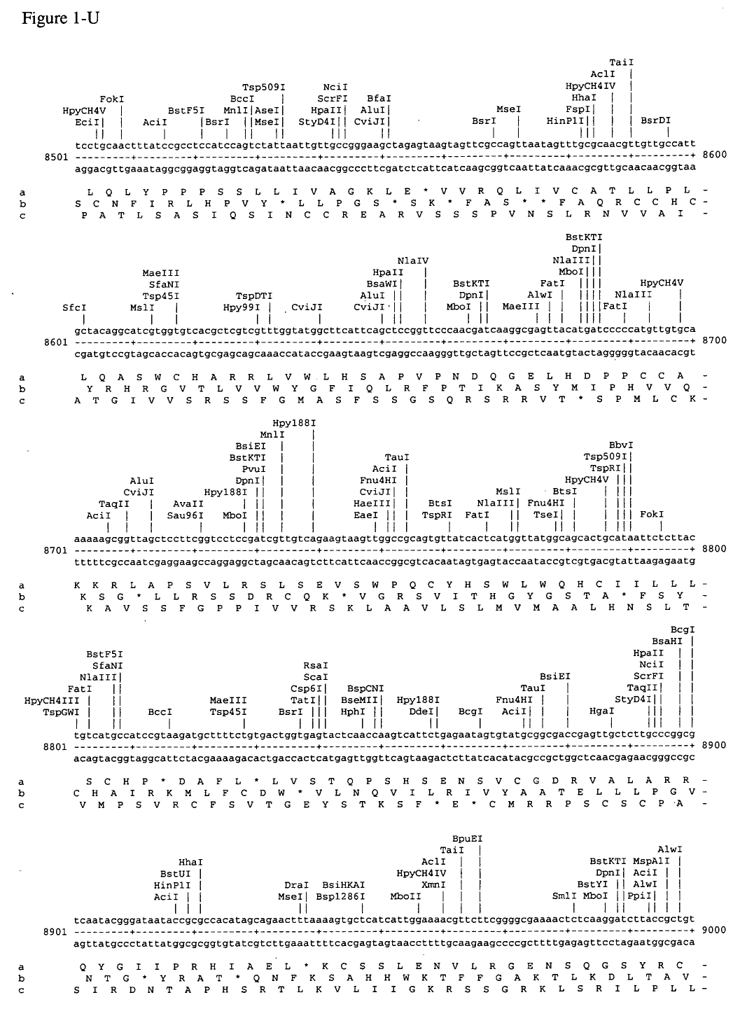

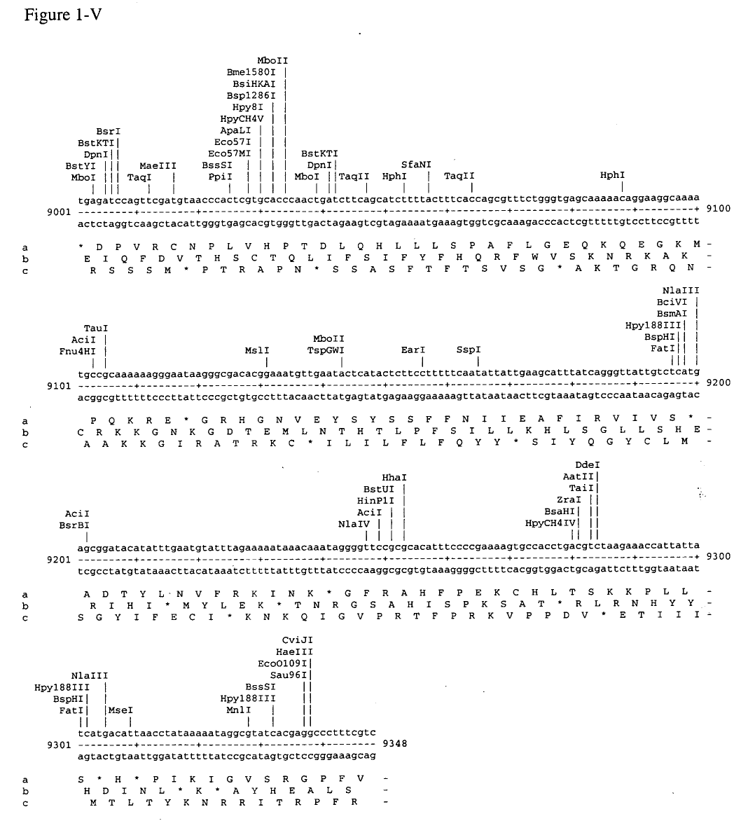

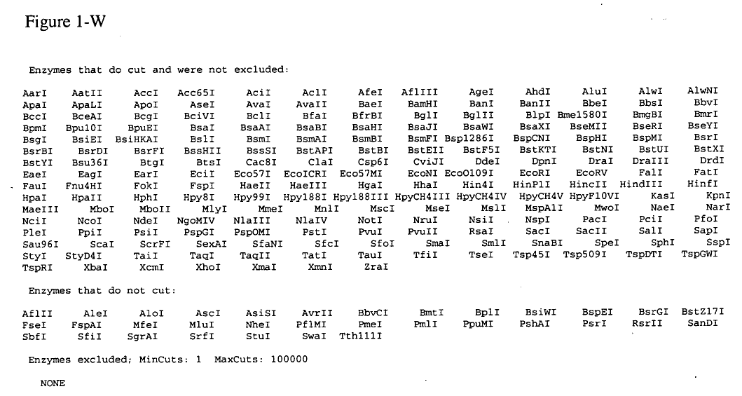

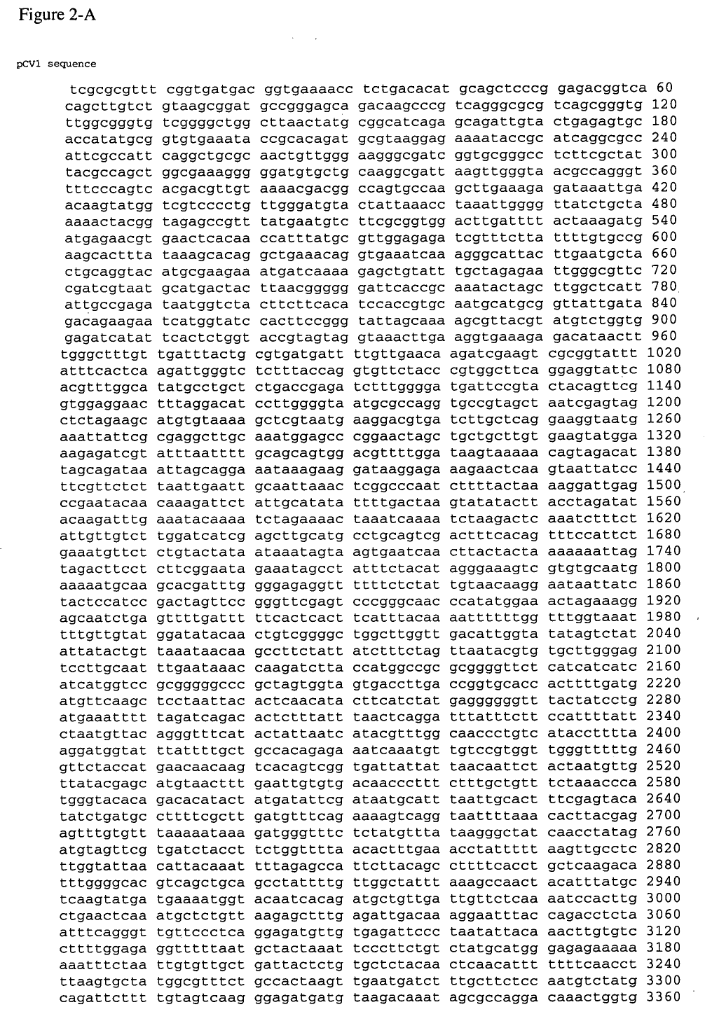

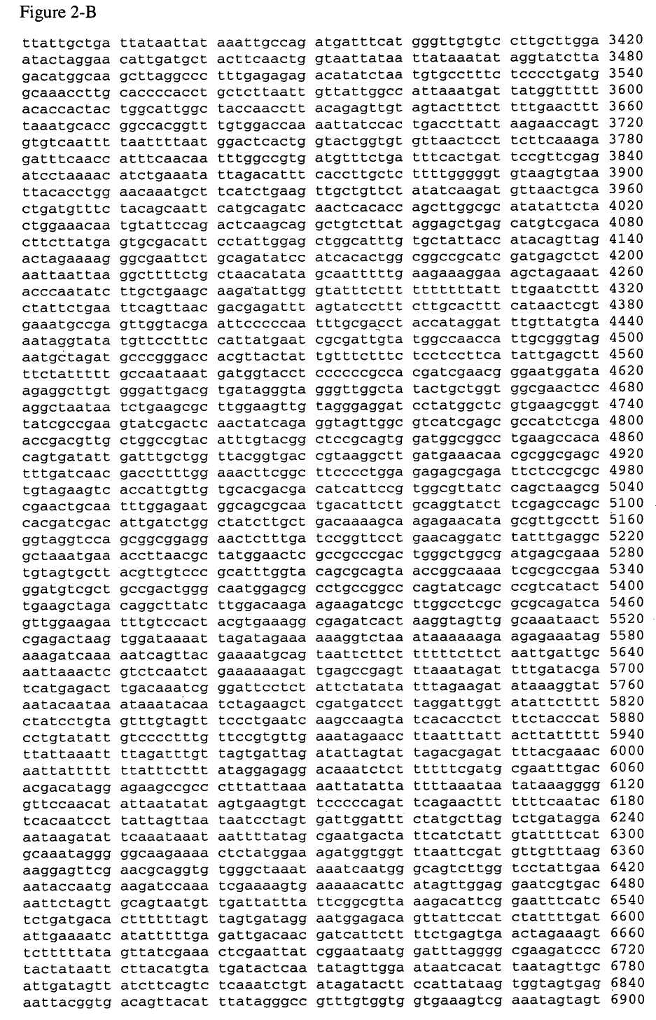

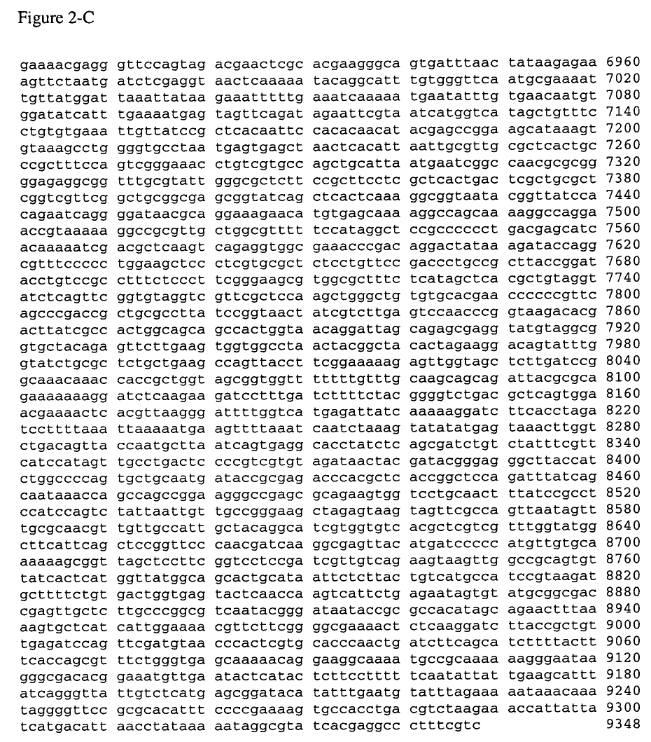

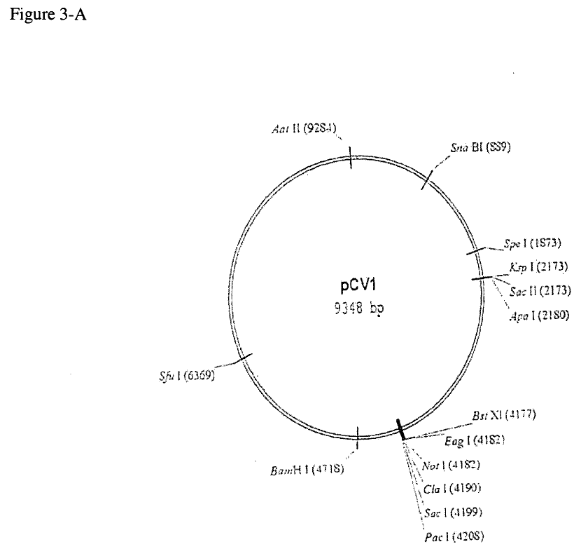

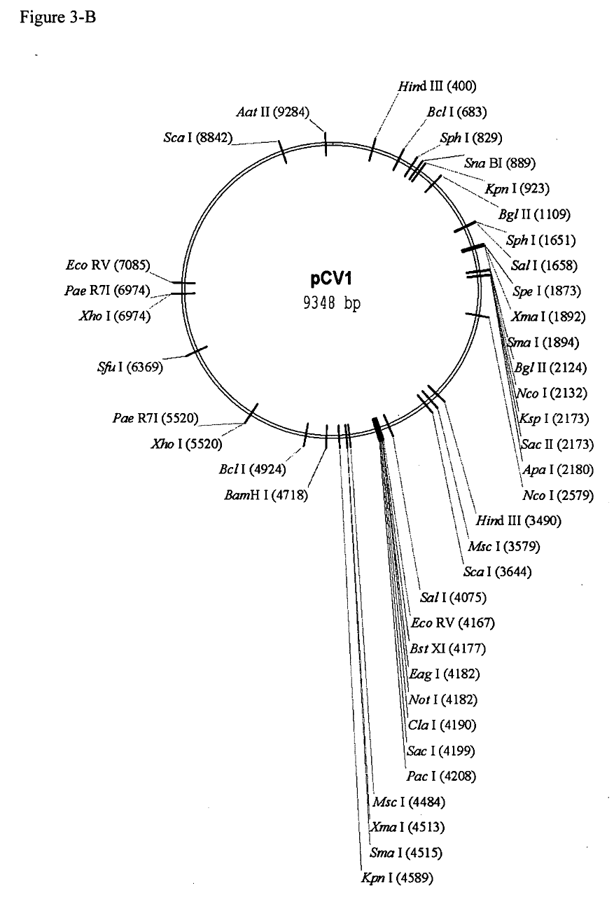

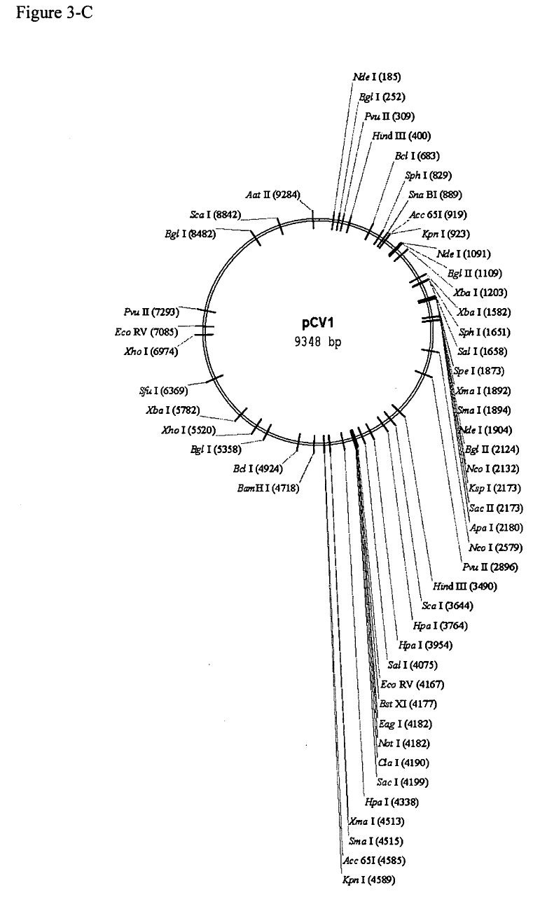

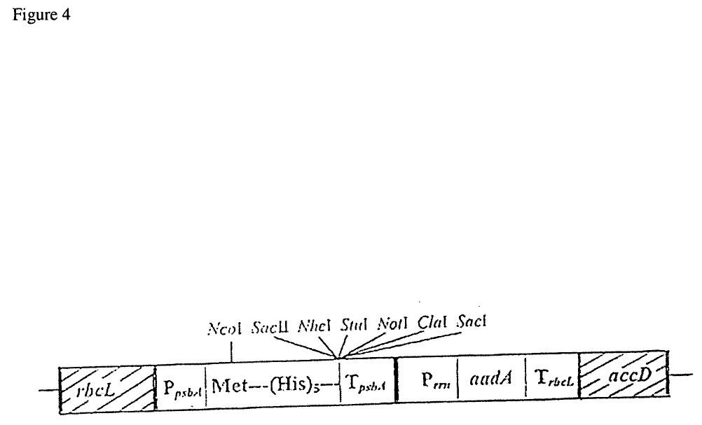

The 2-kb SpeI-NotI fragment encoding S1 minus its start codon and signal peptide from pCRII-S1 was cloned into the NheI and NotI sites of pMLVHisA (FIG. 4). The plasmid pCRII-S1 was derived from pCRII (Invitrogen) and contains a PCR-amplified fragment of S1. In the resultant derivative, designated pCV1 (FIG. 1), amino acids 12-658 of S1 are fused in-frame to seventeen pMLVHisA-derived residues, including the ‘ATG’ start codon and a (His)₅-tag. Plasmid pMLVHisA is designed with a (His)₅-tag enabling recognition of the recombinant protein using antisera against the (His)₅-tag in western blot analysis. Also, the (His)₅-tag enables easy purification of the recombinant protein, if and when required, using Ni-NTA Agarose (Qiagen) affinity columns. The rice plastid psbA promoter drives expression of the recombinant protein and the terminator is also psbA-derived. The presence of flanking rbcL and accD sequences from the tobacco plastid genome enables homologous recombination to occur, resulting in the incorporation of S1 (and aadA) into the plastid genome of the target plant.

Since plant chloroplast genome sequences are highly-conserved, the same construct was used for plastid transformation of tobacco and tomato, both belonging to the same family Solanaceae.

Construction of pCV2, a Nuclear Transformation Vector for Expression of the S1 Antigen

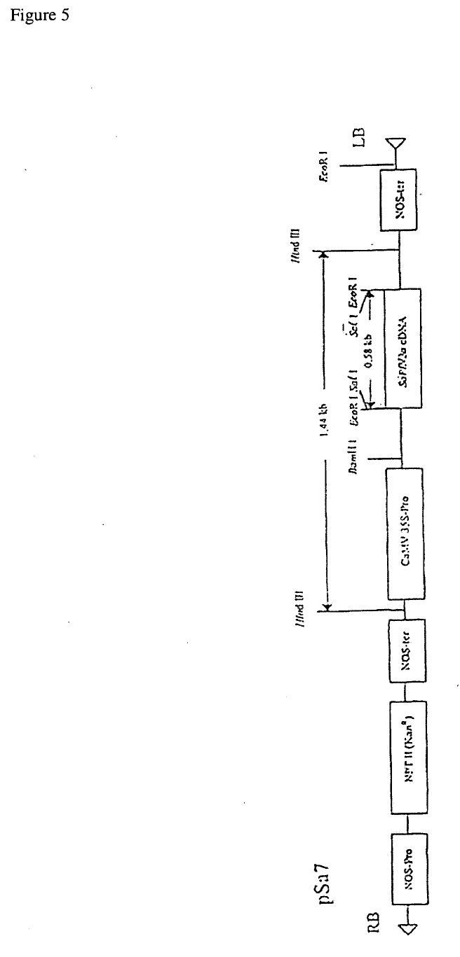

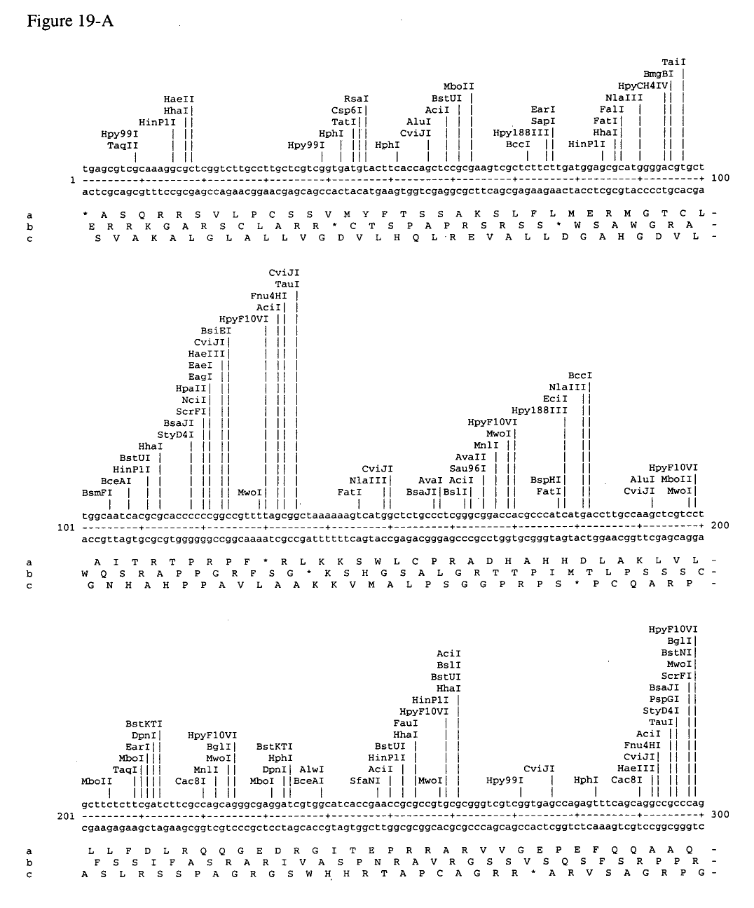

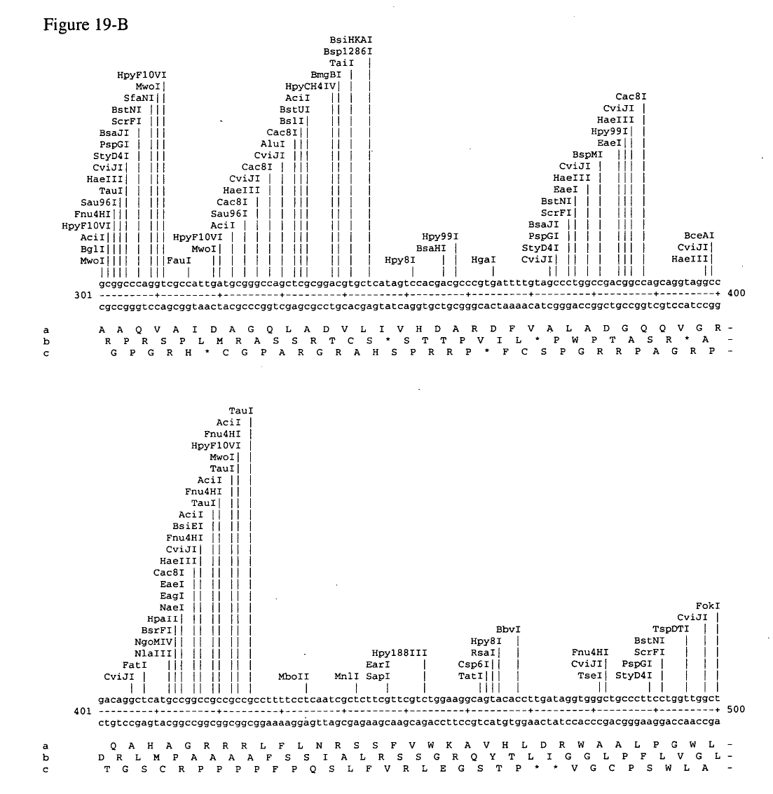

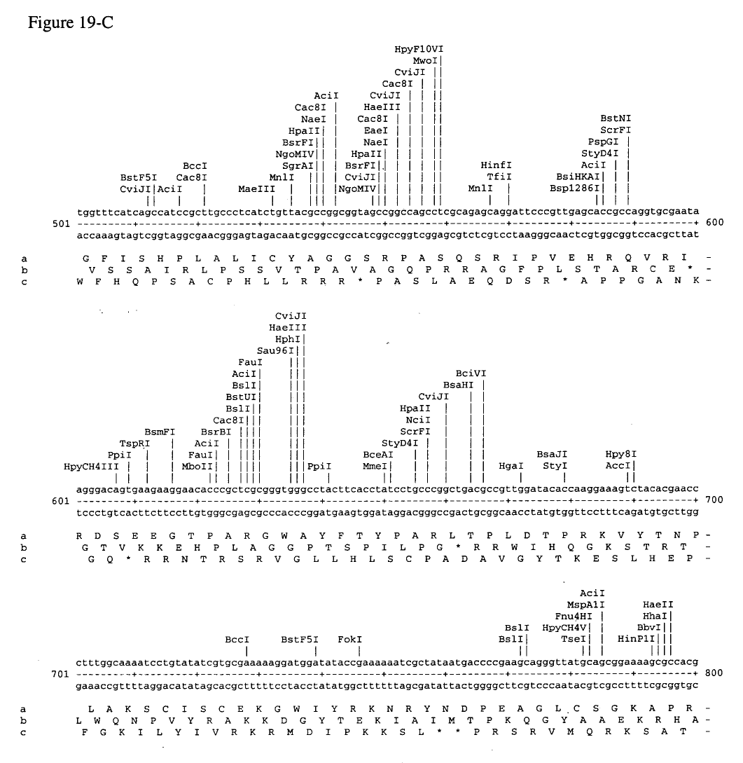

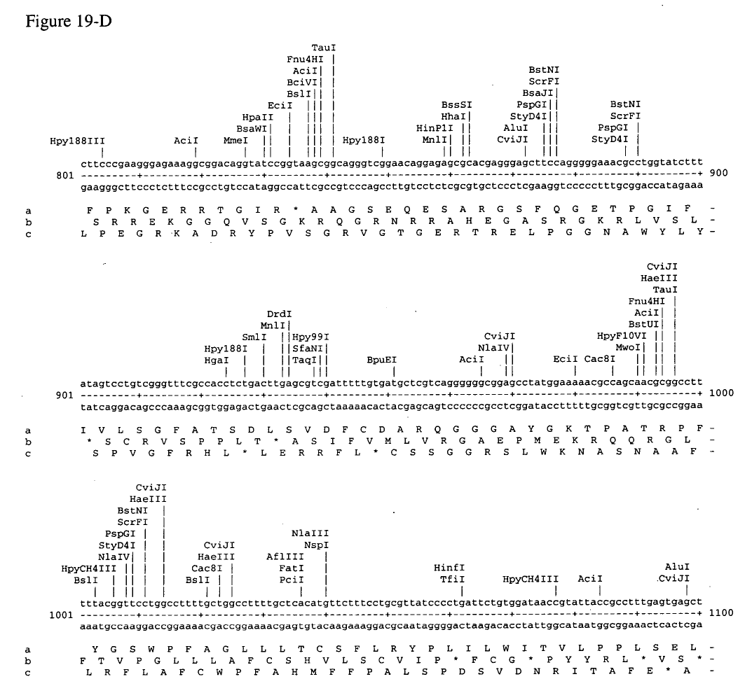

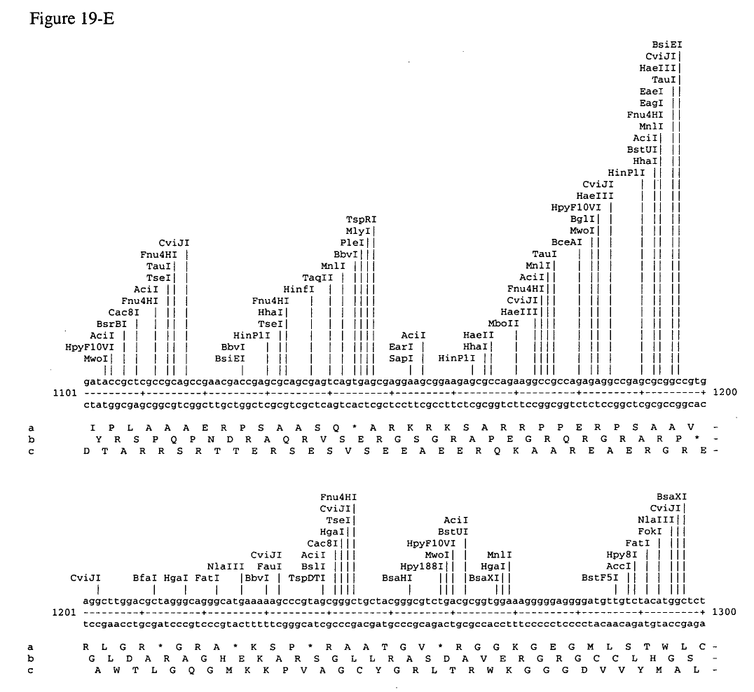

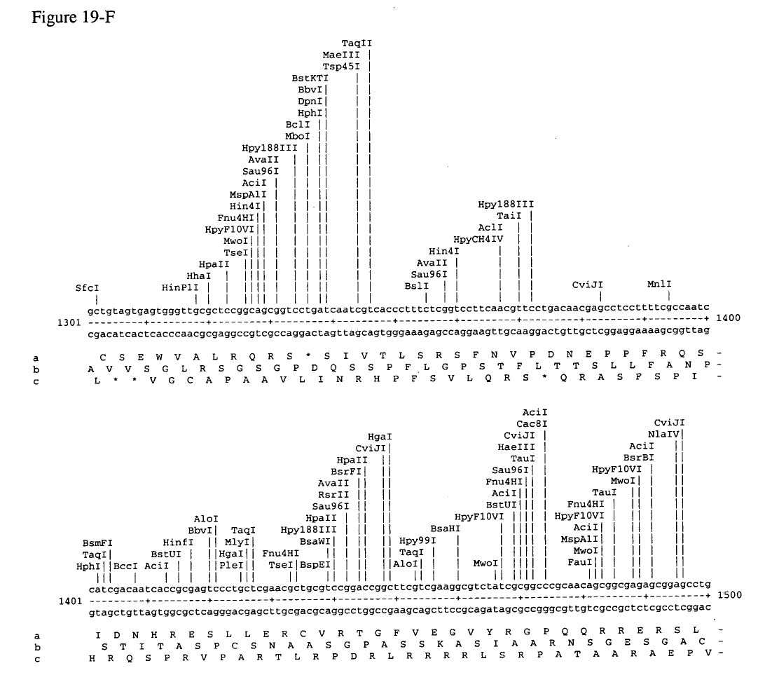

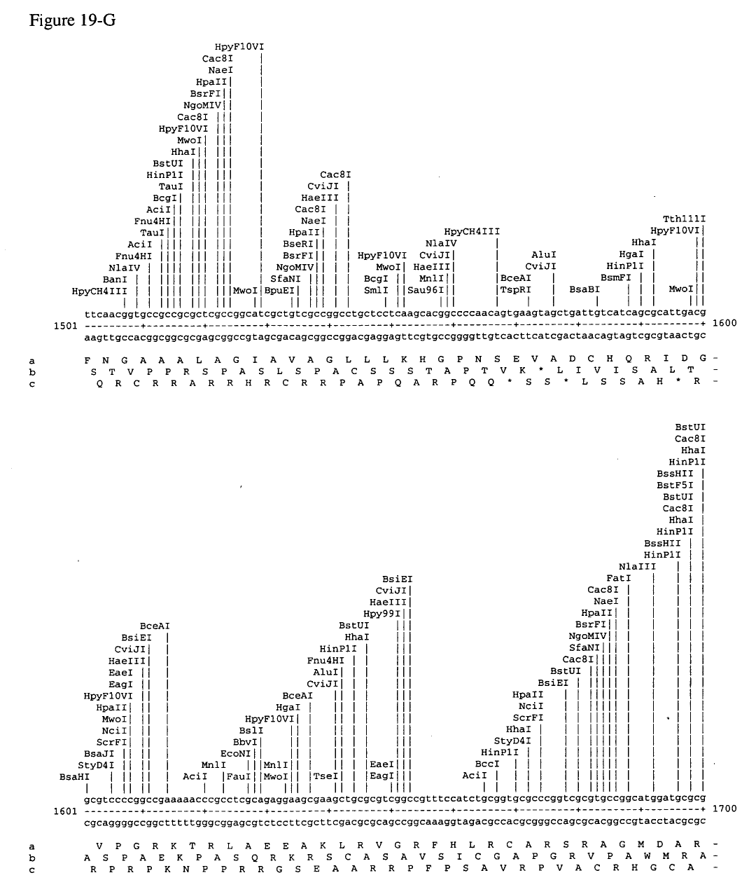

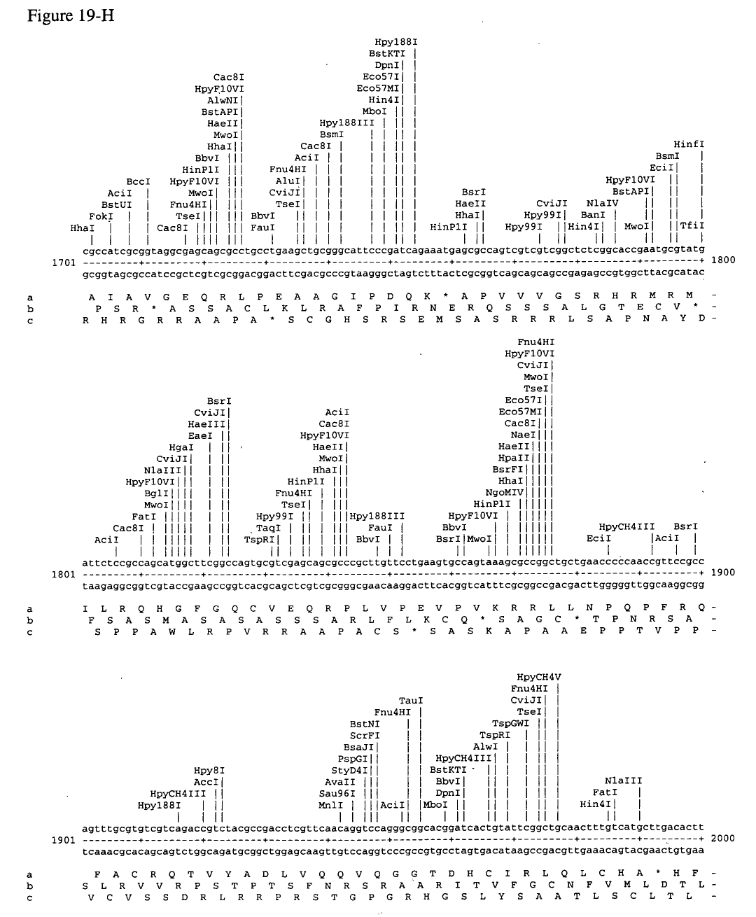

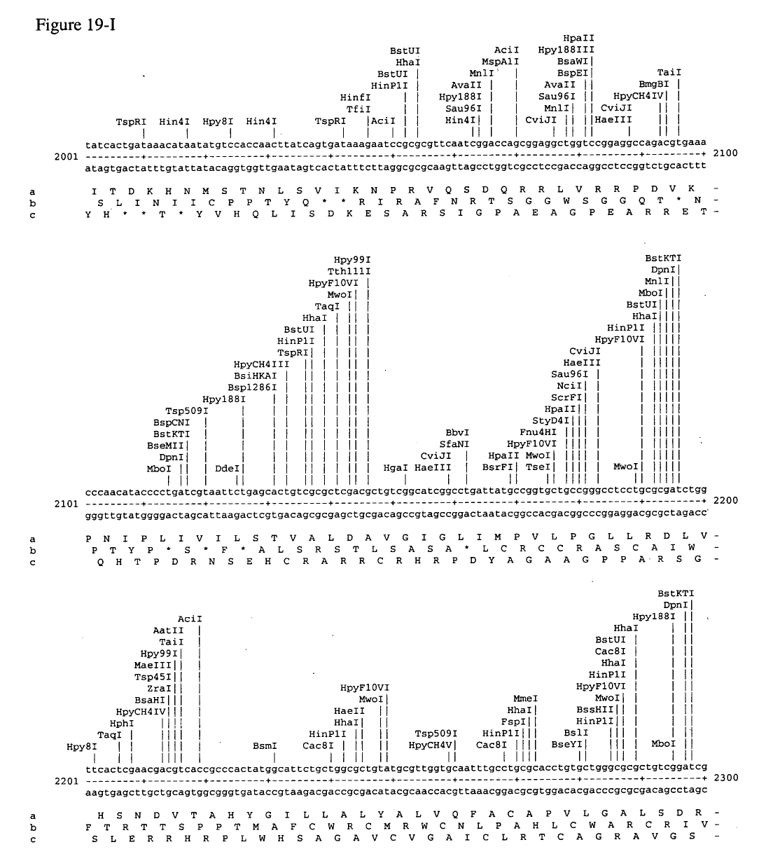

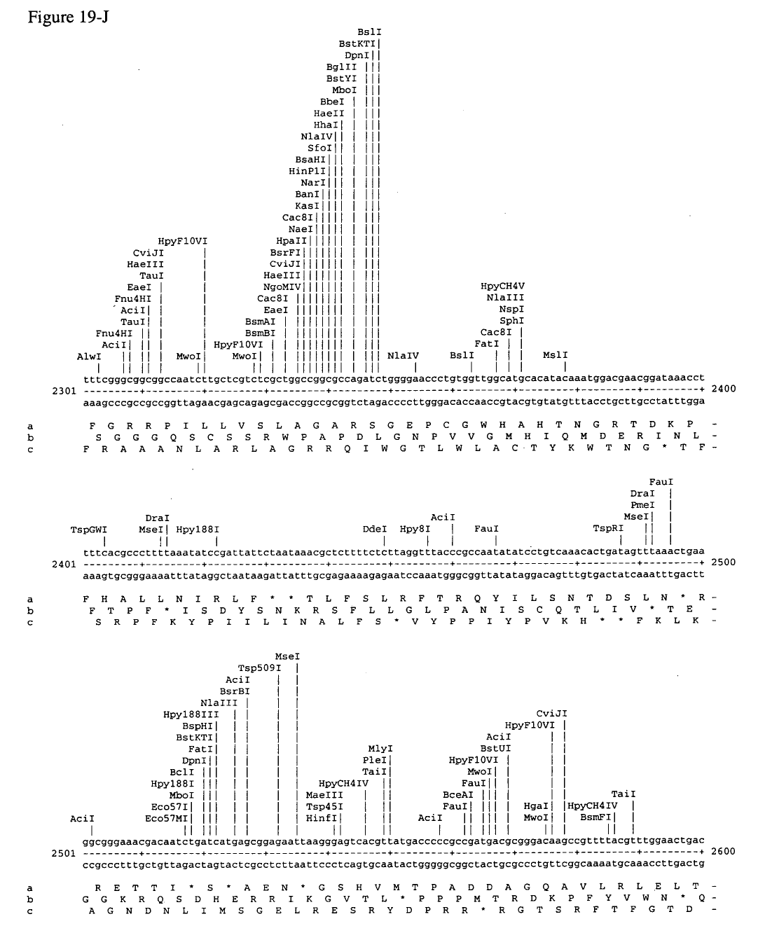

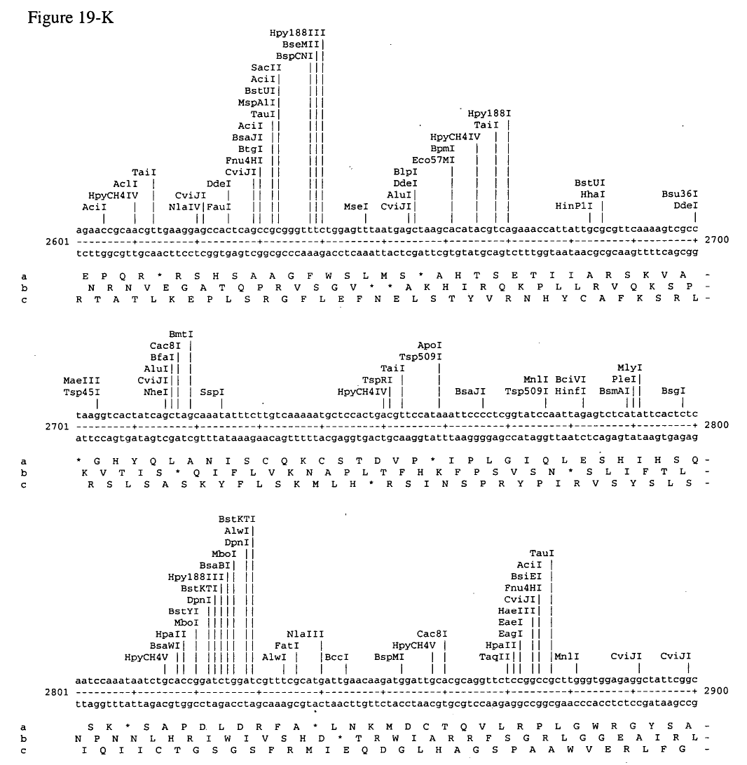

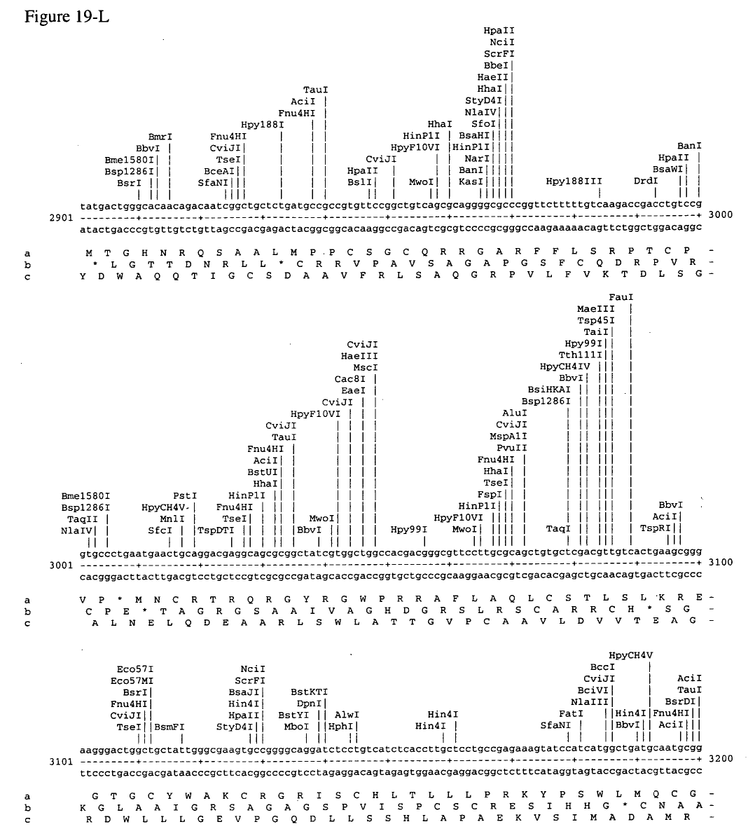

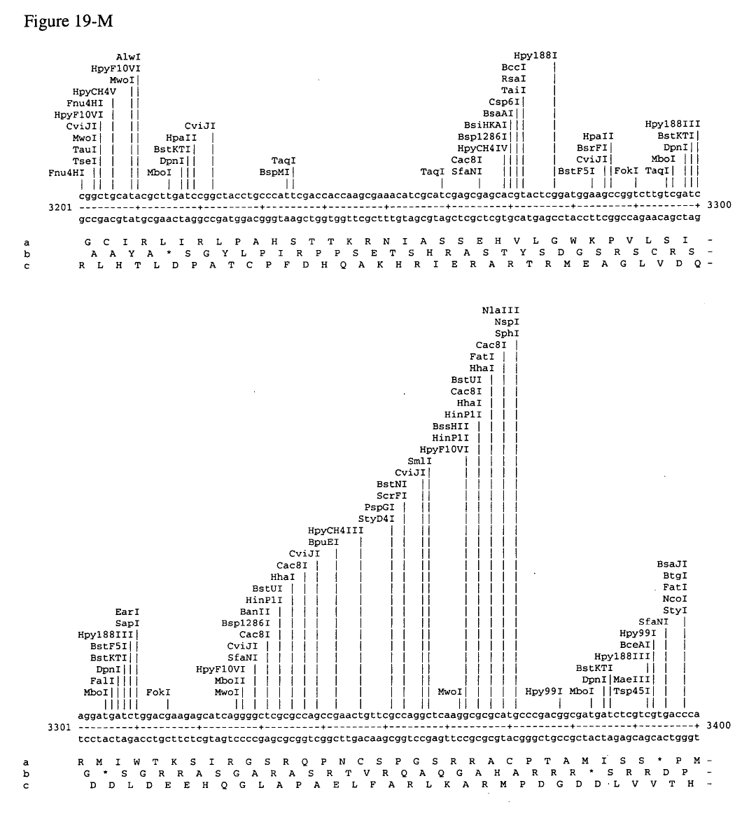

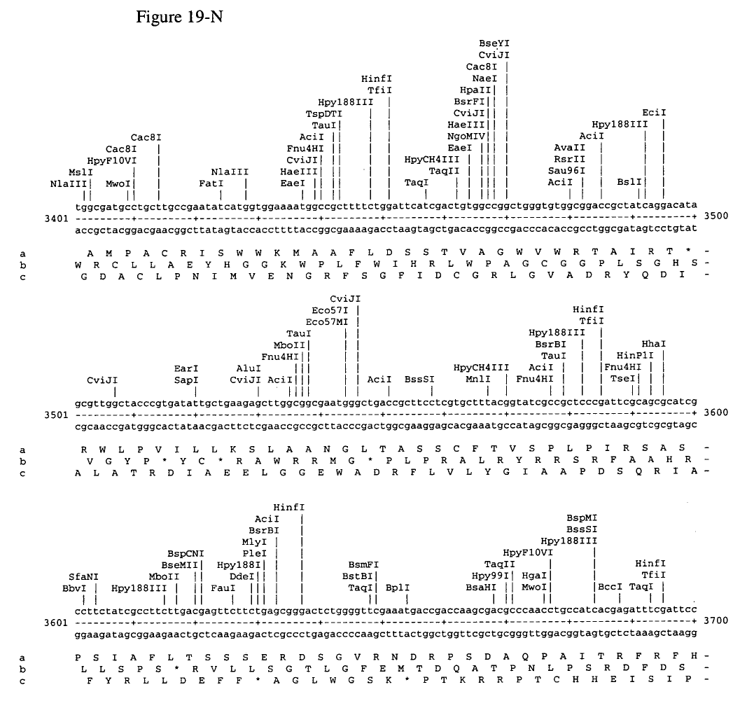

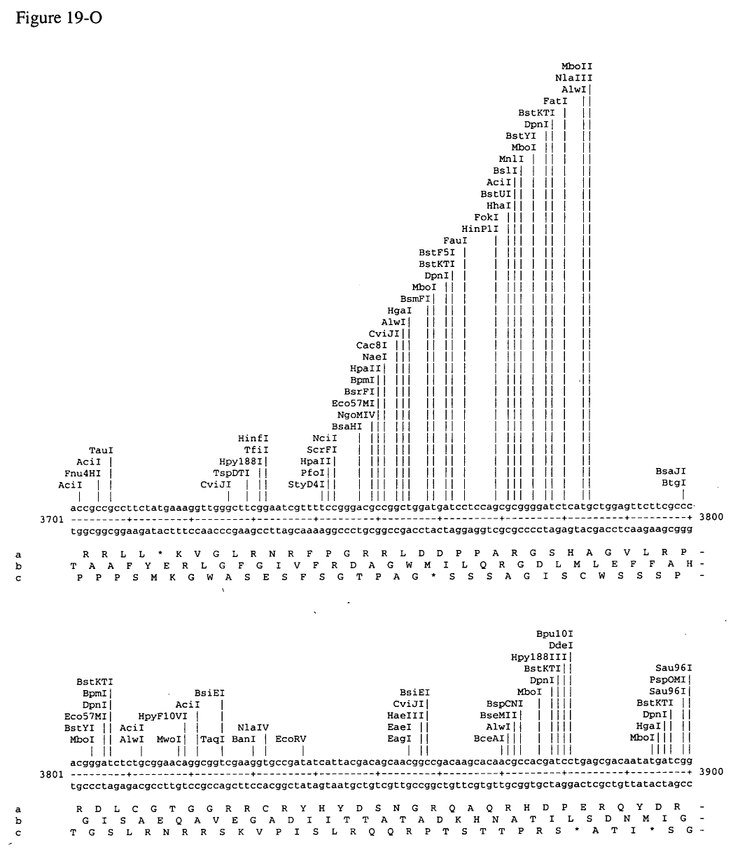

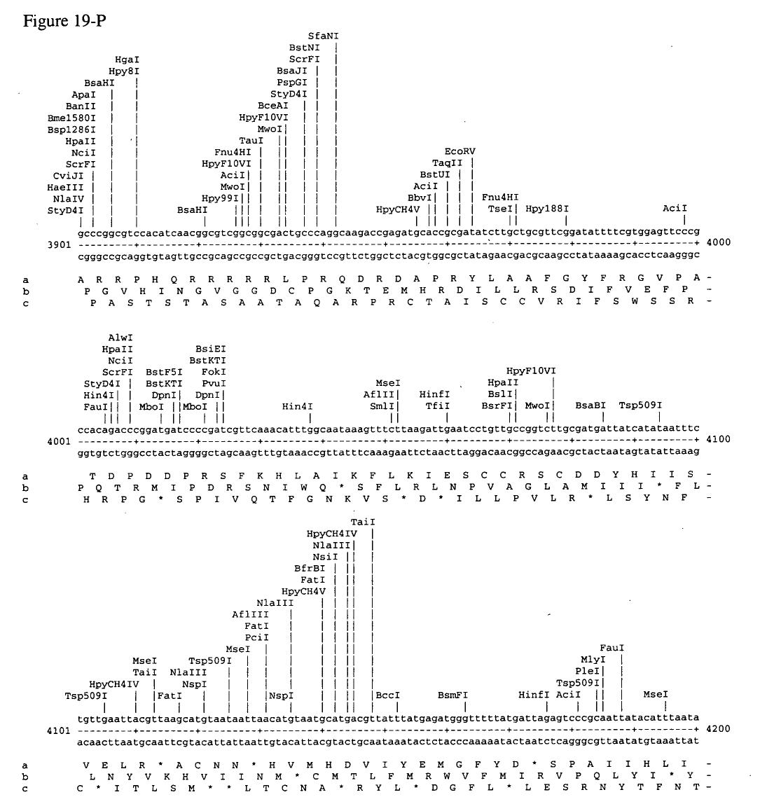

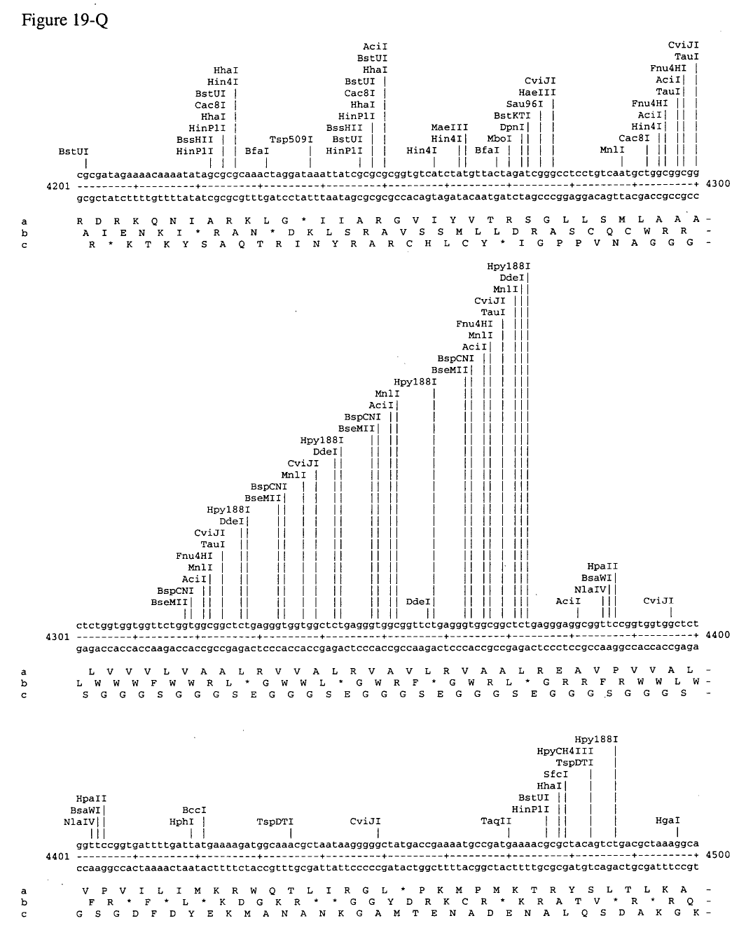

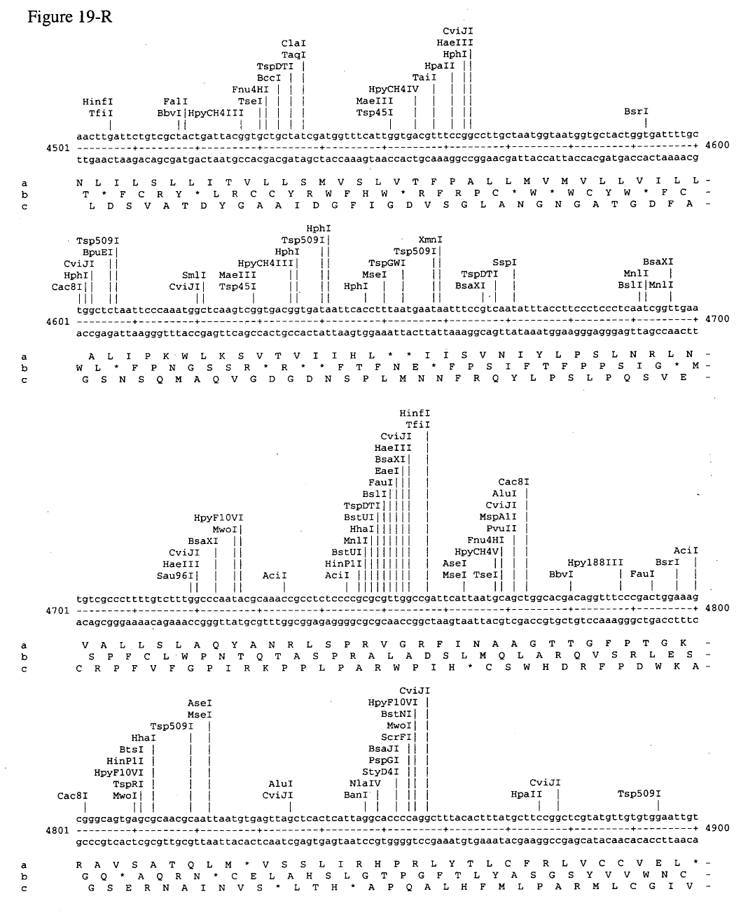

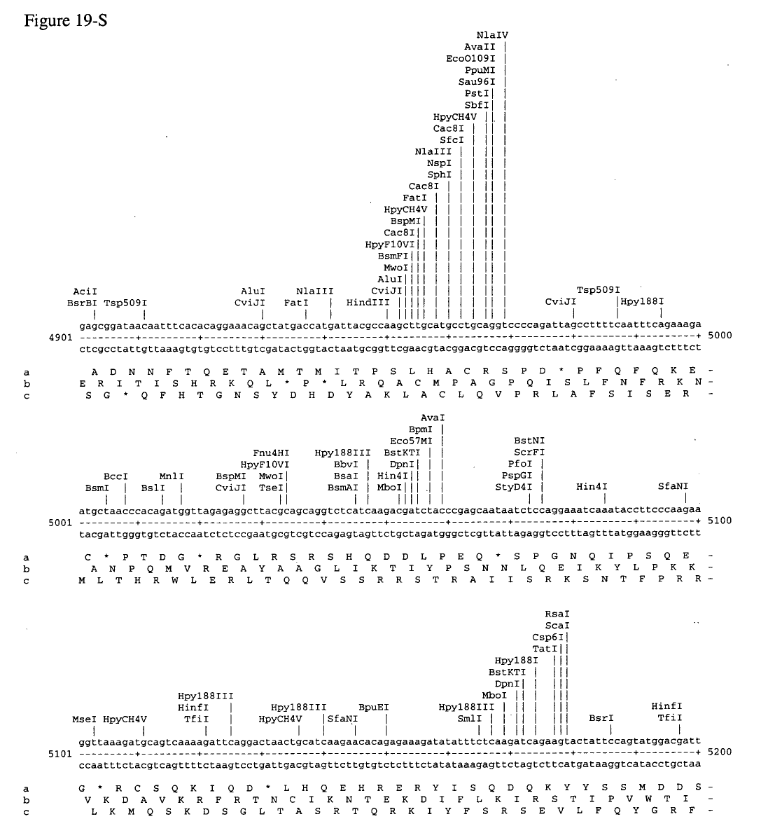

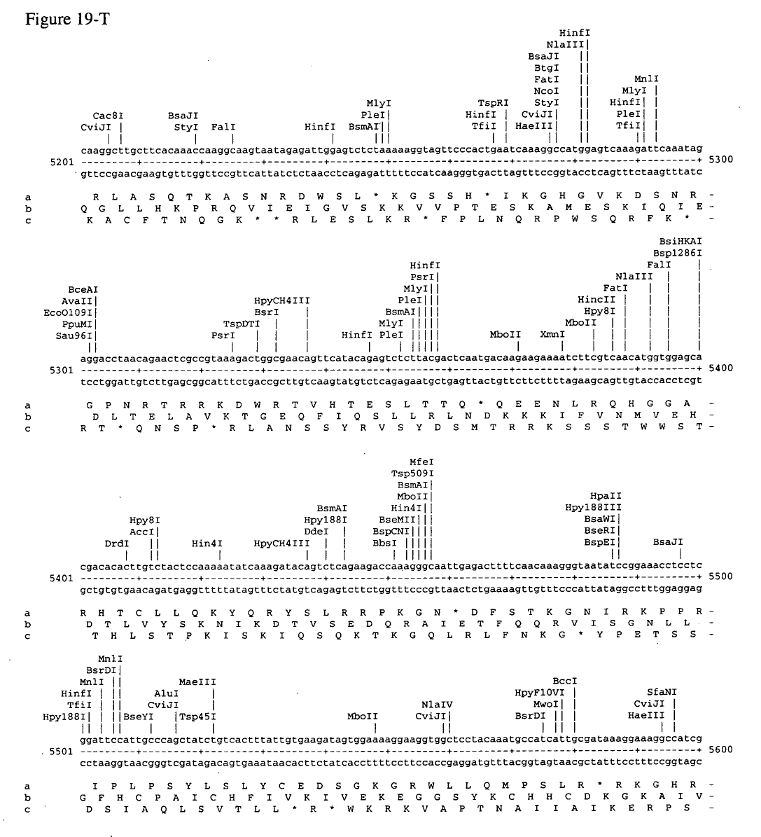

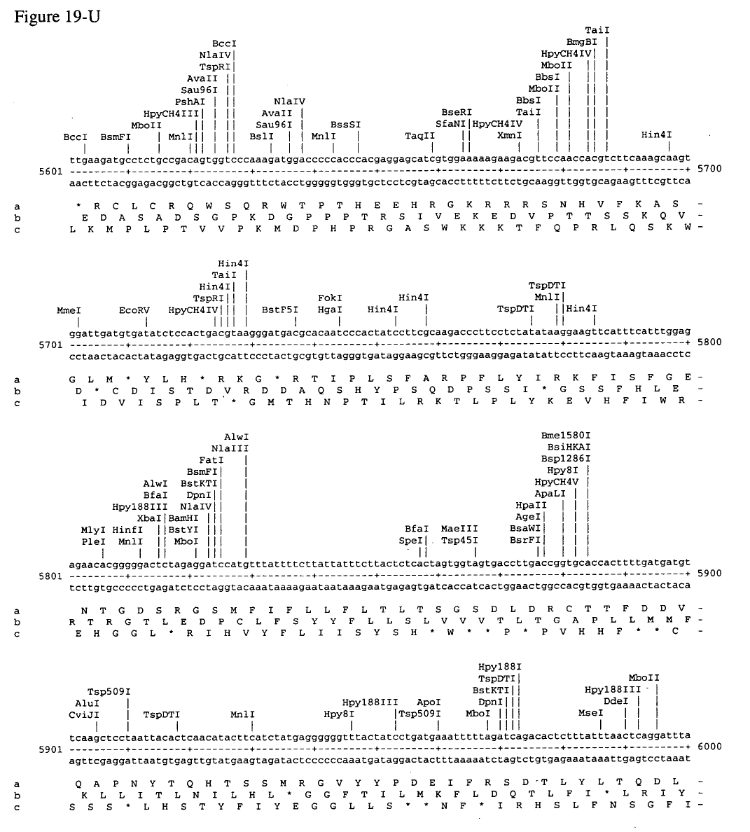

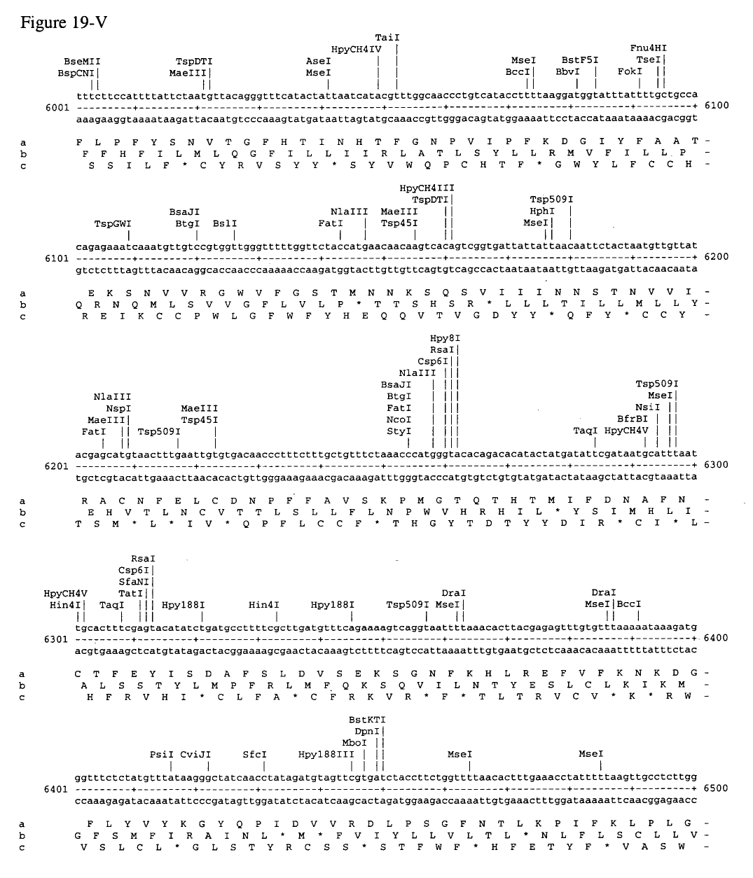

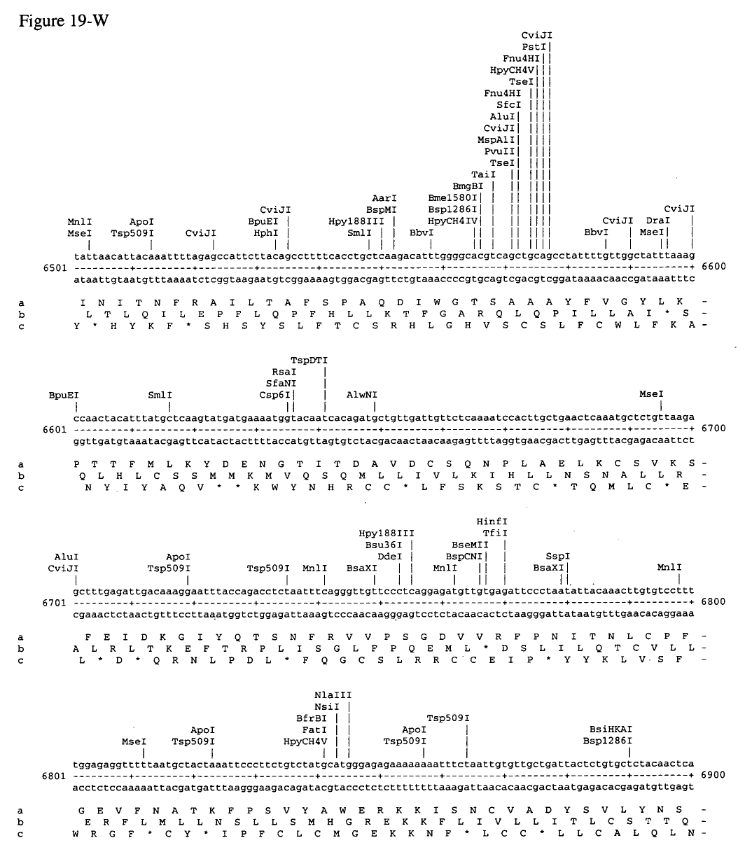

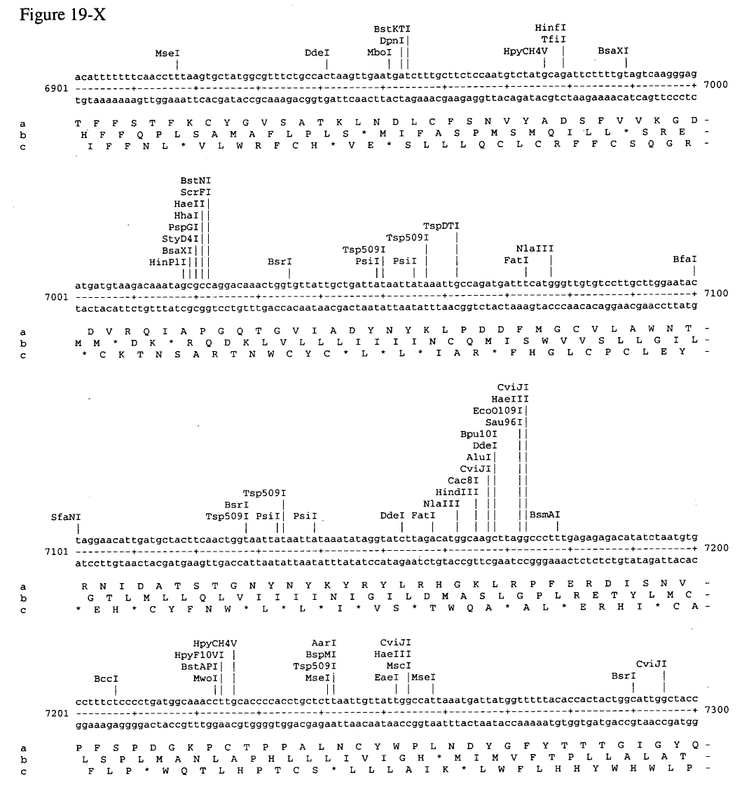

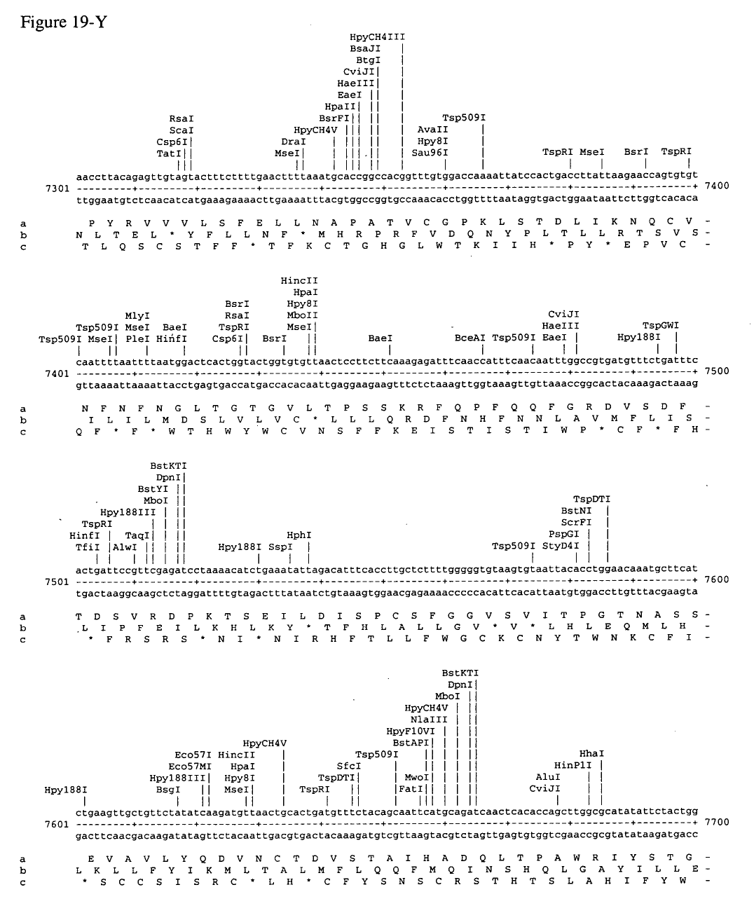

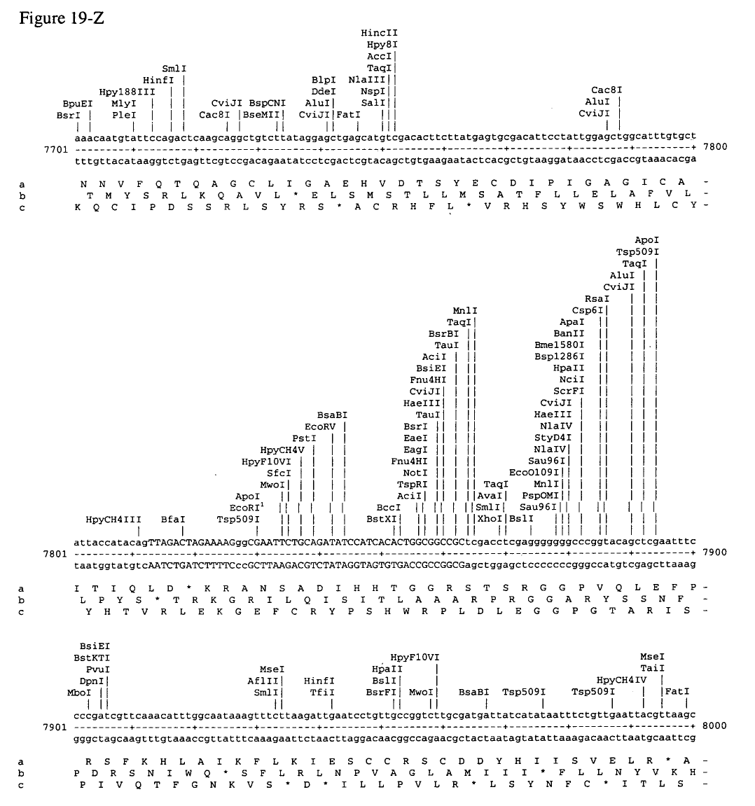

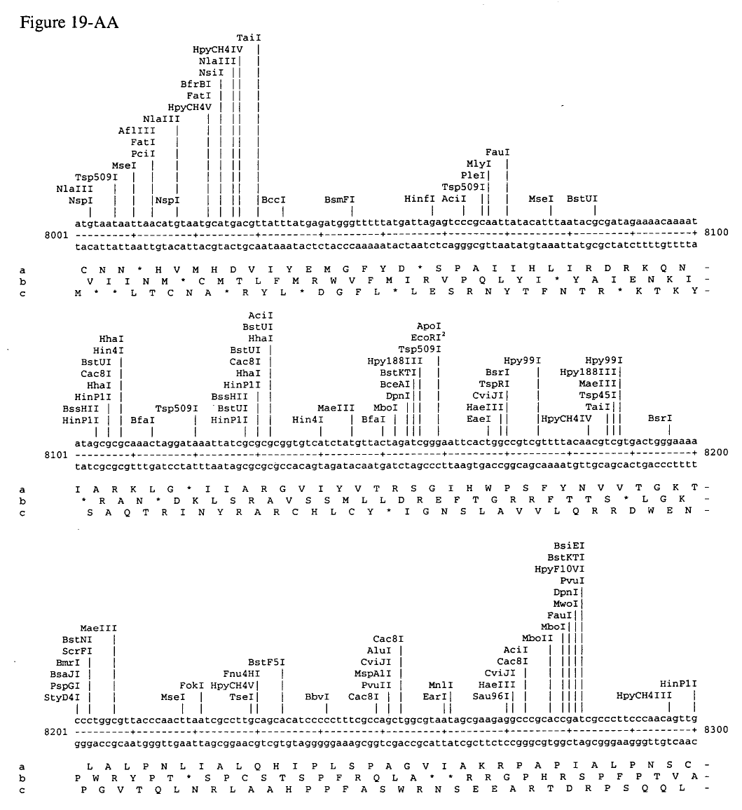

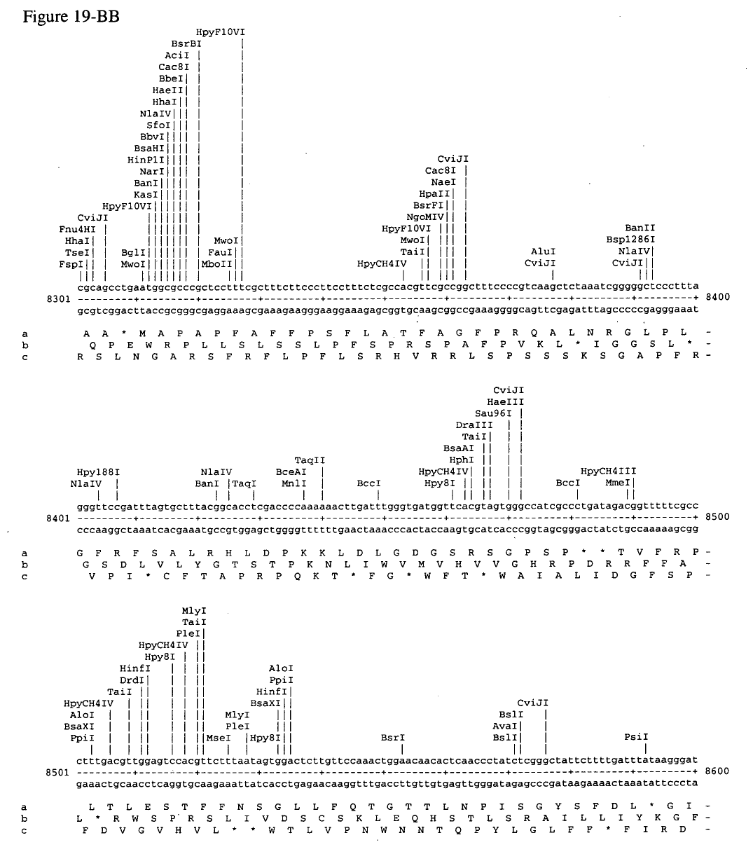

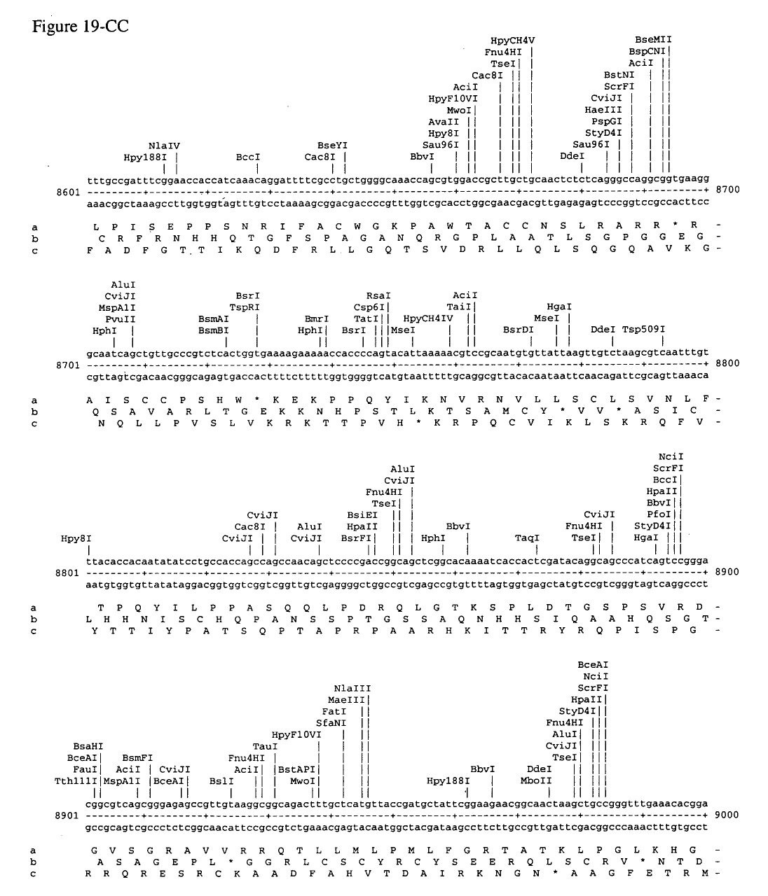

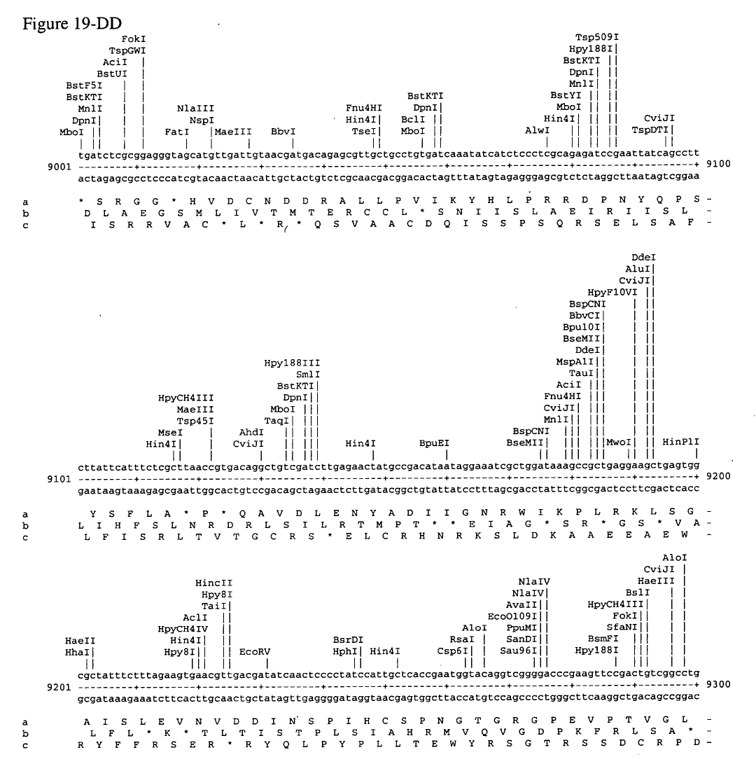

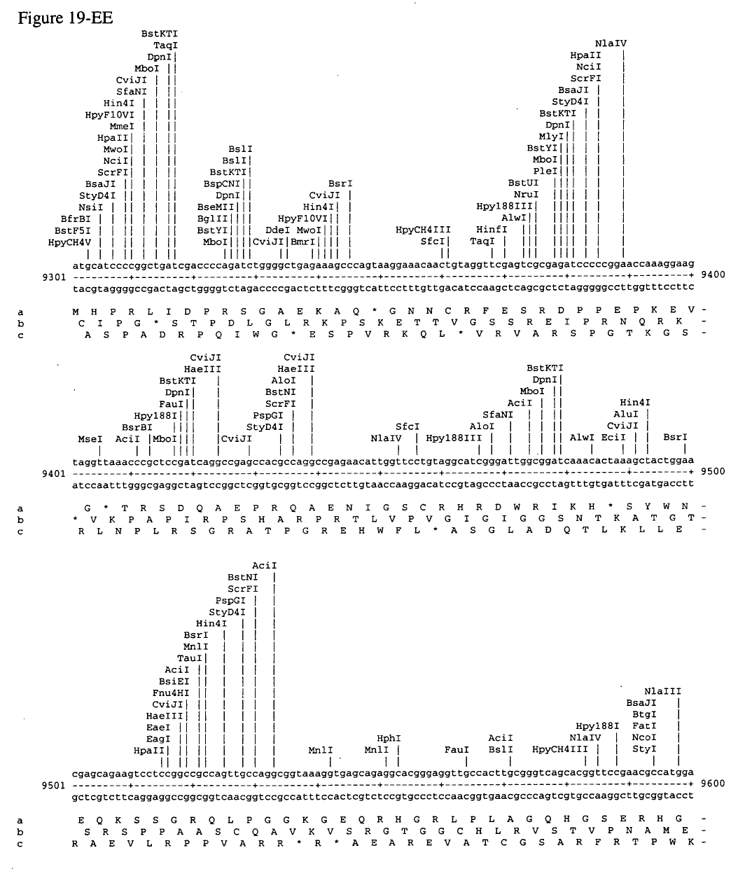

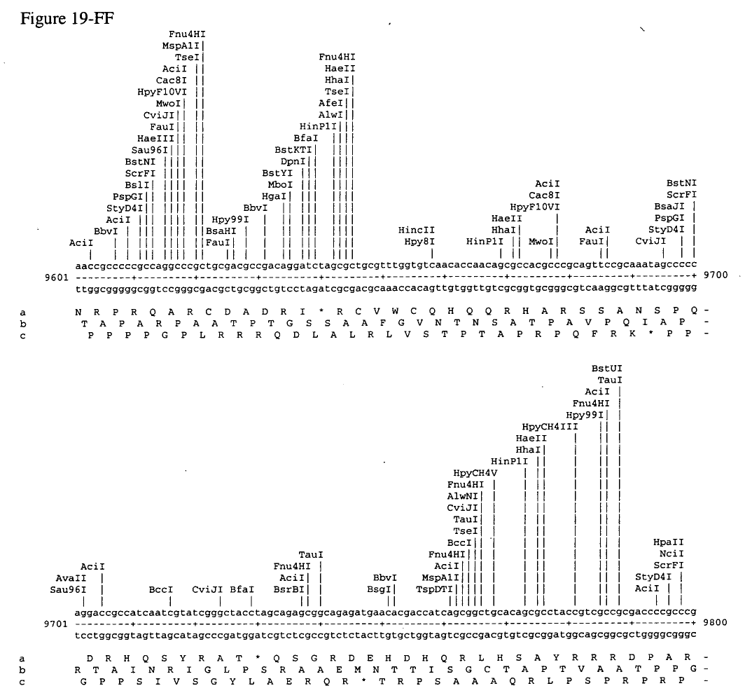

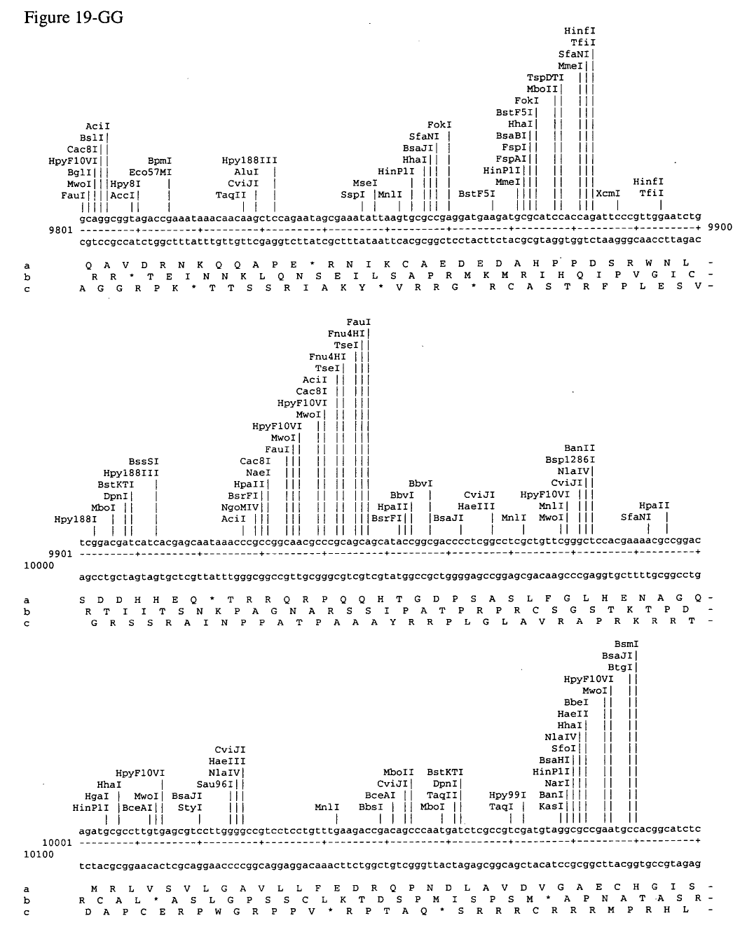

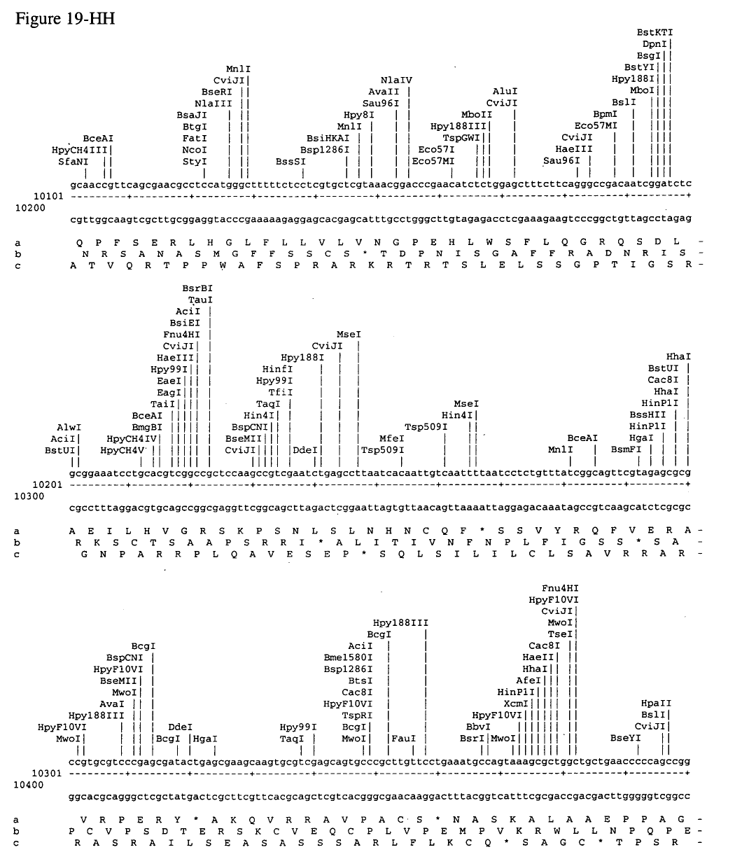

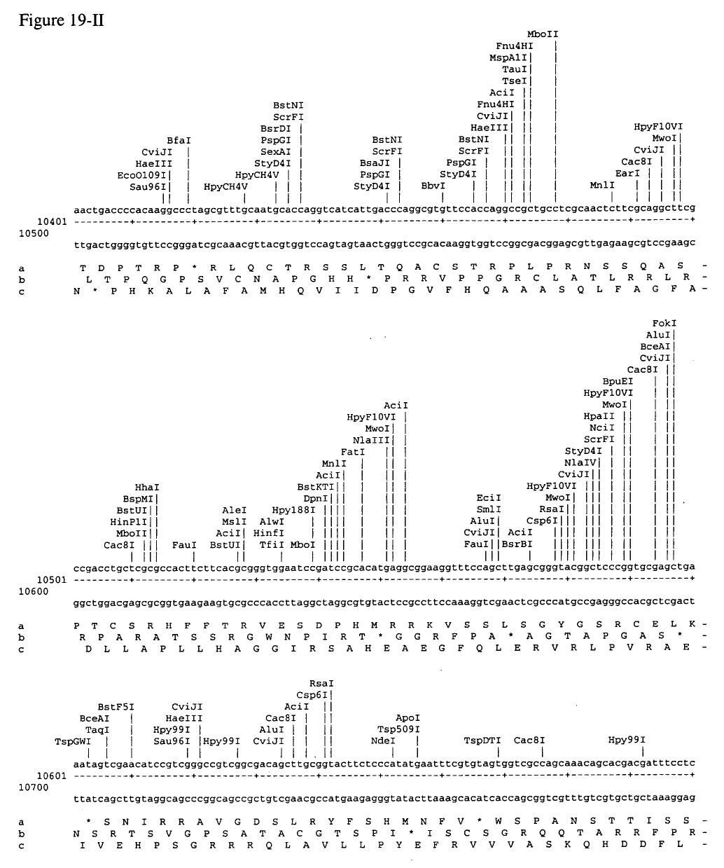

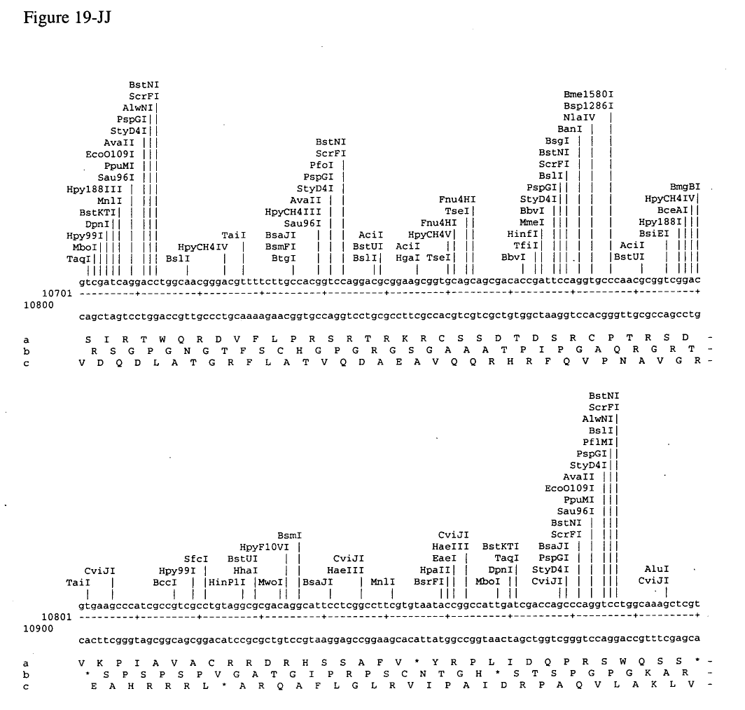

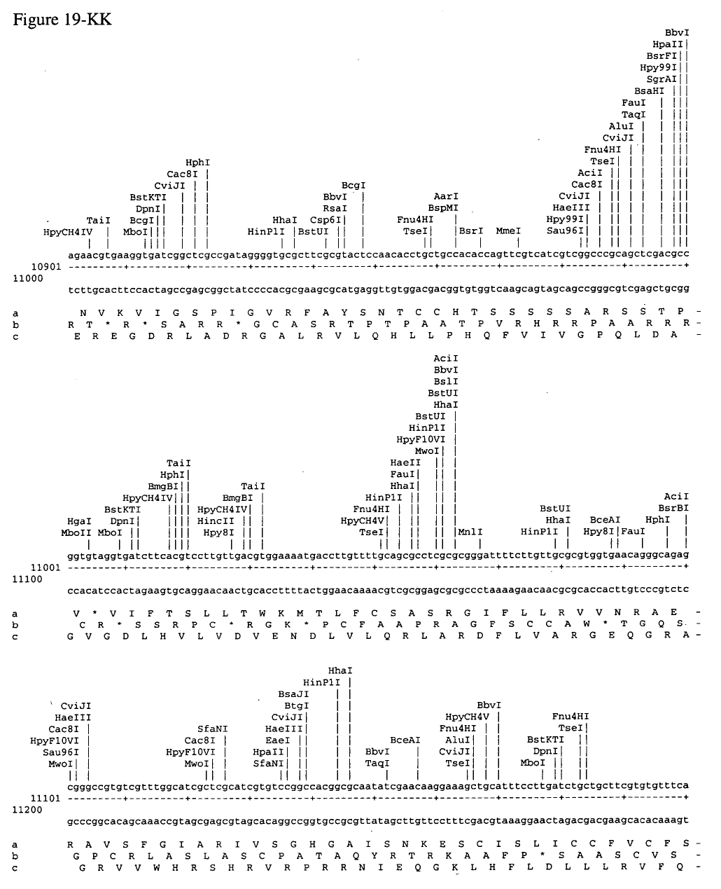

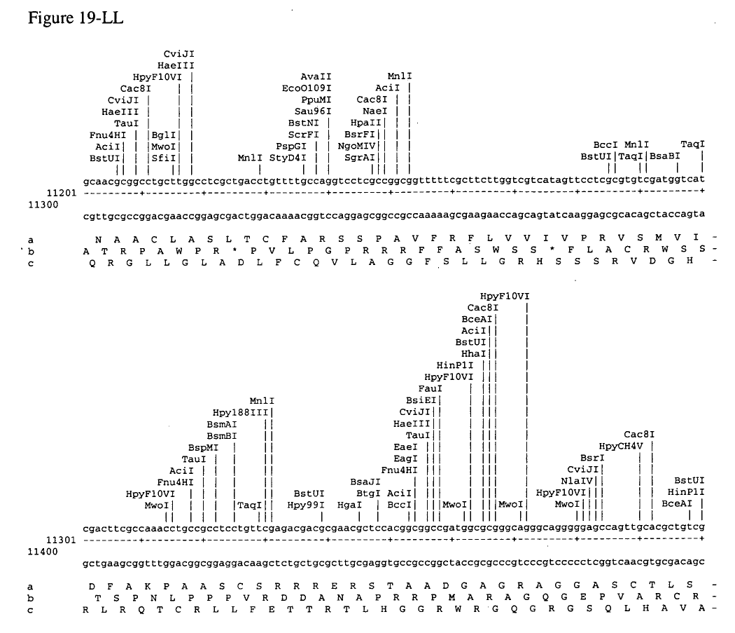

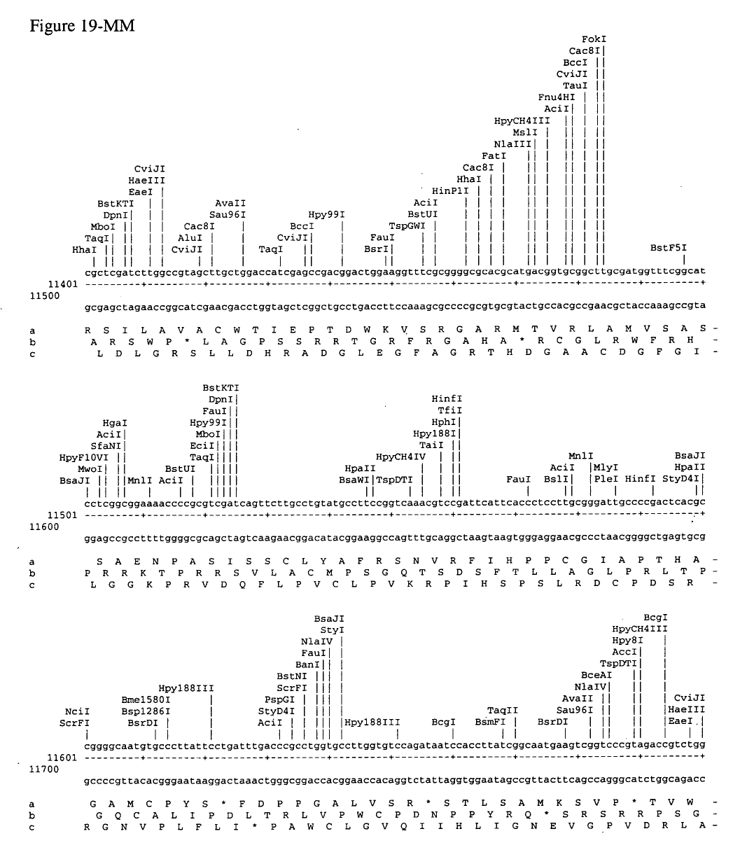

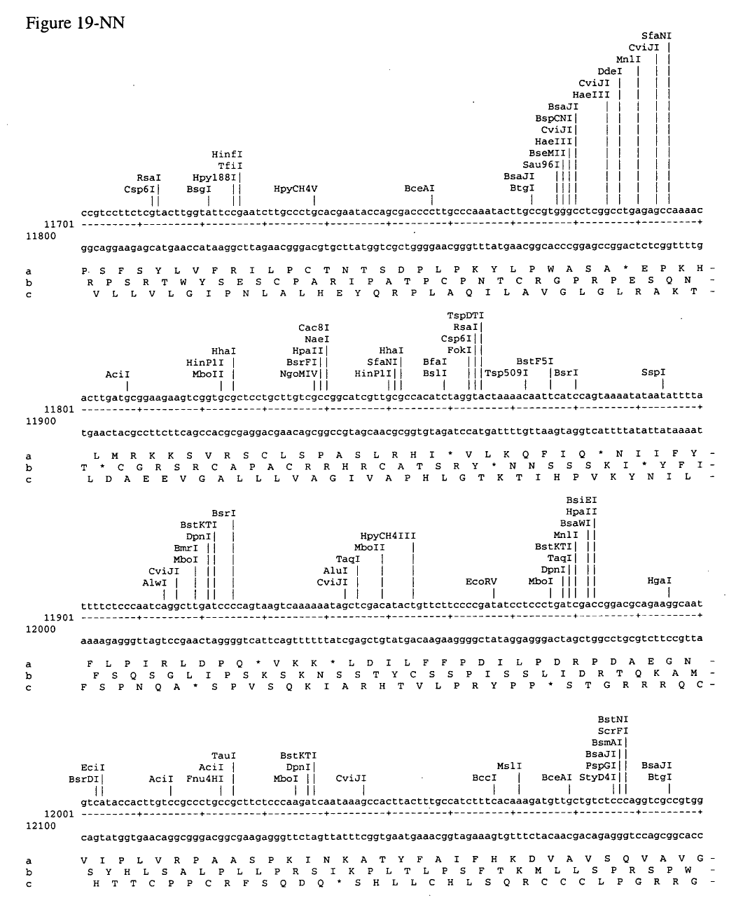

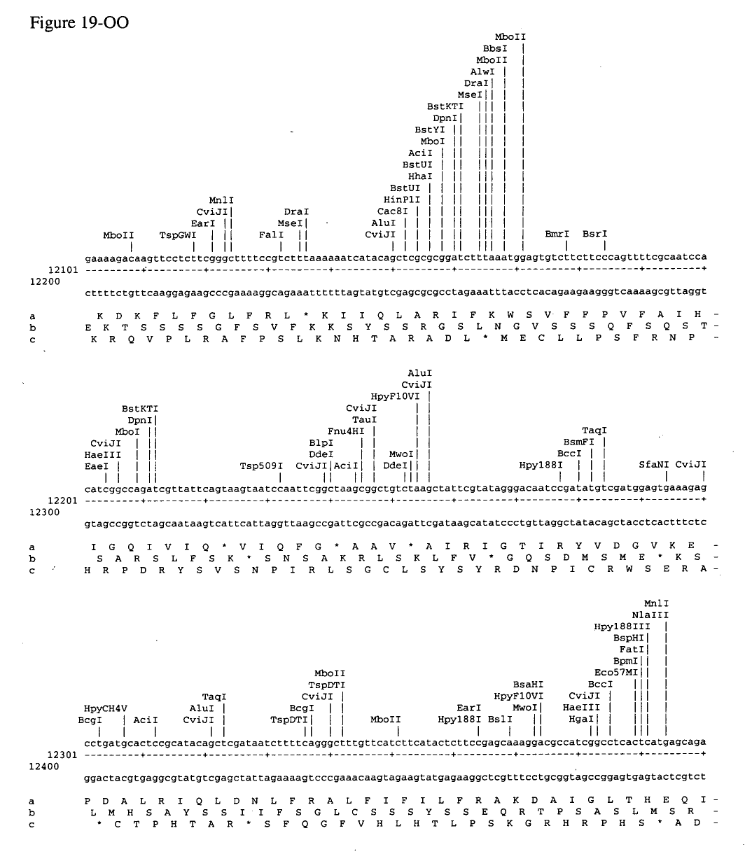

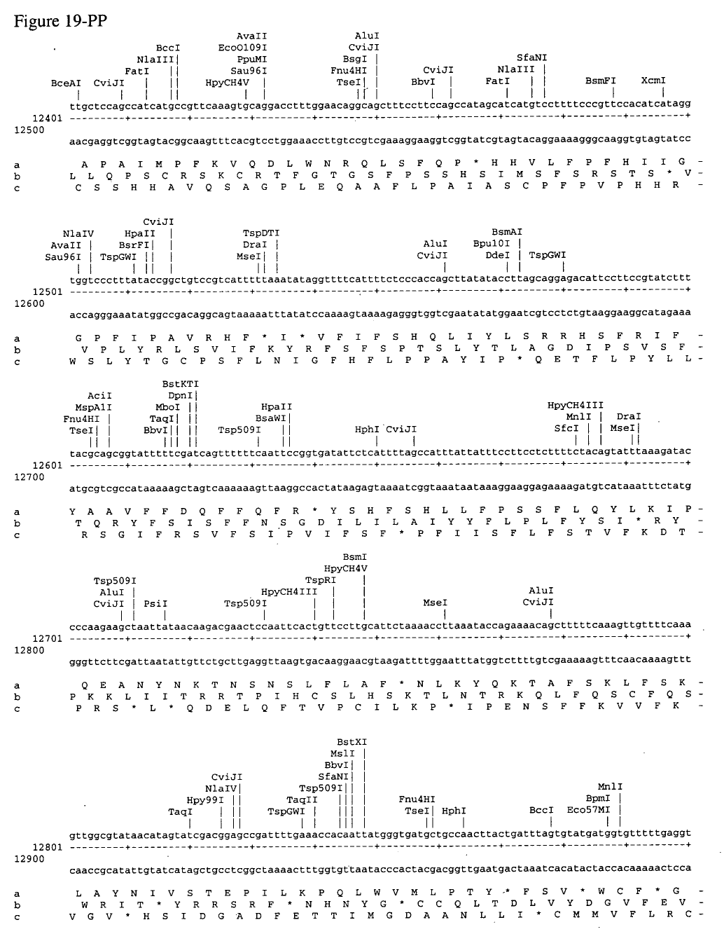

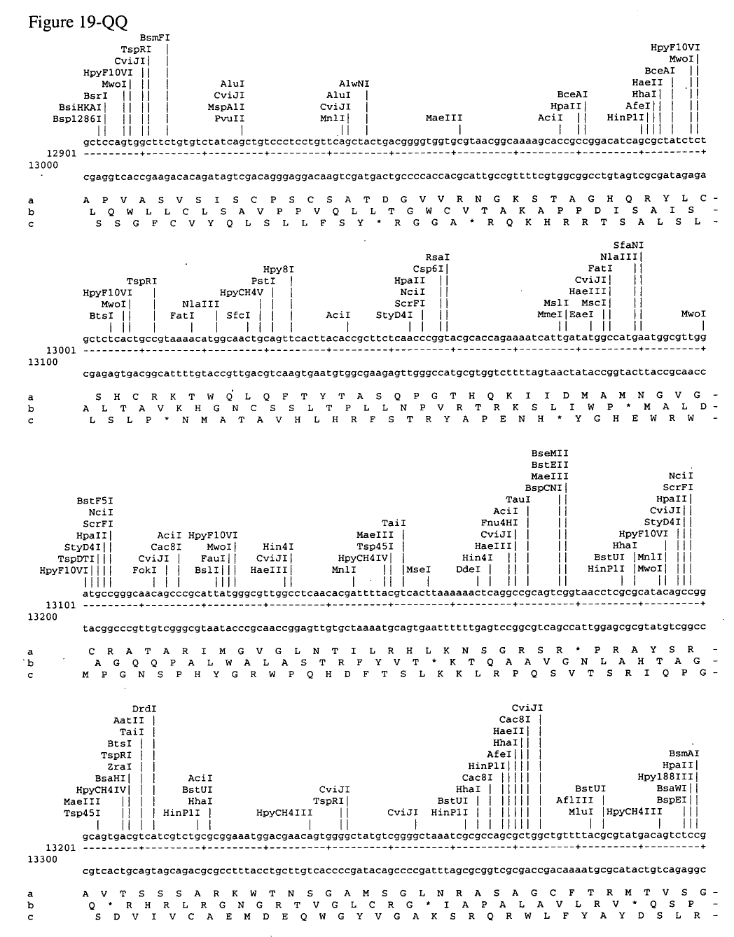

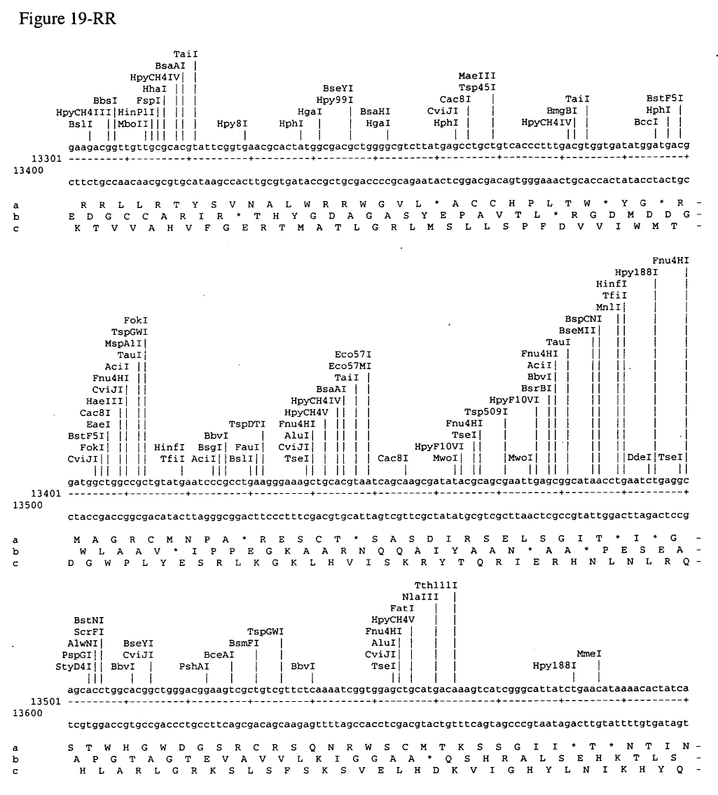

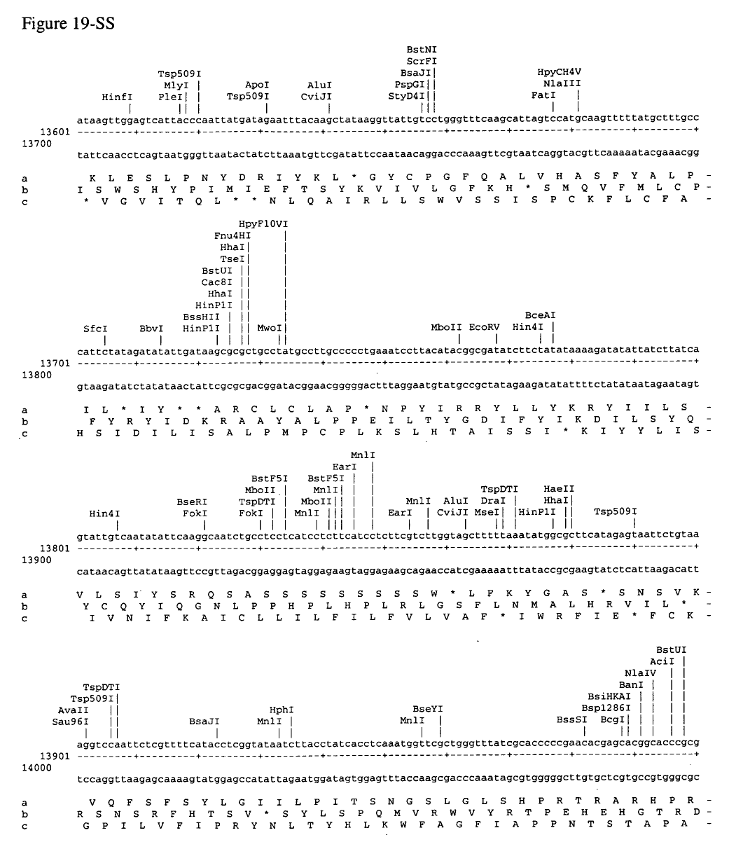

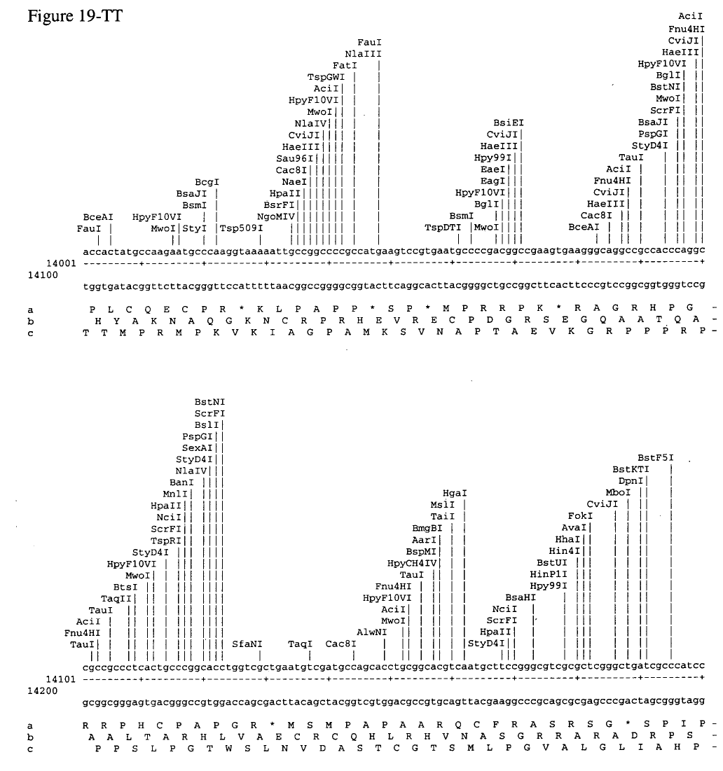

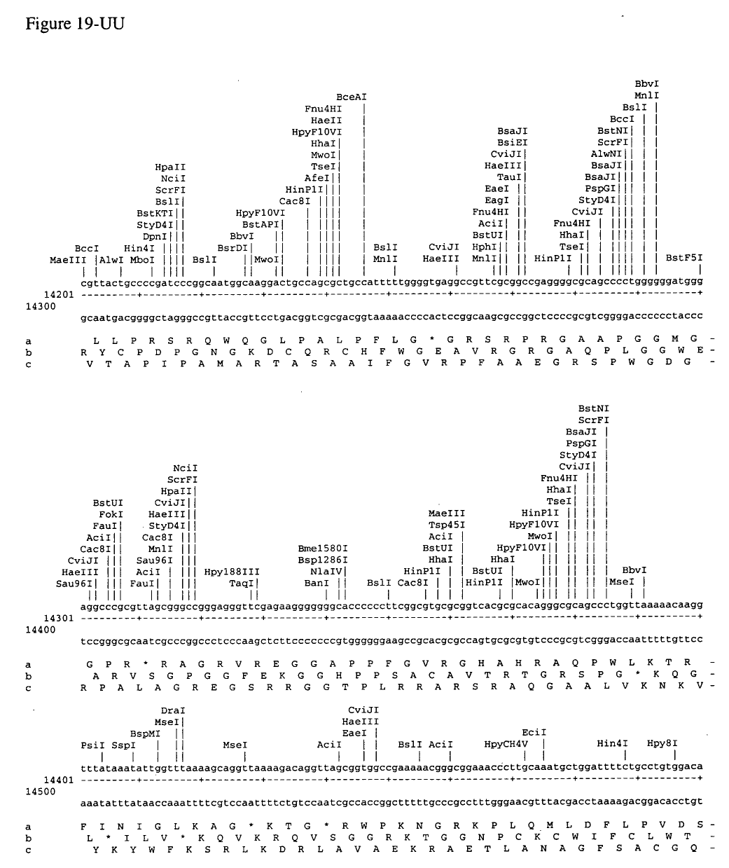

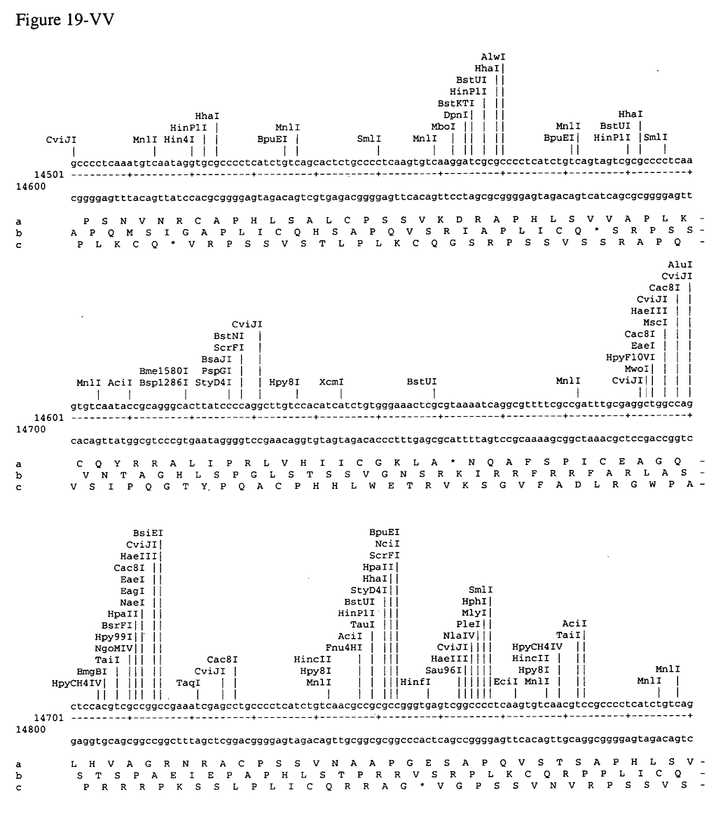

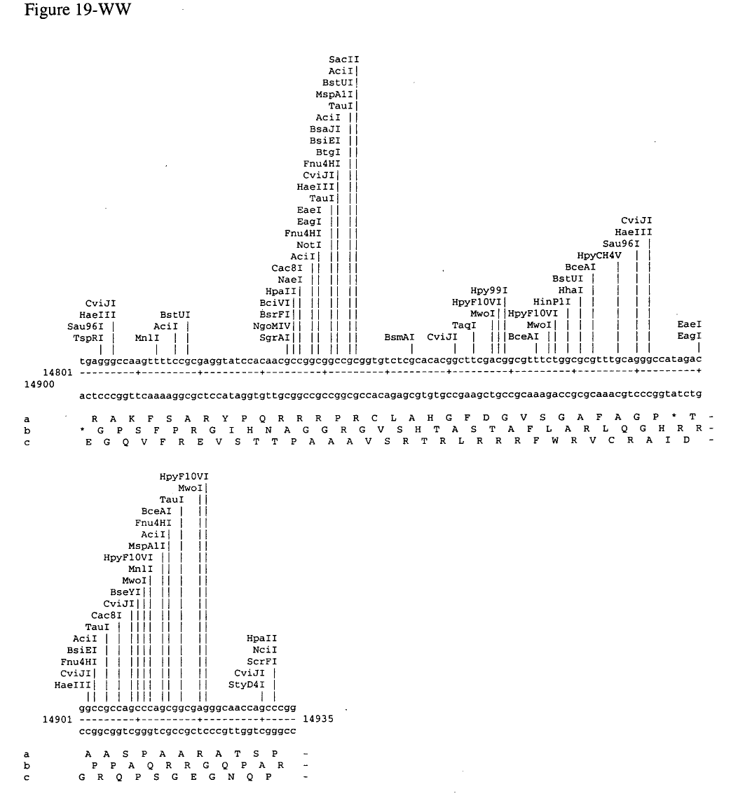

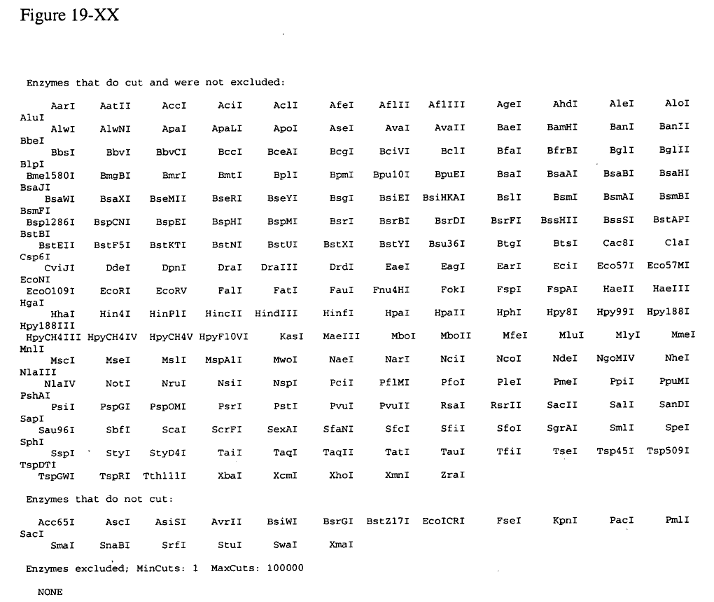

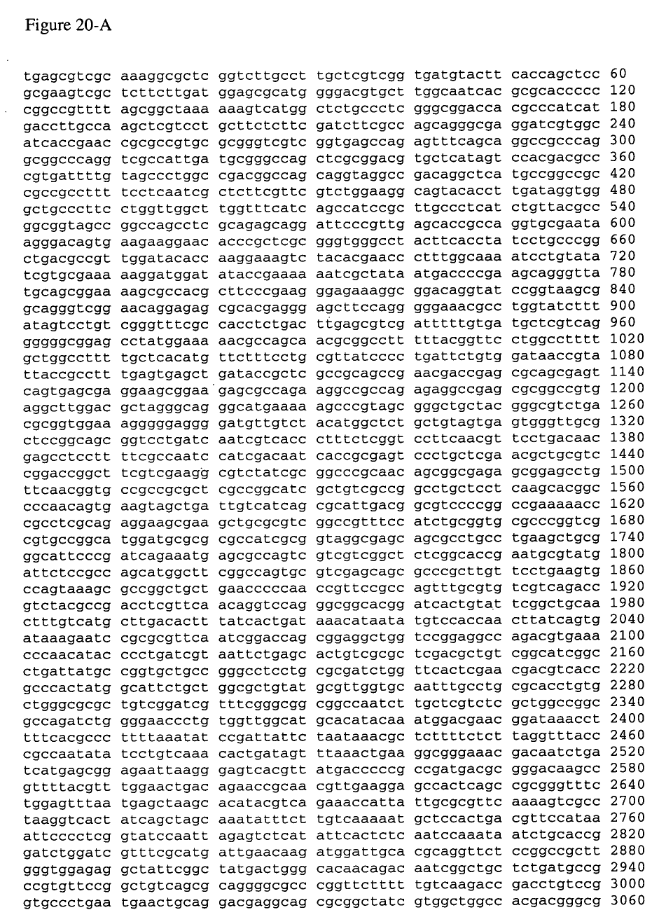

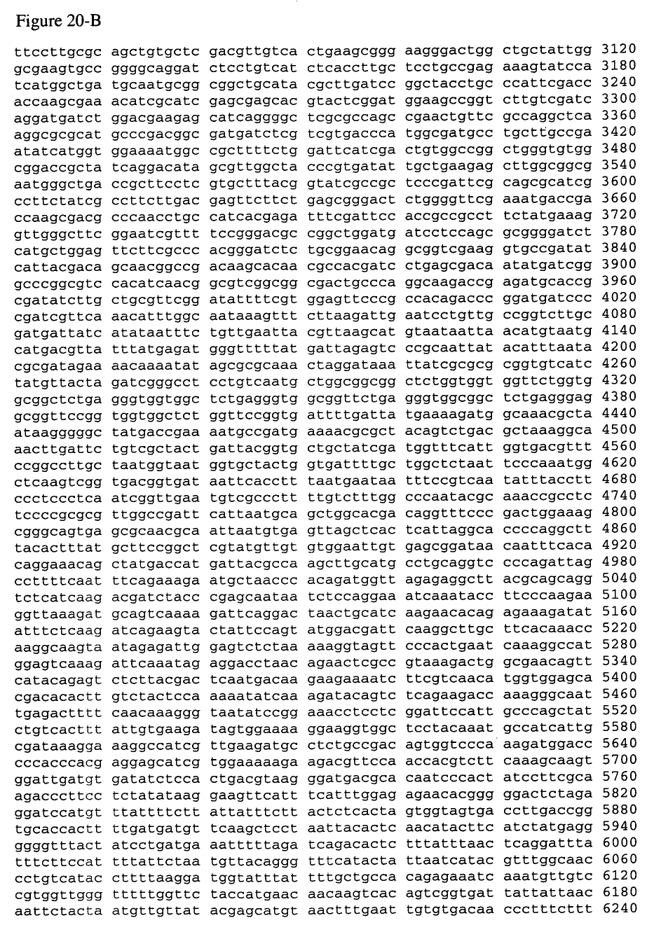

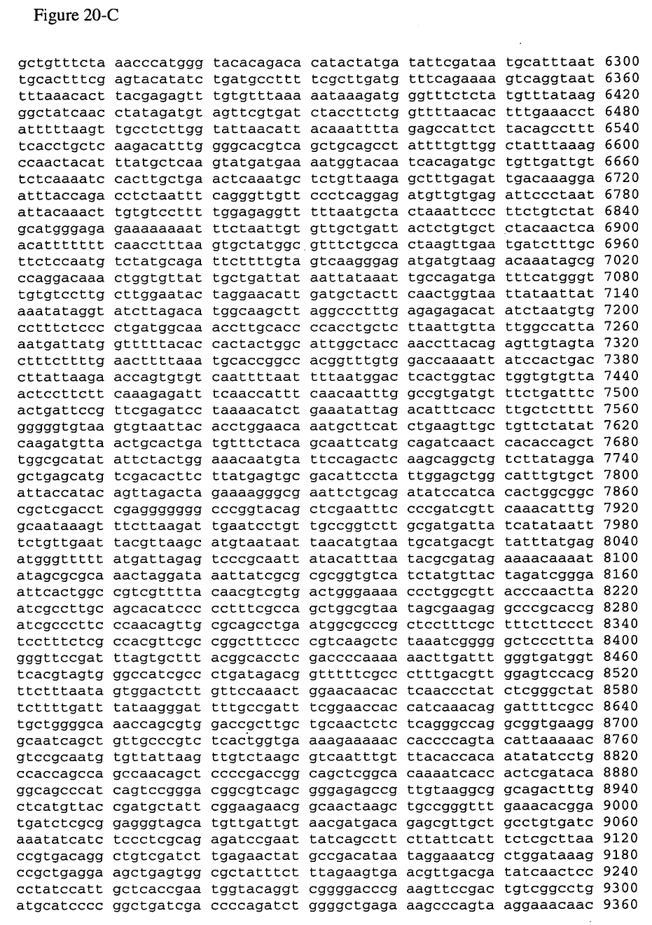

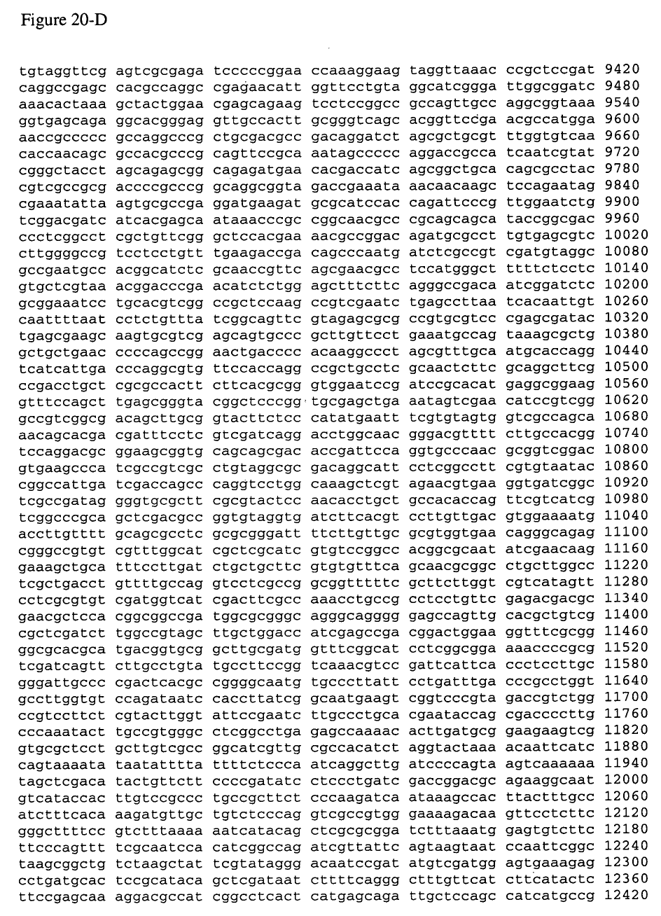

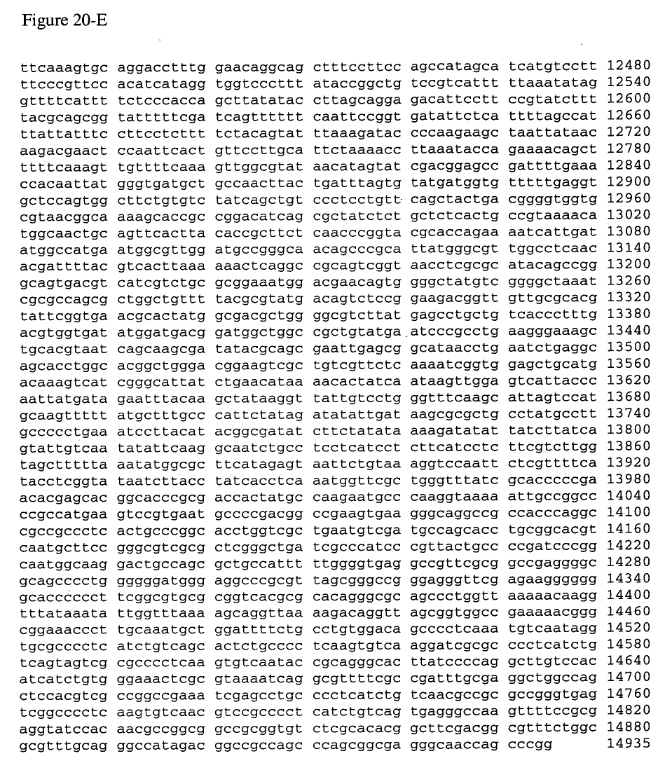

The 2-kb BamHI-XhoI fragment of S1 encoding amino acids 1-658 from pCRII-S1 was cloned into the BamHI and SalI sites of pSa7 (FIG. 5), a plant nuclear transformation vector. In this case, the signal peptide of S1 (amino acids 1-13) is retained because previous reports suggest that the presence of endoplasmic reticulum-targeting signals on plant nuclear-expressed foreign proteins results in improved protein stability (Richter et al., 2000, Nature Biotech 18: 1167-1171; Sojikul et al., 2003, Proc. Natl. Acad. Sci. 100: 2209-2214). In the resultant plasmid, pCV2 (FIG. 19), S1 is placed between the strong and constitutive Cauliflower Mosaic Virus 35S promoter and the nopaline synthase (NOS) terminator. This plasmid was introduced from Escherichia coli to Agrobacterium tumefaciens strain LBA4404 in the presence of E. coli helper strain HB101/pRK2013 by triparental mating (Horsch et al., 1985, Science 227: 1227-1231).

Construction of pCV8, a Plastid Transformation Vector for Expression of the M Antigen

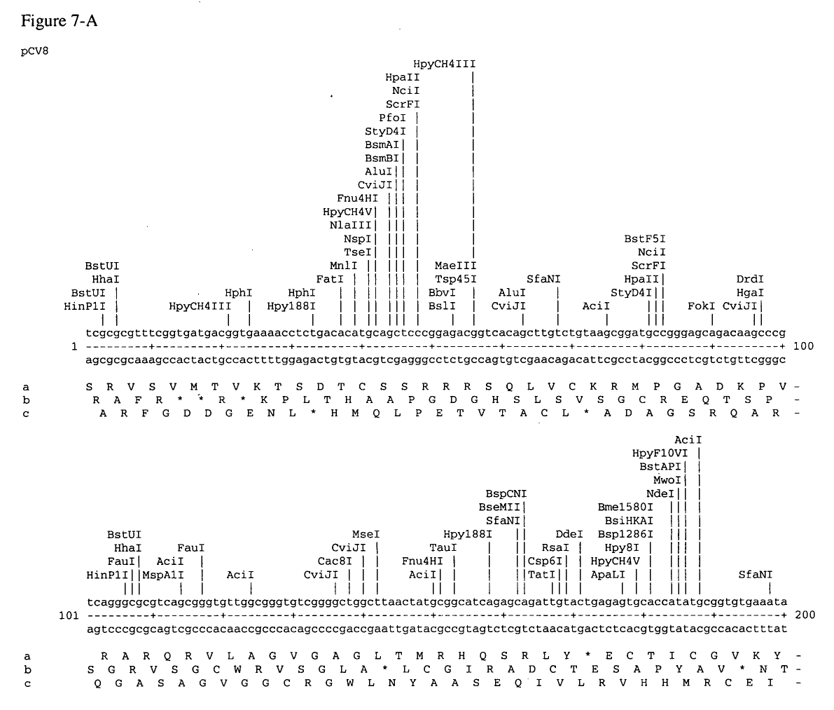

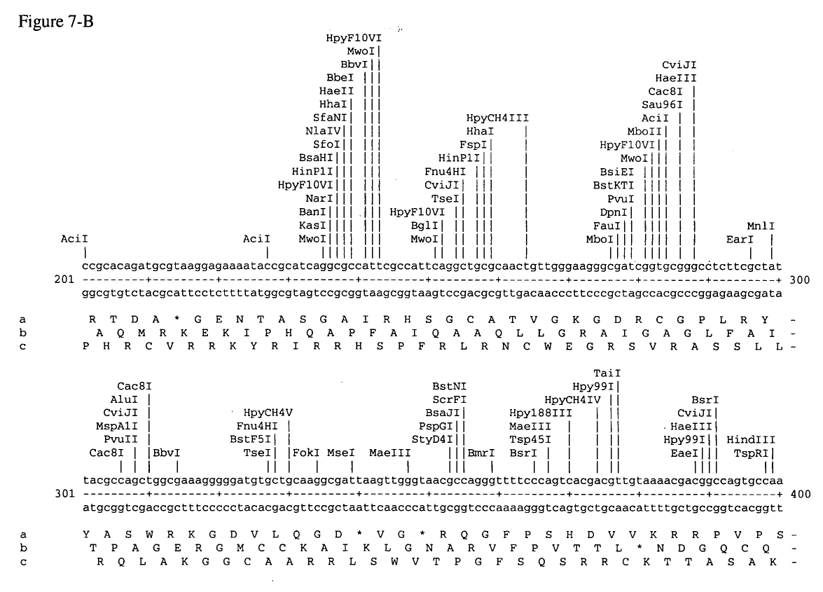

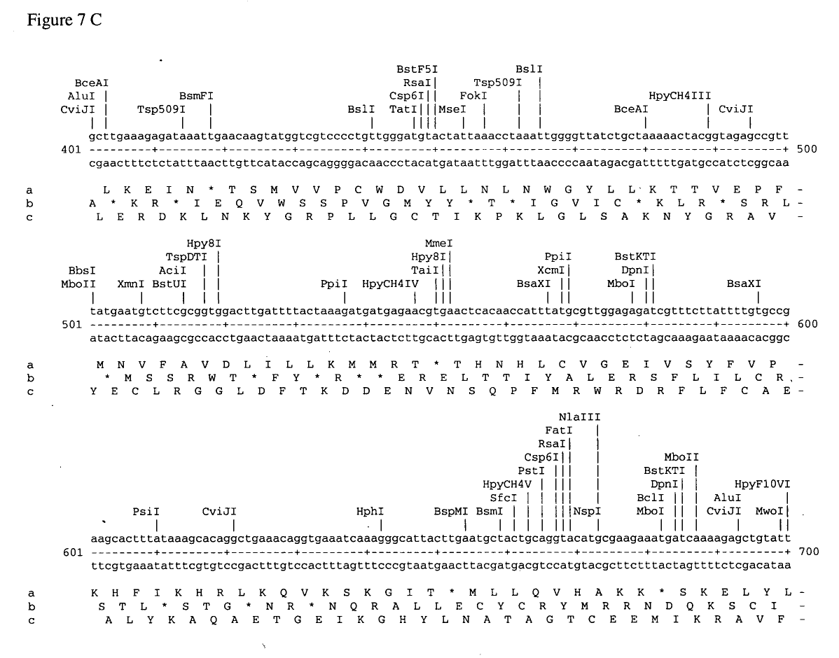

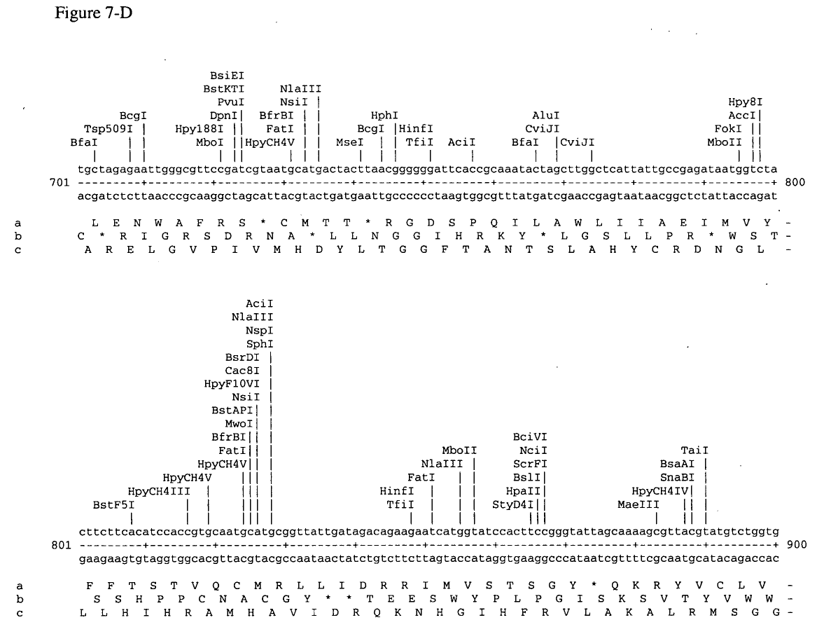

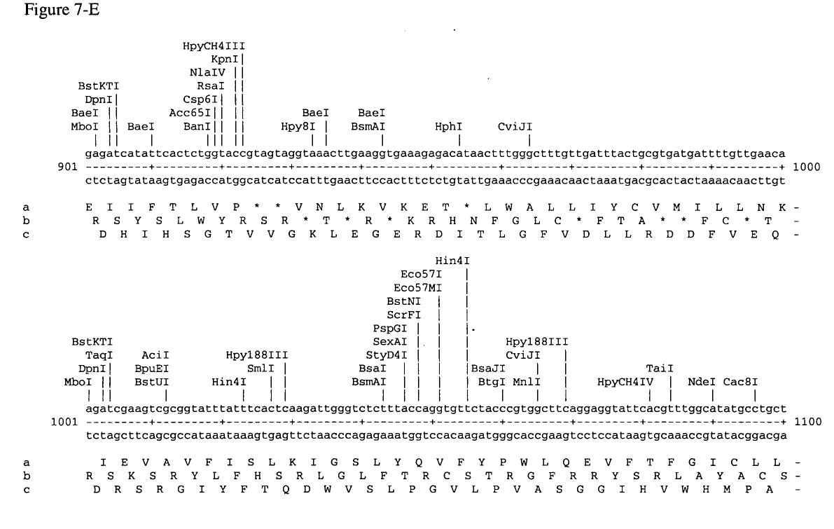

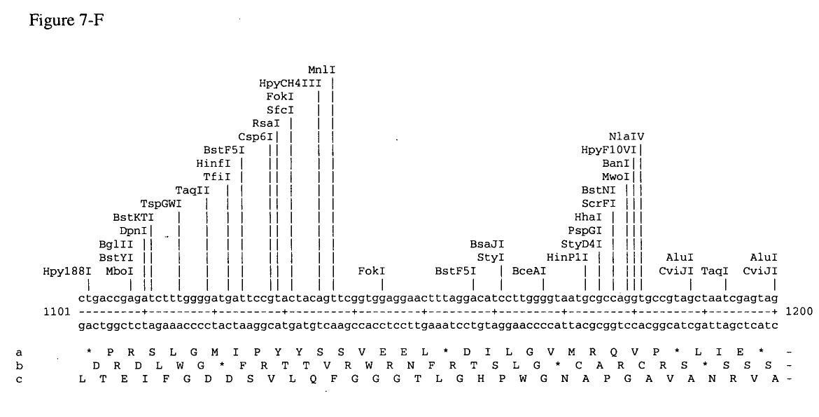

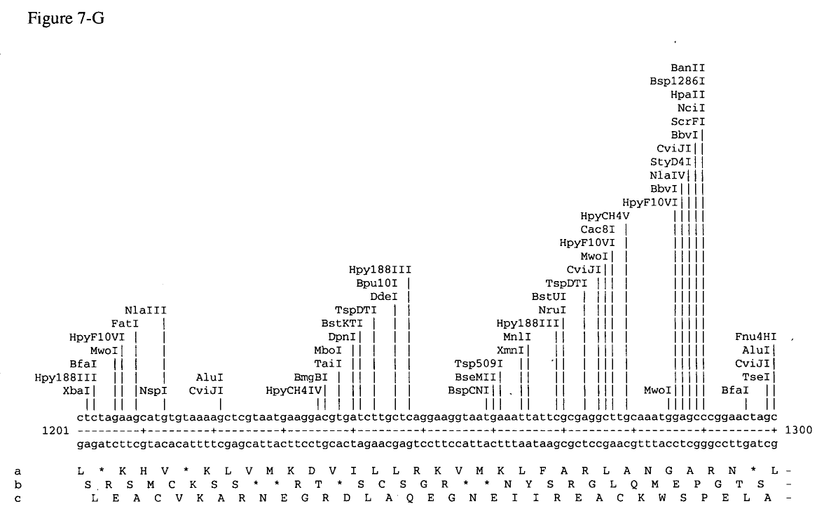

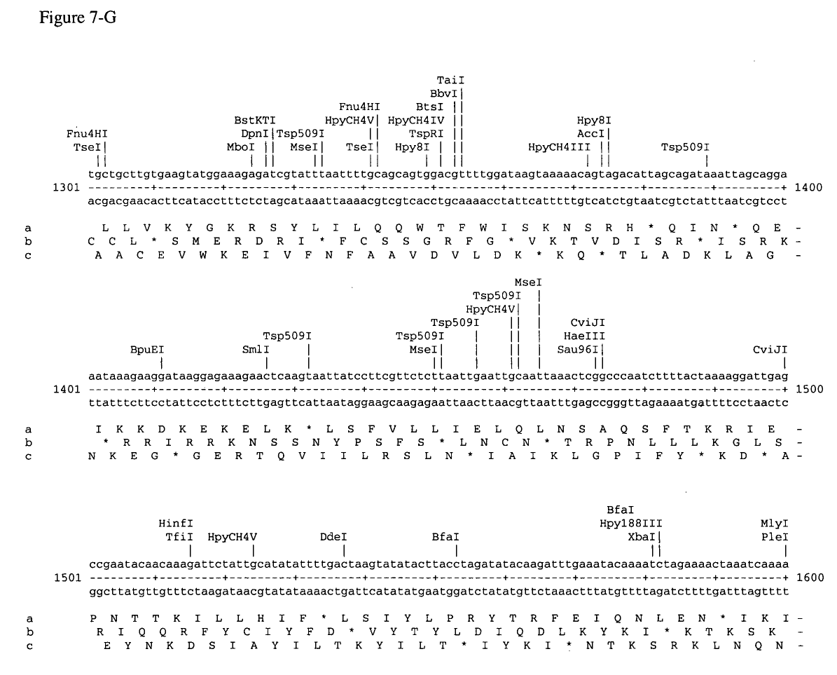

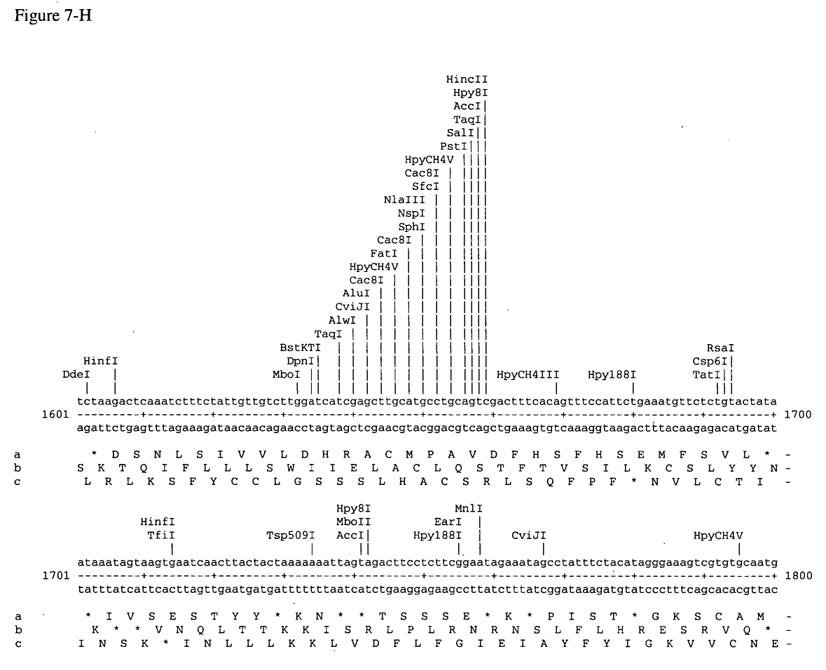

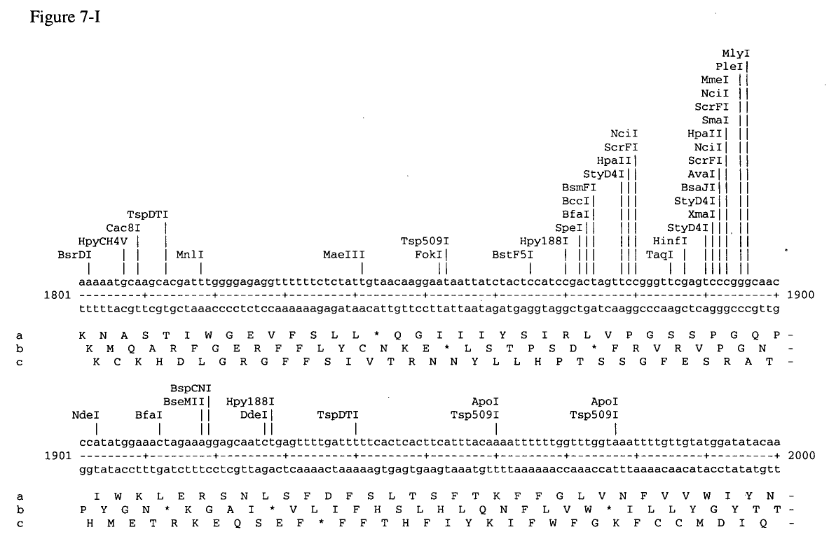

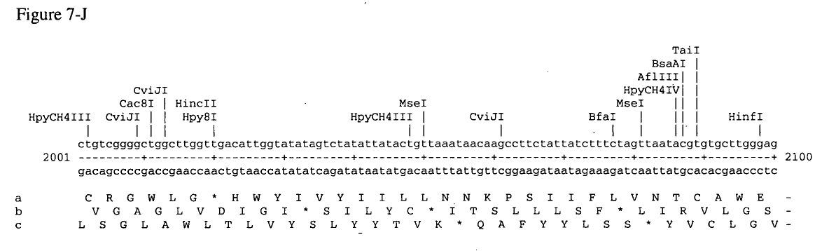

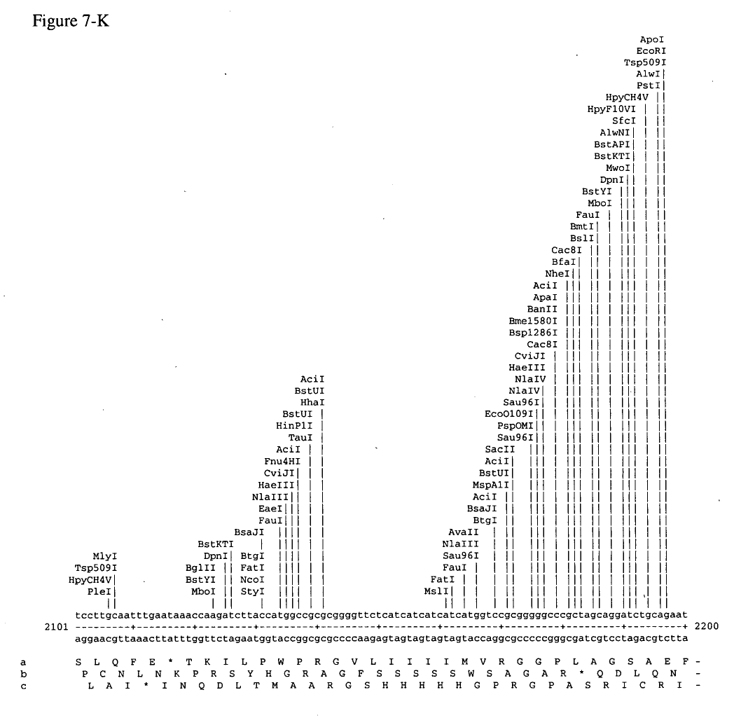

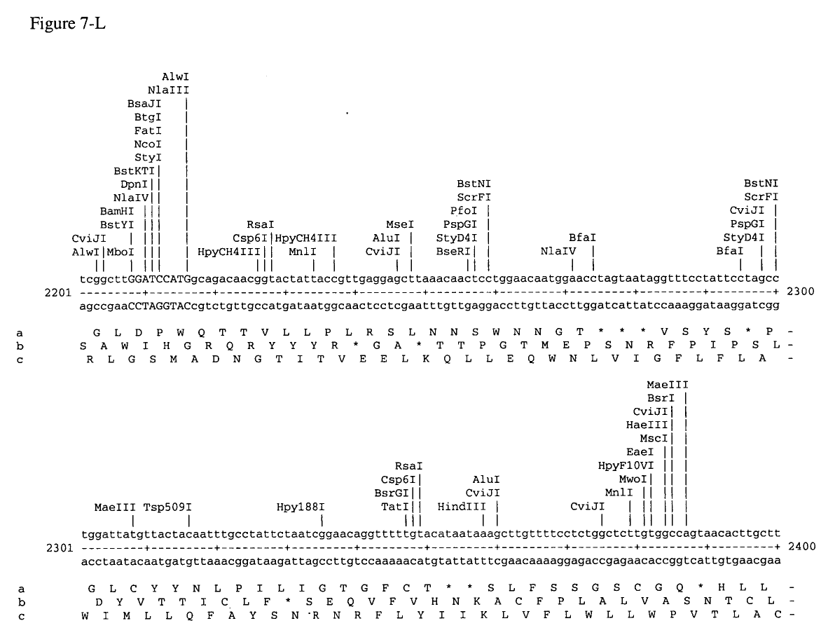

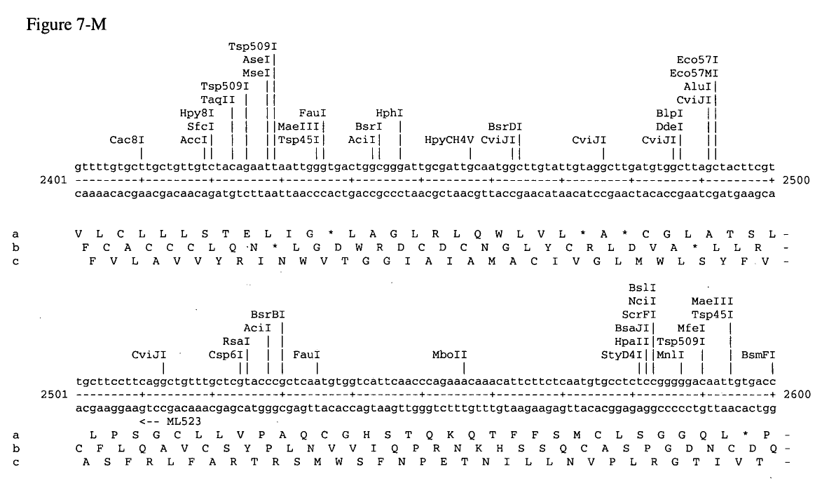

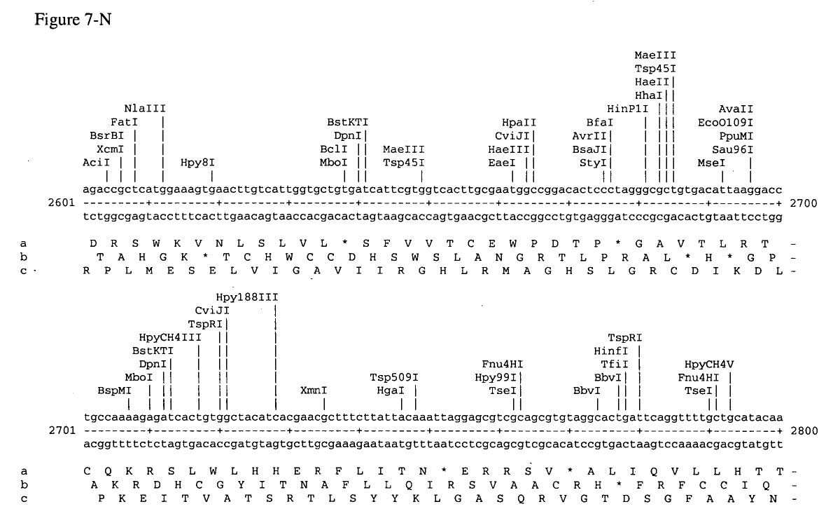

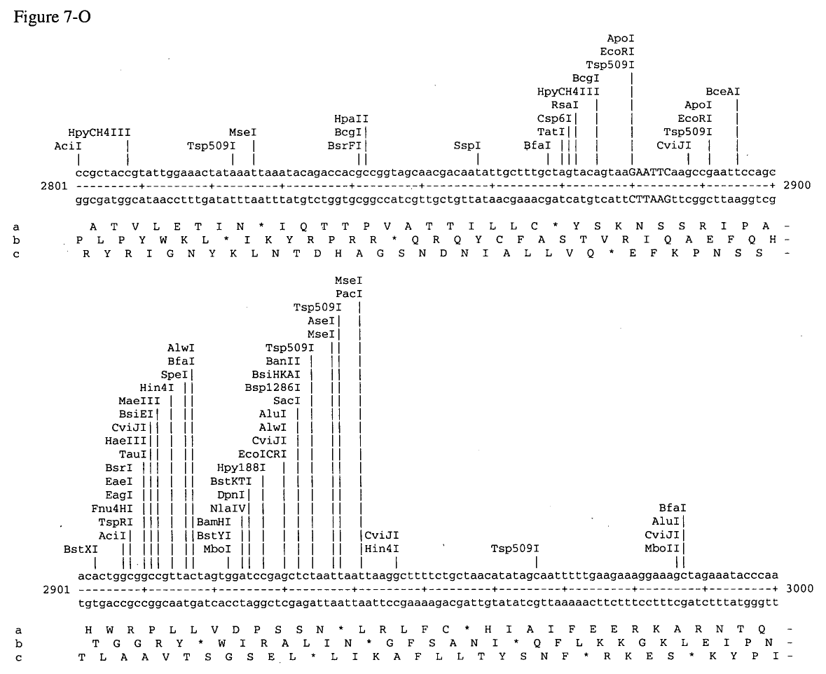

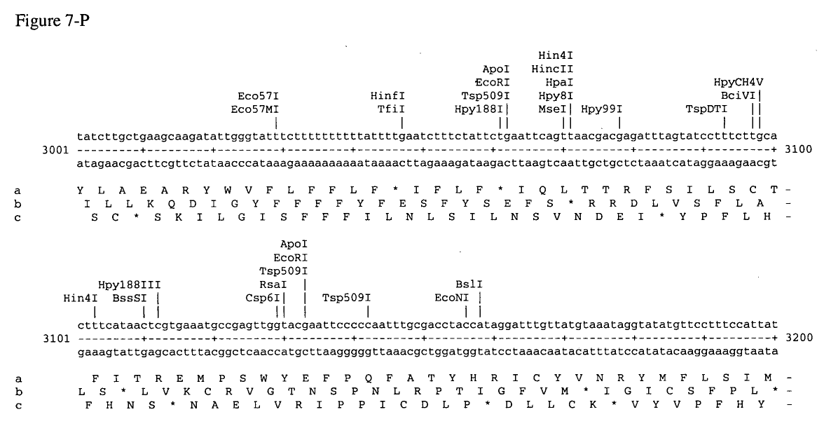

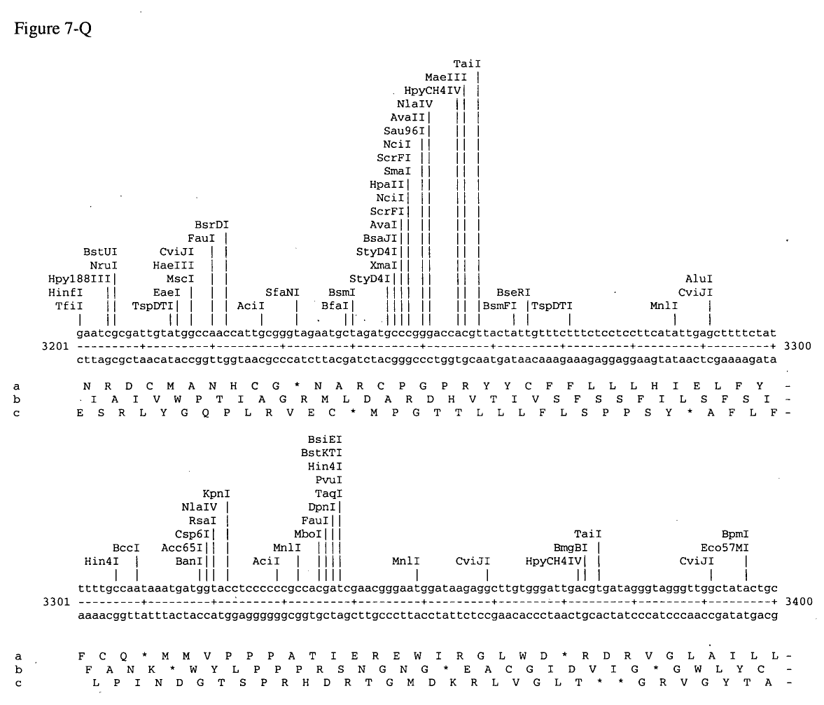

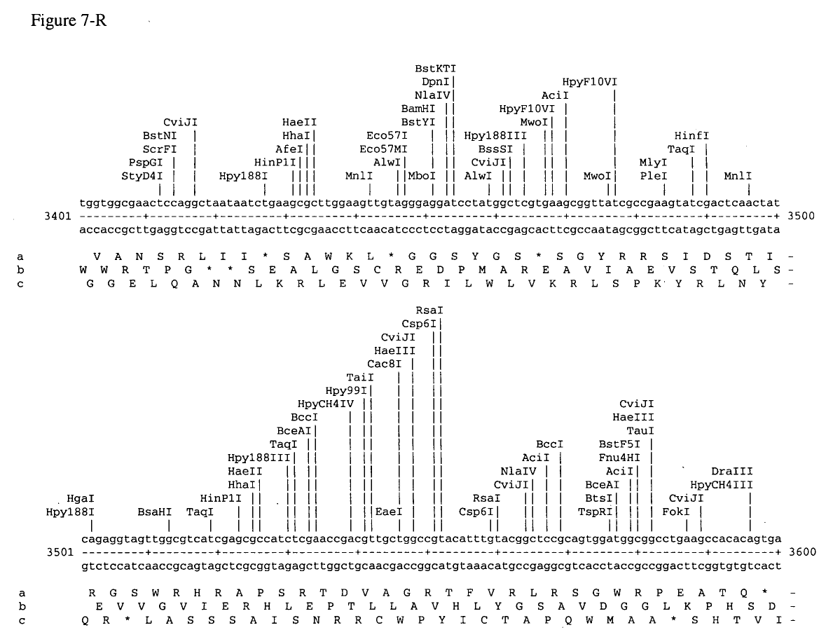

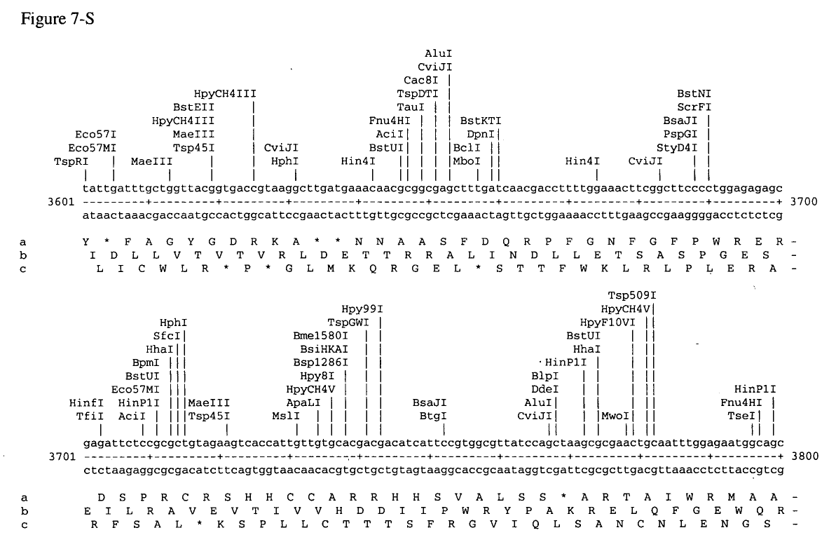

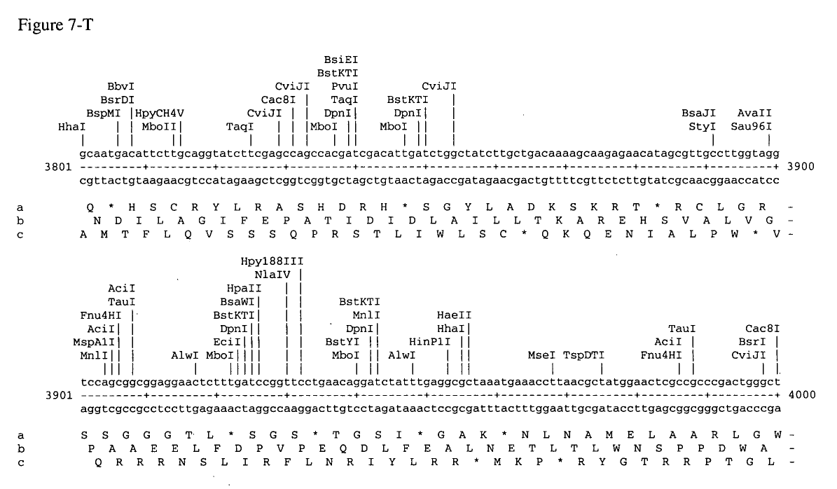

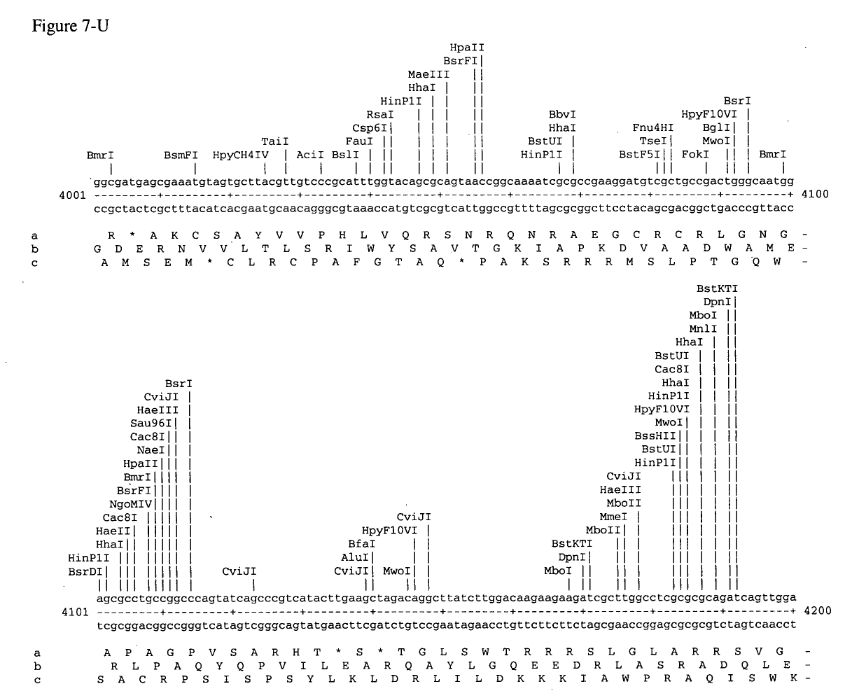

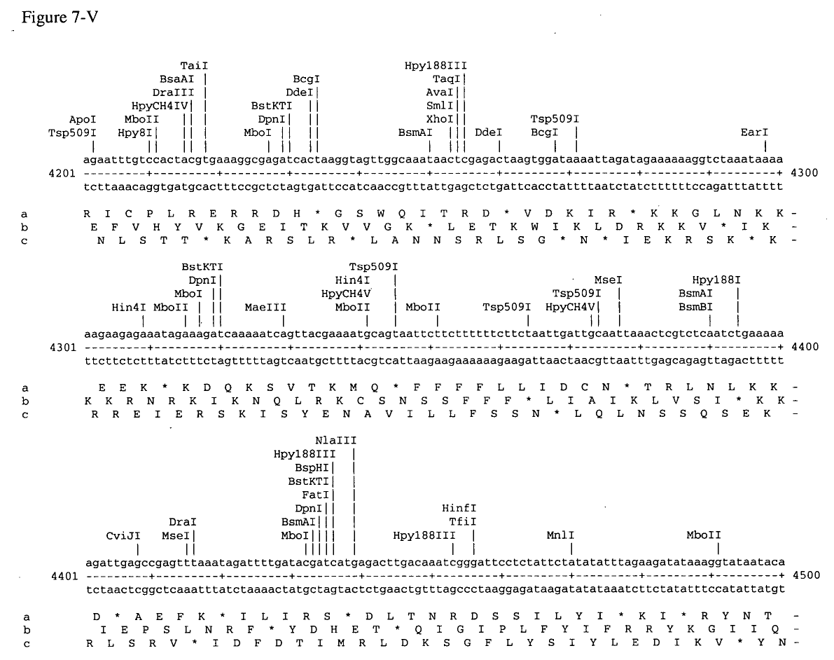

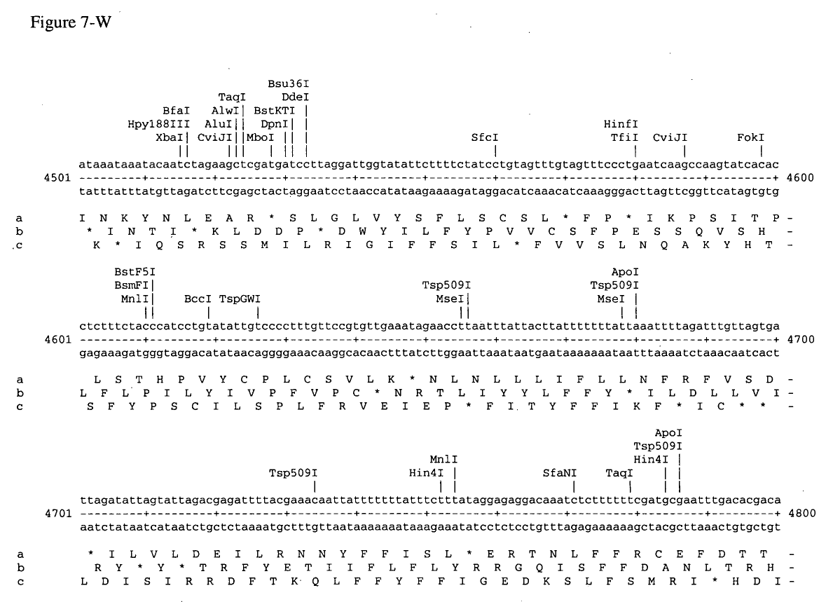

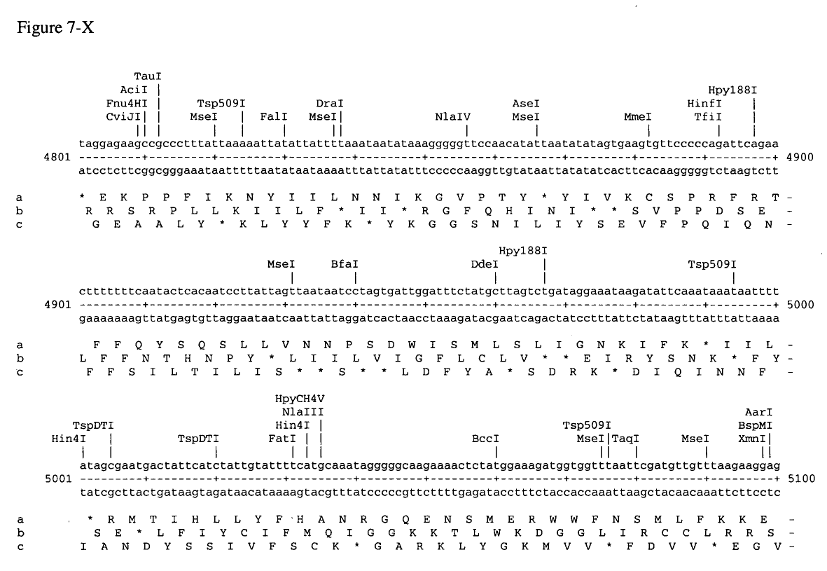

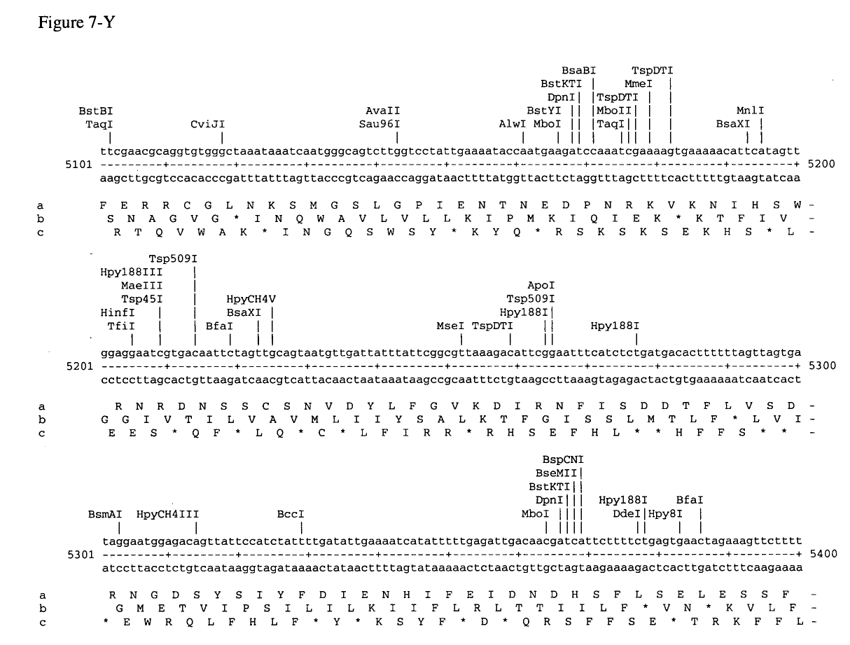

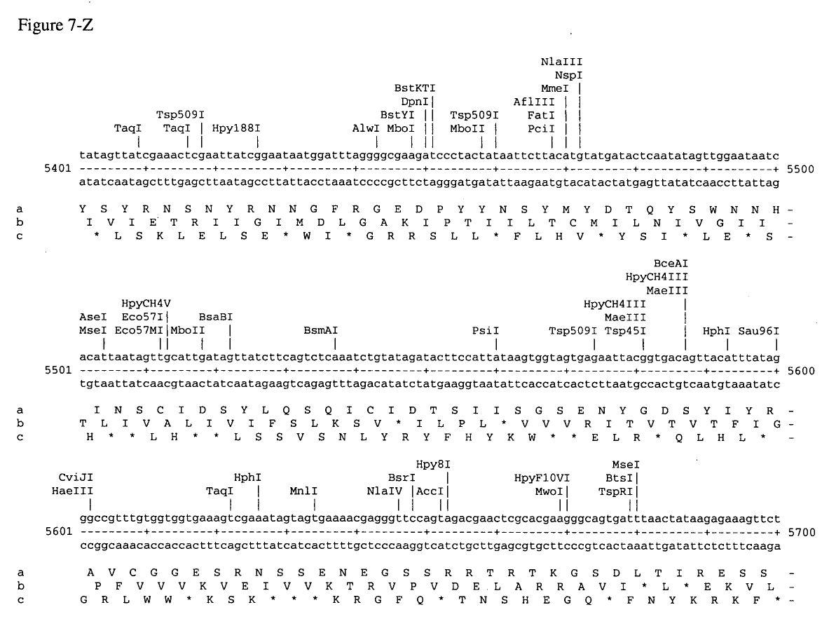

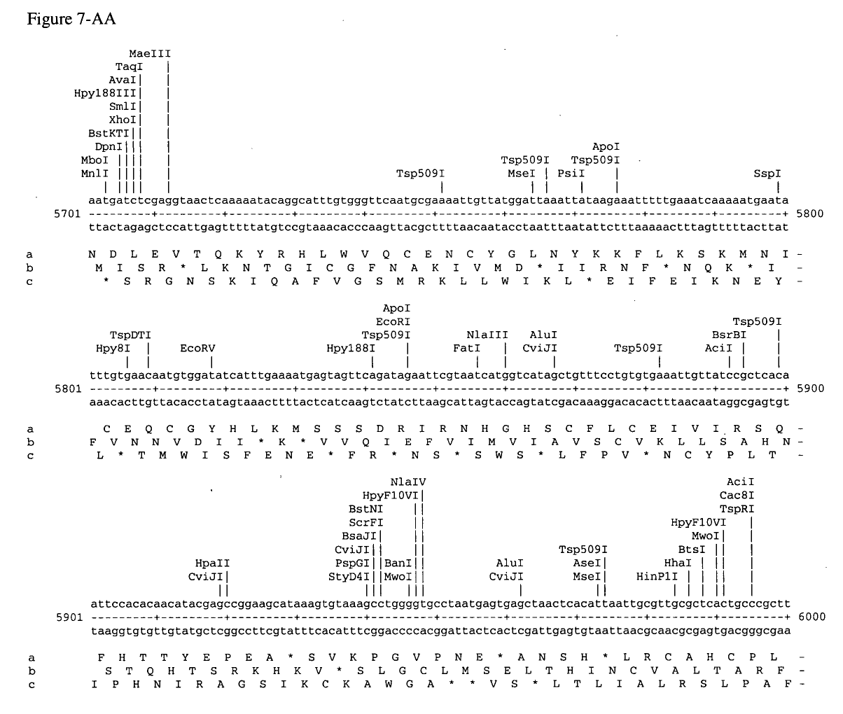

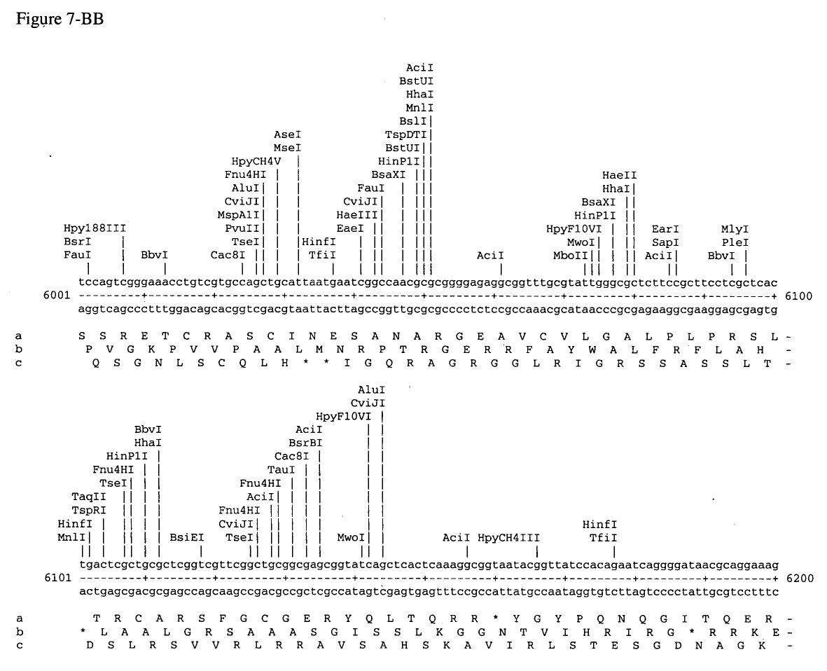

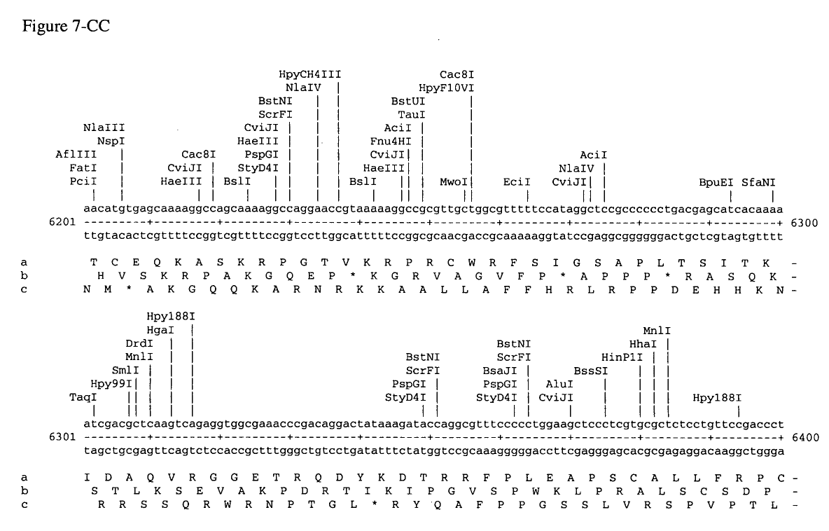

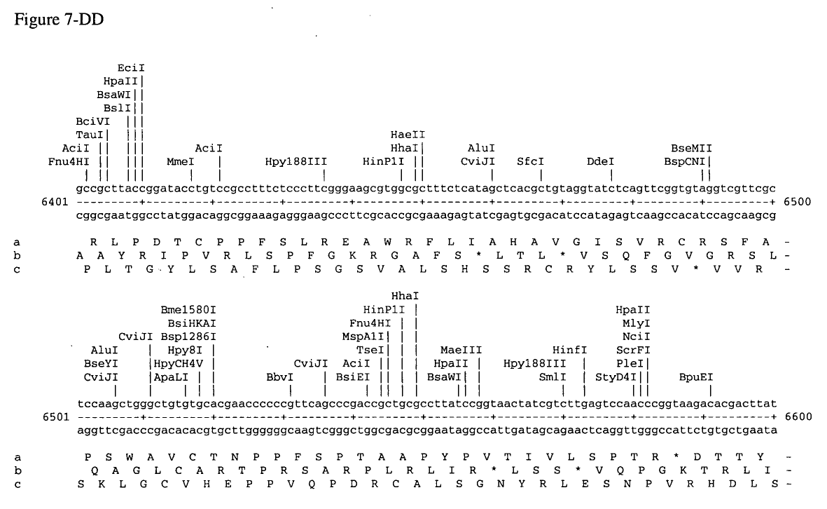

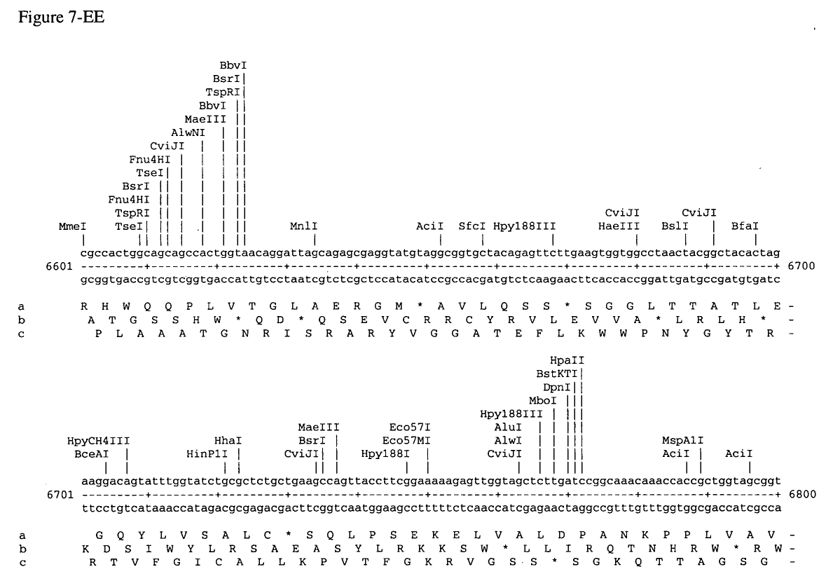

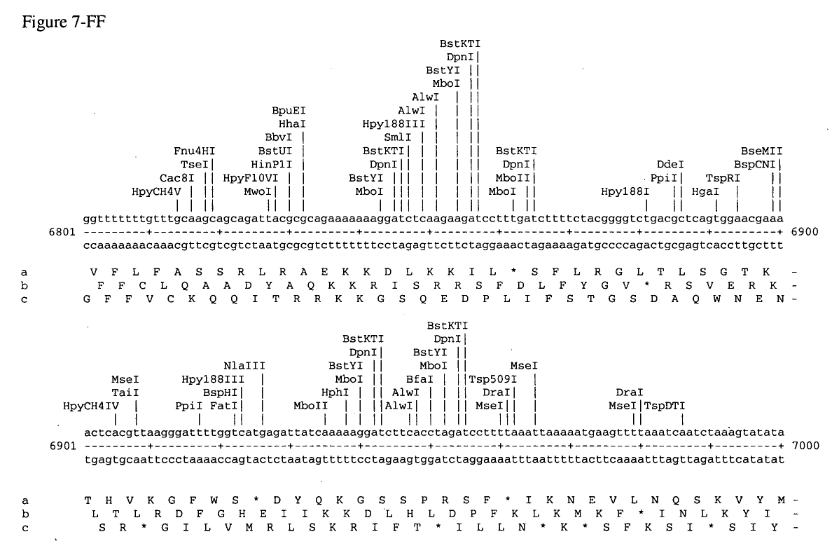

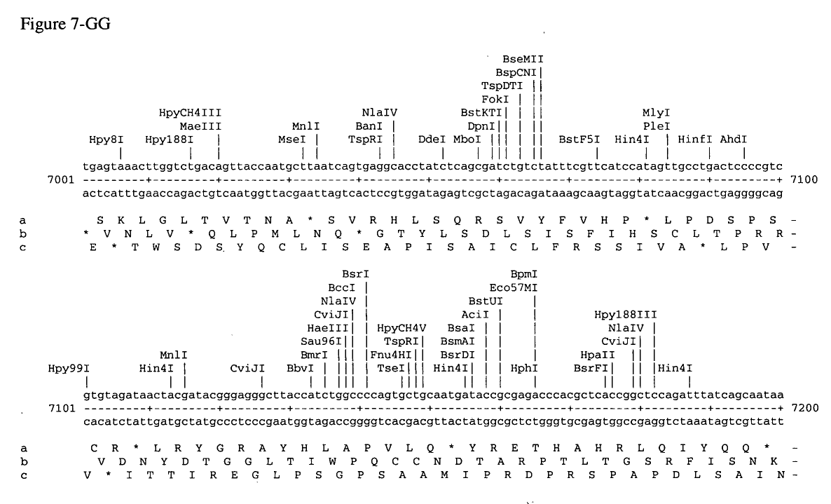

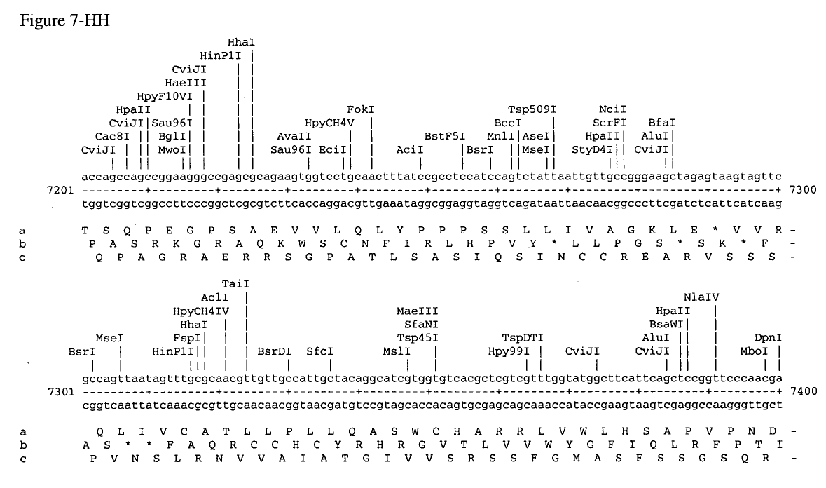

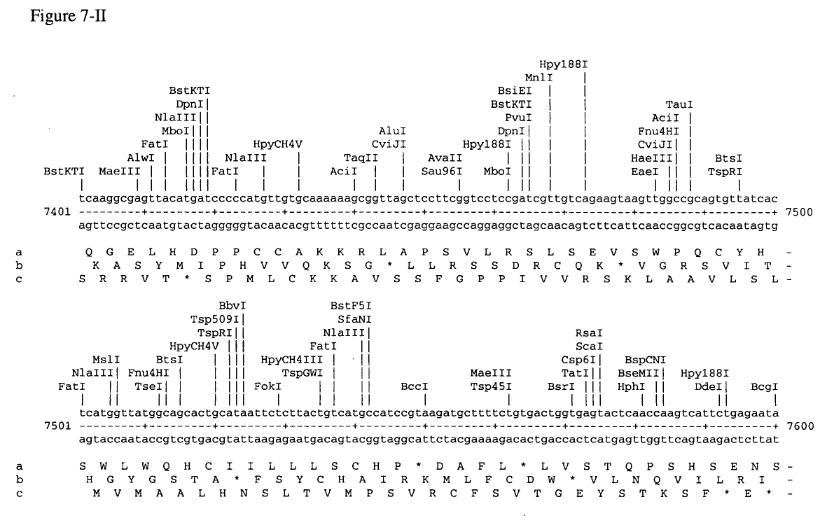

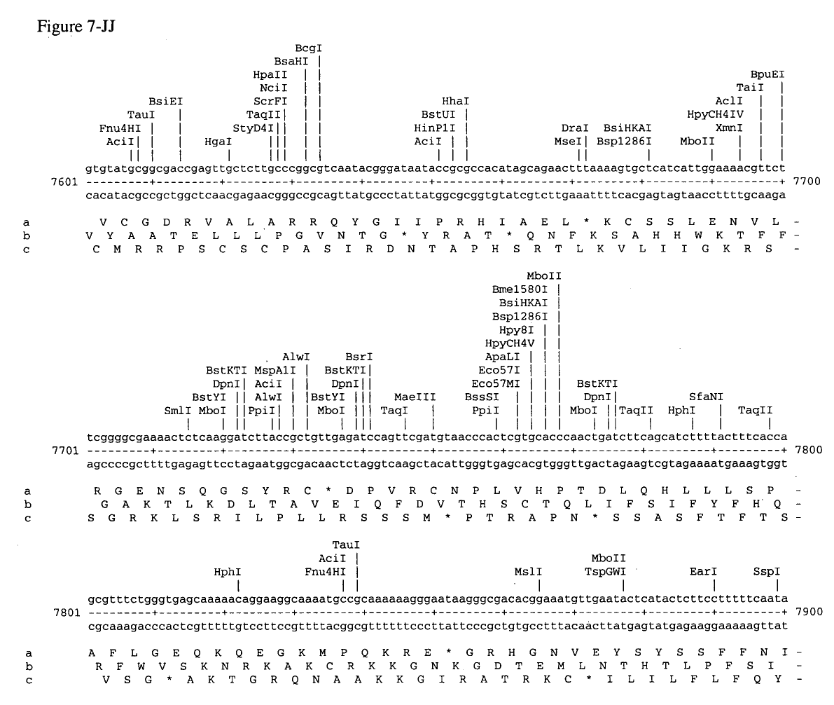

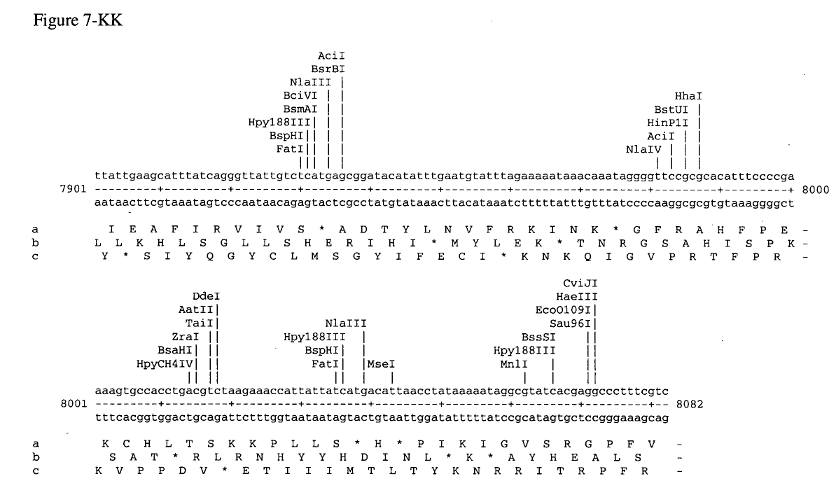

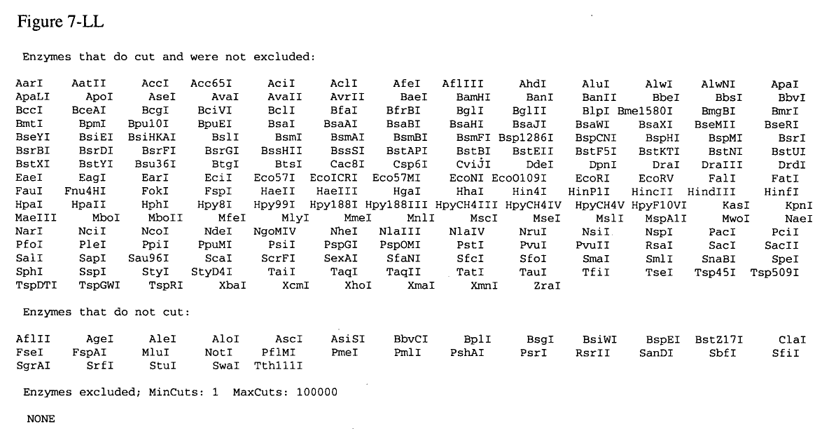

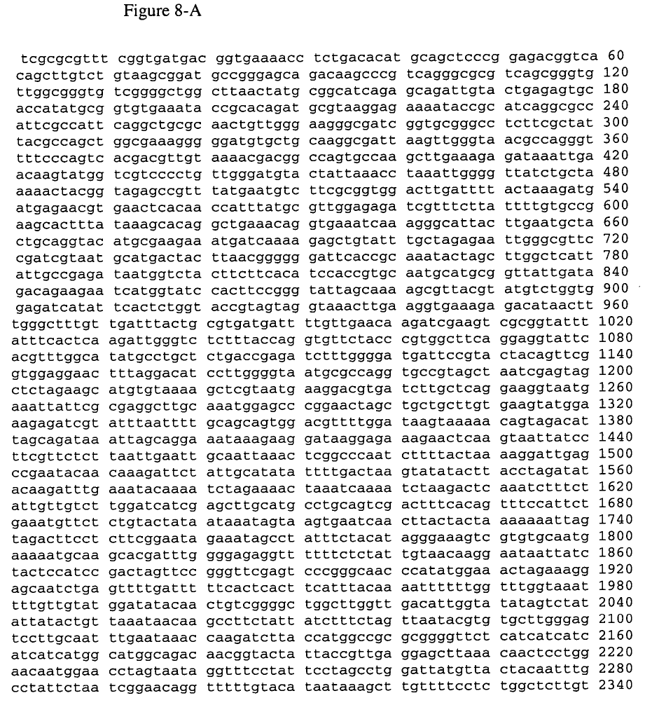

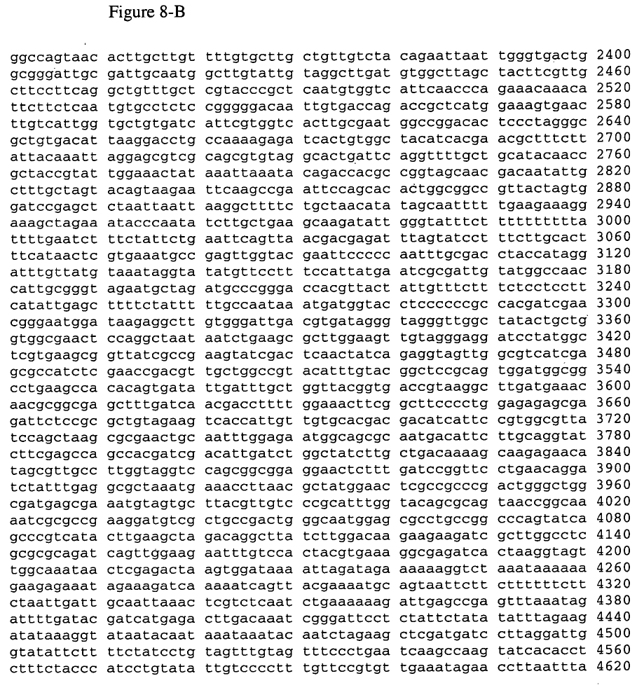

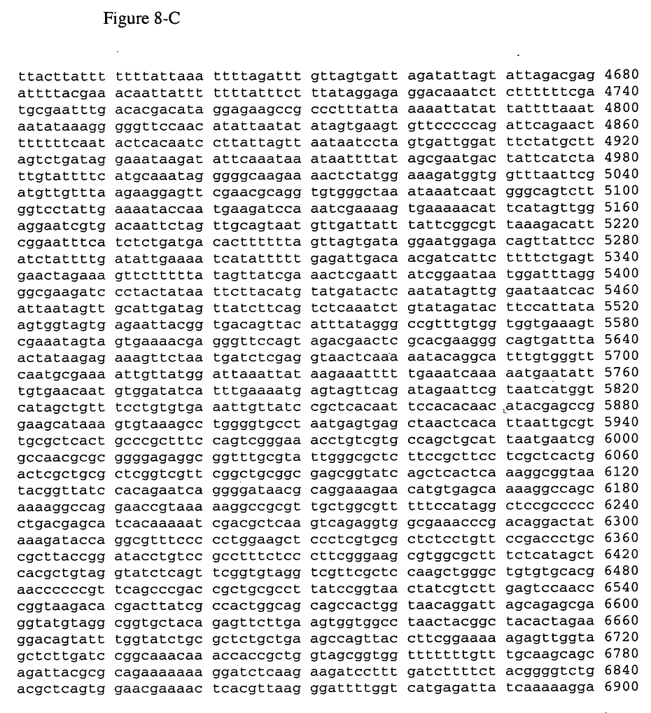

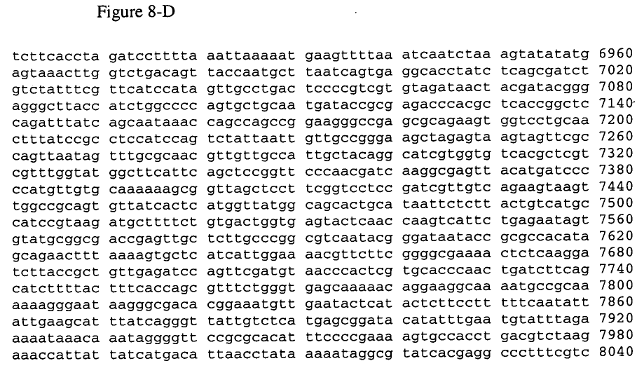

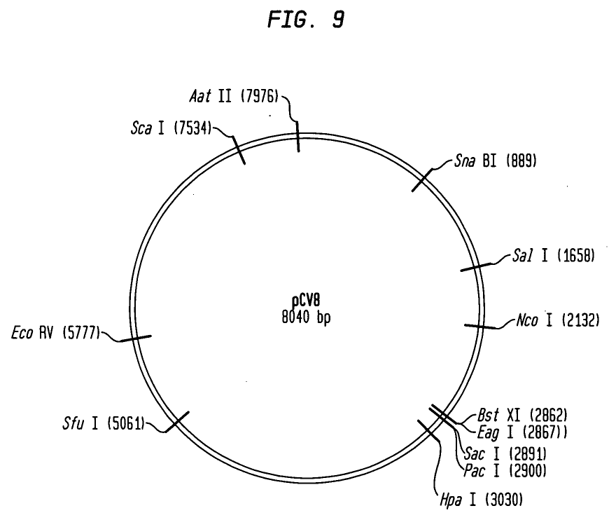

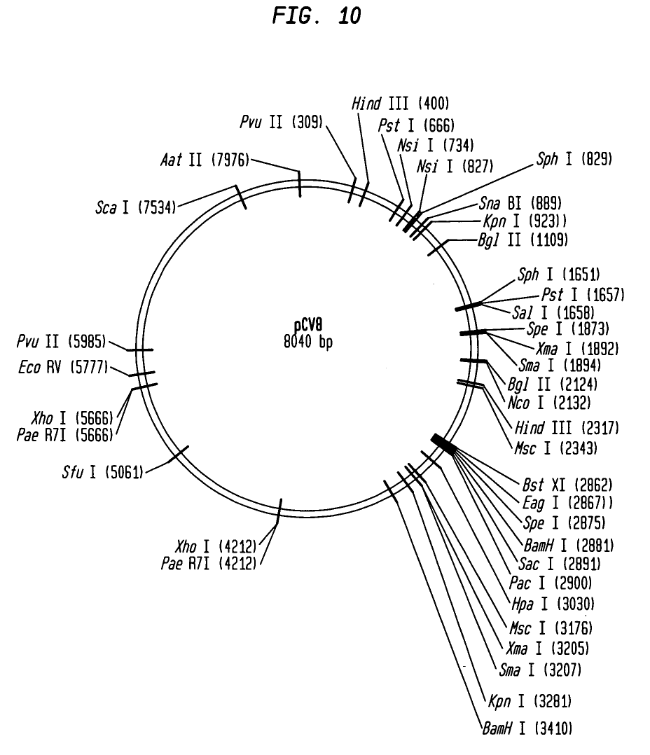

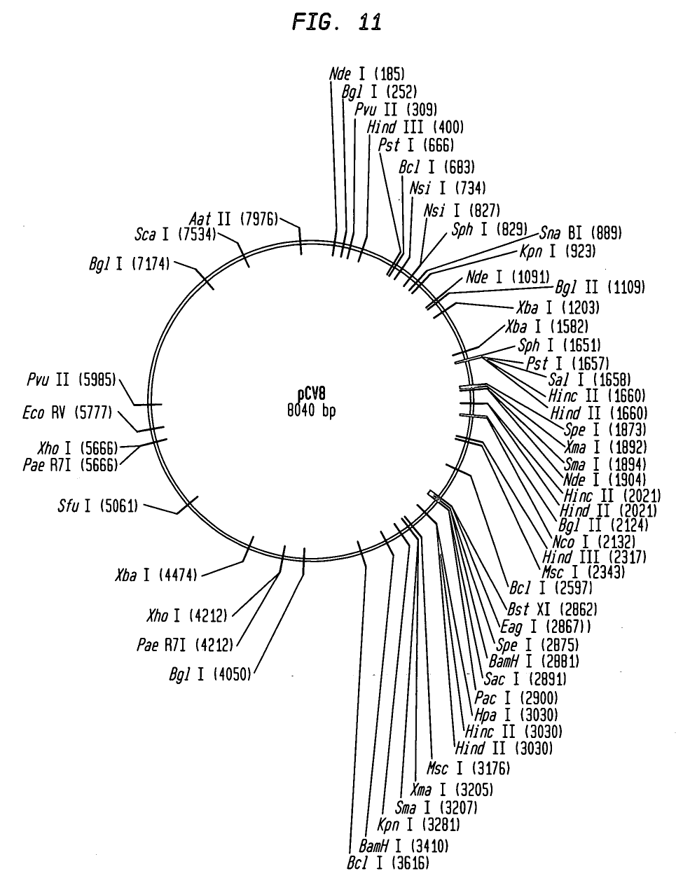

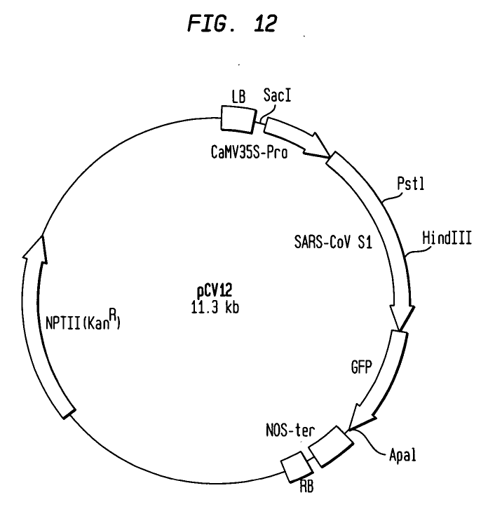

The 0.7-kb NcoI-SacI fragment encoding M from plasmid pCRII-M was cloned into the NcoI and SacI sites of pMLVHisA (FIG. 4) to generate pCV7. The plasmid pCRII-M was derived from pCRII (Invitrogen) and contains a PCR-amplified fragment of M. Subsequently, plasmid pCV7 was digested with NcoI and a (His)₆-tag was introduced into this site using annealed oligomers ML527 (5′-CATGGCCGCGCGGGGTTCTCATCATCATCATCATCATGG-3′; SEQ ID NO:10) and ML528 (5′-CATGCCATGATGATGATGATGATGAGAACCCCGCGCGGC-3′; SEQ ID NO:11) to create pCV8 (FIG. 7). The resultant M expressed from pCV8 is a (His)₆-tagged protein.

Construction of pCV4, a Nuclear Transformation Vector for Expression of the M Antigen

The 0.7-kb BamHI fragment encoding M from plasmid pCRII-M was cloned into the BamHI site of plant nuclear transformation vector pSal3. Plasmid pSal3 was derived from plasmid pSa7 (FIG. 5) by removal of a 0.58-kb SalI fragment of SaPIN2a cDNA followed by religation.

Construction of pCV6, a Plastid Transformation Vector for Co-Expression of the S1 and M Antigens

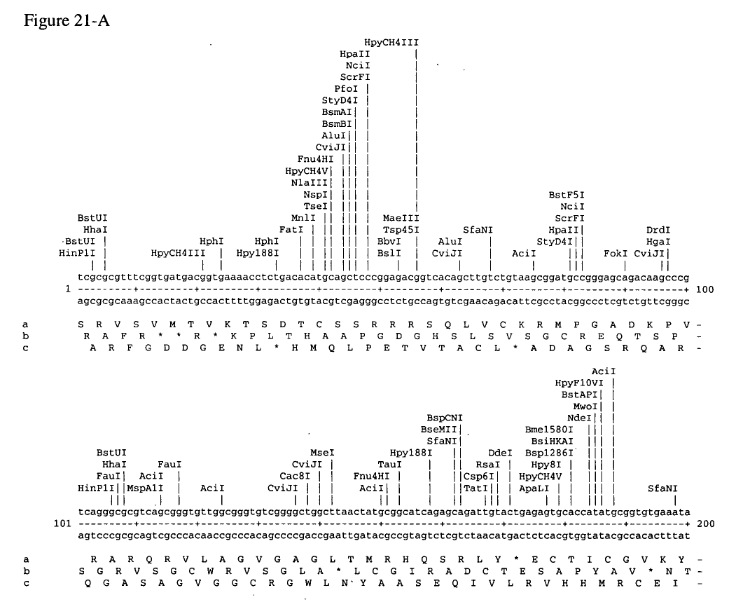

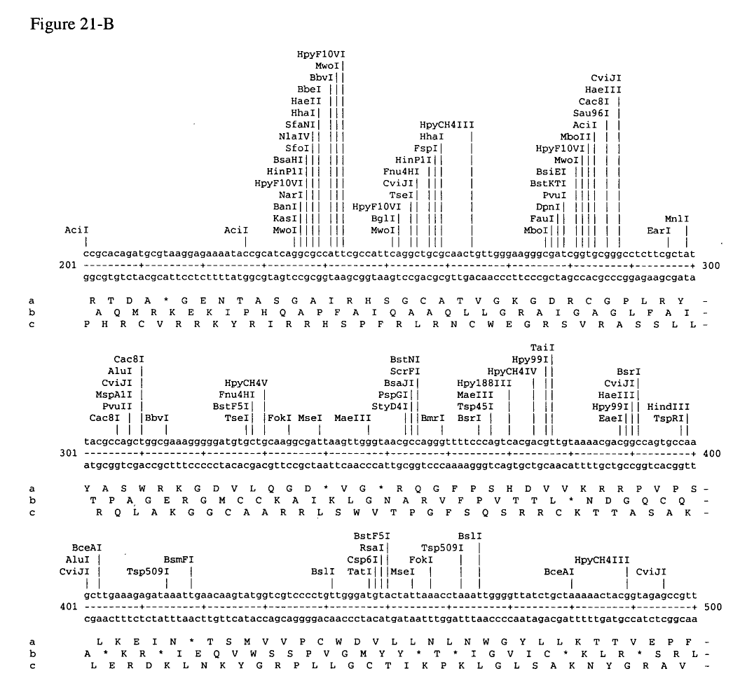

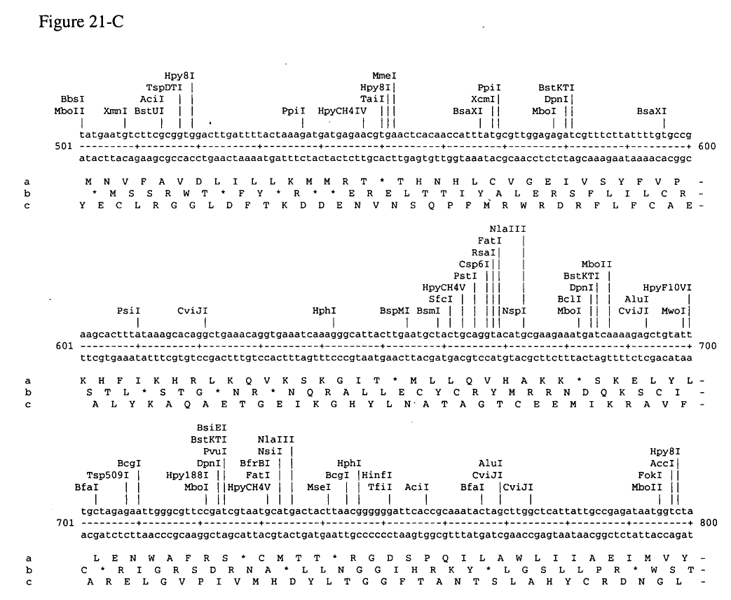

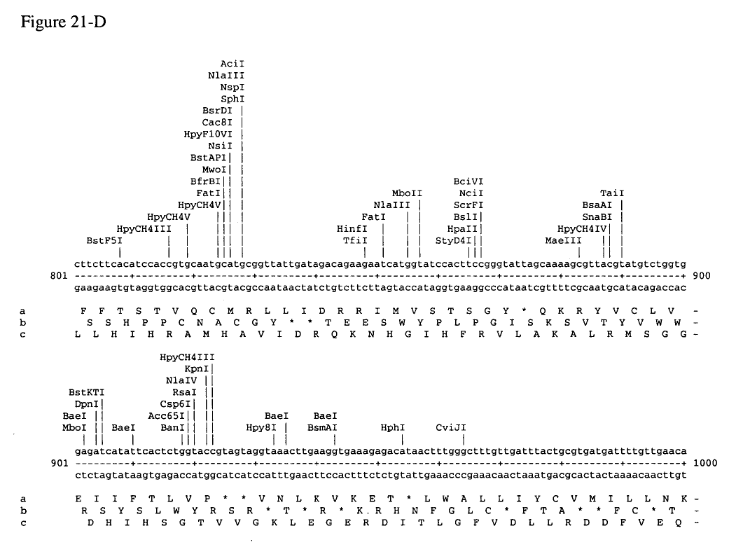

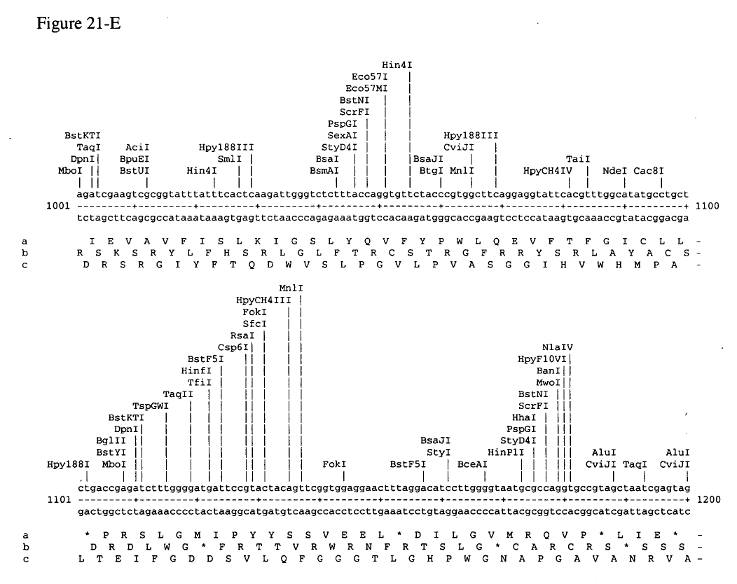

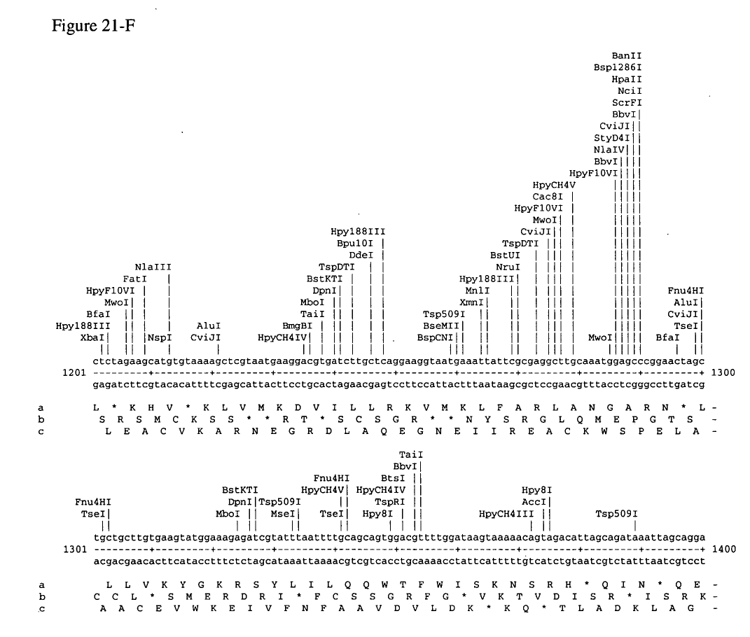

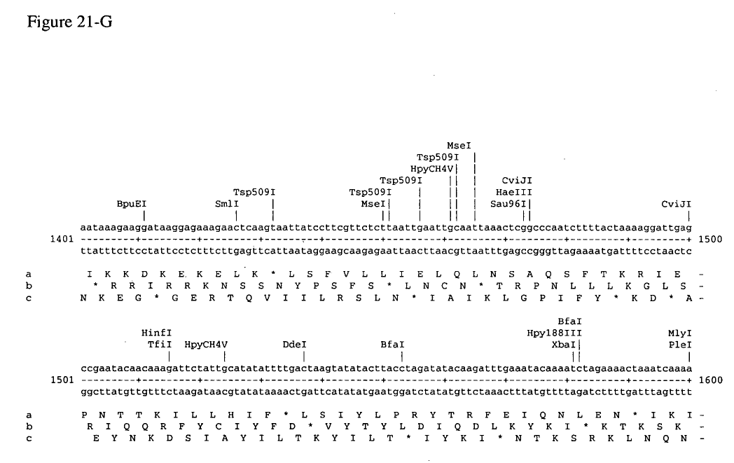

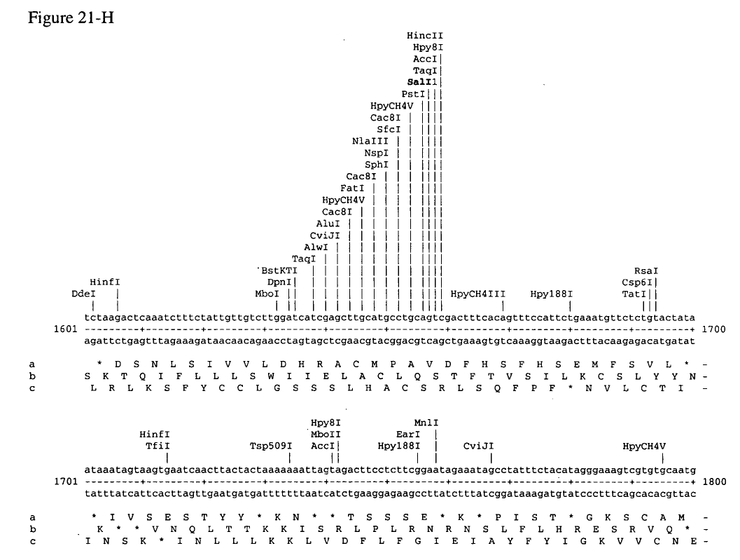

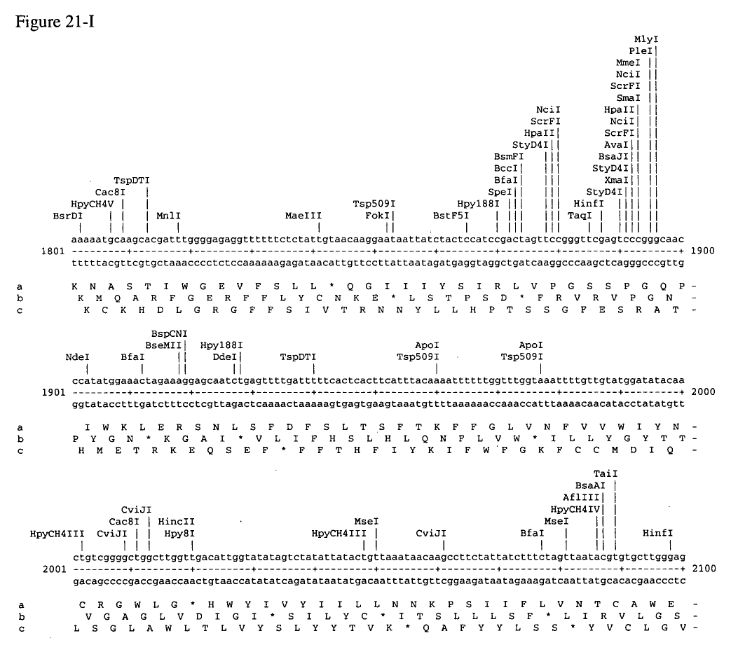

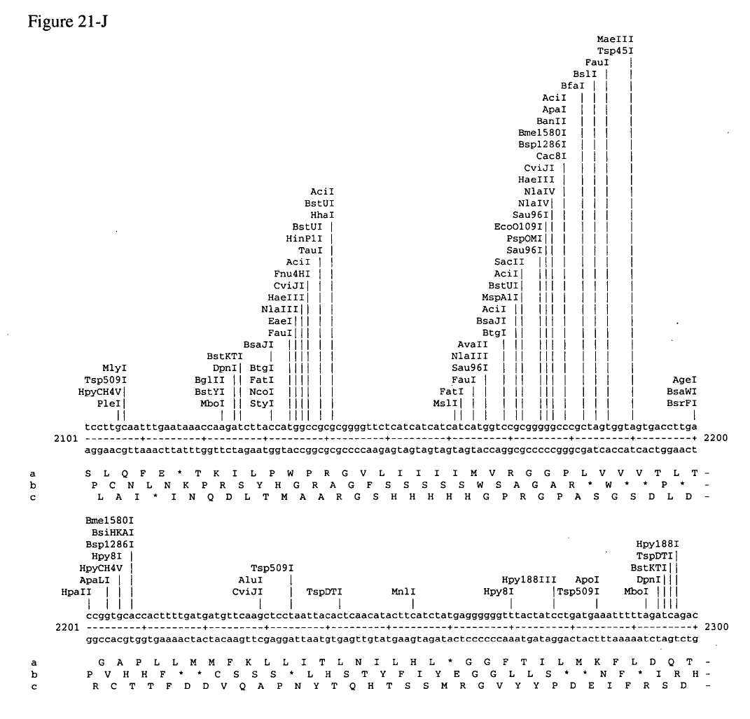

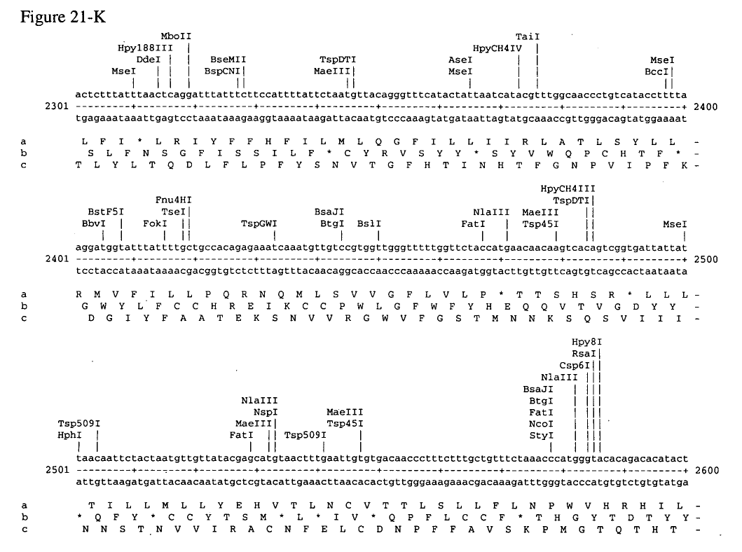

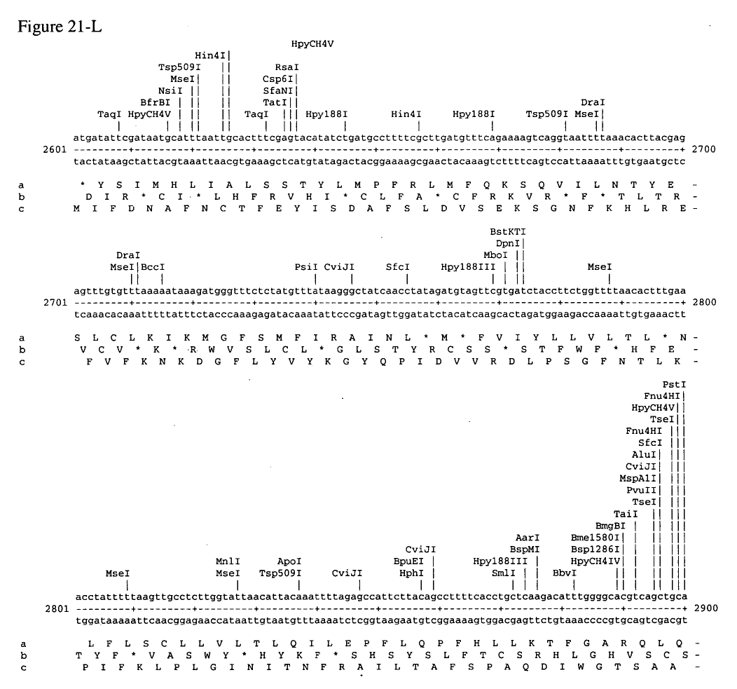

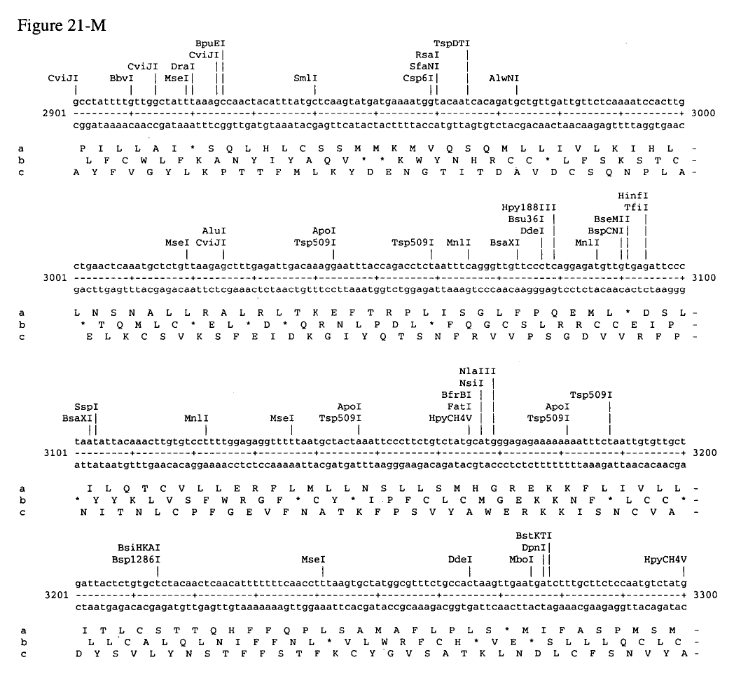

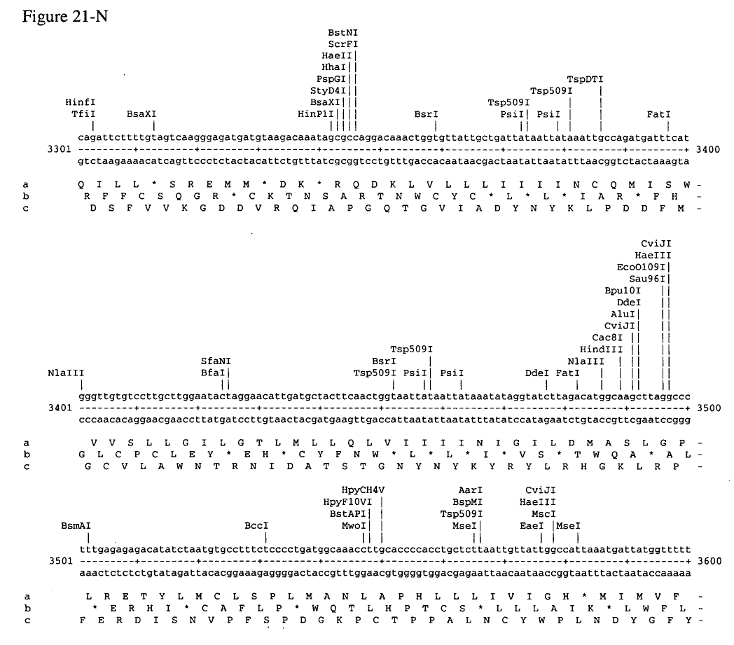

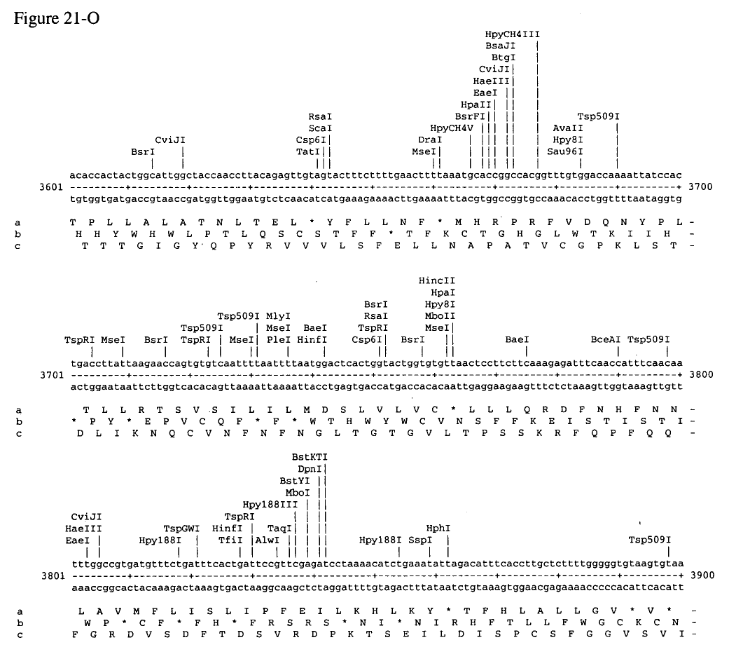

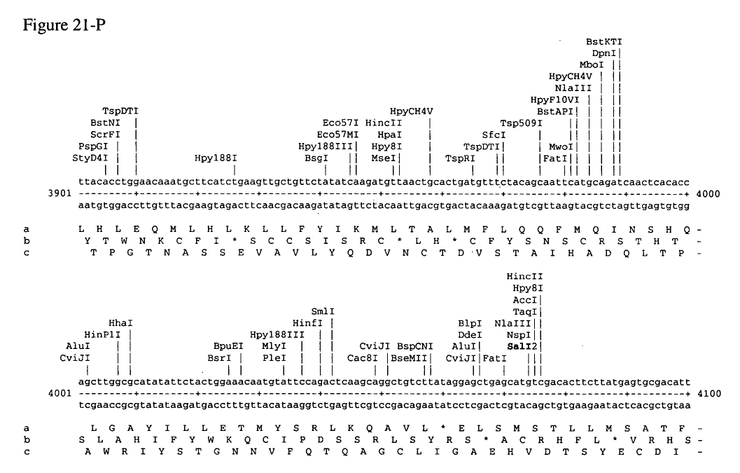

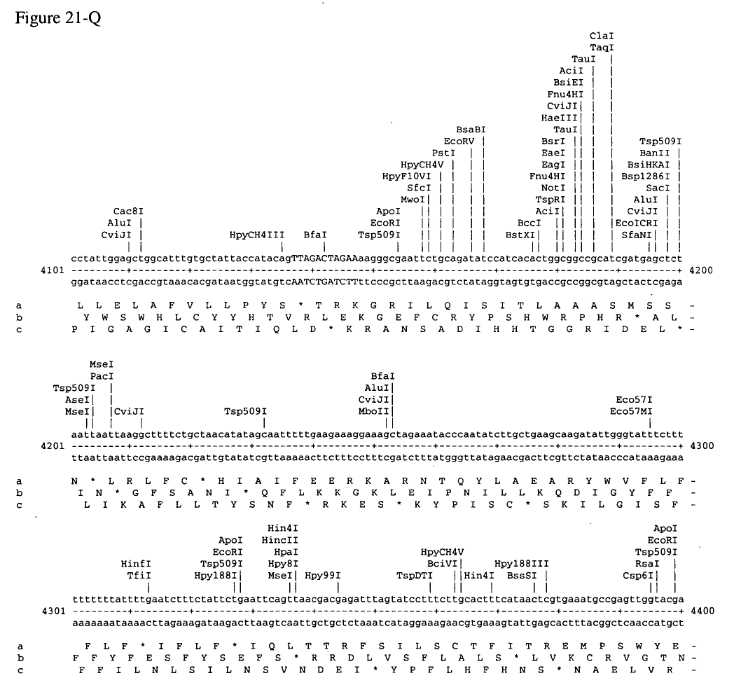

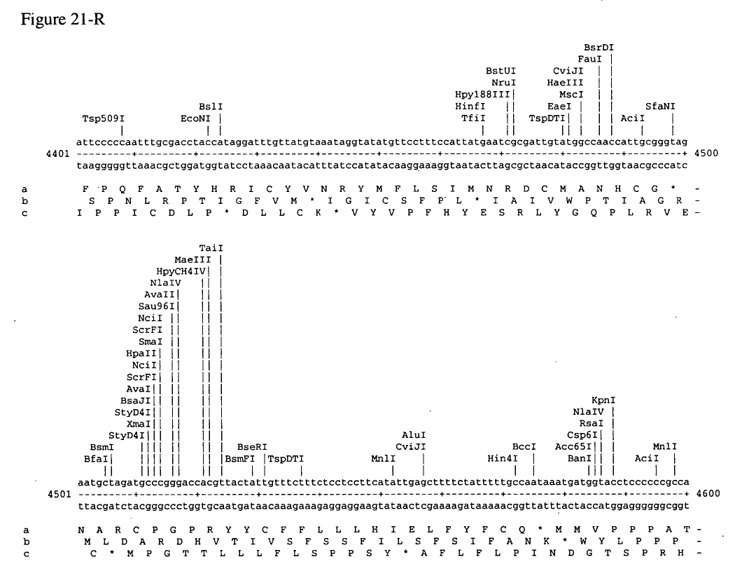

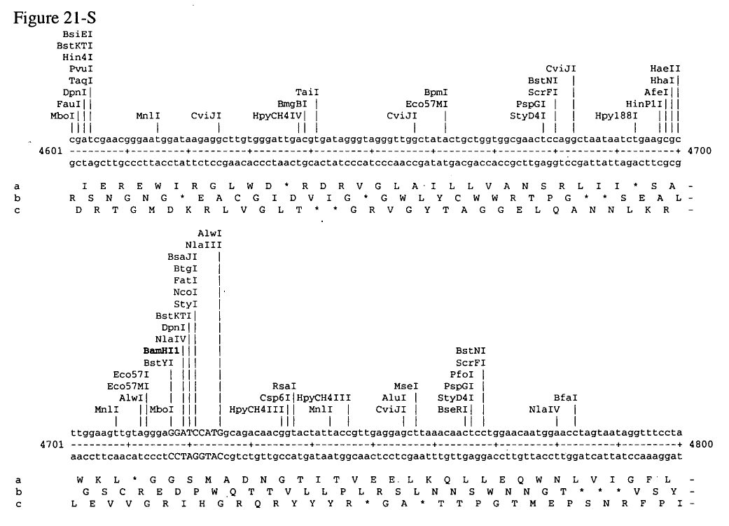

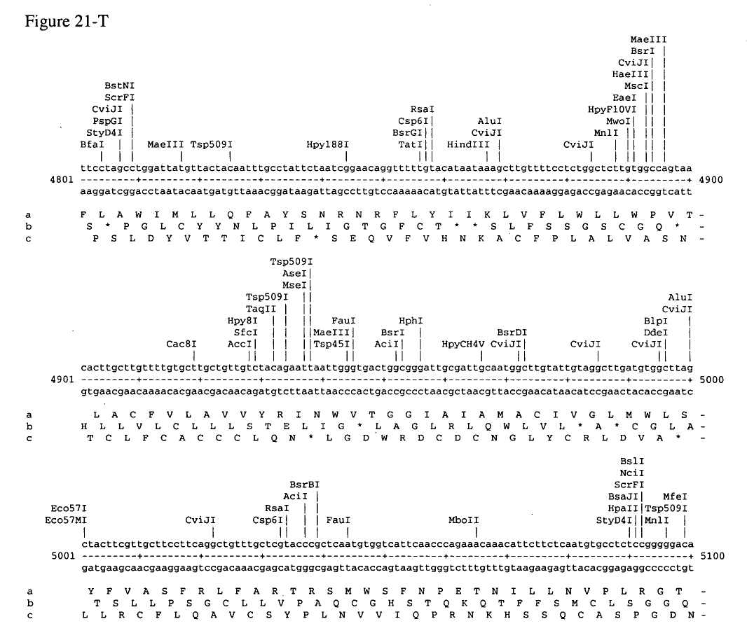

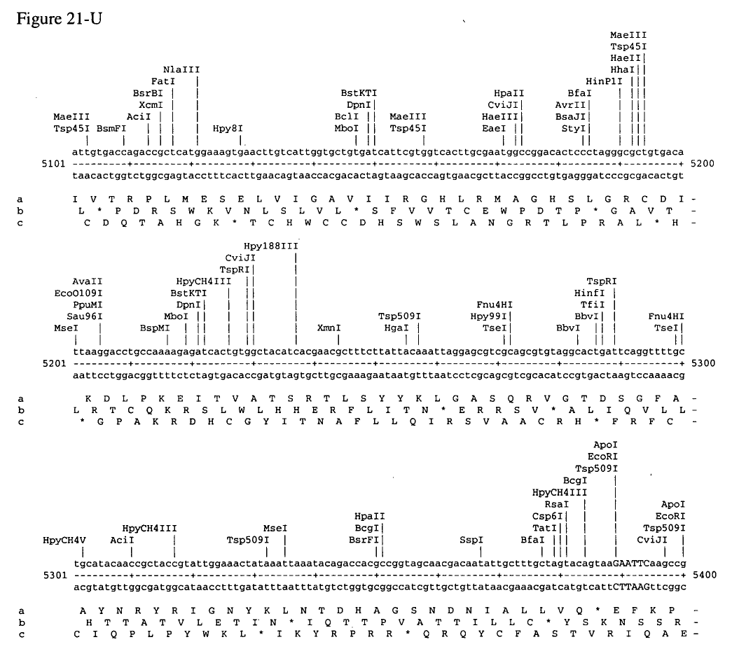

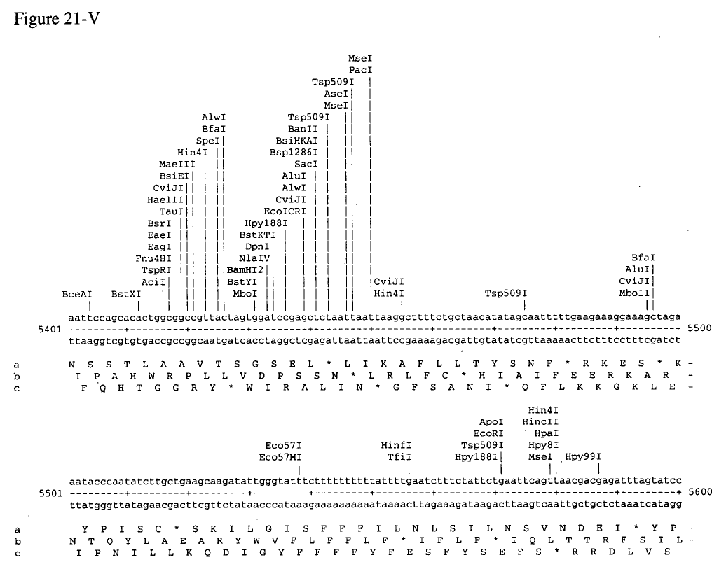

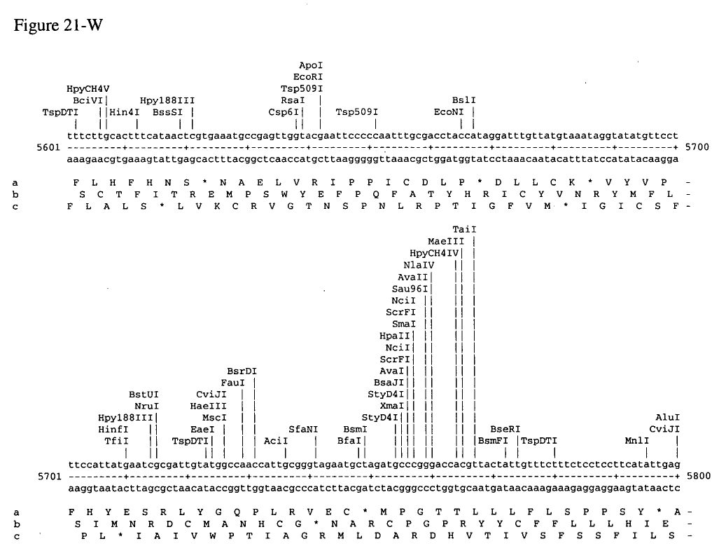

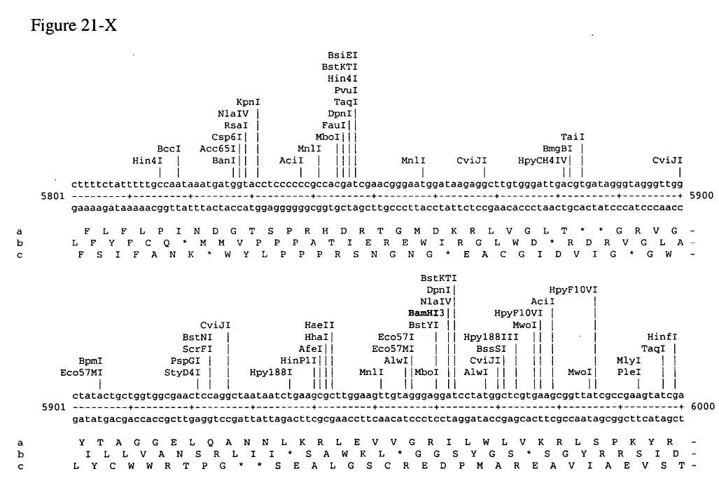

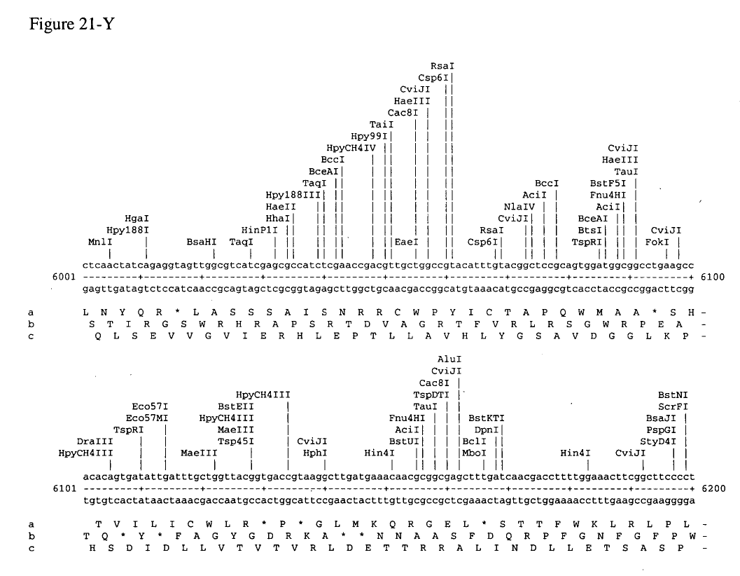

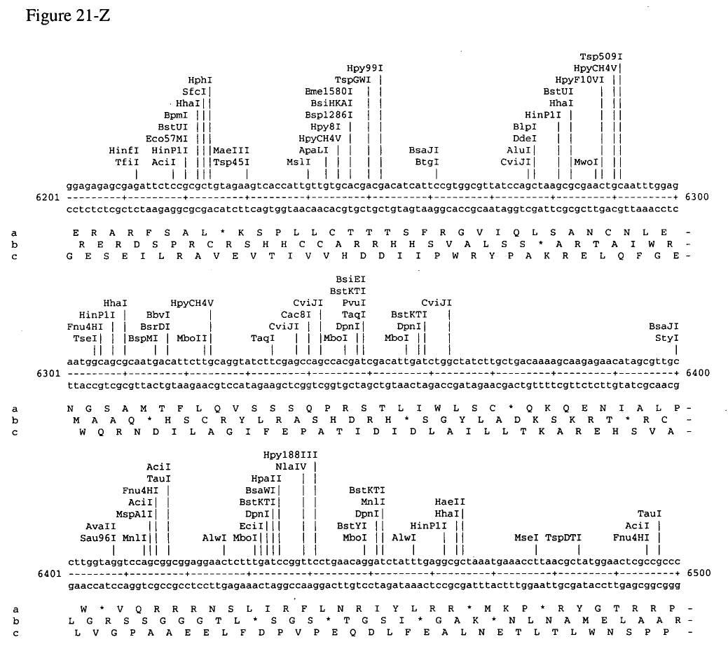

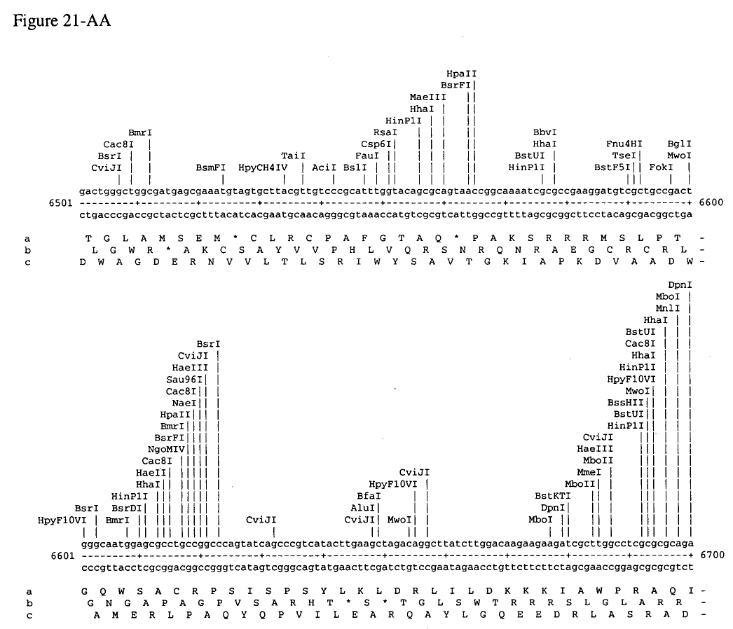

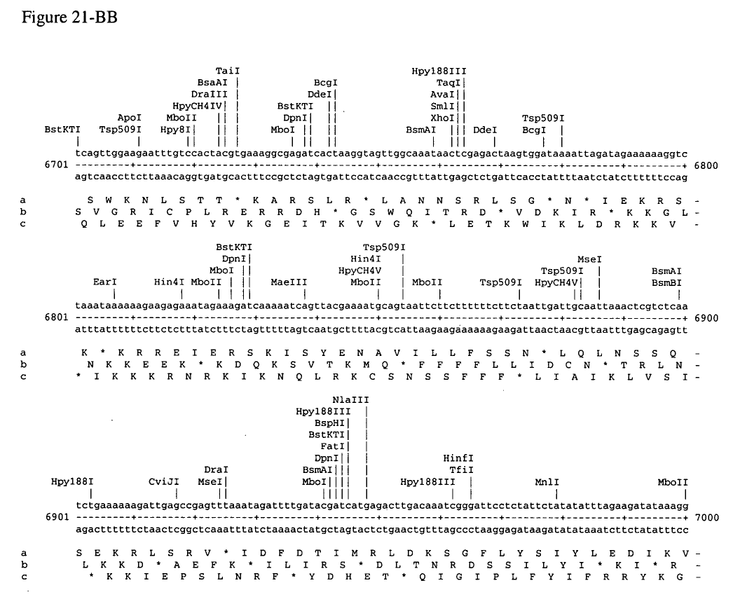

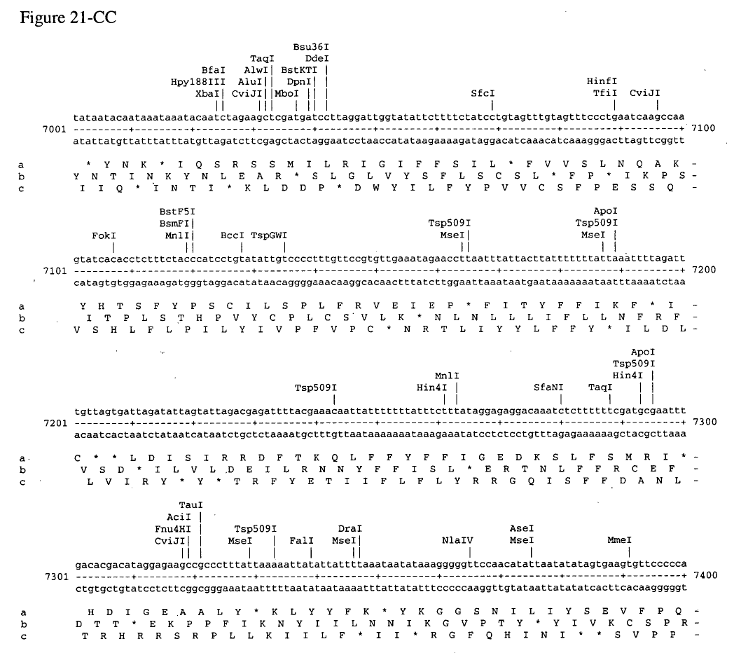

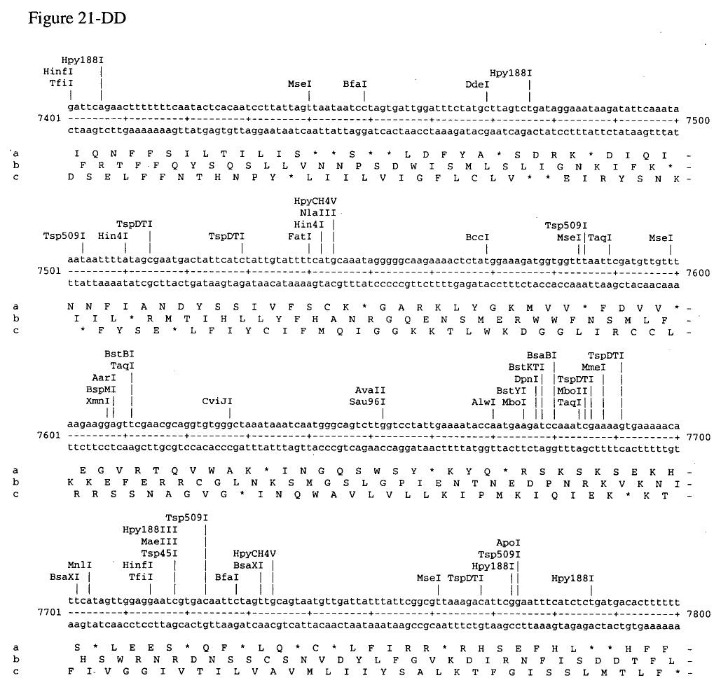

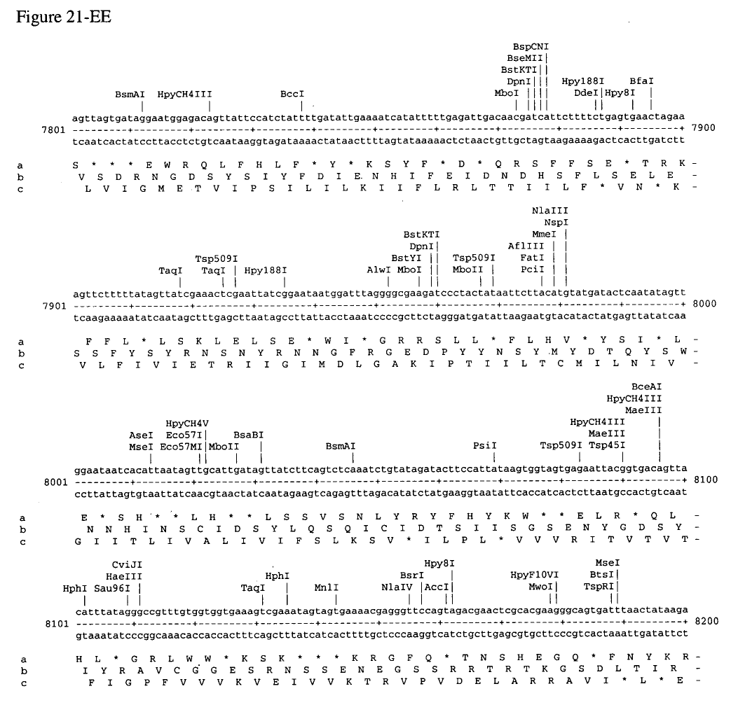

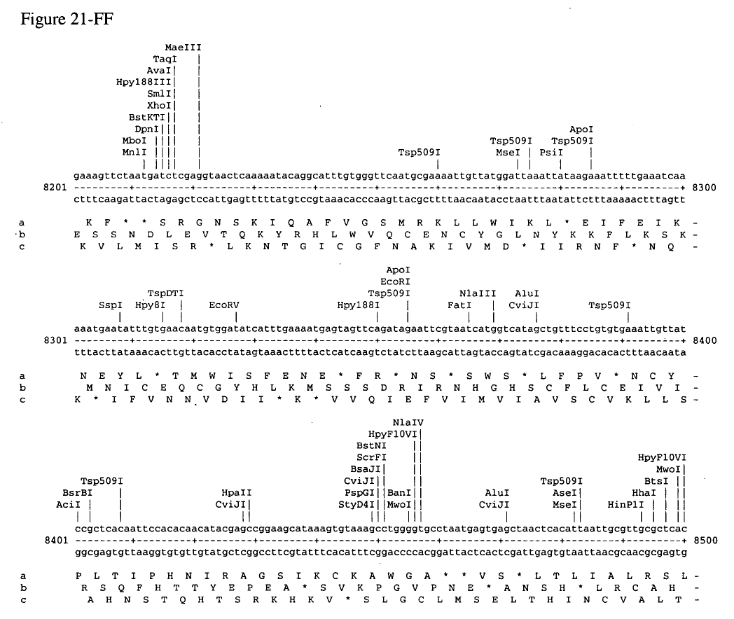

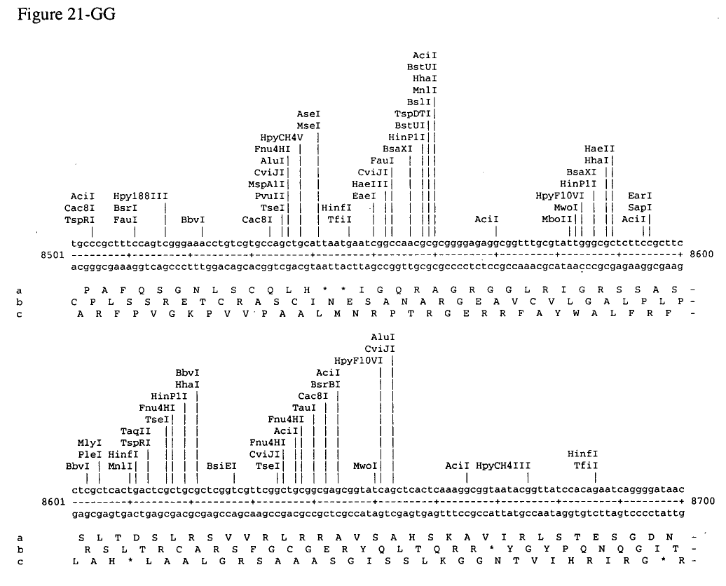

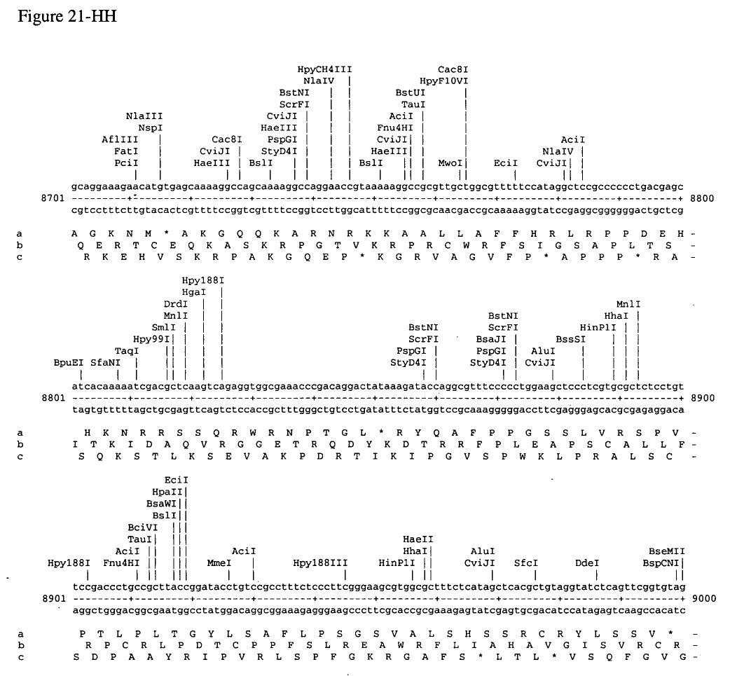

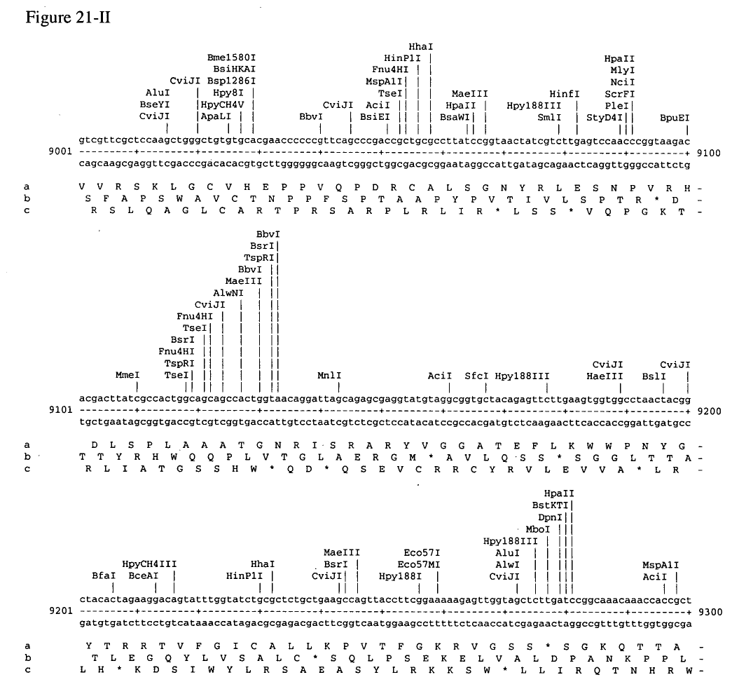

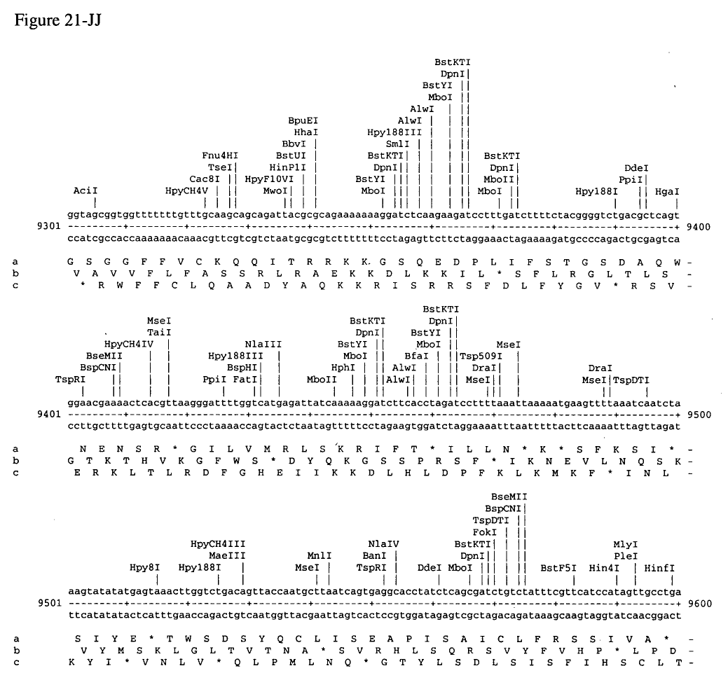

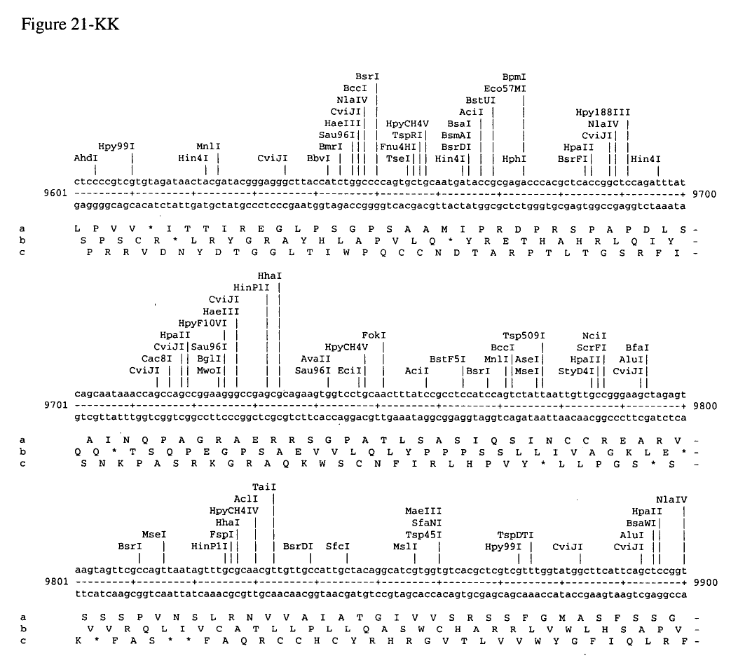

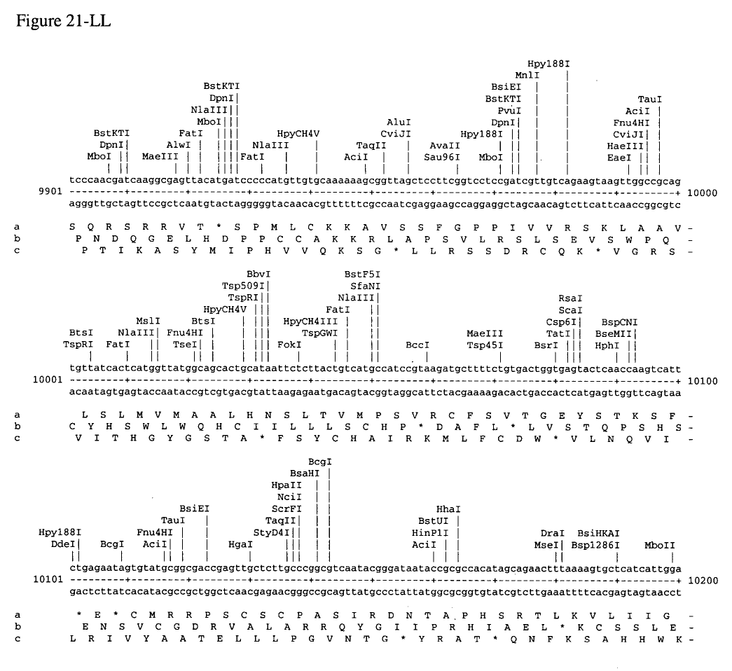

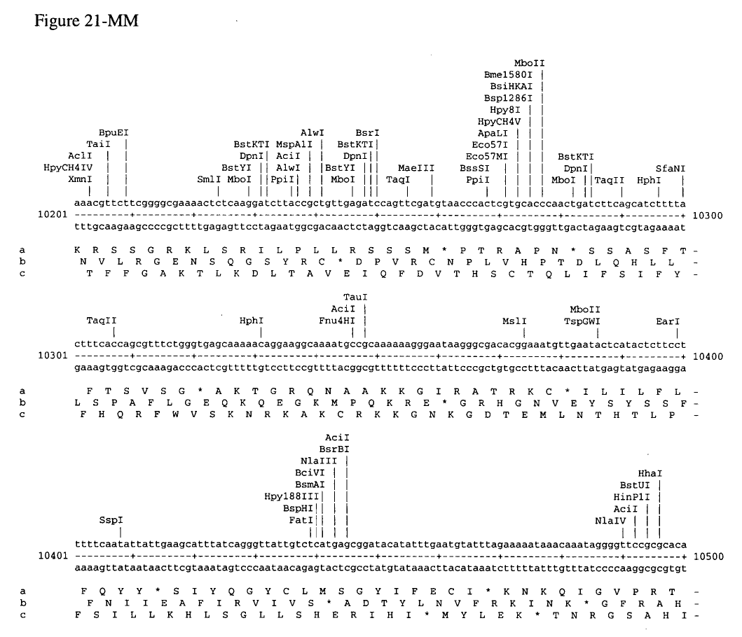

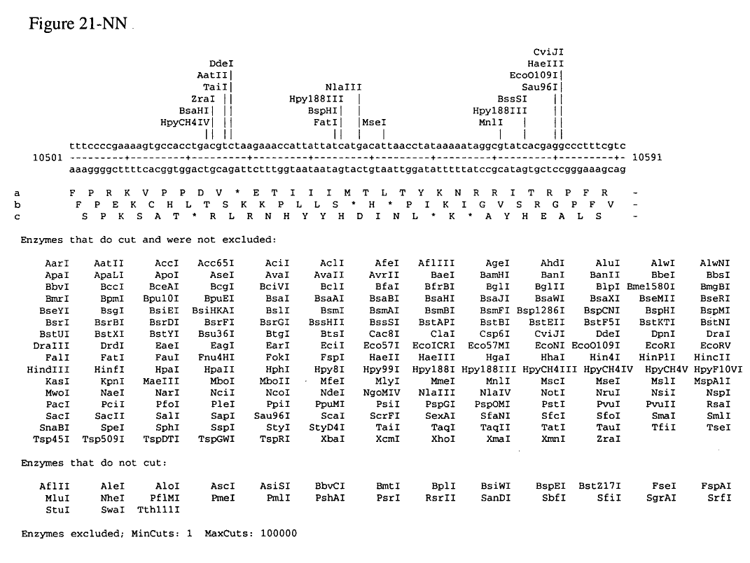

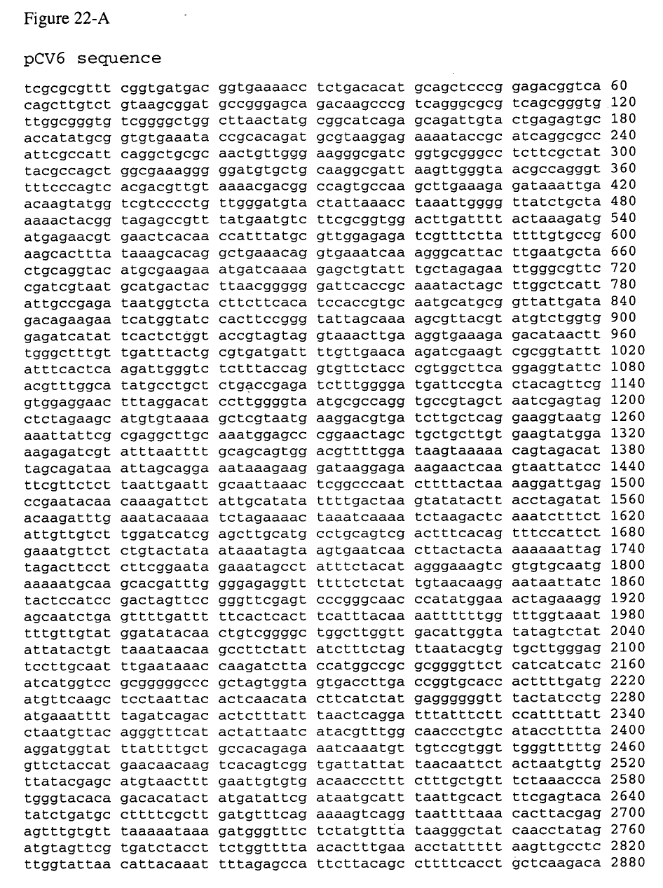

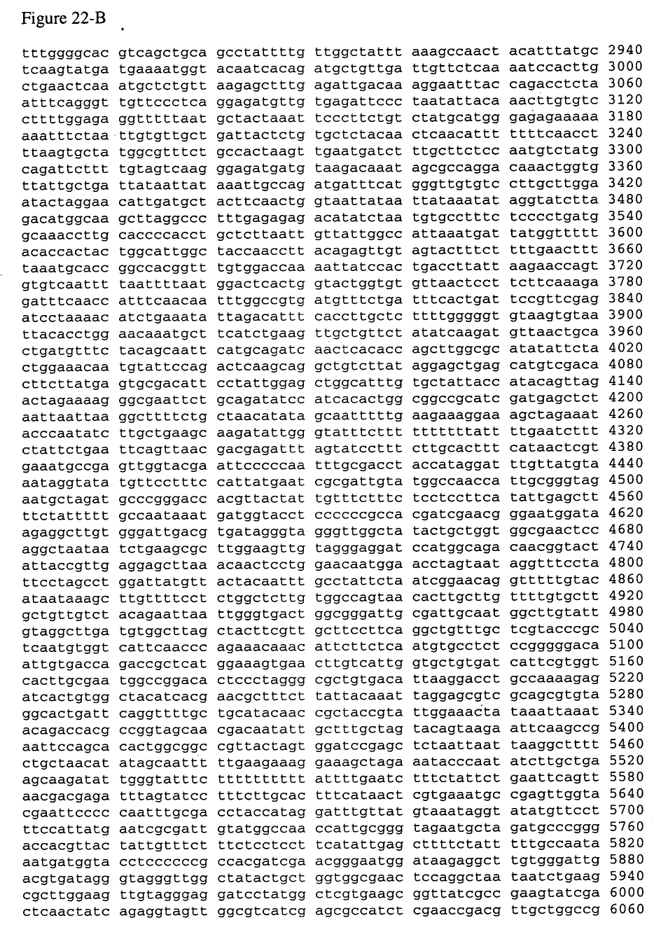

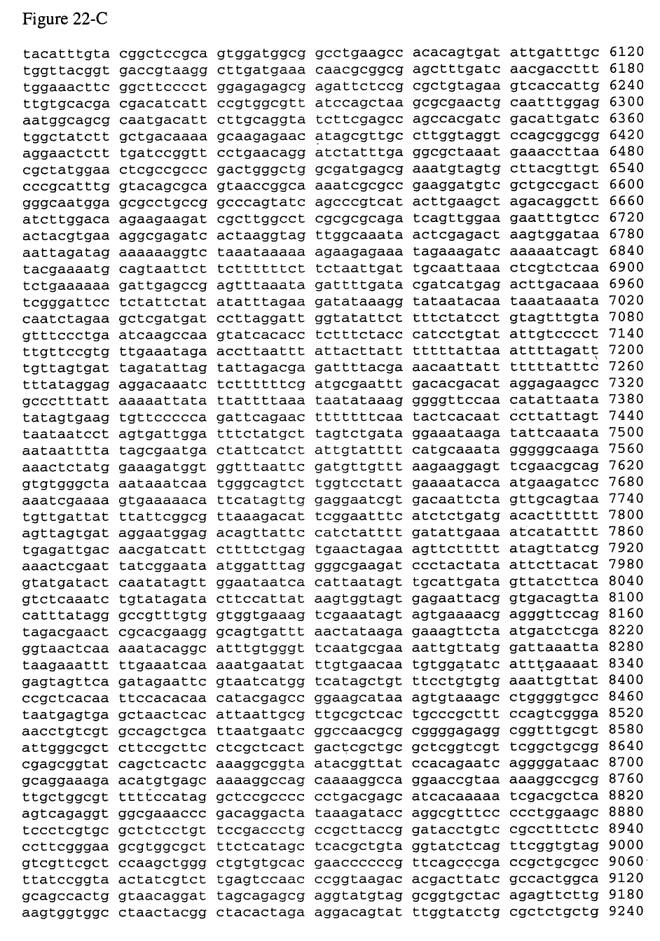

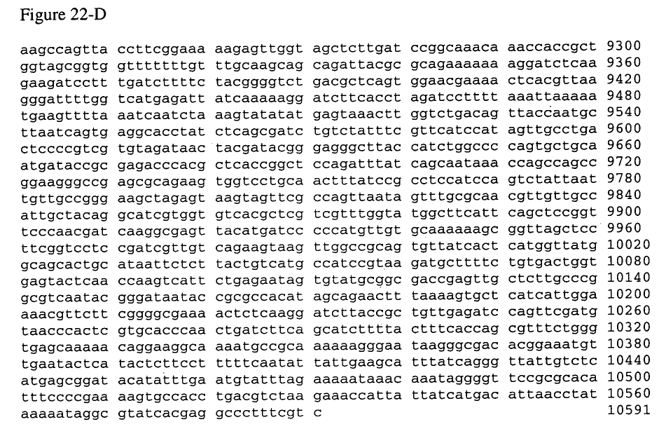

A 1.2-kb partial BamHI fragment consisting of the M-gene fused to the psbA terminator and the rrn promoter, in this order, from plasmid pCV3 was cloned into the BamHI unique site of plasmid pCV1 to generate plastid transformation vector pCV6 (FIG. 21) that co-expresses the S1 and M antigens. Plasmid pCV3 was obtained by cloning a 0.7-kb EcoRV-SacI fragment encoding M from pCRII-M in the StuI-SacI sites of pMLVHisA.

Codon Usage Optimization of SARS-CoV S1 Gene by Site Directed Mutagenesis

Plasmid pCRII-S1 was used as a mutagenesis template for generating derivatives of codon usage optimization. Mutagenesis was carried out by PCR with PfuTurbo DNA polymerase using the ‘QuikChange Multi site-directed mutagenesis kit’ (Stratagene), according to the manufacturer's specifications. Table 1 shows the oligonucleotides used for site-directed mutagenesis. The amplification procedure included denaturation at 95° C. for 1 min, followed by 30 cycles of denaturation (95° C. for 1 min), annealing (55° C. for 1 min) and extension (65° C. for 12 min). The amplified product was treated with DpnI to remove template DNA and the mutated DNA was used in transformation of E. coli XL10-Gold cells. DNA sequencing analysis was performed to confirm each mutation. A total of 13 nucleotide changes (as summarized in Table 1) incorporated in an “optimized” S1 was then used for cloning in plastid and nuclear transformation vectors.

TABLE 1

Oligonucleotides used for site-directed
mutagenesis of S1 for codon optimization

| Af- | | |
|---|---|---|
| fect | | |
| ed | | |
| Res- | | |
| idue | Sequence of primer | |
| | Forward primers: | |
| R18 | 5′-GGTAGTGACCTTGAC [symbol] TGCACCACTT | SEQ ID NO:12 |
| | TTGAT-3′; | |
| T75 | 5′-GGGTTTCATACTATTAATCAT [symbol] TTTG | SEQ ID NO:13 |
| | GCAACCCTGTCATAC-3′; | |
| S113 | 5′-CCATGAACAACAAGTCACAG [symbol] GTGAT | SEQ ID NO:14 |
| | TATTATTAACAATTCTACT-3′; | |
| S169 | 5′-AGTACATATCTGATGCCTTT [symbol] CTTGA | SEQ ID NO:15 |
| | TGTTTCAGAAAAGTC-3′; | |
| L209 | 5′-CCTATAGATGTAGTTCGTGAT [symbol] CCTT | SEQ ID NO:16 |
| | CTGGTTTTAACACTTTG-3′; | |
| T247 | 5′-CAAGACATTTGGGGC [symbol] TCAGCTGCAG | SEQ ID NO:17 |
| | CCTAT-3′; | |
| A398 | 5′-GATGATGTAAGACAAATA [symbol] CCAGGAC | SEQ ID NO:18 |
| | AAACTGG-3′; | |
| P507 | 5′-TCTTTTGAACTTTTAAATGCA [symbol] GCCA | SEQ ID NO:19 |
| | CGGTTTGTGGACC-3′; | |
| T509 | 5′-CTTTTAAATGCACCTGCC [symbol] GTTTGTG | SEQ ID NO:20 |
| | GACCAAAATTATC-3′; | |
| L597 | 5′-CTTCATCTGAAGTTGCTGTT [symbol] ATCAA | SEQ ID NO:21 |
| | GATGTTAACTGCAC-3′; | |
| R620 | 5′-CAACTCACACCAGCTTGG [symbol] ATATATT | SEQ ID NO:22 |
| | CTACTGGAAACAATG-3′; | |
| | Reverse primers: | |
| R18 | 5′-ATCAAAAGTGGTGCA [symbol] GTCAAGGTCA | SEQ ID NO:23 |
| | CTACC-3′; | |
| R620 | 5′-CATTGTTTCCAGTAGAATATAT [symbol] CCA | SEQ ID NO:24 |
| | AGCTGGTGTGAGTTG-3′; | |

*nucleotides in italics are mutated. The affected codons are underlined.

Plant Transformation
Plastid Transformation of Tobacco and Tomato for Expression of the S1 and/or M Antigens

Each plastid transformation construct (pCV1, pCV6 or pCV8, and its derivatives containing an S1 with nucleotide (but not amino acid) changes to optimize codon usage for expression in plants) was introduced by particle gun bombardment into tobacco (Staub and Maliga, 1994, Plant Journal 6: 547-553) and tomato (Ruf et al., 2001, Nature Biotech 19: 870-875).

Plastid Transformation of Lycopersicon esculentum cv. UC82B

Plastid transformation was carried out following Ruf et al., 2001, by bombardment of young leaves with tungsten particles coated with the desired DNA (pCV1, pCV6 or pCV8, and its derivatives containing an S1 with nucleotide (but not amino acid) changes to optimize codon usage for expression in plants).

Aseptical seeds were germinated on Murashige and Skoog (MS) medium (Murashige and Skoog, 1962, Physiol. Plant. 15: 473-497), supplemented with agar (5 g/liter) and sucrose (30 g/liter). For bombardment, young leaves from outgrowing axillary meristem were placed abaxial side facing up on RMOP medium containing MS salts, 6-benzylaminopurine (1 mg/liter), α-naphthalene acetic acid (0.1 mg/liter), thiamine (1 mg/liter), myo-inositol (100 mgaiter), agar (5 g/liter) at pH 5.7 and sucrose (30 g/liter). Bombardment was carried out using the particle delivery system, PDS1000-He (Bio-Rad) and its accessories. Tungsten particles (M 17) were coated with desired DNA and leaves bombarded. After bombardment, leaves were cut into small pieces and placed on RMOP selection medium containing spectinomycin dihydrochloride (250 mg/liter). Greenish yellow calli were passaged for few more cycles in the selection medium to obtain homoplastomic plastid-containing calli. From this calli plant regeneration will be achieved.

Plastid Transformation of N. tabacum cv. xanthi

Plastid transformation was carried out following Svab and Maliga (Svab et al., 1993, Proc. Natl. Acad. Acad. Sci. USA 90: 913-917) by bombardment of leaves with tungsten particles coated with the desired DNA (pCV1, pCV6 or pCV8, and its derivatives containing an S1 with nucleotide (but not amino acid) changes to optimize codon usage for expression in plants).

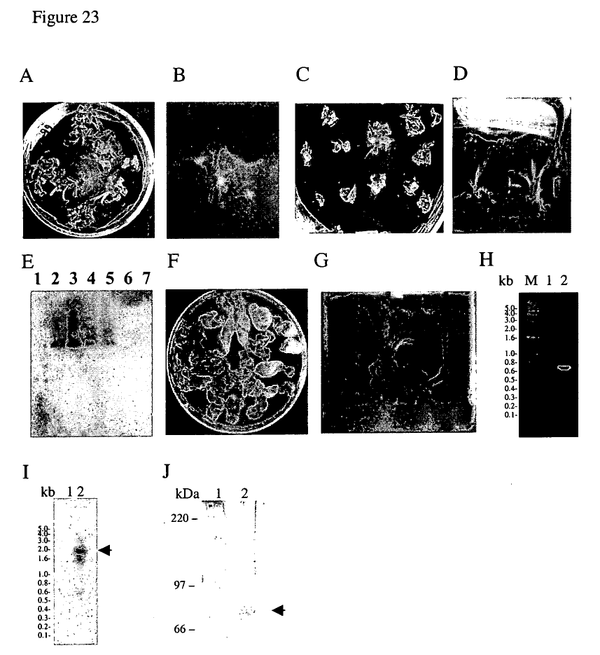

Aseptical seeds were germinated on Murashige and Skoog (MS) medium (Murashige and Skoog, 1962, Physiol. Plant. 15: 473-497), supplemented with agar (5 g/liter) and sucrose (30 g/liter). For bombardment, leaves were placed abaxial side facing up on RMOP medium containing MS salts, 6-benzylaminopurine (1 mg/liter), α-naphthalene acetic acid (0.1 mg/liter), thiamine (1 mg/liter), myo-inositol (100 mg/liter), agar (5 g/liter) at pH 5.7 and sucrose (30 g/liter). Bombardment was carried out using the particle delivery system, PDS 1000-He (Bio-Rad) and its accessories. Tungsten particles (M 17) were coated with desired DNA and leaves bombarded. After bombardment, leaves were cut into small pieces and placed on RMOP selection medium containing spectinomycin dihydrochloride (500 mg/liter). The regenerated plantlets obtained following plastid transformation of tobacco using vector pCV1 (FIG. 23F-23G) were passaged for five more cycles in selection medium to obtain homoplastomic plastid-containing plants. Results of PCR analysis (FIG. 23H) using S1 primers ML560 (5′-CAGAGAGAGTTTCCCGGATTC-3′; SEQ ID NO:25) and ML567 (5′-CAACCTATAGATGTAGTTCG-3′; SEQ ID NO:26) and of northern blot analysis (FIG. 231) using an ³²P-radiolabeled S1 probe are shown. FIG. 23J shows western blot analysis of transplastomic tobacco expressing a modified S1 (with nucleotide changes as stipulated in Table 1) using Ni-NTA conjugate in detection of a His-tagged S1 protein. The arrow indicates the expected 73-kDa (His)₅-S1 band.

Nuclear Transformation of Tobacco and Lettuce for Expression of S1 or M Antigen

Production of transgenic tobacco and transgenic lettuce expressing S1 or M antigen by nuclear transformation was carried out using Agrobacterium-mediated transformation (Horsch et al., 1985, Science 227: 1227-1231) with plasmids pCV2 [and its derivative containing an S1 with nucleotide (but not amino acid) changes to optimize codon usage for expression in plants] and pCV4, respectively.

Following Agrobacterium-mediated transformation, shoots of tobacco (FIG. 23A) and lettuce (FIG. 23C) were rooted and the regenerated shoots (FIG. 23, B, D) were used in PCR analysis using primers 35S and NOS-ter that are located within the CaMV 35S promoter and the NOS-terminator, respectively. Southern blot analysis using a ³²P-labeled S1 probe of these PCR-amplified DNA is shown in FIG. 23E. A 2.1-kb hybridizing band confirms the presence of S1-containing transgenic lines (FIG. 23E; lanes 2-5).

Nuclear Transformation of Tobacco for Expression of S1:GFP Antigen

Production of transgenic tobacco expressing a protein fusion consisting of the SARS-CoV S1 protein fused with green fluorescent protein (GFP) was carried out using Agrobacterium-mediated transformation (Horsch et al. supra.) with plasmid pCV12.

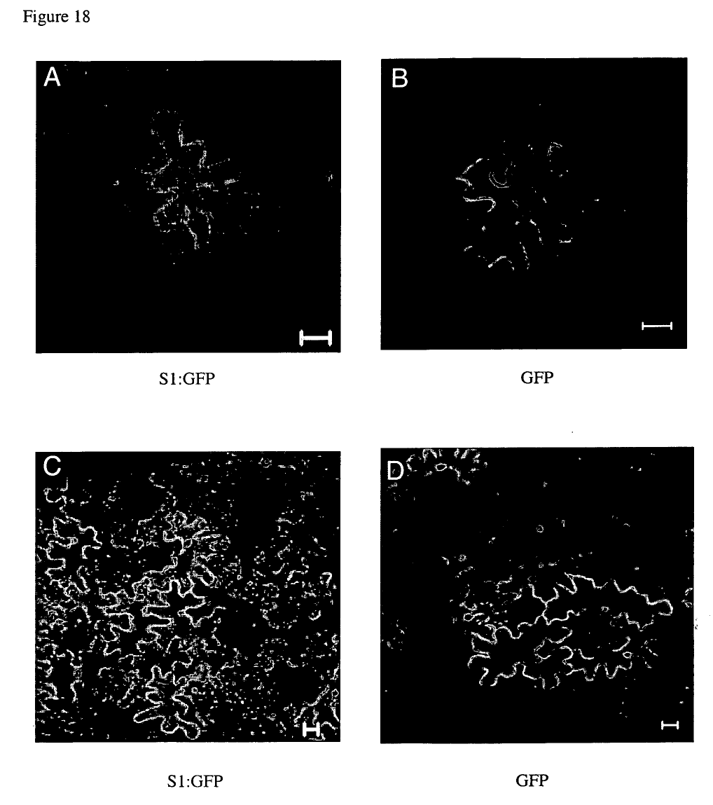

Two days after agroinfiltration, representative tobacco leaf epidermal cells were selected and observed by confocal microscopy (Yang Y. et al., In vivo analysis of plant promoters and transcription factors by agroinfiltration of tobacco leaves. Plant J. 2000; 22: 543-551) of Agrobacterium tumefaciens LBA4404 harboring plasmid pCV12 expressing S1:GFP fusion protein (FIG. 18, A, C) or LBA4404 harboring pGDG expressing GFP alone (FIG. 18, B, D). Bar represents 20 μm.

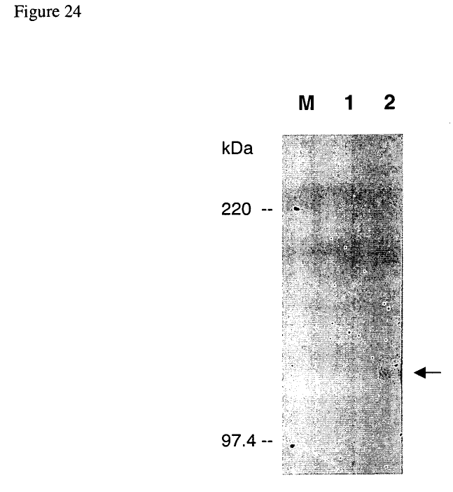

Western blot analysis was performed using antibodies against GFP and showed transient expression of S1:GFP in the tobacco leaves following agroinfiltration. Total protein (200 μg) extracted from tobacco leaves, infiltrated with plasmid pGDG expressing GFP alone (FIG. 23, lane 1) or plasmid pCV12 expressing S1:GFP fusion (FIG. 23, lane 2), were separated on a 8% SDS-PAGE gel, blotted onto Hybond-C filters according to Sambrook et al. (1989. Molecular Cloning: A Laboratory Manual. Cold Spring Harbor Laboratory Press, Cold Spring Harbor), and cross-reacted with antibodies against GFP (Clontech). The results are shown in FIG. 24, wherein arrow indicates cross-reacting S1:GFP band (calculated size 99.1 kDa) and M is the molecular mass marker.

Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference.

Citation or discussion of a reference herein shall not be construed as an admission that such is prior art to the present invention.

1. A vector comprising an isolated nucleic acid which nucleic acid sequence is set forth in SEQ ID NO:708 or 709, or a fragment, derivative or analog thereof.

2. The vector in accordance with claim 1, wherein the vector is a plastid transformation vector.

3. The vector in accordance with claim 2, wherein the vector is pCV1, pCV6, pCV8, or a derivative thereof containing an S1 with at least one nucleotide change.

4. The vector in accordance with claim 1, wherein the vector is a nuclear transformation vector.

5. The vector in accordance with claim 4, wherein the vector is pCV2, pCV4, pCV12, or a derivative thereof containing an S1 with at least one nucleotide change.

6. The vector in accordance with claim 1, wherein the isolated nucleic acid encodes the S protein of the SARS-CoV virus, the M protein of the SARS-CoV virus, or both.

7. The vector in accordance with claim 1, wherein the isolated nucleic acid comprises the nucleic acid sequence as set forth in SEQ ID NO:4023, 4025, or a fragment, derivative or analog thereof.

8. The vector in accordance with claim 1, wherein the isolated nucleic acid encodes the amino acid sequence as set forth in SEQ ID NO:4024 or 4026, or a fragment, derivative or analog thereof.

9. The vector in accordance with claim 1, wherein the isolated nucleic acid hybridizes under high stringency conditions to the nucleic acid sequence of SEQ ID NO:4023, 4025, 708, or 709, or a complement thereof.

10. A plant cell comprising an isolated nucleic acid which nucleic acid sequence is set forth in SEQ ID NO:708, 709, or a fragment, derivative or analog thereof.

11. The plant cell in accordance with claim 10, wherein the isolated nucleic acid encodes the S protein of the SARS-CoV virus, the M protein of the SARS-CoV virus, or both.

12. The plant cell in accordance with claim 10, wherein the isolated nucleic acid comprises the nucleic acid sequence as set forth in SEQ ID NO:4023, 4025, or a fragment, derivative or analog thereof.

13. The plant cell in accordance with claim 10, wherein the isolated nucleic acid encodes the amino acid sequence as set forth in SEQ ID NO:4024 or 4026, or a fragment, derivative or analog thereof.

14. The plant cell in accordance with claim 10, wherein the isolated nucleic acid hybridizes under high stringency conditions to the nucleic acid sequence of SEQ ID NO:4023, 4025, 708, or 709, or a complement thereof.

15. The plant cell in accordance with claim 10, wherein the plant cell is isolated from a plant selected from the group consisting of tobacco, lettuce, tomato, potato, banana, corn, rice, cereals, wheat, maize, barley, apple, pear, strawberry, carrot, sugar beets, yam, kiwifruit, or spinach.

16. A plant cell comprising the vector in accordance with claim 1.

17. A method of immunization against SARS comprising administering to a subject a plant, wherein the plant comprises an isolated nucleic acid which nucleic acid sequence is set forth in SEQ ID NO:708, 709, or a fragment, derivative, analog, or vector thereof.

18. The method in accordance to claim 17, wherein the isolated nucleic acid encodes the S protein of the SARS-CoV virus, the M protein of the SARS-CoV virus, or both.

19. The method in accordance to claim 17, wherein the isolated nucleic acid comprises the nucleic acid sequence as set forth in SEQ ID NO:4023, 4025, or a fragment, derivative or analog thereof.

20. The method in accordance to claim 17, wherein the isolated nucleic acid encodes the amino acid sequence as set forth in SEQ ID NO:4024 or 4026, or a fragment, derivative or analog thereof.

21. The method in accordance to claim 17, wherein the isolated nucleic acid hybridizes under high stringency conditions to the nucleic acid sequence of SEQ ID NO:4023, 4025, 708, or 709, or a complement thereof.

22. The method in accordance to claim 17, where the administering to the subject is oral administration.

23. The method in accordance to claim 17, wherein subject is human.

24. A method of immunization against SARS comprising administering to a subject a vector in accordance with claim 1 and an acceptable pharmaceutical carrier.

25. The method in accordance to claim 24, wherein the subject is human.

26. A plant comprising the isolated nucleic acid which nucleic acid sequence is set forth in SEQ ID NO:708, 709, or a fragment, derivative or analog thereof.

27. The plant in accordance with claim 26, which is selected from the group consisting of tobacco, lettuce, tomato, potato, banana, corn, rice, cereals, wheat, maize, barley, apple, pear, strawberry, carrot, sugar beets, yam, kiwifruit, or spinach.

28. A composition comprising a SARS-CoV viral antigen produced by the plant cell of claim 10.

29. A composition comprising a SARS-CoV viral antigen produced by the plant cell of claim 16.

30. A composition comprising a SARS-CoV viral antigen produced by the plant of claim 26.

31. A method of detecting an antibody to a SARS-CoV viral antigen in a sample comprising:
(a) contacting the sample with the composition of claim 28; and
(b) detecting the presence of an antibody bound to the composition of claim 28, thereby detecting an antibody to a SARS-CoV viral antigen.

32. The method in accordance with claim 31, wherein the sample is a biological fluid.

33. The method in accordance with claim 32, wherein the biological fluid is blood, serum, plasma, saliva, urine, stool, sputum, nasopharyngeal aspirates, cells or tissues.

34. A method of detecting an antibody to a SARS-CoV viral antigen in a sample comprising:
(a) contacting the sample with the composition of claim 29; and
(b) detecting the presence of an antibody bound to the composition of claim 29, thereby detecting an antibody to a SARS-CoV viral antigen.

35. The method in accordance with claim 34, wherein the sample is a biological fluid.

36. The method in accordance with claim 35, wherein the biological fluid is blood, serum, plasma, saliva, urine, stool, sputum, nasopharyngeal aspirates, cells or tissues.

37. A method of detecting an antibody to a SARS-CoV viral antigen in a sample comprising:
(a) contacting the sample with the composition of claim 30; and
(b) detecting the presence of an antibody bound to the composition of claim 30, thereby detecting an antibody to a SARS-CoV viral antigen.

38. The method in accordance with claim 37, wherein the sample is a biological fluid.

39. The method in accordance with claim 38, wherein the biological fluid is blood, serum, plasma, saliva, urine, stool, sputum, nasopharyngeal aspirates, cells or tissues.
